(19) United States
(12) Patent Application Publication
(10) Pub. No.: US 2022/0315913 A1
(43) Pub. Date: Oct. 6, 2022

(54) NOVEL CRISPR DNA TARGETING ENZYMES AND SYSTEMS

(71) Applicant: ARBOR BIOTECHNOLOGIES, INC., Cambridge, MA (US)

(72) Inventors: David A. Scott, Cambridge, MA (US); David R. Cheng, Boston, MA (US); Winston X. Yan, Boston, MA (US)

(21) Appl. No.: 17/619,165

(22) PCT Filed: Jun. 12, 2020
(86) PCT No.: PCT/US2020/037585

§ 371 Date: Dec. 14, 2021

Related U.S. Application Data
(60) Provisional application No. 62/861,582, filed on Jun. 14, 2019.
(60) Provisional application No. 62/872,157, filed on Jul. 9, 2019.
(60) Provisional application No. 62/873,606, filed on Jul. 12, 2019.
(60) Provisional application No. 62/873,602, filed on Jul. 12, 2019.

(51) Int. Cl. C12N 15/10; C07K 14/195; C12N 9/22

(52) U.S. Cl. CPC C12N 15/102; C07K 14/195; C12N 9/22; C12N 15/907

(57) ABSTRACT

The disclosure describes novel systems, methods, and compositions for the manipulation of nucleic acids in a targeted fashion. The disclosure describes non-naturally occurring, engineered CRISPR systems, components, and methods for targeted modification of nucleic acids such as DNA. Each system includes one or more protein components and one or more nucleic acid components that together target nucleic acids.

### RELATED APPLICATIONS

This application claims priority to U.S. Provisional Application 62/861,582 filed on Jun. 14, 2019, U.S. Provisional Application 62/872,157 filed on Jul. 9, 2019, U.S. Provisional Application 62/873,606 filed on Jul. 12, 2019, and U.S. Provisional Application 62/873,602 filed on Jul. 12, 2019, the entire contents of each of which are hereby incorporated by reference.

### SEQUENCE LISTING

The instant application contains a Sequence Listing which has been submitted electronically in ASCII format and is hereby incorporated by reference in its entirety. Said ASCII copy, created on Jun. 12, 2020, is named A2186-7021WO_SL.txt and is 901,689 bytes in size.

### FIELD OF THE INVENTION

The present disclosure relates to systems, methods, and compositions used for the control of gene expression involving sequence targeting and nucleic acid editing, which uses vector systems related to Clustered Regularly Interspaced Short Palindromic Repeats (CRISPR) and components thereof.

### BACKGROUND

Recent application of advances in genome sequencing technologies and analysis have yielded significant insights into the genetic underpinning of biological activities in many diverse areas of nature, ranging from prokaryotic biosynthetic pathways to human pathologies. To fully understand and evaluate the vast quantities of information produced by genetic sequencing technologies, equivalent increases in the scale, efficacy, and ease of technologies for genome and epigenome manipulation are needed. These novel genome and epigenome engineering technologies will accelerate the development of novel applications in numerous areas, including biotechnology, agriculture, and human therapeutics.

Clustered Regularly Interspaced Short Palindromic Repeats (CRISPR) and the CRISPR-associated (Cas) genes, collectively known as the CRISPR-Cas or CRISPR/Cas systems, are currently understood to provide immunity to bacteria and archaea against phage infection. The CRISPR-Cas systems of prokaryotic adaptive immunity are an extremely diverse group of proteins effectors, non-coding elements, as well as loci architectures, some examples of which have been engineered and adapted to produce important biotechnologies.

The components of the system involved in host defense include one or more effector proteins capable of modifying DNA or RNA and an RNA guide element that is responsible to targeting these protein activities to a specific sequence on the phage DNA or RNA. The RNA guide is composed of a CRISPR RNA (crRNA) and may require an additional trans-activating RNA (tracrRNA) to enable targeted nucleic acid manipulation by the effector protein(s). The crRNA consists of a direct repeat responsible for protein binding to the crRNA and a spacer sequence that is complementary to the desired nucleic acid target sequence. CRISPR systems can be reprogrammed to target alternative DNA or RNA targets by modifying the spacer sequence of the crRNA.

CRISPR-Cas systems can be broadly classified into two classes: Class 1 systems are composed of multiple effector proteins that together form a complex around a crRNA, and Class 2 systems consist of a single effector protein that complexes with the crRNA to target DNA or RNA substrates. The single-subunit effector composition of the Class 2 systems provides a simpler component set for engineering and application translation, and have thus far been an important source of programmable effectors. Thus, the discovery, engineering, and optimization of novel Class 2 systems may lead to widespread and powerful programmable technologies for genome engineering and beyond.

Citation or identification of any document in this application is not an admission that such document is available as prior art to the present invention.

CRISPR-Cas systems are adaptive immune systems in archaea and bacteria that defend the species against foreign genetic elements. The characterization and engineering of Class 2 CRISPR-Cas systems, exemplified by CRISPR-Cas9, have paved the way for a diverse array of biotechnology applications in genome editing and beyond. Nevertheless, there remains a need for additional programmable effectors and systems for modifying nucleic acids and polynucleotides (i.e., DNA, RNA, or any hybrid, derivative, or modification) beyond the current CRISPR-Cas systems that enable novel applications through their unique properties.

### SUMMARY

This disclosure provides non-naturally-occurring, engineered systems and compositions for new single-effector Class 2 CRISPR-Cas systems, together with methods for computational identification from genomic databases, development of the natural loci into engineered systems, and experimental validation and application translation. These new effectors are divergent in sequence to orthologs and homologs of existing Class 2 CRISPR effectors, and also have unique domain organizations. They provide additional features that include, but are not limited to, 1) novel DNA/RNA editing properties and control mechanisms, 2) smaller size for greater versatility in delivery strategies, 3) genotype triggered cellular processes such as cell death, and 4) programmable RNA-guided DNA insertion, excision, and mobilization.

Adding the novel DNA-targeting systems described herein to the toolbox of techniques for genome and epigenome manipulation enables broad applications for specific, programmed perturbations.

This disclosure relates to new CRISPR-Cas systems including newly discovered enzymes and other components used to create minimal systems that can be used in non-natural environments. e.g., in bacteria other than those in which the system was initially discovered.

In one aspect, the disclosure provides engineered, non-naturally occurring Clustered Regularly Interspaced Short Palindromic Repeat (CRISPR)-Cas systems of CLUST.121143 including an RNA guide including a direct repeat sequence and a spacer sequence capable of hybridizing to a target nucleic acid, or a nucleic acid encoding the RNA guide; and a CRISPR-associated protein or a nucleic acid encoding the CRISPR-associated protein, where the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence provided in TABLE 2 (e.g., SEQ ID NOs: 1-17); where the CRISPR-associated protein is capable of binding to the RNA guide and of targeting the target nucleic acid sequence complementary to the spacer sequence. In some embodiments, the CRISPR-associated protein has at least one (e.g., one, two, or three) RuvC domain.

In some embodiments of any of the systems described herein, the CRISPR associated protein is the CLUST.121143 3300014839 effector protein. In some embodiments, the CRISPR-associated protein is capable of recognizing a protospacer adjacent motif (PAM), and the target nucleic acid including a PAM that includes the nucleic acid sequence 5′-TTG-3′.

In some embodiments of any of the systems described herein, the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence provided in Table 3. In some embodiments of any of the systems described herein, the direct repeat sequence comprises a nucleotide sequence that is provided in Table 3. In some embodiments of any of the systems described herein, the spacer sequence of the RNA guide includes between about 22 to about 40 nucleotides (e.g., 26 to 35 nucleotides).

In certain embodiments of any of the systems provided herein, the target nucleic acid is a DNA. In some embodiments of any of the systems described herein, the target nucleic acid includes a protospacer adjacent motif (PAM).

In certain embodiments of any of the systems provided herein, the targeting of the target nucleic acid by the CRISPR-associated protein and RNA guide results in a modification (e.g., a single-stranded or a double-stranded cleavage event) in the target nucleic acid. In some embodiments, the modification results in a deletion event. In some embodiments, the modification results in an insertion event. In some embodiments, the modification results in cell toxicity.

In some embodiments, the CRISPR associated protein has non-specific (i.e., “collateral”) nuclease (e.g., DNAse or DNAse) activity. In certain embodiments of any of the systems provided herein, the system further includes a donor template nucleic acid (e.g., a DNA or an RNA).

In some embodiments any of the systems provided herein is within a cell (e.g., a eukaryotic cell (e.g., a mammalian cell) or a prokaryotic cell (e.g., a bacterial cell).

In some embodiments of any of the systems provided herein, the RNA guide includes a tracrRNA, a modulator RNA, or both. In some embodiments of any of the systems provided herein, the system includes a tracrRNA. In some embodiments of any of the systems provided herein, the system includes a modulator RNA. In some embodiments of any of the systems provided herein, the system does not include a tracrRNA.

In some embodiments of any of the systems provided herein, the CRISPR-associated protein comprises a split RuvC domain. In some embodiments, the CRISPR-associated protein comprises a catalytic residue (e.g., aspartic acid or glutamic acid). In some embodiments, the CRISPR-associated protein cleaves the target nucleic acid. In some embodiments, the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor. In some embodiments, the nucleic acid encoding the CRISPR-associated protein is codon-optimized for expression in a cell. In some embodiments, the nucleic acid encoding the CRISPR-associated protein is operably linked to a promoter. In some embodiments, the nucleic acid encoding the CRISPR-associated protein is in a vector. In some embodiments, the vector comprises a retroviral vector, a lentiviral vector, a phage vector, an adenoviral vector, an adeno-associated vector, or a herpes simplex vector. In some embodiments, the system is present in a delivery composition comprising a nanoparticle, a liposome, an exosome, a microvesicle, or a gene-gun.

In another aspect, the disclosure provides a method of binding a system described herein to a target nucleic acid in a cell, where the method comprises: (a) providing the system; and (b) delivering the system to the cell, wherein the cell comprises the target nucleic acid, wherein the CRISPR-associated protein binds to the RNA guide, and wherein the spacer sequence binds to the target nucleic acid.

In another aspect, the disclosure provides methods of targeting and editing a target nucleic acid including contacting the target nucleic acid with any of the systems described herein. In another aspect, the disclosure provides methods of editing a target nucleic acid including contacting the target nucleic acid with any of the systems described herein.

In one aspect, the disclosure provides engineered, non-naturally occurring Clustered Regularly Interspaced Short Palindromic Repeat (CRISPR)-Cas systems of CLUST.196682 including: an RNA guide including a direct repeat sequence and a spacer sequence capable of hybridizing to a target nucleic acid, or a nucleic acid encoding the RNA guide; and a CRISPR-associated protein or a nucleic acid encoding the CRISPR-associated protein, where the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence provided in Table 8; where the CRISPR-associated protein is capable of binding to the RNA guide and of targeting the target nucleic acid sequence complementary to the spacer sequence.

In some embodiments of any of the systems described herein, the CRISPR-associated protein includes at least one (e.g., one, two, or three) RuvC domain.

In some embodiments of any of the systems described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence provided in Table 9. In some embodiments of any of the systems described herein, the direct repeat sequence includes a nucleotide sequence that is provided in Table 9.

In some embodiments of any of the systems described herein, the CRISPR-associated protein is a CLUST.196682 3300025638 effector protein. In some embodiments of any of the systems described herein, the CRISPR-associated protein is capable of recognizing a protospacer adjacent motif (PAM), and the target nucleic acid includes a PAM including the nucleic acid sequence 5′-CG-3′.

In some embodiments of any of the systems described herein, the spacer sequence of the RNA guide includes between about 17 nucleotides to about 44 nucleotides. In some embodiments of any of the systems described herein, the spacer sequence of the RNA guide includes between 26 and 38 nucleotides.

In some embodiments of any of the systems described herein, the target nucleic acid is a DNA. In some embodiments of any of the systems described herein, the target nucleic acid includes a PAM. In some embodiments of any of the systems described herein, the CRISPR associated protein has non-specific nuclease activity.

In some embodiments of any of the systems described herein, the targeting of the target nucleic acid by the CRISPR-associated protein and RNA guide results in a modification in the target nucleic acid. In some embodiments of any of the systems described herein, the modification in the target nucleic acid is a double-stranded cleavage event or a single-stranded cleavage event. In some embodiments of any of the systems described herein, the modification in the target nucleic acid results in an insertion event or a deletion event. In some embodiments of any of the systems described herein, the modification results in cell toxicity or cell death.

In some embodiments of any of the systems described herein, the systems further include a donor template nucleic acid. In some embodiments of any of the systems described herein, the donor template nucleic acid is a DNA or an RNA.

In some embodiments of any of the systems described herein, the RNA guide includes a tracrRNA, a modulator RNA, or both. In some embodiments of any of the systems described herein, the systems further include a tracrRNA and/or a modulator RNA. In some embodiments of any of the systems provided herein, the system does not include a tracrRNA.

In some embodiments of any of the systems described herein, the systems are within a cell. In some embodiments of any of the systems described herein, the systems are within a eukaryotic cell or a prokaryotic cell.

In some embodiments of any of the systems provided herein, the CRISPR-associated protein comprises a split RuvC domain. In some embodiments, the CRISPR-associated protein comprises a catalytic residue (e.g., aspartic acid or glutamic acid). In some embodiments, the CRISPR-associated protein cleaves the target nucleic acid. In some embodiments, the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor. In some embodiments, the nucleic acid encoding the CRISPR-associated protein is codon-optimized for expression in a cell. In some embodiments, the nucleic acid encoding the CRISPR-associated protein is operably linked to a promoter. In some embodiments, the nucleic acid encoding the CRISPR-associated protein is in a vector. In some embodiments, the vector comprises a retroviral vector, a lentiviral vector, a phage vector, an adenoviral vector, an adeno-associated vector, or a herpes simplex vector. In some embodiments, the system is present in a delivery composition comprising a nanoparticle, a liposome, an exosome, a microvesicle, or a gene-gun.

In another aspect, the disclosure provides a method of binding a system described herein to a target nucleic acid in a cell, where the method comprises: (a) providing the system; and (b) delivering the system to the cell, wherein the cell comprises the target nucleic acid, wherein the CRISPR-associated protein binds to the RNA guide, and wherein the spacer sequence binds to the target nucleic acid.

In one aspect, the disclosure provides methods of targeting and editing a target nucleic acid, the methods including contacting the target nucleic acid with any of the systems described herein. In one aspect, the disclosure provides methods of editing a target nucleic acid, the methods including contacting the target nucleic acid with any of the systems described herein.

In one aspect, the disclosure provides methods of targeting the insertion of a payload nucleic acid at a site of a target nucleic acid, the methods including contacting the target nucleic acid with a system described herein.

In one aspect, the disclosure provides engineered, non-naturally occurring Clustered Regularly Interspaced Short Palindromic Repeat (CRISPR)-Cas systems of CLUST.089537 including: an RNA guide including a direct repeat sequence and a spacer sequence capable of hybridizing to a target nucleic acid, or a nucleic acid encoding the RNA guide; and a CRISPR-associated protein or a nucleic acid encoding the CRISPR-associated protein, where the CRISPR-associated protein includes an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence provided in Table 12; where the CRISPR-associated protein is capable of binding to the RNA guide and of targeting the target nucleic acid sequence complementary to the spacer sequence.

CLUST.089537 and CLUST.085589 can be used interchangeably.

In some embodiments of any of the systems described herein, the CRISPR-associated protein includes at least one (e.g., one, two, or three) RuvC domain.

In some embodiments of any of the systems described herein, the direct repeat sequence includes a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence provided in Table 13. In some embodiments of any of the systems described herein, the direct repeat sequence includes a nucleotide sequence that is provided in Table 13.

In some embodiments of any of the systems described herein, the CRISPR-associated protein is a CLUST.089537 CAAACX010000652 effector protein. In some embodiments of any of the systems described herein, the CRISPR-associated protein is capable of recognizing a protospacer adjacent motif (PAM), and the target nucleic acid includes a PAM including the nucleic acid sequence 5′-TTC-3′.

In some embodiments of any of the systems described herein, the spacer sequence of the RNA guide includes between about 20 nucleotides to about 40 nucleotides. In some embodiments of any of the systems described herein, the spacer sequence of the RNA guide includes between 25 and 37 nucleotides.

In some embodiments of any of the systems described herein, the target nucleic acid is a DNA. In some embodiments of any of the systems described herein, the target nucleic acid includes a PAM. In some embodiments of any of the systems described herein, the CRISPR associated protein has non-specific nuclease activity.

In some embodiments of any of the systems described herein, the targeting of the target nucleic acid by the CRISPR-associated protein and RNA guide results in a modification in the target nucleic acid. For example, in any of the systems described herein, the modification in the target nucleic acid is a double-stranded cleavage event. In other embodiments of any of the systems described herein, the modification in the target nucleic acid is a single-stranded cleavage event, an insertion event, or a deletion event. In some embodiments of any of the systems described herein, the modification results in cell toxicity or cell death.

In some embodiments of any of the systems described herein, the systems further include a donor template nucleic acid. For example, the donor template nucleic acid can be a DNA or an RNA.

In some embodiments of any of the systems described herein, the RNA guide includes a tracrRNA, a modulator RNA, or both. In some embodiments of any of the systems described herein, the systems further include a tracrRNA and/or a modulator RNA. In some embodiments of any of the systems provided herein, the system does not include a tracrRNA.

In some embodiments of any of the systems described herein, the systems are within a cell, e.g., a eukaryotic cell or a prokaryotic cell.

In some embodiments of any of the systems provided herein, the CRISPR-associated protein comprises a split RuvC domain. In some embodiments, the CRISPR-associated protein comprises a catalytic residue (e.g., aspartic acid or glutamic acid). In some embodiments, the CRISPR-associated protein cleaves the target nucleic acid. In some embodiments, the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor. In some embodiments, the nucleic acid encoding the CRISPR-associated protein is codon-optimized for expression in a cell. In some embodiments, the nucleic acid encoding the CRISPR-associated protein is operably linked to a promoter. In some embodiments, the nucleic acid encoding the CRISPR-associated protein is in a vector. In some embodiments, the vector comprises a retroviral vector, a lentiviral vector, a phage vector, an adenoviral vector, an adeno-associated vector, or a herpes simplex vector. In some embodiments, the system is present in a delivery composition comprising a nanoparticle, a liposome, an exosome, a microvesicle, or a gene-gun.

In another aspect, the disclosure provides a method of binding a system described herein to a target nucleic acid in a cell, where the method comprises: (a) providing the system; and (b) delivering the system to the cell, wherein the cell comprises the target nucleic acid, wherein the CRISPR-associated protein binds to the RNA guide, and wherein the spacer sequence binds to the target nucleic acid.

In one aspect, the disclosure provides methods of targeting and editing a target nucleic acid, the methods including contacting the target nucleic acid with any of the systems described herein. In one aspect, the disclosure provides methods of editing a target nucleic acid, the methods including contacting the target nucleic acid with any of the systems described herein.

In some embodiments of any of the systems provided herein, the contacting comprises directly contacting or indirectly contacting. In some embodiments of any of the systems provided herein, contacting indirectly comprises administering one or more nucleic acids encoding an RNA guide or CRISPR-associated protein described herein under conditions that allow for production of the RNA guide and/or CRISPR-related protein. In some embodiments of any of the systems provided herein, contacting includes contacting in vivo or contacting in vitro. In some embodiments of any of the systems provided herein, contacting a target nucleic acid with the system comprises contacting a cell comprising the nucleic acid with the system under conditions that allow the CRISPR-related protein and guide RNA to reach the target nucleic acid. In some embodiments of any of the systems provided herein, contacting a cell in vivo with the system comprises administering the system to the subject that comprises the cell, under conditions that allow the CRISPR-related protein and guide RNA to reach the cell or be produced in the cell.

### BRIEF FIGURE DESCRIPTION

The figures are a series of schematics and nucleic acid and amino acid sequences that represent the results of locus analysis of various protein clusters.

FIG. 1 is a schematic sequence representation that shows conserved effector (e_A) and CRISPR array elements for representative CLUST.121143 loci.

FIG. 2 is a series of sequences that show the multiple sequence alignment of examples of CRISPR direct repeat elements for CLUST.121143. FIG. 2 discloses SEQ ID NOs: 825, 101, 101, 101, 101, 101, 101, 106, 106, 106, 106, 102, 103, 103, 104, 104, 104, 107, 107, 107, 107, 107, 107, 108, 108, 108, 108, 108, 108, 108, 110, 109 and 105, respectively, in order of appearance.

FIG. 3 is a schematic representation of a phylogenetic tree of CLUST.121143 effector proteins.

FIG. 4 is a schematic representation of a multiple sequence alignment of CLUST.121143 effector proteins, with the locations of the conserved catalytic residues of the RuvC domain indicated; and the conserved catalytic residues of Zinc finger domain indicated.

FIG. 5 is a graph that shows the degree of depletion activity of the engineered compositions for spacers targeting pACYC184 and direct repeat transcriptional orientations. To quantify depletion, an enrichment ratio was calculated as R_{treated}/Rᵢₙₚᵤₜ for each direct repeat and spacer. The normalized input read count is computed as:

Rᵢₙₚᵤₜ=# reads containing DR+spacer/total reads

where the reads counts are obtained from next-generation sequencing of the plasmid DNA library expressing a CLUST.121143 effector and associated crRNA prior to transformation. The normalized treated read count is computed as:

R_{treated}=(1+# reads containing DR+spacer)/total # reads

where the read counts are obtained from next-generation sequencing of the plasmid DNA extracted from the surviving cells expressing CLUST.121143 effector and associated crRNA after antibiotic screening. A strongly depleted target has an enrichment less than 1/10. The degree of depletion for CLUST.121143 3300014839 with the direct repeat in the “forward” orientation (5′-CTTT . . . AGAG-[spacer]-3′) and with the direct repeat in the “reverse” orientation (5′-CTCT . . . AAAG-[spacer]-3′) are depicted in the figure.

FIG. 6 is a graph that shows the degree of depletion activity for the negative control, in which the screen is repeated with the sequence encoding the CLUST.121143 effector is deleted from the effector plasmid.

FIGS. 7A and 7B are graphic representations that show the density of depleted and non-depleted targets for CLUST.121143 3300014839 by location on the pACYC184 plasmid and E. coli strain E. Cloni. Targets on the top strand and bottom strand are shown separately, and in relation to the orientation of the annotated genes. FIGS. 7A and 7B both disclose SEQ ID NO: 826.

FIG. 8 is a weblogo of the sequences flanking depleted targets for CLUST.121143 3300014839. FIG. 8 discloses SEQ ID NO: 826.

FIG. 9 is a schematic sequence representation that shows conserved effector (e_A) and CRISPR array elements for representative CLUST.196682 loci.

FIG. 10 is a series of sequences that show multiple sequence alignment of examples of CRISPR direct repeat elements for CLUST.196682. FIG. 10 discloses SEQ ID NOs: 827, 714, 714, 714, 733, 701, 704, 705, 705, 703, 734, 706, 743, 720, 720, 740, 740, 731, 731, 742, 742, 828, 732, 730, 730 and 741, respectively, in order of appearance.

FIG. 11 is a schematic representation of a phylogenetic tree of CLUST.196682 effector proteins.

FIG. 12 is a schematic representation of a multiple sequence alignment of CLUST.196682 effector proteins, with the locations of the conserved catalytic residues of the RuvC domain indicated; and the conserved catalytic residues of Zinc finger domain indicated.

FIGS. 13A and 13B are graphic representations that show the density of depleted and non-depleted targets for CLUST.196682 3300025638 by location on the pACYC184 plasmid and E. coli strain E. Cloni. Targets on the top strand and bottom strand are shown separately, and in relation to the orientation of the annotated genes. FIGS. 13A and 13B both disclose SEQ ID NO: 829.

FIG. 14 is a weblogo of the sequences flanking depleted targets for CLUST.196682 3300025638. FIG. 14 discloses SEQ ID NO: 829.

FIG. 15 is a schematic sequence representation that shows conserved effector (e_A) and CRISPR array elements for representative CLUST.089537 loci.

FIG. 16 is a series of sequences that show multiple sequence alignment of examples of CRISPR direct repeat elements for CLUST.089537. FIG. 16 discloses SEQ ID NOs: 830, 402, 429, 403, 419, 431, 436, 432, 441, 404, 455, 446, 447, 411, 414, 415, 440, 437, 438, 430, 412, 425, 448, 452, 406, 407, 422, 442, 443, 424, 409, 413, 420, 418, 426, 444, 416, 445, 434, 401, 410, 449, 454, 417, 427, 423, 450, 428, 439, 435, 453, 421, 408, 405, 433 and 451, respectively, in order of appearance.

FIG. 17 is a schematic representation of a phylogenetic tree of CLUST.089537 effector proteins.

FIG. 18 is a schematic representation of a multiple sequence alignment of CLUST.089537 effector proteins, with the locations of the conserved catalytic residues of the RuvC domain indicated; and the conserved catalytic residues of Zinc finger domain indicated.

FIG. 19 is a graph that shows the degree of depletion activity of the engineered compositions for spacers targeting pACYC184 and direct repeat transcriptional orientations. To quantify depletion, an enrichment ratio was calculated as R_{treated}/Rᵢₙₚᵤₜ for each direct repeat and spacer. The normalized input read count is computed as:

Rᵢₙₚᵤₜ=# reads containing DR+spacer/total reads

where the reads counts are obtained from next-generation sequencing of the plasmid DNA library expressing a CLUST.089537 effector and associated crRNA prior to transformation. The normalized treated read count is computed as:

R_{treated}=(1+# reads containing DR+spacer)/total # reads

where the read counts are obtained from next-generation sequencing of the plasmid DNA extracted from the surviving cells expressing CLUST.089537 effector and associated crRNA after antibiotic screening. A strongly depleted target has an enrichment less than 1/10. The degree of depletion for CLUST.089537 CAAACX010000652 with the direct repeat in the “forward” orientation (5′-GTGC . . . ACAG-[spacer]-3′) and with the direct repeat in the “reverse” orientation (5′-CTGT . . . GCAC-[spacer]-3′) are depicted in the figure.

FIGS. 20A and 20B are graphic representations that show the density of depleted and non-depleted targets for CLUST.089537 CAAACX010000652 by location on the pACYC184 plasmid and E. coli strain E. Cloni. Targets on the top strand and bottom strand are shown separately, and in relation to the orientation of the annotated genes. FIGS. 20A and 20B both disclose SEQ ID NO: 831.

FIG. 21 is a weblogo of the sequences flanking depleted targets for CLUST.089537 CAAACX010000652. FIG. 21 discloses SEQ ID NO: 831.

### DETAILED DESCRIPTION

The broad natural diversity of CRISPR-Cas defense systems contains a wide range of activity mechanisms and functional elements that can be harnessed for programmable biotechnologies. In a natural system, these mechanisms and parameters enable efficient defense against foreign DNA and viruses while providing self vs. non-self discrimination to avoid self-targeting. In an engineered system, the same mechanisms and parameters also provide a diverse toolbox of molecular technologies and define the boundaries of the targeting space. For instance, systems Cas9 and Cas13a have canonical DNA and RNA endonuclease activity and their targeting spaces are defined by the protospacer adjacent motif (PAM) on targeted DNA and protospacer flanking sites (PFS) on targeted RNA, respectively.

The methods described herein have been used to discover additional mechanisms and parameters within single subunit Class 2 effector systems that can expand the capabilities of RNA-programmable nucleic acid manipulation.

In one aspect, the disclosure relates to the use of computational methods and algorithms to search for and identify novel protein families that exhibit a strong co-occurrence pattern with certain other features within naturally occurring genome sequences. In certain embodiments, these computational methods are directed to identifying protein families that co-occur in close proximity to CRISPR arrays. However, the methods disclosed herein are useful in identifying proteins that naturally occur within close proximity to other features, both non-coding and protein-coding (e.g., fragments of phage sequences in non-coding areas of bacterial loci; or CRISPR Cas1 proteins). It is understood that the methods and calculations described herein may be performed on one or more computing devices.

In some embodiments, a set of genomic sequences is obtained from genomic or metagenomic databases. The databases comprise short reads, or contig level data, or assembled scaffolds, or complete genomic sequences of organisms. Likewise, the database may comprise genomic sequence data from prokaryotic organisms, or eukaryotic organisms, or may include data from metagenomic environmental samples. Examples of database repositories include the National Center for Biotechnology Information (NCBI) RefSeq, NCBI GenBank, NCBI Whole Genome Shotgun (WGS), and the Joint Genome Institute (JGI) Integrated Microbial Genomes (IMG).

In some embodiments, a minimum size requirement is imposed to select genome sequence data of a specified minimum length. In certain exemplary embodiments, the minimum contig length may be 100 nucleotides, 500 nt, 1 kb, 1.5 kb, 2 kb, 3 kb, 4 kb, 5 kb, 10 kb, 20 kb, 40 kb, or 50 kb.

In some embodiments, known or predicted proteins are extracted from the complete or a selected set of genome sequence data. In some embodiments, known or predicted proteins are taken from extracting coding sequence (CDS) annotations provided by the source database. In some embodiments, predicted proteins are determined by applying a computational method to identify proteins from nucleotide sequences. In some embodiments, the GeneMark Suite is used to predict proteins from genome sequences. In some embodiments, Prodigal is used to predict proteins from genome sequences. In some embodiments, multiple protein prediction algorithms may be used over the same set of sequence data with the resulting set of proteins de-duplicated.

In some embodiments, CRISPR arrays are identified from the genome sequence data. In some embodiments, PILER-CR is used to identify CRISPR arrays. In some embodiments, CRISPR Recognition Tool (CRT) is used to identify CRISPR arrays. In some embodiments, CRISPR arrays are identified by a heuristic that identifies nucleotide motifs repeated a minimum number of times (e.g., 2, 3, or 4 times), where the spacing between consecutive occurrences of a repeated motif does not exceed a specified length (e.g., 50, 100, or 150 nucleotides). In some embodiments, multiple CRISPR array identification tools may be used over the same set of sequence data with the resulting set of CRISPR arrays de-duplicated.

In some embodiments, proteins in close proximity to CRISPR arrays are identified. In some embodiments, proximity is defined as a nucleotide distance, and may be within 20 kb, 15 kb, or 5 kb. In some embodiments, proximity is defined as the number of open reading frames (ORFs) between a protein and a CRISPR array, and certain exemplary distances may be 10, 5, 4, 3, 2, 1, or 0 ORFs. The proteins identified as being within close proximity to a CRISPR array are then grouped into clusters of homologous proteins. In some embodiments, blastclust is used to form protein clusters. In certain other embodiments, mmseqs2 is used to form protein clusters.

To establish a pattern of strong co-occurrence between the members of a protein cluster with CRISPR arrays, a BLAST search of each member of the protein family may be performed over the complete set of known and predicted proteins previously compiled. In some embodiments, UBLAST or mmseqs2 may be used to search for similar proteins. In some embodiments, a search may be performed only for a representative subset of proteins in the family.

In some embodiments, the clusters of proteins within close proximity to CRISPR arrays are ranked or filtered by a metric to determine co-occurrence. One exemplary metric is the ratio of the number of elements in a protein cluster against the number of BLAST matches up to a certain E value threshold. In some embodiments, a constant E value threshold may be used. In other embodiments, the E value threshold may be determined by the most distant members of the protein cluster. In some embodiments, the global set of proteins is clustered and the co-occurrence metric is the ratio of the number of elements of the CRISPR associated cluster against the number of elements of the containing global cluster(s).

In some embodiments, a manual review process is used to evaluate the potential functionality and the minimal set of components of an engineered system based on the naturally occurring locus structure of the proteins in the cluster. In some embodiments, a graphical representation of the protein cluster may assist in the manual review, and may contain information including pairwise sequence similarity, phylogenetic tree, source organisms/environments, predicted functional domains, and a graphical depiction of locus structures. In some embodiments, the graphical depiction of locus structures may filter for nearby protein families that have a high representation. In some embodiments, representation may be calculated by the ratio of the number of related nearby proteins against the size(s) of the containing global cluster(s). In certain exemplary embodiments, the graphical representation of the protein cluster may contain a depiction of the CRISPR array structures of the naturally occurring loci. In some embodiments, the graphical representation of the protein cluster may contain a depiction of the number of conserved direct repeats versus the length of the putative CRISPR array, or the number of unique spacer sequences versus the length of the putative CRISPR array. In some embodiments, the graphical representation of the protein cluster may contain a depiction of various metrics of co-occurrence of the putative effector with CRISPR arrays predict new CRISPR-Cas systems and identify their components.

In some embodiments, a complex between an RNA guide and a CRISPR-associated protein described herein is an activated CRISPR complex that has bound to or has modified a target nucleic acid.

### Definitions

The term “cleavage event,” as used herein, refers to a DNA break in a target nucleic acid created by a nuclease of a CRISPR system described herein. In some embodiments, the cleavage event is a double-stranded DNA break. In some embodiments, the cleavage event is a single-stranded DNA break.

The term “CRISPR-Cas system” as used herein refers to nucleic acids and/or proteins involved in the expression of, or directing the activity of, CRISPR-Cas effectors, including sequences encoding CRISPR-Cas effectors, RNA guides, and other sequences and transcripts from a CRISPR locus.

The term “CRISPR array” as used herein refers to the nucleic acid (e.g., DNA) segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The term “CRISPR repeat,” or “CRISPR direct repeat,” or “direct repeat,” as used herein, refers to multiple short direct repeating sequences, which show very little or no sequence variation within a CRISPR array.

The term “CRISPR RNA” or “crRNA” as used herein refers to an RNA molecule comprising a guide sequence used by a CRISPR effector to specifically target a nucleic acid sequence. Typically, crRNAs contain a sequence that mediates target recognition and a sequence that forms a duplex with a tracrRNA. The crRNA: tracrRNA duplex binds to a CRISPR effector. The term “donor template nucleic acid,” as used herein refers to a nucleic acid molecule that can be used by one or more cellular proteins to alter the structure of a target nucleic acid after a CRISPR enzyme described herein has altered a target nucleic acid. In some embodiments, the donor template nucleic acid is a double-stranded nucleic acid. In some embodiments, the donor template nucleic acid is a single-stranded nucleic acid. In some embodiments, the donor template nucleic acid is linear. In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome).

The term “CRISPR-Cas effector,” “CRISPR effector,” “effector,” “CRISPR-associated protein,” or “CRISPR enzyme” as used herein refers to a protein that carries out an enzymatic activity or that binds to a target site on a nucleic acid specified by an RNA guide. In some embodiments, a CRISPR effector has endonuclease activity, nickase activity, exonuclease activity, transposase activity, and/or excision activity.

The term “guide RNA” or “gRNA” as used herein refers to an RNA molecule capable of directing a CRISPR effector having nuclease activity to target and cleave a specified target nucleic acid.

The term “RNA guide” as used herein refers to any RNA molecule that facilitates the targeting of a protein described herein to a target nucleic acid. Exemplary “RNA guides” include, but are not limited to, crRNAs, as well as crRNAs fused to either tracrRNAs and/or modulator RNAs. In some embodiments, an RNA guide includes both a crRNA and a tracrRNA. In some embodiments, an RNA guide includes a crRNA and a modulator RNA. In some embodiments, an RNA guide includes a crRNA, a tracrRNA, and a modulator RNA.

The term “modulator RNA” as described herein refers to any RNA molecule that modulates (e.g., increases or decreases) an activity of a CRISPR-Cas effector or a nucleoprotein complex that includes a CRISPR-Cas effector. In some embodiments, a modulator RNA modulates a nuclease activity of a CRISPR-Cas effector or a nucleoprotein complex that includes a CRISPR-Cas effector.

As used herein, the term “target nucleic acid” refers to a specific nucleic acid sequence that is to be modified by a CRISPR system described herein. In some embodiments, the target nucleic acid comprises a gene. In some embodiments, the target nucleic acid comprises a non-coding region (e.g., a promoter). In some embodiments, the target nucleic acid is single-stranded. In some embodiments, the target nucleic acid is double-stranded.

The terms “trans-activating crRNA” or “tracrRNA” as used herein refer to an RNA including a sequence that forms a structure required for a CRISPR effector to bind to a specified target nucleic acid.

A “transcriptionally-active site” as used herein refers to a site in a nucleic acid sequence comprising promoter regions at which transcription is initiated and actively occurring.

The term “collateral RNAse activity,” as used herein in reference to a CRISPR enzyme, refers to non-specific RNAse activity of a CRISPR enzyme after the enzyme has modified a specifically targeted nucleic acid.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### Pooled-Screening

To efficiently validate the activity of the engineered novel CRISPR-Cas systems and simultaneously evaluate in an unbiased manner different activity mechanisms and functional parameters, a new pooled-screening approach in E. coli. First, from the computational identification of the conserved protein and noncoding elements of the novel CRISPR-Cas system. DNA synthesis and molecular cloning was used to assemble the separate components into a single artificial expression vector, which in one embodiment is based on a pET-28a+ backbone. In a second embodiment, the effectors and noncoding elements are transcribed on a single mRNA transcript, and different ribosomal binding sites are used to translate individual effectors.

Second, the natural crRNA and targeting spacers were replaced with a library of unprocessed crRNAs containing non-natural spacers targeting a second plasmid, pACYC184. This crRNA library was cloned into the vector backbone containing the protein effectors and noncoding elements (e.g., pET-28a+), and then subsequently transformed the library into E. coli along with the pACYC184 plasmid target. Consequently, each resulting E. coli cell contains no more than one targeting spacer. In an alternate embodiment, the library of unprocessed crRNAs containing non-natural spacers additionally target E. coli essential genes, drawn from resources such as those described in Baba et al. (2006) Mol. Syst. Biol. 2: 2006.0008; and Gerdes et al. (2003) J. Bacteriol. 185(19): 5673-84, the entire contents of each of which are incorporated herein by reference. In this embodiment, positive, targeted activity of the novel CRISPR-Cas systems that disrupts essential gene function results in cell death or growth arrest. In some embodiments, the essential gene targeting spacers can be combined with the pACYC184 targets to add another dimension to the assay.

Third, the E. coli were grown under antibiotic selection. In one embodiment, triple antibiotic selection is used: kanamycin for ensuring successful transformation of the pET-28a+ vector containing the engineered CRISPR-Cas effector system, and chloramphenicol and tetracycline for ensuring successful co-transformation of the pACYC184 target vector. Since pACYC184 normally confers resistance to chloramphenicol and tetracycline, under antibiotic selection, positive activity of the novel CRISPR-Cas system targeting the plasmid will eliminate cells that actively express the effectors, noncoding elements, and specific active elements of the crRNA library. Examining the population of surviving cells at a later time point compared to an earlier time point results in a depleted signal compared to the inactive crRNAs. In some embodiments, double antibiotic selection is used. For example, withdrawal of either chloramphenicol or tetracycline to remove selective pressure can provide novel information about the targeting substrate, sequence specificity, and potency. In some embodiments, only kanamycin is used to ensure successful transformation of the pET-28a+ vector containing the engineered CRISPR-Cas effector system. This embodiment is suitable for libraries containing spacers targeting E. coli essential genes, as no additional selection beyond kanamycin is needed to observe growth alterations. In this embodiment, chloramphenicol and tetracycline dependence is removed, and their targets (if any) in the library provides an additional source of negative or positive information about the targeting substrate, sequence specificity, and potency.

Since the pACYC184 plasmid contains a diverse set of features and sequences that may affect the activity of a CRISPR-Cas system, mapping the active crRNAs from the pooled screen onto pACYC184 provides patterns of activity that can be suggestive of different activity mechanisms and functional parameters in a broad, hypothesis-agnostic manner. In this way, the features required for reconstituting the novel CRISPR-Cas system in a heterologous prokaryotic species can be more comprehensively tested and studied.

The key advantages of the in vivo pooled-screen described herein include:

(1) Versatility—Plasmid design allows multiple effectors and/or noncoding elements to be expressed; library cloning strategy enables both transcriptional directions of the computationally predicted crRNA to be expressed;

(2) Comprehensive tests of activity mechanisms & functional parameters—Evaluates diverse interference mechanisms, including DNA or RNA cleavage; examines co-occurrence of features such as transcription, plasmid DNA replication; and flanking sequences for crRNA library can be used to reliably determine PAMs with complexity equivalence of 4N's;

(3) Sensitivity—pACYC184 is a low copy plasmid, enabling high sensitivity for CRISPR-Cas activity since even modest interference rates can eliminate the antibiotic resistance encoded by the plasmid; and

(4) Efficiency—Optimized molecular biology steps to enable greater speed and throughput RNA-sequencing and protein expression samples can be directly harvested from the surviving cells in the screen.

The novel CRISPR-Cas families described herein were evaluated using this in vivo pooled-screen to evaluate their operational elements, mechanisms and parameters, as well as their ability to be active and reprogrammed in an engineered system outside of their natural cellular environment.

### Class 2 CRISPR-Cas Effectors Having a RuvC Domain

In one aspect, the disclosure provides a Class 2 CRISPR-Cas systems referred to herein as CLUST.121143. This Class 2 CRISPR-Cas system contains an isolated CRISPR-associated protein having a RuvC domain.

In one aspect, the disclosure provides a Class 2 CRISPR-Cas systems referred to herein as CLUST.196682. This Class 2 CRISPR-Cas system contains an isolated CRISPR-associated protein having a RuvC domain.

In one aspect, the disclosure provides a Class 2 CRISPR-Cas systems referred to herein as CLUST.089537. This Class 2 CRISPR-Cas system contains an isolated CRISPR-associated protein having a RuvC domain.

In some embodiments, the CRISPR-associated protein and the RNA guide form a “binary” complex that may include other components. The binary complex is activated upon binding to a nucleic acid substrate that is complementary to a spacer sequence in the RNA guide (i.e., a sequence-specific substrate or target nucleic acid). In some embodiments, the sequence-specific substrate is a double-stranded DNA. In some embodiments, the sequence-specific substrate is a single-stranded DNA. In some embodiments, the sequence-specific substrate is a single-stranded RNA. In some embodiments, the sequence-specific substrate is a double-stranded RNA. In some embodiments, the sequence-specificity requires a complete match of the spacer sequence in the RNA guide (e.g., crRNA) to the target substrate. In other embodiments, the sequence specificity requires a partial (contiguous or non-contiguous) match of the spacer sequence in the RNA guide (e.g., crRNA) to the target substrate.

In some embodiments, the binary complex becomes activated upon binding to the target substrate. In some embodiments, the activated complex exhibits “multiple turnover” activity, whereby upon acting on (e.g., cleaving) the target substrate the activated complex remains in an activated state. In some embodiments, the activated binary complex exhibits “single turnover” activity, whereby upon acting on the target substrate the binary complex reverts to an inactive state. In some embodiments, the activated binary complex exhibits non-specific (i.e., “collateral”) cleavage activity whereby the complex cleaves non-target nucleic acids. In some embodiments, the non-target nucleic acid is a DNA (e.g., a single-stranded or a double-stranded DNA). In some embodiments, the non-target nucleic acid is an RNA (e.g., a single-stranded or a double-stranded RNA).

### CRISPR Enzyme Modifications

Deactivated/Inactivated CRISPR Enzymes

Where the CRISPR enzymes described herein have nuclease activity, the CRISPR enzymes can be modified to have diminished nuclease activity, e.g., nuclease inactivation of at least 50%, at least 60%, at least 70%, at least 80%, at least 90%, at least 95%, at least 97%, or 100% as compared with the wild type CRISPR enzymes. The nuclease activity can be diminished by several methods known in the art, e.g., introducing mutations into the nuclease domains of the proteins. In some embodiments, catalytic residues for the nuclease activities are identified, and these amino acid residues can be substituted by different amino acid residues (e.g., glycine or alanine) to diminish the nuclease activity.

The inactivated CRISPR enzymes can comprise or be associated with one or more functional domains (e.g., via fusion protein, linker peptides, “GS” linkers, etc.). These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, and switch activity (e.g., light inducible). In some embodiments, the functional domains are Krappel associated box (KRAB), VP64, VP16, Fok1, P65, HSF1, MyoD1, and biotin-APEX.

The positioning of the one or more functional domains on the inactivated CRISPR enzymes is one that allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., Fok1) is positioned to cleave or partially cleave the target. In some embodiments, the functional domain is positioned at the N-terminus of the CRISPR enzyme. In some embodiments, the functional domain is positioned at the C-terminus of the CRISPR enzyme. In some embodiments, the inactivated CRISPR enzyme is modified to comprise a first functional domain at the N-terminus and a second functional domain at the C-terminus.

Split Enzymes

The present disclosure also provides a split version of the CRISPR enzymes described herein. The split version of the CRISPR enzymes may be advantageous for delivery. In some embodiments, the CRISPR enzymes are split to two parts of the enzymes, which together substantially comprises a functioning CRISPR enzyme.

The split can be done in a way that the catalytic domain(s) are unaffected. The CRISPR enzymes may function as a nuclease or may be inactivated enzymes, which are essentially RNA-binding proteins with very little or no catalytic activity (e.g., due to mutation(s) in its catalytic domains).

In some embodiments, the nuclease lobe and α-helical lobe are expressed as separate polypeptides. Although the lobes do not interact on their own, the guide RNA recruits them into a ternary complex that recapitulates the activity of full-length CRISPR enzymes and catalyzes site-specific DNA cleavage. The use of a modified guide RNA abrogates split-enzyme activity by preventing dimerization, allowing for the development of an inducible dimerization system. The split enzyme is described, e.g., in Wright, Addison V., et al. “Rational design of a split-Cas9 enzyme complex,” Proc. Nat'l. Acad. Sci., 112.10 (2015): 2984-2989, which is incorporated herein by reference in its entirety.

In some embodiments, the split enzyme can be fused to a dimerization partner, e.g., by employing rapamycin sensitive dimerization domains. This allows the generation of a chemically inducible CRISPR enzyme for temporal control of CRISPR enzyme activity. The CRISPR enzymes can thus be rendered chemically inducible by being split into two fragments and rapamycin-sensitive dimerization domains can be used for controlled reassembly of the CRISPR enzymes.

The split point is typically designed in silico and cloned into the constructs. During this process, mutations can be introduced to the split enzyme and non-functional domains can be removed. In some embodiments, the two parts or fragments of the split CRISPR enzyme (i.e., the N-terminal and C-terminal fragments), can form a full CRISPR enzyme, comprising, e.g., at least 70%, at least 80%, at least 90%, at least 95%, or at least 99% of the sequence of the wild-type CRISPR enzyme.

Self-Activating or Inactivating Enzymes

The CRISPR enzymes described herein can be designed to be self-activating or self-inactivating. In some embodiments, the CRISPR enzymes are self-inactivating. For example, the target sequence can be introduced into the CRISPR enzyme coding constructs. Thus, the CRISPR enzymes can cleave the target sequence, as well as the construct encoding the enzyme thereby self-inactivating their expression. Methods of constructing a self-inactivating CRISPR system is described, e.g., in Epstein, Benjamin E., and David V. Schaffer. “Engineering a Self-Inactivating CRISPR System for AAV Vectors,” Mol. Ther., 24 (2016): S50, which is incorporated herein by reference in its entirety.

In some other embodiments, an additional guide RNA, expressed under the control of a weak promoter (e.g., 7SK promoter), can target the nucleic acid sequence encoding the CRISPR enzyme to prevent and/or block its expression (e.g., by preventing the transcription and/or translation of the nucleic acid). The transfection of cells with vectors expressing the CRISPR enzyme, guide RNAs, and guide RNAs that target the nucleic acid encoding the CRISPR enzyme can lead to efficient disruption of the nucleic acid encoding the CRISPR enzyme and decrease the levels of CRISPR enzyme, thereby limiting the genome editing activity.

In some embodiments, the genome editing activity of the CRISPR enzymes can be modulated through endogenous RNA signatures (e.g., miRNA) in mammalian cells. The CRISPR enzyme switch can be made by using a miRNA-complementary sequence in the 5′-UTR of mRNA encoding the CRISPR enzyme. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (Hirosawa, Moe et al. “Cell-type-specific genome editing with a microRNA-responsive CRISPR-Cas9 switch,” Nucl. Acids Res., 2017 July 27; 45(13): e118).

Inducible CRISPR Enzymes

The CRISPR enzymes can be inducible, e.g., light inducible or chemically inducible. This mechanism allows for activation of the functional domain in the CRISPR enzymes. Light inducibility can be achieved by various methods known in the art, e.g., by designing a fusion complex wherein CRY2 PHR/CIBN pairing is used in split CRISPR Enzymes (see, e.g., Konermann et al. “Optical control of mammalian endogenous transcription and epigenetic states,” Nature, 500.7463 (2013): 472). Chemical inducibility can be achieved, e.g., by designing a fusion complex wherein FKBP/FRB (FK506 binding protein/FKBP rapamycin binding domain) pairing is used in split CRISPR Enzymes. Rapamycin is required for forming the fusion complex, thereby activating the CRISPR enzymes (see, e.g., Zetsche, Volz, and Zhang, “A split-Cas9 architecture for inducible genome editing and transcription modulation,” Nature Biotech., 33.2 (2015): 139-142).

Furthermore, expression of the CRISPR enzymes can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless, Stephen J. et al. “Direct and specific chemical control of eukaryotic translation with a synthetic RNA-protein interaction,” Nucl. Acids Res., 40.9 (2012): e64-e64).

Various embodiments of inducible CRISPR enzymes and inducible CRISPR systems are described, e.g., in U.S. Pat. No. 8,871,445, US20160208243, and WO2016205764, each of which is incorporated herein by reference in its entirety.

Functional Mutations

Various mutations or modifications can be introduced into CRISPR enzymes as described herein to improve specificity and/or robustness. In some embodiments, the amino acid residues that recognize the Protospacer Adjacent Motif (PAM) are identified. The CRISPR enzymes described herein can be modified further to recognize different PAMs, e.g., by substituting the amino acid residues that recognize PAM with other amino acid residues. In some embodiments, the CRISPR enzymes can recognize, e.g., 5′-NGG-3′, 5′-YG-3′, 5′-TTTN-3′, or 5′-YTN-3′ PAM, wherein “Y” is a pyrimidine and “N” is any nucleobase.

In some embodiments, at least one Nuclear Localization Signal (NLS) is attached to the nucleic acid sequences encoding the CRISPR enzyme. In some embodiments, at least one Nuclear Export Signal (NES) is attached to the nucleic acid sequences encoding the CRISPR enzyme. In some embodiments, a C-terminal and/or N-terminal NLS or NES is attached for optimal expression and nuclear targeting in eukaryotic cells, e.g., human cells.

In some embodiments, the CRISPR enzymes described herein are mutated at one or more amino acid residues to alter one or more functional activities. For example, in some embodiments, the CRISPR enzyme is mutated at one or more amino acid residues to alter its helicase activity. In some embodiments, the CRISPR enzyme is mutated at one or more amino acid residues to alter its nuclease activity (e.g., endonuclease activity or exonuclease activity). In some embodiments, the CRISPR enzyme is mutated at one or more amino acid residues to alter its ability to functionally associate with a guide RNA. In some embodiments, the CRISPR enzyme is mutated at one or more amino acid residues to alter its ability to functionally associate with a target nucleic acid.

In some embodiments, the CRISPR enzymes described herein are capable of cleaving a target nucleic acid molecule. In some embodiments, the CRISPR enzyme cleaves both strands of the target nucleic acid molecule. However, in some embodiments, the CRISPR enzyme is mutated at one or more amino acid residues to alter its cleaving activity. For example, in some embodiments, the CRISPR enzyme may comprise one or more mutations that render the enzyme incapable of cleaving a target nucleic acid. In other embodiments, the CRISPR enzyme may comprise one or more mutations such that the enzyme is capable of cleaving a single strand of the target nucleic acid (i.e., nickase activity). In some embodiments, the CRISPR enzyme is capable of cleaving the strand of the target nucleic acid that is complementary to the strand that the guide RNA hybridizes to. In some embodiments, the CRISPR enzyme is capable of cleaving the strand of the target nucleic acid that the guide RNA hybridizes to.

In some embodiments, a CRISPR enzyme described herein may be engineered to comprise a deletion in one or more amino acid residues to reduce the size of the enzyme while retaining one or more desired functional activities (e.g., nuclease activity and the ability to interact functionally with a guide RNA). The truncated CRISPR enzyme may be used advantageously in combination with delivery systems having load limitations.

In one aspect, the present disclosure provides nucleic acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the nucleic sequences described herein. In another aspect, the present disclosure also provides amino acid sequences that are at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequences described herein.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as the sequences described herein.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

### Guide RNA Modifications

In some embodiments, an RNA guide described herein comprises a uracil (U). In some embodiments, an RNA guide described herein comprises a thymine (T). In some embodiments, a direct repeat sequence of an RNA guide described herein comprises a uracil (U). In some embodiments, a direct repeat sequence of an RNA guide described herein comprises a thymine (T). In some embodiments, a direct repeat sequence according to any of Tables 3, 9, or 13 comprises a sequence comprising a uracil, in one or more places indicated as thymine in the corresponding sequences in any of Tables 3, 9, or 13.

In some embodiments, the direct repeat comprises only one copy of a sequence that is repeated in an endogenous CRISPR array. In some embodiments, the direct repeat is a full-length sequence adjacent to (e.g., flanking) one or more spacer sequences found in an endogenous CRISPR array. In some embodiments, the direct repeat is a portion (e.g., processed portion) of a full-length sequence adjacent to (e.g., flanking) one or more spacer sequences found in an endogenous CRISPR array.

Spacer Lengths

In some embodiments, the CRISPR-Cas system is the CRISPR-Cas system of CLUST.121143. The spacer length of guide RNAs can range from about 15 to 50 nucleotides. In some embodiments, the spacer length of a guide RNA is at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, or at least 27 nucleotides. In some embodiments, the spacer length is from 15 to 17 nucleotides, from 15 to 23 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 33 nucleotides (e.g., 30, 31, 32, or 33 nucleotides), from 33 to 36 nucleotides (e.g., 33, 34, 35, or 36 nucleotides), from 36 to 40 nucleotides (e.g., 36, 37, 38, 39, or 40 nucleotides), from 40 to 45 nucleotides (e.g., 40, 41, 42, 43, 44, or 45 nucleotides), from 30 or 35 to 40 nucleotides, from 41 to 45 nucleotides, from 45 to 50 nucleotides, or longer. In some embodiments, the spacer length is between 22 to 40 nucleotides, or from 26 to 35 nucleotides. In some embodiments, the direct repeat length of the guide RNA is at least 16 nucleotides, or is from 16 to 20 nucleotides (e.g., 16, 17, 18, 19, or 20 nucleotides). In some embodiments, the direct repeat length of the guide RNA is 19 nucleotides.

In some embodiments, the CRISPR-Cas system is the CRISPR-Cas system of CLUST.196682. The spacer length of guide RNAs can range from about 15 to 50 nucleotides. In some embodiments, the spacer length of a guide RNA is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, or longer. In some embodiments, the direct repeat length of the guide RNA is at least 16 nucleotides, or is from 16 to 20 nucleotides (e.g., 16, 17, 18, 19, or 20 nucleotides). In some embodiments, the direct repeat length of the guide RNA is 19 nucleotides.

In some embodiments, the CRISPR-Cas system is the CRISPR-Cas system of CLUST.089537. The spacer length of guide RNAs can range from about 15 to 50 nucleotides. In some embodiments, the spacer length of a guide RNA is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides, from 15 to 23 nucleotides, from 16 to 22 nucleotides, from 17 to 20 nucleotides, from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 40, or 45 nucleotides), from 30 or 35 to 40 nucleotides, from 41 to 45 nucleotides, from 45 to 50 nucleotides, or longer. In some embodiments, the direct repeat length of the guide RNA is at least 16 nucleotides, or is from 16 to 20 nucleotides (e.g., 16, 17, 18, 19, or 20 nucleotides). In some embodiments, the direct repeat length of the guide RNA is 19 nucleotides.

In some embodiments, the direct repeat comprises only one copy of a sequence that is repeated in an endogenous CRISPR array. In some embodiments, the direct repeat is a full-length sequence adjacent to (e.g., flanking) one or more spacer sequences found in an endogenous CRISPR array. In some embodiments, the direct repeat is a portion (e.g., processed portion) of a full-length sequence adjacent to (e.g., flanking) one or more spacer sequences found in an endogenous CRISPR array.

The guide RNA sequences can be modified in a manner that allows for formation of the CRISPR complex and successful binding to the target, while at the same time not allowing for successful nuclease activity (i.e., without nuclease activity/without causing indels). These modified guide sequences are referred to as “dead guides” or “dead guide sequences.” These dead guides or dead guide sequences may be catalytically inactive or conformationally inactive with regard to nuclease activity. Dead guide sequences are typically shorter than respective guide sequences that result in active RNA cleavage. In some embodiments, dead guides are 5%, 10%, 20%, 30%, 40%, or 50%, shorter than respective guide RNAs that have nuclease activity. Dead guide sequences of guide RNAs can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR systems including a functional CRISPR enzyme as described herein, and a guide RNA (gRNA) wherein the gRNA comprises a dead guide sequence whereby the gRNA is capable of hybridizing to a target sequence such that the CRISPR system is directed to a genomic locus of interest in a cell without detectable cleavage activity.

A detailed description of dead guides is described, e.g., in WO 2016094872, which is incorporated herein by reference in its entirety.

Inducible Guides

Guide RNAs can be generated as components of inducible systems. The inducible nature of the systems allows for spatiotemporal control of gene editing or gene expression. In some embodiments, the stimuli for the inducible systems include, e.g., electromagnetic radiation, sound energy, chemical energy, and/or thermal energy.

In some embodiments, the transcription of guide RNA can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in their entirety.

Chemical Modifications

Chemical modifications can be applied to the guide RNA's phosphate backbone, sugar, and/or base. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3:154). Additionally, RNA is amenable to both 5′ and 3′ end conjugations with a variety of functional moieties including fluorescent dyes, polyethylene glycol, or proteins.

A wide variety of modifications can be applied to chemically synthesized guide RNA molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the guide RNA includes one or more phosphorothioate modifications. In some embodiments, the guide RNA includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965 B2; each which is incorporated by reference in its entirety.

Sequence Modifications

The sequences and the lengths of the guide RNAs, tracrRNAs, and crRNAs described herein can be optimized. In some embodiments, the optimized length of guide RNA can be determined by identifying the processed form of tracrRNA and/or crRNA, or by empirical length studies for guide RNAs, tracrRNAs, crRNAs, and the tracrRNA tetraloops.

The guide RNAs can also include one or more aptamer sequences. Aptamers are oligonucleotide or peptide molecules that can bind to a specific target molecule. The aptamers can be specific to gene effectors, gene activators, or gene repressors. In some embodiments, the aptamers can be specific to a protein, which in turn is specific to and recruits/binds to specific gene effectors, gene activators, or gene repressors. The effectors, activators, or repressors can be present in the form of fusion proteins. In some embodiments, the guide RNA has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCbl2r, ϕCb23r, 7s, and PRR1. Accordingly, in some embodiments, the aptamer is selected from binding proteins specifically binding any one of the adaptor proteins as described herein. In some embodiments, the aptamer sequence is a MS2 loop. A detailed description of aptamers can be found, e.g., in Nowak et al., “Guide RNA engineering for versatile Cas9 functionality,” Nucl. Acid. Res., 2016 Nov. 16; 44(20):9555-9564; and WO 2016205764, which are incorporated herein by reference in their entirety.

Guide: Target Sequence Matching Requirements

In classic CRISPR systems, the degree of complementarity between a guide sequence and its corresponding target sequence can be about 50%, 60%, 75%, 80%, 85%, 90%, 95%, 97.5%, 99%, or 100%. In some embodiments, the degree of complementarity is 100%. The guide RNAs can be about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required provided that there is sufficient complementarity to be functional. Modulations of cleavage efficiency can be exploited by introduction of mismatches, e.g., one or more mismatches, such as 1 or 2 mismatches between spacer sequence and target sequence, including the position of the mismatch along the spacer/target. Typically, the more central (i.e., not at the 3′ or 5′ ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

### Methods of Using CRISPR Systems

The CRISPR systems described herein have a wide variety of utilities including modifying (e.g., deleting, inserting, translocating, inactivating, or activating) a target polynucleotide in a multiplicity of cell types. The CRISPR systems have a broad spectrum of applications in, e.g., DNA/RNA detection (e.g., specific high sensitivity enzymatic reporter unlocking (SHERLOCK)), tracking and labeling of nucleic acids, enrichment assays (extracting desired sequence from background), detecting circulating tumor DNA, preparing next generation library, drug screening, disease diagnosis and prognosis, and treating various genetic disorders.

In another aspect, the disclosure provides methods of targeting the insertion or excision of a payload nucleic acid at a site of a target nucleic acid, wherein the methods include contacting the target nucleic acid with any of the systems described herein.

In another aspect, the disclosure provides methods of non-specifically degrading single-stranded DNA upon recognition of a DNA target nucleic acid, the method including contacting the target nucleic acid with any of the systems described herein.

In another aspect, the disclosure provides methods of detecting a target nucleic acid (e.g., DNA or RNA) in a sample, the methods including contacting the sample with any of the systems described herein and a labeled reporter nucleic acid, where hybridization of the crRNA to the target nucleic acid causes cleavage of the labeled reporter nucleic acid, and measuring a detectable signal produced by cleavage of the labeled reporter nucleic acid, thereby detecting the presence of the target nucleic acid in the sample.

In yet another aspect, the disclosure provides methods of treating a condition or disease in a subject in need thereof, the methods including administering to the subject any of the systems described herein, where the spacer sequence is complementary to between 22 to 40 nucleotides of a target nucleic acid associated with the condition or disease; where the CRISPR-associated protein associates with the RNA guide to form a complex; where the complex binds to a target nucleic acid sequence that is complementary to the nucleotides of the spacer sequence; and where upon binding of the complex to the target nucleic acid sequence the CRISPR-associated protein cleaves the target nucleic acid, thereby treating the condition or disease in the subject.

In one aspect, the disclosure provides methods of treating a condition or disease in a subject in need thereof, the methods including administering to the subject any of the systems described herein, where the spacer sequence is complementary to between 17 to 44 nucleotides of a target nucleic acid associated with the condition or disease; where the CRISPR-associated protein associates with the RNA guide to form a complex; where the complex binds to a target nucleic acid sequence that is complementary to the nucleotides of the spacer sequence; and where, upon binding of the complex to the target nucleic acid sequence, the CRISPR-associated protein cleaves the target nucleic acid, thereby treating the condition or disease in the subject.

In one aspect, the disclosure provides methods of treating a condition or disease in a subject in need thereof, the methods including administering to the subject any of the systems described herein, where the spacer sequence is complementary to between 20 to 40 nucleotides of a target nucleic acid associated with the condition or disease; where the CRISPR-associated protein associates with the RNA guide to form a complex; where the complex binds to a target nucleic acid sequence that is complementary to the nucleotides of the spacer sequence; and where upon binding of the complex to the target nucleic acid sequence the CRISPR-associated protein cleaves the target nucleic acid, thereby treating the condition or disease in the subject.

In another aspect, the disclosure provides methods for using any of the systems disclosed herein as a medicament (e.g., for use in the treatment or prevention of a condition or disease).

In some embodiments, the condition or disease is an infectious disease or cancer (e.g., Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer).

In yet another aspect, the disclosure provides methods for using any of the systems disclosed herein in a method (e.g., an in vitro or ex vivo method) of editing a target nucleic acid, e.g., targeting and editing a target nucleic acid; non-specifically degrading single-stranded DNA upon recognition of a DNA target nucleic acid; targeting and nicking a non-spacer complementary strand of a double-stranded target DNA upon recognition of a spacer complementary strand of the double-stranded target DNA; targeting and cleaving a double-stranded target DNA; detecting a target nucleic acid in a sample; specifically editing a double-stranded nucleic acid; base editing a double-stranded nucleic acid; inducing genotype-specific or transcriptional-state-specific cell death or dormancy in a cell; creating an indel in a double-stranded target DNA; inserting a sequence into a double-stranded target DNA, or deleting or inverting a sequence in a double-stranded target DNA.

DNA/RNA Detection

In one aspect, the CRISPR systems described herein can be used in DNA/RNA detection. Single effector RNA-guided DNases can be reprogrammed with CRISPR RNAs (crRNAs) to provide a platform for specific single-stranded DNA (ssDNA) sensing. Upon recognition of its DNA target, activated Type V single effector DNA-guided DNases engage in “collateral” cleavage of nearby non-targeted ssDNAs. This crRNA-programmed collateral cleavage activity allows the CRISPR systems to detect the presence of a specific DNA by nonspecific degradation of labeled ssDNA.

The collateral ssDNA activity can be combined with a reporter in DNA detection applications such as a method called the DNA Endonuclease-Targeted CRISPR trans reporter (DETECTR) method, which achieves attomolar sensitivity for DNA detection (see, e.g., Chen et al., Science, 360(6387):436-439, 2018), which is incorporated herein by reference in its entirety. One application of using the enzymes described herein is to degrade non-specific ssDNA in an in vitro environment. A “reporter” ssDNA molecule linking a fluorophore and a quencher can also be added to the in vitro system, along with an unknown sample of DNA (either single-stranded or double-stranded). Upon recognizing the target sequence in the unknown piece of DNA, the effector complex cleaves the reporter ssDNA resulting in a fluorescent readout.

In other embodiments, the SHERLOCK method (Specific High Sensitivity Enzymatic Reporter UnLOCKing) also provides an in vitro nucleic acid detection platform with attomolar (or single-molecule) sensitivity based on nucleic acid amplification and collateral cleavage of a reporter ssDNA, allowing for real-time detection of the target. Methods of using CRISPR in SHERLOCK are described in detail, e.g., in Gootenberg, et al. “Nucleic acid detection with CRISPR-Cas13a/C2c2,” Science, 356(6336):438-442 (2017), which is incorporated herein by reference in its entirety.

In some embodiments, the CRISPR systems described herein can be used in multiplexed error-robust fluorescence in situ hybridization (MERFISH). These methods are described in, e.g., Chen et al., “Spatially resolved, highly multiplexed RNA profiling in single cells,” Science, 2015 Apr. 24; 348(6233):aaa6090, which is incorporated herein by reference in its entirety.

Tracking and Labeling of Nucleic Acids

Cellular processes depend on a network of molecular interactions among proteins, RNAs, and DNAs. Accurate detection of protein-DNA and protein-RNA interactions is key to understanding such processes. In vitro proximity labeling techniques employ an affinity tag combined with, a reporter group, e.g., a photoactivatable group, to label polypeptides and RNAs in the vicinity of a protein or RNA of interest in vitro. After UV irradiation, the photoactivatable groups react with proteins and other molecules that are in close proximity to the tagged molecules, thereby labelling them. Labelled interacting molecules can subsequently be recovered and identified. The RNA targeting effector proteins can for instance be used to target probes to selected RNA sequences. These applications can also be applied in animal models for in vivo imaging of diseases or difficult-to culture cell types. The methods of tracking and labeling of nucleic acids are described, e.g., in U.S. Pat. No. 8,795,965; WO 2016205764; and WO 2017070605; each of which is incorporated herein by reference in its entirety.

High-Throughput Screening

The CRISPR systems described herein can be used for preparing next generation sequencing (NGS) libraries. For example, to create a cost-effective NGS library, the CRISPR systems can be used to disrupt the coding sequence of a target gene, and the CRISPR enzyme transfected clones can be screened simultaneously by next-generation sequencing (e.g., on the Ion Torrent PGM system). A detailed description regarding how to prepare NGS libraries can be found, e.g., in Bell et al., “A high-throughput screening strategy for detecting CRISPR-Cas9 induced mutations using next-generation sequencing,” BMC Genomics, 15.1 (2014): 1002, which is incorporated herein by reference in its entirety.

Engineered Microorganisms

Microorganisms (e.g., E. coli, yeast, and microalgae) are widely used for synthetic biology. The development of synthetic biology has a wide utility, including various clinical applications. For example, the programmable CRISPR systems can be used to split proteins of toxic domains for targeted cell death, e.g., using cancer-linked RNA as target transcript. Further, pathways involving protein-protein interactions can be influenced in synthetic biological systems with, e.g., fusion complexes with the appropriate effectors such as kinases or enzymes.

In some embodiments, guide RNA sequences that target phage sequences can be introduced into the microorganism. Thus, the disclosure also provides methods of vaccinating a microorganism (e.g., a production strain) against phage infection.

In some embodiments, the CRISPR systems provided herein can be used to engineer microorganisms, e.g., to improve yield or improve fermentation efficiency. For example, the CRISPR systems described herein can be used to engineer microorganisms, such as yeast, to generate biofuel or biopolymers from fermentable sugars, or to degrade plant-derived lignocellulose derived from agricultural waste as a source of fermentable sugars. More particularly, the methods described herein can be used to modify the expression of endogenous genes required for biofuel production and/or to modify endogenous genes, which may interfere with the biofuel synthesis. These methods of engineering microorganisms are described e.g., in Verwaal et al., “CRISPR/Cpf1 enables fast and simple genome editing of Saccharomyces cerevisiae,” Yeast, 2017 Sep. 8. doi: 10.1002/yea.3278; and Hlavova et al., “Improving microalgae for biotechnology—from genetics to synthetic biology,” Biotechnol. Adv., 2015 Nov. 1; 33:1194-203, both of which are incorporated herein by reference in their entirety.

Application in Plants

The CRISPR systems described herein have a wide variety of utility in plants. In some embodiments, the CRISPR systems can be used to engineer genomes of plants (e.g., improving production, making products with desired post-translational modifications, or introducing genes for producing industrial products). In some embodiments, the CRISPR systems can be used to introduce a desired trait to a plant (e.g., with or without heritable modifications to the genome), or regulate expression of endogenous genes in plant cells or whole plants.

In some embodiments, the CRISPR systems can be used to identify, edit, and/or silence genes encoding specific proteins, e.g., allergenic proteins (e.g., allergenic proteins in peanuts, soybeans, lentils, peas, green beans, and mung beans). A detailed description regarding how to identify, edit, and/or silence genes encoding proteins is described, e.g., in Nicolaou et al., “Molecular diagnosis of peanut and legume allergy,” Curr. Opin. Allergy Clin. Immunol., 11(3):222-8 (2011), and WO 2016205764 A1; both of which are incorporated herein by reference in their entirety.

Gene Drives

Gene drive is the phenomenon in which the inheritance of a particular gene or set of genes is favorably biased. The CRISPR systems described herein can be used to build gene drives. For example, the CRISPR systems can be designed to target and disrupt a particular allele of a gene, causing the cell to copy the second allele to fix the sequence. Because of the copying, the first allele will be converted to the second allele, increasing the chance of the second allele being transmitted to the offspring. A detailed method regarding how to use the CRISPR systems described herein to build gene drives is described, e.g., in Hammond et al., “A CRISPR-Cas9 gene drive system targeting female reproduction in the malaria mosquito vector Anopheles gambiae,” Nat. Biotechnol., 2016 January; 34(1):78-83, which is incorporated herein by reference in its entirety.

Pooled-Screening

As described herein, pooled CRISPR screening is a powerful tool for identifying genes involved in biological mechanisms such as cell proliferation, drug resistance, and viral infection. Cells are transduced in bulk with a library of guide RNA (gRNA)-encoding vectors described herein, and the distribution of gRNAs is measured before and after applying a selective challenge. Pooled CRISPR screens work well for mechanisms that affect cell survival and proliferation, and they can be extended to measure the activity of individual genes (e.g., by using engineered reporter cell lines). Arrayed CRISPR screens, in which only one gene is targeted at a time, make it possible to use RNA-seq as the readout. In some embodiments, the CRISPR systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods., 2017 March; 14(3):297-301, which is incorporated herein by reference in its entirety.

Saturation Mutagenesis (“Bashing”)

The CRISPR systems described herein can be used for in situ saturating mutagenesis. In some embodiments, a pooled guide RNA library can be used to perform in situ saturating mutagenesis for particular genes or regulatory elements. Such methods can reveal critical minimal features and discrete vulnerabilities of these genes or regulatory elements (e.g., enhancers). These methods are described, e.g., in Canver et al., “BCL11A enhancer dissection by Cas9-mediated in situ saturating mutagenesis,” Nature, 2015 Nov. 12; 527(7577):192-7, which is incorporated herein by reference in its entirety.

Therapeutic Applications

The CRISPR systems described herein can have various therapeutic applications. In some embodiments, the new CRISPR systems can be used to treat various diseases and disorders, e.g., genetic disorders (e.g., monogenetic diseases), diseases that can be treated by nuclease activity (e.g., Pcsk9 targeting, Duchenne Muscular Dystrophy (DMD), BCL11a targeting).

In some embodiments, the methods described here are used to treat a subject, e.g., a mammal, such as a human patient. The mammalian subject can also be a domesticated mammal, such as a dog, cat, horse, monkey, rabbit, rat, mouse, cow, goat, or sheep.

In some of the therapeutic methods described herein, the condition or disease is selected from the group consisting of Cystic Fibrosis, Duchenne Muscular Dystrophy, Becker Muscular Dystrophy, Alpha-1-antitrypsin Deficiency, Pompe Disease, Myotonic Dystrophy, Huntington Disease, Fragile X Syndrome, Friedreich's ataxia, Amyotrophic Lateral Sclerosis, Frontotemporal Dementia, Hereditary Chronic Kidney Disease, Hyperlipidemia, Hypercholesterolemia, Leber Congenital Amaurosis, Sickle Cell Disease, and Beta Thalassemia. In some embodiments of the methods described herein (and compositions for use in such methods), the condition or disease is a cancer or an infectious disease.

In some embodiments, the condition or disease is cancer, and wherein the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In another aspect, the disclosure provides the use of a system described herein in a method selected from the group consisting of RNA sequence specific interference; RNA sequence-specific gene regulation; screening of RNA, RNA products, lncRNA, non-coding RNA, nuclear RNA, or mRNA; mutagenesis; inhibition of RNA splicing; fluorescence in situ hybridization; breeding; induction of cell dormancy; induction of cell cycle arrest; reduction of cell growth and/or cell proliferation; induction of cell anergy; induction of cell apoptosis; induction of cell necrosis; induction of cell death; or induction of programmed cell death.

The methods can include the condition or disease being infectious, and wherein the infectious agent is selected from the group consisting of human immunodeficiency virus (HIV), herpes simplex virus-1 (HSV1), and herpes simplex virus-2 (HSV2).

In some embodiments, the CRISPR systems described herein can be used to edit a target nucleic acid to modify the target nucleic acid (e.g., by inserting, deleting, or mutating one or more amino acid residues). For example, in some embodiments the CRISPR systems described herein comprise an exogenous donor template nucleic acid (e.g., a DNA molecule or an RNA molecule), which comprises a desirable nucleic acid sequence. Upon resolution of a cleavage event induced with the CRISPR system described herein, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the CRISPR systems described herein may be used to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA). Methods of designing exogenous donor template nucleic acids are described, for example, in PCT Publication No. WO 2016094874 A1, the entire contents of which are expressly incorporated herein by reference.

In one aspect, the CRISPR systems described herein can be used for treating a disease caused by overexpression of RNAs, toxic RNAs and/or mutated RNAs (e.g., splicing defects or truncations). For example, expression of the toxic RNAs may be associated with the formation of nuclear inclusions and late-onset degenerative changes in brain, heart, or skeletal muscle. In some embodiments, the disorder is myotonic dystrophy. In myotonic dystrophy, the main pathogenic effect of the toxic RNAs is to sequester binding proteins and compromise the regulation of alternative splicing (see, e.g., Osborne et al., “RNA-dominant diseases,” Hum. Mol. Genet., 2009 Apr. 15; 18(8):1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR systems as described herein can target overexpressed RNA or toxic RNA, e.g., the DMPK gene or any of the mis-regulated alternative splicing in DM1 skeletal muscle, heart, or brain.

The CRISPR systems described herein can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR systems described herein is summarized in Cooper et al., “RNA and disease,” Cell, 136.4 (2009): 777-793, and WO 2016205764 A1, both of which are incorporated herein by reference in their entirety. Those of skill in this field will understand how to use the new CRISPR systems to treat these diseases.

The CRISPR systems described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR systems described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis.

The CRISPR systems described herein can further be used for antiviral activity, in particular against RNA viruses. The effector proteins can target the viral RNAs using suitable guide RNAs selected to target viral RNA sequences.

Furthermore, in vitro RNA sensing assays can be used to detect specific RNA substrates. The RNA targeting effector proteins can be used for RNA-based sensing in living cells. Examples of applications are diagnostics by sensing of, for examples, disease-specific RNAs.

A detailed description of therapeutic applications of the CRISPR systems described herein can be found, e.g., in U.S. Pat. No. 8,795,965, EP 3009511, WO 2016205764, and WO 2017070605; each of which is incorporated herein by reference in its entirety.

Delivery of CRISPR Systems

Through this disclosure and the knowledge in the art, the CRISPR systems described herein, or components thereof, nucleic acid molecules thereof, or nucleic acid molecules encoding or providing components thereof, can be delivered by various delivery systems such as vectors, e.g., plasmids, viral delivery vectors. The new CRISPR enzymes and/or any of the RNAs (e.g., guide RNAs) can be delivered using suitable vectors, e.g., plasmids or viral vectors, such as adeno-associated viruses (AAV), lentiviruses, adenoviruses, and other viral vectors, or combinations thereof. The proteins and one or more guide RNAs can be packaged into one or more vectors, e.g., plasmids or viral vectors.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 A1 and U.S. Pat. No. 8,454,972 B2, both of which are incorporated herein by reference in their entirety.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR enzymes, operably linked to the promoter; (iii) a selectable marker; (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, the delivery is via liposomes or lipofectin formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Further means of introducing one or more components of the new CRISPR systems to the cell is by using cell-penetrating peptides (CPP). In some embodiments, a cell penetrating peptide is linked to the CRISPR enzymes. In some embodiments, the CRISPR enzymes and/or guide RNAs are coupled to one or more CPPs to transport them inside cells effectively (e.g., plant protoplasts). In some embodiments, the CRISPR enzymes and/or guide RNA(s) are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hillbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764 A1; each of which is incorporated herein by reference in its entirety.

Various delivery methods for the CRISPR systems described herein are also described, e.g., in U.S. Pat. No. 8,795,965, EP 3009511, WO 2016205764, and WO 2017070605; each of which is incorporated herein by reference in its entirety.

### EXAMPLES

The invention is further described in the following examples, which do not limit the scope of the invention described in the claims.

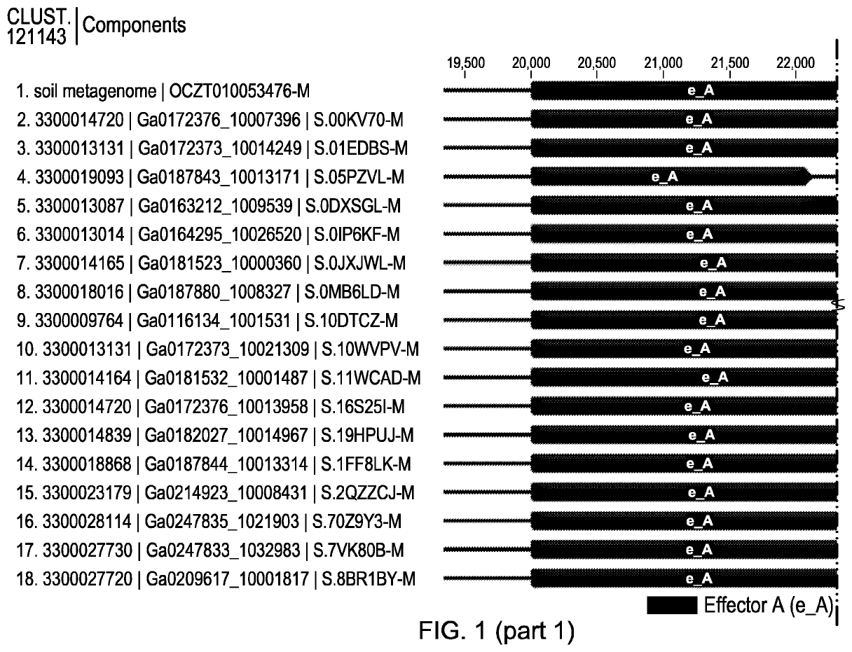

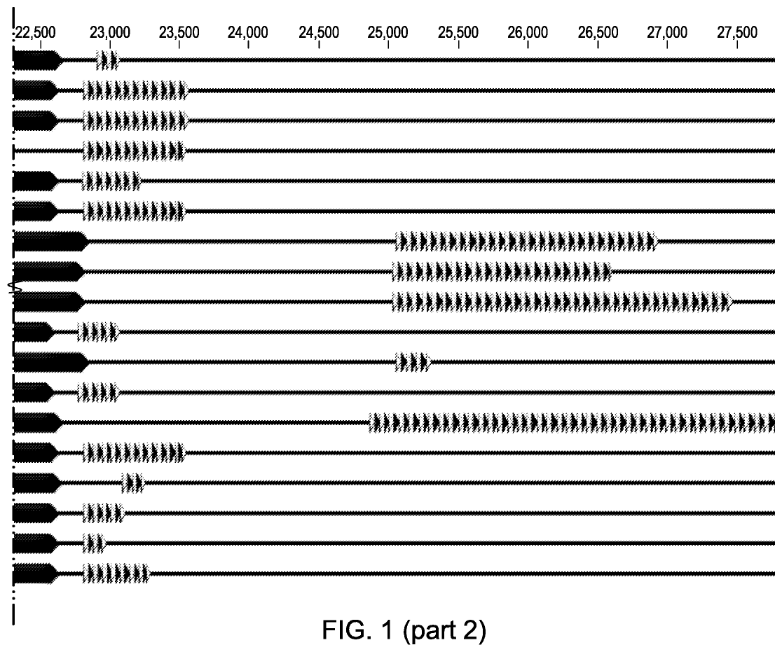

### Example 1—Identification of Minimal Components for CLUST.121143 CRISPR-Cas System (FIGS. 1-4)

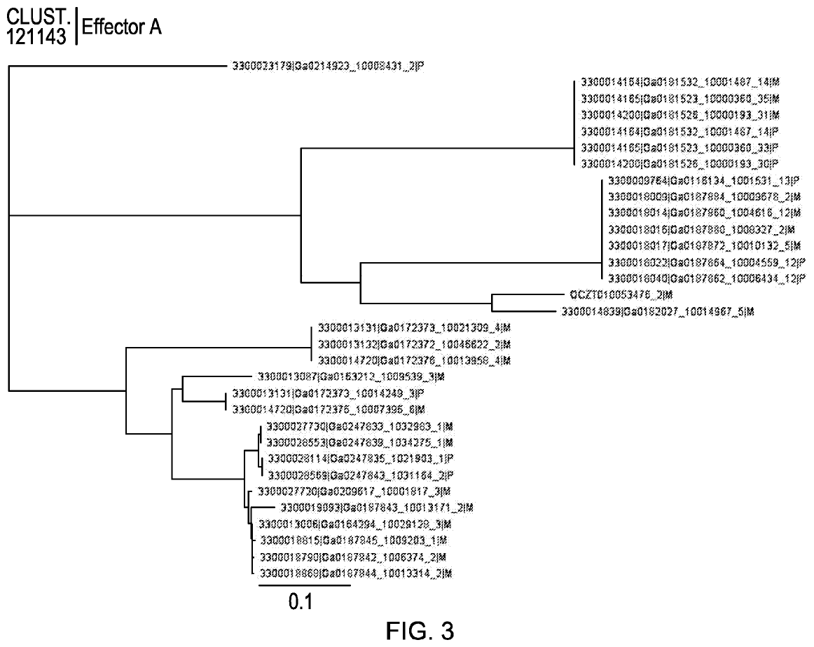

This protein family describes a large single effector associated with CRISPR systems found in uncultured metagenomic sequences collected from freshwater environments (FIG. 3). CLUST.121143 effectors include the examples of proteins detailed in TABLES 1 and 2, below. Examples of direct repeat sequences for these systems are shown in TABLE 3.

Examples of naturally occurring loci containing this effector complex are depicted in FIG. 1, indicating that for CLUST.121143 loci, the effector protein co-occurs with a CRISPR array. No other families of large proteins were identified within a bi-directional 15 kb window that co-occur with the effector protein or a CRISPR array.

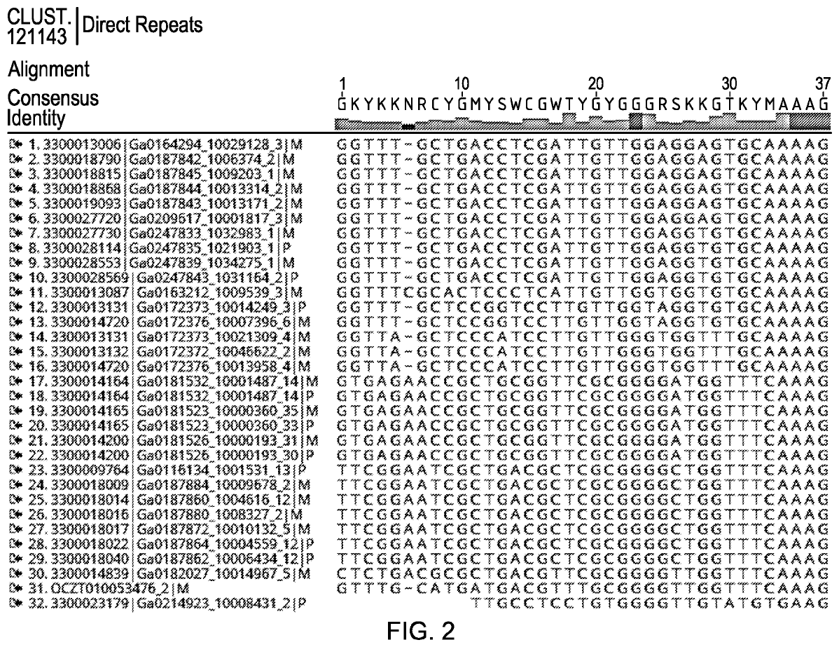

The direct repeat sequence for CLUST.121143 exhibits a conserved motif of 5′-TGCAAAAG-3′ or 5′-TTTCAAAG-4′ proximal to the 3′ end (FIG. 2).

FIG. 3 is a schematic representation of a phylogenetic tree of CLUST.121143 effectors, showing that the family exhibits sequence diversity.

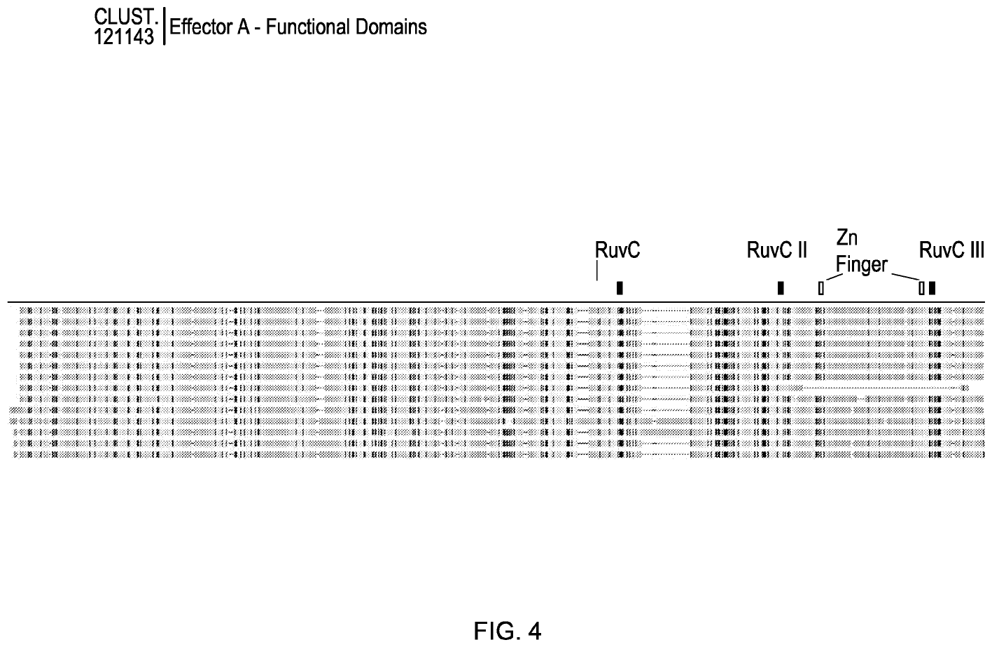

FIG. 4 is a schematic representation of a multiple sequence alignment of CLUST.121143 effector proteins revealing the location of the conserved catalytic residues of the RuvC domain, indicated by the colors Magenta, Blue, and Red.
Optionally, the CLUST.121 143 CRISPR systems described herein include a transactivating RNA (tracrRNA) with a DR homology as detailed in TABLE 4 and a complete tracrRNA contained in the DR homology loci detailed in TABLE 5. Optionally, the system includes a tracrRNA that is a subset of a non-coding sequence listed in TABLE 6.
Optionally, the CLUST.121 143 CRISPR systems described herein include an RNA modulator encoded by a non-coding sequence (or fragment thereof) listed in TABLE 6.

TABLE 1
Representative CLUST.121143 Effector Proteins

| | | # | | | effector |
|---|---|---|---|---|---|
| species | effector accession | spacers | cas1 | cas2 | size |
| aquatic-freshwater | 3300013006\|Ga0164294_10029128_3\|M | 11 | N | N | 878 |
| (3300013006\|Ga0164294_10029128) | | | | | |
| aquatic-freshwater | 3300013087\|Ga0163212_1009539_3\|M | 6 | N | N | 877 |
| (3300013087\|Ga0163212_1009539) | | | | | |
| aquatic-freshwater | 3300013131\|Ga0172373_10014249_3\|P | 11 | N | N | 877 |
| (3300013131\|Ga0172373_10014249) | | | | | |
| aquatic-freshwater | 3300013131\|Ga0172373_10021309_4\|M | 4 | N | N | 867 |
| (3300013131\|Ga0172373_10021309) | | | | | |
| aquatic-freshwater | 3300013132\|Ga0172372_10046622_2\|M | 4 | N | N | 867 |
| (3300013132\|Ga0172372_10046622) | | | | | |
| aquatic-freshwater | 3300014720\|Ga0172376_10007396_6\|M | 11 | N | N | 877 |
| (3300014720\|Ga0172376_10007396) | | | | | |
| aquatic-freshwater | 3300014720\|Ga0172376_10013958_4\|M | 4 | N | N | 867 |
| (3300014720\|Ga0172376_10013958) | | | | | |
| aquatic-freshwater | 3300018790\|Ga0187842_1006374_2\|M | 11 | N | N | 878 |
| (3300018790\|Ga0187842_1006374) | | | | | |
| aquatic-freshwater | 3300018815\|Ga0187845_1009203_1\|M | 11 | N | N | 878 |
| (3300018815\|Ga0187845_1009203) | | | | | |
| aquatic-freshwater | 3300018868\|Ga0187844_10013314_2\|M | 11 | N | N | 878 |
| (3300018868\|Ga0187844_10013314) | | | | | |
| aquatic-freshwater | 3300019093\|Ga0187843_10013171_2\|M | 11 | N | N | 708 |
| (3300019093\|Ga0187843_10013171) | | | | | |
| aquatic-freshwater | 3300023179\|Ga0214923_10008431_2\|P | 2 | N | N | 885 |
| (3300023179\|Ga0214923_10008431) | | | | | |
| aquatic-freshwater | 3300027730\|Ga0247833_1032983_1\|M | 2 | N | N | 878 |
| (3300027730\|Ga0247833_1032983) | | | | | |
| aquatic-freshwater | 3300028114\|Ga0247835_1021903_1\|P | 4 | N | N | 878 |
| (3300028114\|Ga0247835_1021903) | | | | | |
| aquatic-freshwater | 3300028553\|Ga0247839_1034275_1\|M | 2 | N | N | 878 |
| (3300028553\|Ga0247839_1034275) | | | | | |
| aquatic-freshwater | 3300028569\|Ga0247843_1031164_2\|P | 5 | N | N | 878 |
| (3300028569\|Ga0247843_1031164) | | | | | |
| aquatic-freshwater-bog | 3300014164\|Ga0181532_10001487_14\|M | 3 | Y | Y | 951 |
| (3300014164\|Ga0181532_10001487) | | | | | |
| aquatic-freshwater-bog | 3300014164\|Ga0181532_10001487_14\|P | 3 | Y | Y | 1000 |
| (3300014164\|Ga0181532_10001487) | | | | | |
| aquatic-freshwater-bog | 3300014165\|Ga0181523_10000360_35\|M | 26 | Y | Y | 951 |
| (3300014165\|Ga0181523_10000360) | | | | | |
| aquatic-freshwater-bog | 3300014165\|Ga0181523_10000360_33\|P | 26 | Y | Y | 1000 |
| (3300014165\|Ga0181523_10000360) | | | | | |
| aquatic-freshwater-bog | 3300014200\|Ga0181526_10000193_31\|M | 26 | Y | Y | 951 |
| (3300014200\|Ga0181526_10000193) | | | | | |
| aquatic-freshwater-bog | 3300014200\|Ga0181526_10000193_30\|P | 26 | Y | Y | 1000 |
| (3300014200\|Ga0181526_10000193) | | | | | |
| aquatic-freshwater-freshwater and | 3300027720\|Ga0209617_10001817_3\|M | 7 | N | N | 878 |
| sediment | | | | | |
| (3300027720\|Ga0209617_10001817) | | | | | |
| aquatic-freshwater-peatland | 3300009764\|Ga0116134_1001531_13\|P | 34 | Y | Y | 941 |
| (3300009764\|Ga0116134_1001531) | | | | | |
| aquatic-freshwater-peatland | 3300018009\|Ga0187884_10009678_2\|M | 8 | Y | Y | 941 |
| (3300018009\|Ga0187884_10009678) | | | | | |
| aquatic-freshwater-peatland | 3300018014\|Ga0187860_1004616_12\|M | 34 | Y | Y | 941 |
| (3300018014\|Ga0187860_1004616) | | | | | |
| aquatic-freshwater-peatland | 3300018016\|Ga0187880_1008327_2\|M | 22 | Y | Y | 941 |
| (3300018016\|Ga0187880_1008327) | | | | | |
| aquatic-freshwater-peatland | 3300018017\|Ga0187872_10010132_5\|M | 6 | Y | Y | 941 |
| (3300018017\|Ga0187872_10010132) | | | | | |
| aquatic-freshwater-peatland | 3300018022\|Ga0187864_10004559_12\|P | 34 | Y | Y | 941 |
| (3300018022\|Ga0187864_10004559) | | | | | |
| aquatic-freshwater-peatland | 3300018040\|Ga0187862_10006434_12\|P | 34 | Y | Y | 941 |
| (3300018040\|Ga0187862_10006434) | | | | | |
| soil metagenome (OCZT010053476) | OCZT010053476_2\|M | 2 | N | N | 887 |
| terrestrial-soil-fen | 3300014839\|Ga0182027_10014967_5\|M | 49 | Y | Y | 888 |
| (3300014839\|Ga0182027_10014967) | | | | | |

TABLE 2
Amino acid sequences of Representative CLUST.121143 Effector Proteins

>3300013006|Ga0164294_10029128_3|M
[aquatic-freshwater]
MNHKSTVKASVRHLTRHALQKPGEESDPGETTKTLKFRVDAKGHTRTRLDQIFTAQRQFVRDLLNKLEGMWDKDPQRFIRMVES
SASDPFESKTSCYGWMMTKFLTGSDLPEHLSRSAATASVATLSGNMKSFVTRRENVLKDMAVVIERNMENCEAGLKDLCAELKI
DRFKSPPSVDLESINAKTVEKYNEWVAAARVWCNLILAQKHKIPRGDAFFPTRLKAYPGFPGSVIHKEATSFATAIKELRKCLA
DLKQSHQKLFGALSNEEWSLVLERFPAPEGTGGKGMRHQLGKTLAVLGKKHPEWKPRQYIEELVAGLERGADSMAKHLENRQFS
DRPAAIKLINLLNNACVVLMEPLRVKSLYKEYFEQDTPRRNAFGQARGSLAEPTDQADSLQILGFSLMDEVRGICRYDGFLAFA
GDKEKDQWAFFFDPDGPGGMGIADPVKHPKGSTDSGYIGFGTMGGSRKRAQAAEKRLVRERLWIRRDRKPLQLPLHFGTRQGRE
YLWNFDHGLKKSSDPWMLTNGRLLRIYPPNRPQFSDYFLTITLQKDTPPLAISSGEKLVGVDRGEQNPASFAVVDAKGRLLERG
NVGEKYTAKIRQFAEMKSDLQRTAGGYTRKLRAKERNTASALGGLVMRELLDLLVRHPGALVFERLSSGITTRGGRGTLMGNMH
YERILSGVEGKLAEAGCYKAPSAAKYRKGITPFLRFVNPAYTSATCSACGEVFSSSWYKKTAATISKESPGSWSVTLPNGKKLQ
LPCSYTSYMRGRGEKTWNTAERLEELFKDRTWSDLPPSTRKTVEGLVARLTHHRPKRGEFVCPSCGHKDDADLQAALNIARKSI
WVSSLPPRQAKQSEGEDRRSTEEPWRVWYKKQIMEKWS (SEQ ID NO: 1)
>3300013087|Ga0163212_1009539_3|M
[aquatic-freshwater]
MIHKSTVKASVRHLTRHALDKPGEGDPGETTKTLKFRIDAKGHTRTRLDQIFSAQRQFVRDLLNKLEGMWDKDPERFIRMARSS
PSEPFEKKTSCAGWMLTKFLTGSDLPKHLSRQSARATLSPLSRNMKSFITRRKNVLKDMAVVIGRNRDNWDSGLKELCAELKVD
QFKGPPSVDPDKLNAKTVEKYNEWVAAARVWCNLILAQKHKIPRGDAFFPTRLKAYPGFPGSVIHKEAVPFSTAIEELRKCLAD
LKRSQLKLFDGLTAEEWVLVLERFPATEGEGGKGMRHQLGKTLAVLGKKHSDWKPRQLVEELVAGLERGADSLVNHLKDRDFSD
RPAAIKLINLLNNACVVLMEPLRVKNLYREYFEQDTPRRNAFGQARGSLAEPTDQADSLEIEGFSLKKGDAGSCNFDGLLAVTP
KDGKDQWSFFFDPEGPGGMGIAEITKHPRGSTTTEHLSFGSSGGSRKKAQASEKKLVRERLWIRRDRKPLQLPLQFGTRQGREY
LWNFDHGLKKSSDPWLLANGRLLRVYPINRPQFSQYNLTITLQKDTPPLALSAGEMLIGVDRGEVNPASFAVVDAKGKLLKCGN
VGEKYTAKIRQLAEMKSDLQRTAGGYTRKLRAKERNTASALGGLVIRELLDLLVRHPGDLVFERLTSGITTRGGRGTLMGNMHY
ERIMSGVEGKLAEAGCYKVPRATKFRSKDTRFVRFVHPAYTSATCSACGDVFSSGWYKKTAATIAKESPGSWSVTLPSGKKIQL
PSSYTYYMRGRGEKTWNTAERLEELFKGRKWSDLPPSTRKTVEGLVARLIHHRPKRGEFVCPSCGHKDDADLQAALNIARKSIW
VRSLPPRQAKQSEGEDRRSTEEPWREWYKKQIREKWA (SEQ ID NO: 2)
>3300013131|Ga0172373_10014249_3|P
[aquatic-freshwater]
MIHKSTVKASVRHLTRHALDKPGEGDPGETTKTLKFRIDAKDHTRTRLDQIFNAQRQFVRDLLNKLEGMWDKDPERFIRMVRSS
ASEPFEKKTSCAGWMLTKFLTGSDLPEHLSRSSATASVATLSGNMKSFITRRENVLKDMAVVIERNRENWDSGLKELCAELKID
QFKAPPSVDPDKLNAKTVEKYNEWVAAARVWCNLILAQKHKIPRGDAFFPTRLKAYPGFPGSVIHKEGIAFDTAIKELRKCLAD
LKRSHLKLFDGLTVEEWGLVLERFPAPEGEGGKGMRHQLGKTLAVLGKKHSDWKPRQLAEELVTGLERGADSLANHLKDRDFSD
RPAAIKLINLLNNACVVLMEPLRVKNLYREYFEQDTPRRNAFGQARGSLAEPTDQADSLQIMGFAIRKTPEGHSGFDGFLAVSQ
KEGKDIWSFFFDPDGPGGMGLCDASKHPRGSTTTDQIGFGTSGGSRKRAQAAEKRLVRERLWIRRDRKPLQLPLQFGTRQGREY
LWNFDHGLKKSSDPWMLTNGRLLRIYPPNRPQFSQYYLTIALQKDTPPPALTAGEMLIGVDRGEQNPASYAVTDGKGKLLAKGN
VGESYSAKIRQFAEMKSEMQRTAGGYTRKLRAKERNTASALGGLVIRELLDLLVRHPGALVFERLTSGITTRGGRGTLMGNMHY
ERILSGVEGKLAEAGCYKAPSAAKYRKGITPFLRFVNPAYTSATCSACGDVFSSGWYRKAAATISRESSGSWSVTLPSGKKVQL
PSSYTSYMRGRGEKTWNTAERLEELFKGRKWSDLPPSTRKTVDGLVARLIHHRPKRGEFVCPSCARKDDADLQAALNIARKSIW
VSSLPHRQAKQSEGEDRRSTEEPWREWYKKRIREKWA (SEQ ID NO: 3)
>3300013131|Ga0172373_10021309_4|M
[aquatic-freshwater]
MIHKSTVKASVRHLTRHALDKPGEGDPGKPTTKTLKFRIDAKGHTRTRLDQIFTAQNHFRRDLYIKLEGMWDKDPESFISMVNS
SPSKPFQKKTSCSGWIETKFVTGVALPEHLSRETAAATAVAVGNSLTATFERRKNVISQLAKNIEENRSVWRGNLKELCSELKI
TPFKAPPSIHPAKINAKTVEKYNEWVAAARIWCNLILVQKHRVPRNSACLPVRLKHYPGFPGSVIFKKGASLATAIKEFRKCLA
GLKRSQLKLFDGLTAEEWGLVIDRFPAPEGEGGKGMRHQLGKTLAVLGKKHPDWKPRQLVEELVAGLERGADSLVNHLKDRDFS
DRPAAIKLINLLNNACVVLMEPLRVKNLYRELFEVGTPRRDAFGQARGALAEPTDQSEGLEIRGFSIRTNRPPIGHSSFNGFLA
VRQTEGKDEWSFFFDPDGPGGMGLSGLPPAGSIPTDYIGFGSTGGSRKKAQASEKKLVRERLWIRRDRNPLQLPLQFGKRQGRE
YLWNFDHGLKKSSDPWMLANGRLLRVYPPNQPQFSDYFLTITLQKDTPPLALSAGEMLIGVDQGEVNPASYAVTDAKGKLLTRG
NVGESYSAKIRQFAEMKSAEQRTAGGYTRKLRSKERNTASACGGLVIRELLDLLVRYPGALVFERLSSGIATRGGRGTLMSNMH
YQRITGGIEKKLSEAGCHRITDFKSNFSPFLRMVQPHYTSATCSNCGEVFDSDWYEKLTATIRKSVSGSWVVTLPDGKELWLQG
REEIPWNPTDRLEEIFKGRTFPDLTSRSQKIVQGIVGRLVAFRPKRGEFVCPCCGHTDDADLQAALNIARKSIWLSTLPKRSKK
ETLAEARGSTKPAWKKWYQNRISENWC (SEQ ID NO: 4)
>3300013132|Ga0172372_10046622_2|M
[aquatic-freshwater]
MIHKSTVKASVRHLTRHALDKPGEGDPGKPTTKTLKFRIDAKGHTRTRLDQIFTAQNHFRRDLYIKLEGMWDKDPESFISMVNS
SPSKPFQKKTSCSGWIETKFVTGVALPEHLSRETAAATAVAVGNSLTATFERRKNVISQLAKNIEENRSVWRGNLKELCSELKI
TPFKAPPSIHPAKINAKTVEKYNEWVAAARIWCNLILVQKHRVPRNSACLPVRLKHYPGFPGSVIFKKGASLATAIKEFRKCLA
GLKRSQLKLFDGLTAEEWGLVIDRFPAPEGEGGKGMRHQLGKTLAVLGKKHPDWKPRQLVEELVAGLERGADSLVNHLKDRDFS
DRPAAIKLINLLNNACVVLMEPLRVKNLYRELFEVGTPRRDAFGQARGALAEPTDQSEGLEIRGFSIRTNRPPIGHSSFNGFLA
VRQTEGKDEWSFFFDPDGPGGMGLSGLPPAGSIPTDYIGFGSTGGSRKKAQASEKKLVRERLWIRRDRNPLQLPLQFGKRQGRE
YLWNFDHGLKKSSDPWMLANGRLLRVYPPNQPQFSDYFLTITLQKDTPPLALSAGEMLIGVDQGEVNPASYAVTDAKGKLLTRG
NVGESYSAKIRQFAEMKSAEQRTAGGYTRKLRSKERNTASACGGLVIRELLDLLVRYPGALVFERLSSGIATRGGRGTLMSNMH
YQRITGGIEKKLSEAGCHRITDFKSNFSPFLRMVQPHYTSATCSNCGEVFDSDWYEKLTATIRKSVSGSWVVTLPDGKELWLQG
REEIPWNPTDRLEEIFKGRTFPDLTSRSQKIVQGIVGRLVAFRPKRGEFVCPCCGHTDDADLQAALNIARKSIWLSTLPKRSKK
ETLAEARGSTKPAWKKWYQNRISENWC (SEQ ID NO: 4)
>3300014720|Ga0172376_10007396_6|M
[aquatic-freshwater]
MIHKSTVKASVRHLTRHALDKPGEGDPGETTKTLKFRIDAKDHTRTRLDQIFNAQRQFVRDLLNKLEGMWDKDPERFIRMVRSS
ASEPFEKKTSCAGWMLTKFLTGSDLPEHLSRSSATASVATLSGNMKSFITRRENVLKDMAVVIERNRENWDSGLKELCAELKID
QFKAPPSVDPDKLNAKTVEKYNEWVAAARVWCNLILAQKHKIPRGDAFFPTRLKAYPGFPGSVIHKEGIAFDTAIKELRKCLAD
LKRSHLKLFDGLTVEEWGLVLERFPAPEGEGGKGMRHQLGKTLAVLGKKHSDWKPRQLAEELVTGLERGADSLANHLKDRDFSD
RPAAIKLINLLNNACVVLMEPLRVKNLYREYFEQDTPRRNAFGQARGSLAEPTDQADSLQIMGFAIRKTPEGHSGFDGFLAVSQ
KEGKDIWSFFFDPDGPGGMGLCDASKHPRGSTTTDQIGFGTSGGSRKRAQAAEKRLVRERLWIRRDRKPLQLPLQFGTRQGREY
LWNFDHGLKKSSDPWMLTNGRLLRIYPPNRPQFSQYYLTIALQKDTPPPALTAGEMLIGVDRGEQNPASYAVTDGKGKLLAKGN
VGESYSAKIRQFAEMKSEMQRTAGGYTRKLRAKERNTASALGGLVIRELLDLLVRHPGALVFERLTSGITTRGGRGTLMGNMHY
ERILSGVEGKLAEAGCYKAPSAAKYRKGITPFLRFVNPAYTSATCSACGDVFSSGWYRKAAATISRESSGSWSVTLPSGKKVQL
PSSYTSYMRGRGEKTWNTAERLEELFKGRKWSDLPPSTRKTVDGLVARLIHHRPKRGEFVCPSCARKDDADLQAALNIARKSIW
VSSLPHRQAKQSEGEDRRSTEEPWREWYKKRIREKWA (SEQ ID NO: 3)
>3300014720|Ga0172376_10013958_4|M
[aquatic-freshwater]
MIHKSTVKASVRHLTRHALDKPGEGDPGKPTTKTLKFRIDAKGHTRTRLDQIFTAQNHFRRDLYIKLEGMWDKDPESFISMVNS
SPSKPFQKKTSCSGWIETKFVTGVALPEHLSRETAAATAVAVGNSLTATFERRKNVISQLAKNIEENRSVWRGNLKELCSELKI
TPFKAPPSIHPAKINAKTVEKYNEWVAAARIWCNLILVQKHRVPRNSACLPVRLKHYPGFPGSVIFKKGASLATAIKEFRKCLA
GLKRSQLKLFDGLTAEEWGLVIDRFPAPEGEGGKGMRHQLGKTLAVLGKKHPDWKPRQLVEELVAGLERGADSLVNHLKDRDFS
DRPAAIKLINLLNNACVVLMEPLRVKNLYRELFEVGTPRRDAFGQARGALAEPTDQSEGLEIRGFSIRTNRPPIGHSSFNGFLA
VRQTEGKDEWSFFFDPDGPGGMGLSGLPPAGSIPTDYIGFGSTGGSRKKAQASEKKLVRERLWIRRDRNPLQLPLQFGKRQGRE
YLWNFDHGLKKSSDPWMLANGRLLRVYPPNQPQFSDYFLTITLQKDTPPLALSAGEMLIGVDQGEVNPASYAVTDAKGKLLTRG
NVGESYSAKIRQFAEMKSAEQRTAGGYTRKLRSKERNTASACGGLVIRELLDLLVRYPGALVFERLSSGIATRGGRGTLMSNMH
YQRITGGIEKKLSEAGCHRITDFKSNFSPFLRMVQPHYTSATCSNCGEVFDSDWYEKLTATIRKSVSGSWVVTLPDGKELWLQG
REEIPWNPTDRLEEIFKGRTFPDLTSRSQKIVQGIVGRLVAFRPKRGEFVCPCCGHTDDADLQAALNIARKSIWLSTLPKRSKK
ETLAEARGSTKPAWKKWYQNRISENWC (SEQ ID NO: 4)
>3300018790|Ga0187842_1006374_2|M
[aquatic-freshwater]
MNHKSTVKASVRHLTRHALQKPGEESDPGETTKTLKFRVDAKGHTRTRLDQIFTAQRQFVRDLLNKLEGMWDKDPQRFIRMVES
SASDPFESKTSCYGWMMTKFLTGSDLPEHLSRSAATASVATLSGNMKSFVTRRENVLKDMAVVIERNMENCEAGLKDLCAELKI
DRFKSPPSVDLESINAKTVEKYNEWVAAARVWCNLILAQKHKIPRGDAFFPTRLKAYPGFPGSVIHKEATSFATAIKELRKCLA
DLKQSHQKLFGALSNEEWSLGLERFPAPEGTGGKGMRHQLGKTLAVLGKKHPEWKPRQYIEELVAGLERGADSMAKHLENRQFS
DRPAAIKLINLLNNACVVLMEPLRVKSLYKEYFEQDTPRRNAFGQARGSLAEPTDQADSLQILGFSLMDEVRGICRYDGFLAFA
GDKEKDQWAFFFDPDGPGGMGIADPVKHPKGSTDSGYIGFGTMGGSRKRAQAAEKRLVRERLWIRRDRKPLQLPLHFGTRQGRE
YLWNFDHGLKKSSDPWMLTNGRLLRIYPPNRPQFSDYFLTITLQKDTPPLAISSGEKLVGVDRGEQNPASFAVVDAKGRLLERG
NVGEKYTAKIRQFAEMKSDLQRTAGGYTRKLRAKERNTASALGGLVMRELLDLLVRHPGALVFERLSSGITTRGGRGTLMGNMH
YERILSGVEGKLAEAGCYKAPSAAKYRKGITPFLRFVNPAYTSATCSACGEVFSSSWYKKTAATISKESPGSWSVTLPNGKKLQ
LPCSYTSYMRGRGEKTWNTAERLEELFKDRTWSDLPPSTRKTVEGLVARLTHHRPKRGEFVCPSCGHKDDADLQAALNIARKSI
WVSSLPPRQAKQSEGEDRRSTEEPWRVWYKKQIMEKWS (SEQ ID NO: 5)
>3300018815|Ga0187845_1009203_1|M
[aquatic-freshwater]
MNHKSTVKASVRHLTRHALQKPGEESDPGETTKTLKFRVDAKGHTRTRLDQIFTAQRQFVRDLLNKLEGMWDKDPQRFIRMVES
SASDPFESKTSCYGWMMTKFLTGSDLPEHLSRSAATASVATLSGNMKSFVTRRENVLKDMAVVIERNMENCEAGLKDLCAELKI
DRFKSPPSVDLESINAKTVEKYNEWVAAARVWCNLILAQKHKIPRGDAFFPTRLKAYPGFPGSVIHKEATSFATAIKELRKCLA
DLKQSHQKLFGALSNEEWGLVLERFPAPEGTGGKGMRHQLGKTLAVLGKKHPEWKPRQYIEELVAGLERGADSMAKHLENRQFS
DRPAAIKLINLLNNACVVLMEPLRVKSLYKEYFEQDTPRRNAFGQARGSLAEPTDQADSLQILGFSLMDEVRGICRYDGFLAFA
GDKEKDQWAFFFDPDGPGGMGIADPVKHPKGSTDSGYIGFGTMGGSRKRAQAAEKRLVRERLWIRRDRKPLQLPLHFGTRQGRE
YLWNFDHGLKKSSDPWMLTNGRLPRIYPPNRPQFSDYFLTITLQKDTPPLAISSGEKLVGVDRGEQNPASFAVVDAKGRLLERG
NVGEKYTAKIRQFAEMKSDLQRTAGGYTRKLRAKERNTASALGGLVMRELLDLLVRHPGALVFERLSSGITTRGGRGTLMGNMH
YERILSGVEGKLAEAGCYKAPSAAKYRKGITPFLRFVNPAYTSATCSACGEVFSSSWYKKTAATISKESPGSWSVTLPNGKKLQ
LPCSYTSYMRGRGEKTWNTAERLEELFKDRTWSDLPPSTRKTVEGLVARLTHHRPKRGEFVCPSCGHKDDADLQAALNIARKSI
WVSSLPPRQAKQSEGEDRRSTEEPWRVWYKKQIMEKWS (SEQ ID NO: 6)
>3300018868|Ga0187844_10013314_2|M
[aquatic-freshwater]
MNHKSTVKASVRHLTRHALQKPGEESDPGETTKTLKFRVDAKGHTRTRLDQIFTAQRQFVRDLLNKLEGMWDKDPQRFIRMVES
SASDPFESKTSCYGWMMTKFLTGSDLPEHLSRSAATASVATLSGNMKSFVTRRENVLKDMAVVIERNMENCEAGLKDLCAESKI
DRFKSPPSVDLESINAKTVEKYNEWVAAARVWCNLILAQKHKIPRGDAFFPTRLKAYPGFPGSVIHKEATSFATAIKELRKCLA
DLKQSHQKLFGALSNEEWSLVLERFPAPEGTGGKGMRHQLGKTLAVLGKKHPEWKPRQYIEELVAGLERGADSMAKHLENRQFS
DRPAAIKLINLLNNACVVLMEPLRVKSLYKEYFEQDTPRRNAFGQARGSLAEPTDQADSLQILGFSLMDEVRGICRYDGFLAFA
GDKEKDQWAFFFDPDGPGGMGIADPVKHPKGSTDSGYIGFGTMGGSRKRAQAAEKRLVRERLWIRRDRKPLQLPLHFGTRQGRE
YLWNFDHGLKKSSDPWMLTNGRLLRIYPPNRPQFSDYFLTITLQKDTPPLAISSGEKLVGVDRGEQNPASFAVVDAKGRLLERG
NVGEKYTAKIRQFAEMKSDLQRTAGGYTRKLRAKERNTASALGGLVMRELLDLLVRHPGALVFERLSSGITTRGGRGTLMGNMH
YERILSGVEGKLAEAGCYKAPSAAKYRKGITPFLRFVNPAYTSATCSACGEVFSSSWYKKTAATISKESPGSWSVTLPNGKKLQ
LPCSYTSYMRGRGEKTWNTAERLEELFKDRTWSDLPPSTRKTVEGLVARLTHHRPKRGEFVCPSCGHKDDADLQAALNIARKSI
WVSSLPPRQAKQSEGEDRRSTEEPWRVWYKKQIMEKWS (SEQ ID NO: 7)
>3300019093|Ga0187843_10013171_2|M
[aquatic-freshwater]
MNHKSTVKASVRHLTRHALQKPGEESDPGETTKTLKFRVDAKGHTRTRLDQIFTAQRQFVRDLLNKLEGMWDKDPQRFIRMVES
SASDPFESKTSCYGWMMTKFLTGSDLPEHLSRSAATASVATLSGNMKSFVTRRENVLKDMAVVIERNMENCEAGLKDLCAELKI
DRFKSPPSVDLESINAKTVEKYNEWVAAARVWCNLILAQKHKIPRGDAFFPTRLKAYPGFPGSVIHKEATYFATAIKELRKCLA
DLKQSHQKLFGALSNEEWSLVLERFPAPEGTGGKGMRHQLGKTLAVLGKKHPEWKPRQYIEELVAGLERGADSMAKHLENRQFS
DRPAAIKLINLLNNACVVLMEPLRVKSLYKEYFEQDTPRRNAFGQARGSLAEPTDQADSLQILGFSLMDEVRGICRYDGFLAFA
GDKEKDQWAFFFDPDGPGGMGIADPVKHPKGSTDSGYIGFGTMGGSRKRAQAAEKRLVRERLWIRRDRKPLQLPLHFGTRQGRE
YLWNFDHGLKKSSDPWMLTNGRLLRIYPHNRPQFSDYFLTITLQKDTPPLAISSGEKLVGVDRGEQNPASFAVVDAKGRLLERG
NVGEKYTAKIRQFAEMKSDLQRTAGGYTRKLRAKERNTASALGGLVMRELLDLLVRHPGALVFERLSSGITTRGGRGTLMGNMH
YERILSGLRASWPRQVATRHPVPPSTAKESRRSCDL (SEQ ID NO: 8)
>3300023179|Ga0214923_10008431_2|P
[aquatic-freshwater]
MLNPAEMQMPSNSIKSIKASVRELYNHALNKQGDGEPGETTKTLKFKIDPSPENQIQLTKIFDAQRAFLRDVLQKLEGMWDKHP
EKIEEMANCSGSKPFEKKTSCYGWLLTKFLTGSDLPENLSRSAATASIGTLAGNLKSFLTRRKNVLKEMANTVSANAKLWDEDL
PIKCKEMDLDLFKAPPKIKVESLNAKTVDEYNEWVASARVWCNLILVQKNKVPRSDACFPKRLKAYPGFPFSVKHKETAKITDA
VKSLQDCLCELKKAHLKAFDSLTEVEWQSVLERFPSPKEVDGKGTRRLIGKTLAALHKKNPNWKPVDIVKELIAGMERSIQSLV
KHLEVREFTDRKAAIKLMNHMNHACVFLLEPLRVRNDYKGLYEKDTPRRNAFGEARGSLSEPTDEADSLQIIGFSLNERGGTRY
DGILAYSGEPGSQKWAFFFDPLGPEGMGLADEGKNPARTTLLPWVGFGTTGGARKKAQAKAKALVRGKIWVSPKRKPMALPLLF
GARQGREYLWNFDRGLKSSSTPWMLTNGRILRIFPYQKPELAEFYLTLTLQKDNPPLALSLGEHLIGVDRGEENPASYALVDAK
GRLVERGHIGQEYSQRIKTFAKEKAELQRSLGGYTRRLRAKERNIASALGGQVIRELMNLLVAKKGALVFENLSSGIVTRGGRG
TLMGNMHYERILSGIEQKLAEAGCYEVPNAKKYRKGVSPFVRFVNPSYTSSTCSKCGEVYSRAWYEKIAPSLERKGAGIWAVKL
GEKHFTLPLSYSYFLRGRGEQHKKTDERLTEILKGKDWAKLSATSKKEIVGLLQSKLVAYRPKRGKFNCPSCEHEDDADLQAAL
NIARKTIWVSTLPPKKAKSGDQDFRGVGQELWAKWYKDRVHKNWA (SEQ ID NO: 9)
>3300027730|Ga0247833_1032983_1|M
[aquatic-freshwater]
MNHKNTVKASVRELTRHALQKPGEESEPGETTKTLKFRVDAKGHTRTRLDQIFTAQRQFVRDLLTKLEGMWDKDPQRFIRMVES
SASDPFESKTSCYGWMMTKFLTGSDLPEHLSRSAATASVATLSGNMKSFVTRRENVLKDMAVVIERNMENCEAGLKDLCAELKI
DRFKSPPSVDLESINAKTVEKYNEWVAAARVWCNLILAQKHKIPRGDAFFPTRLKAYPGFPGSVIHKEATSFATAIKELRKCLA
DLKQSHQKLFDALSTEEWSLVLEKFPAPEGTGGKGMRHQLGKTLAVLGKKHPEWKPRQYIEELVAGLERGADSMAKHLKNRQFS
DRPAAIKLINLLNNACVVLMEPLRVKSLYKEYFEQDTPRRNAFGQARGSLAEPTDQADSLQILGFSLMDEVRGVCRYDGFLAFT
GDKGKDQWAFFFDPDGPGGMGIADPVKHPKGSSDSGYIGFGTMGGSRKRAQAAEKRLVRERLWIRRDRKPLQLPLHFGTRQGRE
YLWNFDHGLKKSSDPWMLTNGRLLRVYPPNRPQLSDYFLTITLQKDTPPLAISSGDKLVGVDRGEQNPASFAVVDDKGRLLERG
NVGEKYTAKIRQFAEMKSDLQRTAGGYTRKLRAKERNTASALGGLVMRELLDLLVRHPGALVFERLSSGITTRGGRGTLMGNMH
YERILSGVEGKLAEAGCYKAPSAAKYRKGITPFLRFVNPAYTSATCSACGEVFSSGWYKKTAATISKESLGSWSVTLPNGKKLQ
LPSSYTSYMRGRGEKTWNTAERLEELFQDRTWSDLPPSTRKTVEGLVARLTHHRPKRGEFVCSSCGHKDDADLQAALNIARKSI
WVSSLPPRQAKQSEGEDRRSTEEPWRVWYKKQIMGKWS (SEQ ID NO: 10)
>3300028114|Ga0247835_1021903_1|P
[aquatic-freshwater]
MNHKNTVKASVRELTRHALQKPGEESEPGETTKTLKFRVDAKGHTRTRLDQIFTAQRQFVRDLLTKLEGMWDKDPQRFIRMVES
SASDPFESKTSCYGWMMTKFLTGSDLPEHLSRSAATASVATLSGNMKSFVTRRENVLKDMAVVIERNMESFEAGLKDLCAELKI
DRFKSPPSVDLESINAKTVEKYNEWVAAARVWCNLILAQKHKIPRGDAFFPTRLKAYPGFPGSVIHKEATSFATAIKELRKCLA
DLKQSHQKLFDALSTEEWSLVLEKFPAPEGTGGKGMRHQLGKTLAVLGKKHPEWKPRQYIEELVAGLERGADSMAKHLKNRQFS
DRPAAIKLINLLNNACVVLMEPLRVKSLYKEYFEQDTPRRNAFGQARGSLAEPTDQADSLQILGFSLMDEVRGVCRYDGFLAFT
GDKGKDQWAFFFDPDGPGGMGIADPVKHPKGSSDSGYIGFGTMGGSRKRAQAAEKRLVRERLWIRRDRKPLQLPLHFGTRQGRE
YLWNFDHGLKKSSDPWMLTNGRLLRVYPPNRPQLSDYFLTITLQKDTPPLAISSGDKLVGVDRGEQNPASFAVVDDKGRLLERG
NVGEKYTAKIRQFAEMKSDLQRTAGGYTRKLRAKERNTASALGGLVMRELLDLLVRHPGALVFERLSSGITTRGGRGTLMGNMH
YERILSGVEGKLAEAGCYKAPSAAKYRKGITPFLRFVNPAYTSATCSACGEVFSSGWYKKTAATISKESLGSWSVTLPNGKKLQ
LPSSYTSYMRGRGEKTWNTAERLEELFQDRTWSDLPPSTRKTVEGLVARLTHHRPKRGEFVCSSCGHKDDADLQAALNIARKSI
WVSSLPPRQAKQSEGEDRRSTEEPWRVWYKKQIMGKWS (SEQ ID NO: 11)
>3300028553|Ga0247839_1034275_1|M
[aquatic-freshwater]
MNHKNTVKASVRELTRHALQKPGEESEPGETTKTLKFRVDAKGHTRTRLDQIFTAQRQFVRDLLTKLEGMWDKDPQRFIRMVES
SASDPFESKTSCYGWMMTKFLTGSDLPEHLSRSAATASVATLSGNMKSFVTRRENVLKDMAVVIERNMENCEAGLKDLCAELKI
DRFKSPPSVDLESINAKTVEKYNEWVAAARVWCNLILAQKHKIPRGDAFFPTRLKAYPGFPGSVIHKEATSFATAIKELRKCLA
DLKQSHQKLFDALSTEEWSLVLEKFPAPEGTGGKGMRHQLGKTLAVLGKKHPEWKPRQYIEELVAGLERGADSMAKHLKNRQFS
DRPAAIKLINLLNNACVVLMEPLRVKSLYKEYFEQDTPRRNAFGQARGSLAEPTDQADSLQILGFSLMDEVRGVCRYDGFLAFT
GDKGKDQWAFFFDPDGPGGMGIADPVKHPKGSSDSGYIGFGTMGGSRKRAQAAEKRLVRERLWIRRDRKPLQLPLHFGTRQGRE
YLWNFDHGLKKSSDPWMLTNGRLLRVYPPNRPQLSDYFLTITLQKDTPPLAISSGDKLVGVDRGEQNPASFAVVDDKGRLLERG
NVGEKYTAKIRQFAEMKSDLQRTAGGYTRKLRAKERNTASALGGLVMRELLDLLVRHPGALVFERLSSGITTRGGRGTLMGNMH
YERILSGVEGKLAEAGCYKAPSAAKYRKGITPFLRFVNPAYTSATCSACGEVFSSGWYKKTAATISKESLGSWSVTLPNGKKLQ
LPSSYTSYMRGRGEKTWNTAERLEELFQDRTWSDLPPSTRKTVEGLVARLTHHRPKRGEFVCSSCGHKDDADLQAALNIARKSI
WVSSLPPRQAKQSEGEDRRSTEEPWRVWYKKQIMGKWS (SEQ ID NO: 10)
>3300028569|Ga0247843_1031164_2|P
[aquatic-freshwater]
MNHKNTVKASVRELTRHALQKPGEESEPGETTKTLKFRVDAKGHTRTRLDQIFTAQRQFVRDLLTKLEGMWDKDPQRFIRMVES
SASDPFESKTSCYGWMMTKFLTGSDLPEHLSRSAATASVATLSGNMKSFVTRRENVLKDMAVVIERNMESFEAGLKDLCAELKI
DRFKSPPSVDLESINAKTVEKYNEWVAAARVWCNLILAQKHKIPRGDAFFPTRLKAYPGFPGSVIHKEATSFATAIKELRKCLA
DLKQSHQKLFDALSTEEWSLVLEKFPAPEGTGGKGMRHQLGKTLAVLGKKHPEWKPRQYIEELVAGLERGADSMAKHLKNRQFS
DRPAAIKLINLLNNACVVLMEPLRVKSLYKEYFEQDTPRRNAFGQARGSLAEPTDQADSLQILGFSLMDEVRGVCRYDGFLAFT
GDKGKDQWAFFFDPDGPGGMGIADPVKHPKGSSDSGYIGFGTMGGSRKRAQAAEKRLVRERLWIRRDRKPLQLPLHFGTRQGRE
YLWNFDHGLKKSSDPWMLTNGRLLRVYPPNRPQLSDYFLTITLQKDTPPLAISSGDKLVGVDRGEQNPASFAVVDDKGRLLERG
NVGEKYTAKIRQFAEMKSDLQRTAGGYTRKLRAKERNTASALGGLVMRELLDLLVRHPGALVFERLSSGITTRGGRGTLMGNMH
YERILSGVEGKLAEAGCYKAPSAAKYRKGITPFLRFVNPAYTSATCSACGEVFSSGWYKKTAATISKESLGSWSVTLPNGKKLQ
LPSSYTSYMRGRGEKTWNTAERLEELFQDRTWSDLPPSTRKTVEGLVARLTHHRPKRGEFVCSSCGHKDDADLQAALNIARKSI
WVSSLPPRQAKQSEGEDRRSTEEPWRVWYKKQIMGKWS (SEQ ID NO: 11)
>3300014164|Ga0181532_10001487_14|M
[aquatic-freshwater-bog]
MPQTIELKLFMNSIHKSVRSLLSLGLSRSASASPQTITRTVKFKINTDIRPELIPVLNRHFDAFEKFRRKVLDELEAWWNKKPD
DFTSMVRATAKERFQDKSSCYGWLYRHFLSGKDLPAGLTREAANSVLWSLKGGLKSFLTRRENVAKELEENYTTNANLWNESII
RIIRDHKLDKPTPPTAPPTVNFKKLTDDQIEKYNGWVGIVRMWGNLILHQQLVQKGLVDREDAKLPRRLKGYPGFPGSQRYLQG
ASLADSLKRLDDITGKFAKDSEKRFAKITSEHWGAVLERFFPPEAKEDETRRRPRTVKQMAAERLHYLCKHNPDWKPEQTAKEV
LAGLFRLADKLHAHLKNNSPHDRRAVIKLANVLNVAAVFSLEPLRASGDYIACFEADEPRRQAFADLRGALAQPGDDTVAIEVT
GFSLSSNSPQYCGLLACREIKPDCDEWAFFYTLDNQDLKIFAKPGPRPPRDFQPTNWHGFATKGSDDEKEIVRGTVWLPTEISK
TKPALALPLAFGARQGREYLWHFDRDLRTRNEWTPLNARLLRVMPAAQESESEKERALRANRADFYVTITFLRLAPPIASVKSK
ELIGVDRGEVVPAAFAVVGATGRFLAKPSRLREFMEYLRQLDNWEKLNLAHPENARENERKPRWEARPDDGFGFIAPEYRDQQR
EFNKLKRELQQTKGGYTRWLRAKERNRARALGGQVMRELLMLCATHKAPLVFESLGSGLATRGGKGTMMSQMQYSRVFTSVEQK
LAEAGLCAIARQPKFRKWDNGFIKLVGPACTSATCSVCGQVHSTSFYATIAATLKESSKGNWQVTLPNGKVRRLPKEYGYWLRG
KGEQTANTDERIHQLLNKPFAELSTTNHNALISLLKSRWLPFRPDQATFQCLACGYSVNADVQAALNIARKHLFRIGREKKSDE
ENEAARRKVMGEWETWYRQKLASVWNK (SEQ ID NO: 12)
>3300014164|Ga0181532_10001487_14|P
[aquatic-freshwater-bog]
MGLGRTFCLGHNLARSTKRREKEDECGNFRGWQNHVGFRLGHRLRIARRMPQTIELKLFMNSIHKSVRSLLSLGLSRSASASPQ
TITRTVKFKINTDIRPELIPVLNRHFDAFEKFRRKVLDELEAWWNKKPDDFTSMVRATAKERFQDKSSCYGWLYRHFLSGKDLP
AGLTREAANSVLWSLKGGLKSFLTRRENVAKELEENYTTNANLWNESIIRIIRDHKLDKPTPPTAPPTVNFKKLTDDQIEKYNG
WVGIVRMWGNLILHQQLVQKGLVDREDAKLPRRLKGYPGFPGSQRYLQGASLADSLKRLDDITGKFAKDSEKRFAKITSEHWGA
VLERFFPPEAKEDETRRRPRTVKQMAAERLHYLCKHNPDWKPEQTAKEVLAGLFRLADKLHAHLKNNSPHDRRAVIKLANVLNV
AAVFSLEPLRASGDYIACFEADEPRRQAFADLRGALAQPGDDTVAIEVTGFSLSSNSPQYCGLLACREIKPDCDEWAFFYTLDN
QDLKIFAKPGPRPPRDFQPTNWHGFATKGSDDEKEIVRGTVWLPTEISKTKPALALPLAFGARQGREYLWHFDRDLRTRNEWTP
LNARLLRVMPAAQESESEKERALRANRADFYVTITFLRLAPPIASVKSKELIGVDRGEVVPAAFAVVGATGRFLAKPSRLREFM
EYLRQLDNWEKLNLAHPENARENERKPRWEARPDDGFGFIAPEYRDQQREFNKLKRELQQTKGGYTRWLRAKERNRARALGGQV
MRELLMLCATHKAPLVFESLGSGLATRGGKGTMMSQMQYSRVFTSVEQKLAEAGLCAIARQPKFRKWDNGFIKLVGPACTSATC
SVCGQVHSTSFYATIAATLKESSKGNWQVTLPNGKVRRLPKEYGYWLRGKGEQTANTDERIHQLLNKPFAELSTTNHNALISLL
KSRWLPFRPDQATFQCLACGYSVNADVQAALNIARKHLFRIGREKKSDEENEAARRKVMGEWETWYRQKLASVWNK (SEQ ID
NO: 13)
>3300014165|Ga0181523_10000360_35|M
[aquatic-freshwater-bog]
MPQTIELKLFMNSIHKSVRSLLSLGLSRSASASPQTITRTVKFKINTDIRPELIPVLNRHFDAFEKFRRKVLDELEAWWNKKPD
DFTSMVRATAKERFQDKSSCYGWLYRHFLSGKDLPAGLTREAANSVLWSLKGGLKSFLTRRENVAKELEENYTTNANLWNESII
RIIRDHKLDKPTPPTAPPTVNFKKLTDDQIEKYNGWVGIVRMWGNLILHQQLVQKGLVDREDAKLPRRLKGYPGFPGSQRYLQG
ASLADSLKRLDDITGKFAKDSEKRFAKITSEHWGAVLERFFPPEAKEDETRRRPRTVKQMAAERLHYLCKHNPDWKPEQTAKEV
LAGLFRLADKLHAHLKNNSPHDRRAVIKLANVLNVAAVFSLEPLRASGDYIACFEADEPRRQAFADLRGALAQPGDDTVAIEVT
GFSLSSNSPQYCGLLACREIKPDCDEWAFFYTLDNQDLKIFAKPGPRPPRDFQPTNWHGFATKGSDDEKEIVRGTVWLPTEISK
TKPALALPLAFGARQGREYLWHFDRDLRTRNEWTPLNARLLRVMPAAQESESEKERALRANRADFYVTITFLRLAPPIASVKSK
ELIGVDRGEVVPAAFAVVGATGRFLAKPSRLREFMEYLRQLDNWEKLNLAHPENARENERKPRWEARPDDGFGFIAPEYRDQQR
EFNKLKRELQQTKGGYTRWLRAKERNRARALGGQVMRELLMLCATHKAPLVFESLGSGLATRGGKGTMMSQMQYSRVFTSVEQK
LAEAGLCAIARQPKFRKWDNGFIKLVGPACTSATCSVCGQVHSTSFYATIAATLKESSKGNWQVTLPNGKVRRLPKEYGYWLRG
KGEQTANTDERIHQLLNKPFAELSTTNHNALISLLKSRWLPFRPDQATFQCLACGYSVNADVQAALNIARKHLFRIGREKKSDE
ENEAARRKVMGEWETWYRQKLASVWNK (SEQ ID NO: 12)
>3300014165|Ga0181523_10000360_33|P
[aquatic-freshwater-bog]
MGLGRTFCLGHNLARSTKRREKEDECGNFRGWQNHVGFRLGHRLRIARRMPQTIELKLFMNSIHKSVRSLLSLGLSRSASASPQ
TITRTVKFKINTDIRPELIPVLNRHFDAFEKFRRKVLDELEAWWNKKPDDFTSMVRATAKERFQDKSSCYGWLYRHFLSGKDLP
AGLTREAANSVLWSLKGGLKSFLTRRENVAKELEENYTTNANLWNESIIRIIRDHKLDKPTPPTAPPTVNFKKLTDDQIEKYNG
WVGIVRMWGNLILHQQLVQKGLVDREDAKLPRRLKGYPGFPGSQRYLQGASLADSLKRLDDITGKFAKDSEKRFAKITSEHWGA
VLERFFPPEAKEDETRRRPRTVKQMAAERLHYLCKHNPDWKPEQTAKEVLAGLFRLADKLHAHLKNNSPHDRRAVIKLANVLNV
AAVFSLEPLRASGDYIACFEADEPRRQAFADLRGALAQPGDDTVAIEVTGFSLSSNSPQYCGLLACREIKPDCDEWAFFYTLDN
QDLKIFAKPGPRPPRDFQPTNWHGFATKGSDDEKEIVRGTVWLPTEISKTKPALALPLAFGARQGREYLWHFDRDLRTRNEWTP
LNARLLRVMPAAQESESEKERALRANRADFYVTITFLRLAPPIASVKSKELIGVDRGEVVPAAFAVVGATGRFLAKPSRLREFM
EYLRQLDNWEKLNLAHPENARENERKPRWEARPDDGFGFIAPEYRDQQREFNKLKRELQQTKGGYTRWLRAKERNRARALGGQV
MRELLMLCATHKAPLVFESLGSGLATRGGKGTMMSQMQYSRVFTSVEQKLAEAGLCAIARQPKFRKWDNGFIKLVGPACTSATC
SVCGQVHSTSFYATIAATLKESSKGNWQVTLPNGKVRRLPKEYGYWLRGKGEQTANTDERIHQLLNKPFAELSTTNHNALISLL
KSRWLPFRPDQATFQCLACGYSVNADVQAALNIARKHLFRIGREKKSDEENEAARRKVMGEWETWYRQKLASVWNK (SEQ ID
NO: 13)
>3300014200|Ga0181526_10000193_31|M
[aquatic-freshwater-bog]
MPQTIELKLFMNSIHKSVRSLLSLGLSRSASASPQTITRTVKFKINTDIRPELIPVLNRHFDAFEKFRRKVLDELEAWWNKKPD
DFTSMVRATAKERFQDKSSCYGWLYRHFLSGKDLPAGLTREAANSVLWSLKGGLKSFLTRRENVAKELEENYTTNANLWNESII
RIIRDHKLDKPTPPTAPPTVNFKKLTDDQIEKYNGWVGIVRMWGNLILHQQLVQKGLVDREDAKLPRRLKGYPGFPGSQRYLQG
ASLADSLKRLDDITGKFAKDSEKRFAKITSEHWGAVLERFFPPEAKEDETRRRPRTVKQMAAERLHYLCKHNPDWKPEQTAKEV
LAGLFRLADKLHAHLKNNSPHDRRAVIKLANVLNVAAVFSLEPLRASGDYIACFEADEPRRQAFADLRGALAQPGDDTVAIEVT
GFSLSSNSPQYCGLLACREIKPDCDEWAFFYTLDNQDLKIFAKPGPRPPRDFQPTNWHGFATKGSDDEKEIVRGTVWLPTEISK
TKPALALPLAFGARQGREYLWHFDRDLRTRNEWTPLNARLLRVMPAAQESESEKERALRANRADFYVTITFLRLAPPIASVKSK
ELIGVDRGEVVPAAFAVVGATGRFLAKPSRLREFMEYLRQLDNWEKLNLAHPENARENERKPRWEARPDDGFGFIAPEYRDQQR
EFNKLKRELQQTKGGYTRWLRAKERNRARALGGQVMRELLMLCATHKAPLVFESLGSGLATRGGKGTMMSQMQYSRVFTSVEQK
LAEAGLCAIARQPKFRKWDNGFIKLVGPACTSATCSVCGQVHSTSFYATIAATLKESSKGNWQVTLPNGKVRRLPKEYGYWLRG
KGEQTANTDERIHQLLNKPFAELSTTNHNALISLLKSRWLPFRPDQATFQCLACGYSVNADVQAALNIARKHLFRIGREKKSDE
ENEAARRKVMGEWETWYRQKLASVWNK (SEQ ID NO: 12)
>3300014200|Ga0181526_10000193_30|P
[aquatic-freshwater-bog]
MGLGRTFCLGHNLARSTKRREKEDECGNFRGWQNHVGFRLGHRLRIARRMPQTIELKLFMNSIHKSVRSLLSLGLSRSASASPQ
TITRTVKFKINTDIRPELIPVLNRHFDAFEKFRRKVLDELEAWWNKKPDDFTSMVRATAKERFQDKSSCYGWLYRHFLSGKDLP
AGLTREAANSVLWSLKGGLKSFLTRRENVAKELEENYTTNANLWNESIIRIIRDHKLDKPTPPTAPPTVNFKKLTDDQIEKYNG
WVGIVRMWGNLILHQQLVQKGLVDREDAKLPRRLKGYPGFPGSQRYLQGASLADSLKRLDDITGKFAKDSEKRFAKITSEHWGA
VLERFFPPEAKEDETRRRPRTVKQMAAERLHYLCKHNPDWKPEQTAKEVLAGLFRLADKLHAHLKNNSPHDRRAVIKLANVLNV
AAVFSLEPLRASGDYIACFEADEPRRQAFADLRGALAQPGDDTVAIEVTGFSLSSNSPQYCGLLACREIKPDCDEWAFFYTLDN
QDLKIFAKPGPRPPRDFQPTNWHGFATKGSDDEKEIVRGTVWLPTEISKTKPALALPLAFGARQGREYLWHFDRDLRTRNEWTP
LNARLLRVMPAAQESESEKERALRANRADFYVTITFLRLAPPIASVKSKELIGVDRGEVVPAAFAVVGATGRFLAKPSRLREFM
EYLRQLDNWEKLNLAHPENARENERKPRWEARPDDGFGFIAPEYRDQQREFNKLKRELQQTKGGYTRWLRAKERNRARALGGQV
MRELLMLCATHKAPLVFESLGSGLATRGGKGTMMSQMQYSRVFTSVEQKLAEAGLCAIARQPKFRKWDNGFIKLVGPACTSATC
SVCGQVHSTSFYATIAATLKESSKGNWQVTLPNGKVRRLPKEYGYWLRGKGEQTANTDERIHQLLNKPFAELSTTNHNALISLL
KSRWLPFRPDQATFQCLACGYSVNADVQAALNIARKHLFRIGREKKSDEENEAARRKVMGEWETWYRQKLASVWNK (SEQ ID
NO: 13)
>3300027720|Ga0209617_10001817_3|M
[aquatic-freshwater-freshwater and sediment]
MNHKSTVKASVRHLTRHALQKPGEESDPGETTKTLKFRVDAKAHTRTRLDQIFTAQRQFVRDLLNKLEGMWDKDPQRFIRMVES
SASDPFESKTSCYGWMMTKFLTGSDLPEHLSRSAATASVATLSGNMKSFVTRRENVLKDMAVVIERNMENCEAGLKDLCAELKI
DRFKSPPSVDLESINAKTVEKYNEWVAAARVWCNLILAQKHKIPRGDAFFPTRLKAYPGFPGSVIHKEATSFATAIKELRKCLA
DLKQSHQKLFDALSNEEWSLVLERFPAPEGTGGKGMRHQLGKTLAVLGKKHPEWKPRQFIEELVAGLERGADSMAKHLENRQFS
DRPAAIKLINLLNNACVVLMEPLRVKSLYKEYFEQDTPRRNAFGQARGSLAEPTDQADSLQILGFSLMDEVRGICRYDGFLAFA
GDKGKDQWAFFFDPDGPGGMGIADPVKHPKGSSDSGYIGFGTMGGSRKRAQAAEKRLVRERLWIRRDRKPLQLPLHFGTRQGRE
YLWNFDHGLKKSSDPWMLTNGRLLRIYPPNRPQFSDYFLTITLQKDTPPLAISSGEKLVGVDRGEQNPASFAVVDAKGRLLERG
NVGEKYTAKIRQFAEMKSDLQRTAGGYTRKLRAKERNTASALGGLVMRELLDLLVRHPGALVFERLSSGITTRGGRGTLMGNMH
YERILSGVEGKLAEAGCYKAPSAAKYRKGITPFLRFVNPAYTSATCSACGEVFSSSWYKKTAATISKESPGSWSVTLPNGKKLQ
LPCSYTSYMRGRGEKTWNTAERLEELFKDRTWSDLPPSTRKTVEGLVARLTHHRPKRGEFVCPSCGHKDDADLQAALNIARKSI
WVSSLPPRQAKQSEGEDRRSTEEPWRVWYKKQIMEKWS (SEQ ID NO: 14)
>3300009764|Ga0116134_1001531_13|P
[aquatic-freshwater-peatland]
MKAKYRSNSIHASLRQLLELGLSKSSSAEPQKITRTAKFKINTDIRPDLVPVLNRHFDAFEKFRRKVLDELEARWNKDQKSFQA
MVQCSAKEPYQKKSSCYAWLDTHFITEAKAVLDLPRKPATSLLYNLSGGLKGFLTRRETVTEDIQKRFNDNLREWNGDLSQLAS
DLKAPLPPAPPNLDFENLTERAIERYNDWVGRTRAWCNLILVQQKKVERRDACLPRYLKGYPGFFGSQRYATTASLAENLKKLE
QEAREQSKKAPTRFAKLSPEIWTAIQERFSPPEGHEAGEKRRPRTAHQTVCLRFAALRAAHPEWTPAQLAGEILAGIFHGTEKL
KKHLAANGFEDRRAVIKLANLYNVVVAFSLDPIRAAGNYISFYKEETPKRNAFGDVRGGLHQPSDESAAIEIMGFGLQKESGKP
LYNGLLVCKKSEKEHDDSWAFLYCHTEGQMFELANEKAKLRGKLLTDWTGFASRGGSRKKAEASAKQLVRGRVWISEKTPPTVM
PLAFGSRQGREYLWHFDRDLREKNEWVLGNGRLLRIMPPGRPNAADFYLTITLERQAPPVAEFIASKLIGIDRGEAVPAAYALI
DREGRWLVNQKRFQEFLKALGTWHDELDEWWKKNKNTPINDRVKRPRLQWTSELEDGFGFVASEYREQQADFNDQKRELQRTQG
GYTRWLRSKERNRARALGGEVTRAVLSLASEHRSPLVLEKLGSALATRGGKGTMMSQMQYERMLTTLEQKLAEVGLYAMPSAPK
YRKLTNGFINFAAPHYTSSTCSSCGQVHDSIFYNELAKKIVRPNDSKWQVTLPSGQTRTLPQTYSYWLKGKGEQTKQTNERLNE
LLGERTVTQLSKSSYMTLVSLLKNSWLPYRPQQADFCCLNCGYTTNADVQGALNIARKFLFRSERGKKTDDAGEEGEAKRGKYK
DDWQTWYRQKLEKVWCK (SEQ ID NO: 15)
>3300018009|Ga0187884_10009678_2|M
[aquatic-freshwater-peatland]
MKAKYRSNSIHASLRQLLELGLSKSSSAEPQKITRTAKFKINTDIRPDLVPVLNRHFDAFEKFRRKVLDELEARWNKDQKSFQA
MVQCSAKEPYQKKSSCYAWLDTHFITEAKAVLDLPRKPATSLLYNLSGGLKGFLTRRETVTEDIQKRFNDNLREWNGDLSQLAS
DLKAPLPPAPPNLDFENLTERAIERYNDWVGRTRAWCNLILVQQKKVERRDACLPRYLKGYPGFFGSQRYATTASLAENLKKLE
QEAREQSKKAPTRFAKLSPEIWTAIQERFSPPEGHEAGEKRRPRTAHQTVCLRFAALRAAHPEWTPAQLAGEILAGIFHGTEKL
KKHLAANGFEDRRAVIKLANLYNVVVAFSLDPIRAAGNYISFYKEETPKRNAFGDVRGGLHQPSDESAAIEIMGFGLQKESGKP
LYNGLLVCKKSEKEHDDSWAFLYCHTEGQMFELANEKAKLRGKLLTDWTGFASRGGSRKKAEASAKQLVRGRVWISEKTPPTVM
PLAFGSRQGREYLWHFDRDLREKNEWVLGNGRLLRIMPPGRPNAADFYLTITLERQAPPVAEFIASKLIGIDRGEAVPAAYALI
DREGRWLVNQKRFQEFLKALGTWHDELDEWWKKNKNTPINDRVKRPRLQWTSELEDGFGFVASEYREQQADFNDQKRELQRTQG
GYTRWLRSKERNRARALGGEVTRAVLSLASEHRSPLVLEKLGSALATRGGKGTMMSQMQYERMLTTLEQKLAEVGLYAMPSAPK
YRKLTNGFINFAAPHYTSSTCSSCGQVHDSIFYNELAKKIVRPNDSKWQVTLPSGQTRTLPQTYSYWLKGKGEQTKQTNERLNE
LLGERTVTQLSKSSYMTLVSLLKNSWLPYRPQQADFCCLNCGYTTNADVQGALNIARKFLFRSERGKKTDDAGEEGEAKRGKYK
DDWQTWYRQKLEKVWCK (SEQ ID NO: 15)
>3300018014|Ga0187860_1004616_12|M
[aquatic-freshwater-peatland]
MKAKYRSNSIHASLRQLLELGLSKSSSAEPQKITRTAKFKINTDIRPDLVPVLNRHFDAFEKFRRKVLDELEARWNKDQKSFQA
MVQCSAKEPYQKKSSCYAWLDTHFITEAKAVLDLPRKPATSLLYNLSGGLKGFLTRRETVTEDIQKRFNDNLREWNGDLSQLAS
DLKAPLPPAPPNLDFENLTERAIERYNDWVGRTRAWCNLILVQQKKVERRDACLPRYLKGYPGFFGSQRYATTASLAENLKKLE
QEAREQSKKAPTRFAKLSPEIWTAIQERFSPPEGHEAGEKRRPRTAHQTVCLRFAALRAAHPEWTPAQLAGEILAGIFHGTEKL
KKHLAANGFEDRRAVIKLANLYNVVVAFSLDPIRAAGNYISFYKEETPKRNAFGDVRGGLHQPSDESAAIEIMGFGLQKESGKP
LYNGLLVCKKSEKEHDDSWAFLYCHTEGQMFELANEKAKLRGKLLTDWTGFASRGGSRKKAEASAKQLVRGRVWISEKTPPTVM
PLAFGSRQGREYLWHFDRDLREKNEWVLGNGRLLRIMPPGRPNAADFYLTITLERQAPPVAEFIASKLIGIDRGEAVPAAYALI
DREGRWLVNQKRFQEFLKALGTWHDELDEWWKKNKNTPINDRVKRPRLQWTSELEDGFGFVASEYREQQADFNDQKRELQRTQG
GYTRWLRSKERNRARALGGEVTRAVLSLASEHRSPLVLEKLGSALATRGGKGTMMSQMQYERMLTTLEQKLAEVGLYAMPSAPK
YRKLTNGFINFAAPHYTSSTCSSCGQVHDSIFYNELAKKIVRPNDSKWQVTLPSGQTRTLPQTYSYWLKGKGEQTKQTNERLNE
LLGERTVTQLSKSSYMTLVSLLKNSWLPYRPQQADFCCLNCGYTTNADVQGALNIARKFLFRSERGKKTDDAGEEGEAKRGKYK
DDWQTWYRQKLEKVWCK (SEQ ID NO: 15)
>3300018016|Ga0187880_1008327_2|M
[aquatic-freshwater-peatland]
MKAKYRSNSIHASLRQLLELGLSKSSSAEPQKITRTAKFKINTDIRPDLVPVLNRHFDAFEKFRRKVLDELEARWNKDQKSFQA
MVQCSAKEPYQKKSSCYAWLDTHFITEAKAVLDLPRKPATSLLYNLSGGLKGFLTRRETVTEDIQKRFNDNLREWNGDLSQLAS
DLKAPLPPAPPNLDFENLTERAIERYNDWVGRTRAWCNLILVQQKKVERRDACLPRYLKGYPGFFGSQRYATTASLAENLKKLE
QEAREQSKKAPTRFAKLSPEIWTAIQERFSPPEGHEAGEKRRPRTAHQTVCLRFAALRAAHPEWTPAQLAGEILAGIFHGTEKL
KKHLAANGFEDRRAVIKLANLYNVVVAFSLDPIRAAGNYISFYKEETPKRNAFGDVRGGLHQPSDESAAIEIMGFGLQKESGKP
LYNGLLVCKKSEKEHDDSWAFLYCHTEGQMFELANEKAKLRGKLLTDWTGFASRGGSRKKAEASAKQLVRGRVWISEKTPPTVM
PLAFGSRQGREYLWHFDRDLREKNEWVLGNGRLLRIMPPGRPNAADFYLTITLERQAPPVAEFIASKLIGIDRGEAVPAAYALI
DREGRWLVNQKRFQEFLKALGTWHDELDEWWKKNKNTPINDRVKRPRLQWTSELEDGFGFVASEYREQQADFNDQKRELQRTQG
GYTRWLRSKERNRARALGGEVTRAVLSLASEHRSPLVLEKLGSALATRGGKGTMMSQMQYERMLTTLEQKLAEVGLYAMPSAPK
YRKLTNGFINFAAPHYTSSTCSSCGQVHDSIFYNELAKKIVRPNDSKWQVTLPSGQTRTLPQTYSYWLKGKGEQTKQTNERLNE
LLGERTVTQLSKSSYMTLVSLLKNSWLPYRPQQADFCCLNCGYTTNADVQGALNIARKFLFRSERGKKTDDAGEEGEAKRGKYK
DDWQTWYRQKLEKVWCK (SEQ ID NO: 15)
>3300018017|Ga0187872_10010132_5|M
[aquatic-freshwater-peatland]
MKAKYRSNSIHASLRQLLELGLSKSSSAEPQKITRTAKFKINTDIRPDLVPVLNRHFDAFEKFRRKVLDELEARWNKDQKSFQA
MVQCSAKEPYQKKSSCYAWLDTHFITEAKAVLDLPRKPATSLLYNLSGGLKGFLTRRETVTEDIQKRFNDNLREWNGDLSQLAS
DLKAPLPPAPPNLDFENLTERAIERYNDWVGRTRAWCNLILVQQKKVERRDACLPRYLKGYPGFFGSQRYATTASLAENLKKLE
QEAREQSKKAPTRFAKLSPEIWTAIQERFSPPEGHEAGEKRRPRTAHQTVCLRFAALRAAHPEWTPAQLAGEILAGIFHGTEKL
KKHLAANGFEDRRAVIKLANLYNVVVAFSLDPIRAAGNYISFYKEETPKRNAFGDVRGGLHQPSDESAAIEIMGFGLQKESGKP
LYNGLLVCKKSEKEHDDSWAFLYCHTEGQMFELANEKAKLRGKLLTDWTGFASRGGSRKKAEASAKQLVRGRVWISEKTPPTVM
PLAFGSRQGREYLWHFDRDLREKNEWVLGNGRLLRIMPPGRPNAADFYLTITLERQAPPVAEFIASKLIGIDRGEAVPAAYALI
DREGRWLVNQKRFQEFLKALGTWHDELDEWWKKNKNTPINDRVKRPRLQWTSELEDGFGFVASEYREQQADFNDQKRELQRTQG
GYTRWLRSKERNRARALGGEVTRAVLSLASEHRSPLVLEKLGSALATRGGKGTMMSQMQYERMLTTLEQKLAEVGLYAMPSAPK
YRKLTNGFINFAAPHYTSSTCSSCGQVHDSIFYNELAKKIVRPNDSKWQVTLPSGQTRTLPQTYSYWLKGKGEQTKQTNERLNE
LLGERTVTQLSKSSYMTLVSLLKNSWLPYRPQQADFCCLNCGYTTNADVQGALNIARKFLFRSERGKKTDDAGEEGEAKRGKYK
DDWQTWYRQKLEKVWCK (SEQ ID NO: 15)
>3300018022|Ga0187864_10004559_12|P
[aquatic-freshwater-peatland]
MKAKYRSNSIHASLRQLLELGLSKSSSAEPQKITRTAKFKINTDIRPDLVPVLNRHFDAFEKFRRKVLDELEARWNKDQKSFQA
MVQCSAKEPYQKKSSCYAWLDTHFITEAKAVLDLPRKPATSLLYNLSGGLKGFLTRRETVTEDIQKRFNDNLREWNGDLSQLAS
DLKAPLPPAPPNLDFENLTERAIERYNDWVGRTRAWCNLILVQQKKVERRDACLPRYLKGYPGFFGSQRYATTASLAENLKKLE
QEAREQSKKAPTRFAKLSPEIWTAIQERFSPPEGHEAGEKRRPRTAHQTVCLRFAALRAAHPEWTPAQLAGEILAGIFHGTEKL
KKHLAANGFEDRRAVIKLANLYNVVVAFSLDPIRAAGNYISFYKEETPKRNAFGDVRGGLHQPSDESAAIEIMGFGLQKESGKP
LYNGLLVCKKSEKEHDDSWAFLYCHTEGQMFELANEKAKLRGKLLTDWTGFASRGGSRKKAEASAKQLVRGRVWISEKTPPTVM
PLAFGSRQGREYLWHFDRDLREKNEWVLGNGRLLRIMPPGRPNAADFYLTITLERQAPPVAEFIASKLIGIDRGEAVPAAYALI
DREGRWLVNQKRFQEFLKALGTWHDELDEWWKKNKNTPINDRVKRPRLQWTSELEDGFGFVASEYREQQADFNDQKRELQRTQG
GYTRWLRSKERNRARALGGEVTRAVLSLASEHRSPLVLEKLGSALATRGGKGTMMSQMQYERMLTTLEQKLAEVGLYAMPSAPK
YRKLTNGFINFAAPHYTSSTCSSCGQVHDSIFYNELAKKIVRPNDSKWQVTLPSGQTRTLPQTYSYWLKGKGEQTKQTNERLNE
LLGERTVTQLSKSSYMTLVSLLKNSWLPYRPQQADFCCLNCGYTTNADVQGALNIARKFLFRSERGKKTDDAGEEGEAKRGKYK
DDWQTWYRQKLEKVWCK (SEQ ID NO: 15)
>3300018040|Ga0187862_10006434_12|P
[aquatic-freshwater-peatland]
MKAKYRSNSIHASLRQLLELGLSKSSSAEPQKITRTAKFKINTDIRPDLVPVLNRHFDAFEKFRRKVLDELEARWNKDQKSFQA
MVQCSAKEPYQKKSSCYAWLDTHFITEAKAVLDLPRKPATSLLYNLSGGLKGFLTRRETVTEDIQKRFNDNLREWNGDLSQLAS
DLKAPLPPAPPNLDFENLTERAIERYNDWVGRTRAWCNLILVQQKKVERRDACLPRYLKGYPGFFGSQRYATTASLAENLKKLE
QEAREQSKKAPTRFAKLSPEIWTAIQERFSPPEGHEAGEKRRPRTAHQTVCLRFAALRAAHPEWTPAQLAGEILAGIFHGTEKL
KKHLAANGFEDRRAVIKLANLYNVVVAFSLDPIRAAGNYISFYKEETPKRNAFGDVRGGLHQPSDESAAIEIMGFGLQKESGKP
LYNGLLVCKKSEKEHDDSWAFLYCHTEGQMFELANEKAKLRGKLLTDWTGFASRGGSRKKAEASAKQLVRGRVWISEKTPPTVM
PLAFGSRQGREYLWHFDRDLREKNEWVLGNGRLLRIMPPGRPNAADFYLTITLERQAPPVAEFIASKLIGIDRGEAVPAAYALI
DREGRWLVNQKRFQEFLKALGTWHDELDEWWKKNKNTPINDRVKRPRLQWTSELEDGFGFVASEYREQQADFNDQKRELQRTQG
GYTRWLRSKERNRARALGGEVTRAVLSLASEHRSPLVLEKLGSALATRGGKGTMMSQMQYERMLTTLEQKLAEVGLYAMPSAPK
YRKLTNGFINFAAPHYTSSTCSSCGQVHDSIFYNELAKKIVRPNDSKWQVTLPSGQTRTLPQTYSYWLKGKGEQTKQTNERLNE
LLGERTVTQLSKSSYMTLVSLLKNSWLPYRPQQADFCCLNCGYTTNADVQGALNIARKFLFRSERGKKTDDAGEEGEAKRGKYK
DDWQTWYRQKLEKVWCK (SEQ ID NO: 15)
>OCZT010053476_2|M
[soil metagenome]
MSKEPRKNSTHASLRQLLELGLSKSSSAEPQKITRTVKFKIHTLARPELVAPLNKHFNAFDQFREKVLAELESWWDKDPDSFTH
MVKCSTKLKFEEKSSCYAWLYKKFLTGAELPPELSRDAANAMLDNLGGGLKSFLTRRATVSLEIKKRYEQNKEEWEKELSLIAK
ERNLKLPEVPREIDFNNLTAKKIDFYNEWVGLVRMWCNLILIQKEKLTRENACLPRYLKSYPGFPGSQRFRKPVTIEQTLAELE
TIAREQAGKASQRFADLKPEVWQAIQERFSPPKKEEAETTPRPRTASQTVSRRFTALIQANPNWKPQQIAEEILAGIFRAADKL
KKHLVTTKYTDRRATIKFANLFNVAVVFAREPFRARGDYVASYEADIARQKAFGDLRGALHQPSDESSAIQISGFSIKDGGTPN
YNGLLVCKSEANGKDSWAFLYCHQEGQIFQLADEKAKLRGKKLSEWTGFASRGGSRKKAEASPKQLVKSSVWVSEKNPPTVLPL
AFGTRLGREYLWHFDRNLRTKSEWVLGNGRLLRVMPPGRTDLAEFYLAITMERETPPVASVVANKLIGIDRGEAVPASYAVVDG
EERLLDSGRIAENYREEQRAFNKSKRELQSERGGYTRTLRSKERNRARALGGEVTRKLLSLAAEHCAPLVLENLNSSLATRGGK
NTMMSQMQYERVFTALEQKLAEAGLYSLPSAPKFRKGDNAFIKLVGPAYTSQTCSACGHVHWAEFYEKILDTLAPCGEKQWQVT
VEGKARTLPESYTYWLRGKGEQTKNTHERIIELLKDRPVSKLNKTDRKSLVSLFKNRWMPYRPKQADFQCVVCGHKMNADEQGA
LNIARKFLFASGRGKKSGEMTEAERRKVRGEWEKWYKEKLATVWKKQ (SEQ ID NO: 16)
>3300014839|Ga0182027_10014967_5|M
[terrestrial-soil-fen]
MSKEYRPDSIHASLRHLLELGLSKTGSSEPQKITRTVKFKVHTLARPELVAPLNQHFNAFDEFRHKVLADLESWWDKAPDSFTR
MVKSSTKAKYEEKSSCYAWLYRKYLTGAELPTELSRDAANAMLDNLAGSLKSFLTRRATVSLEIKKRYEQNREEWEKELSLVAK
EKNLTLPKPPPEIDFDHLNVMAIGLYNEWVGLVRMWCNLILIQKEKLTRENACLPRYLKSYPGFPGSQRFRKPVTIEQTFGELE
AAARDQAGNATNRFANVKPEVWQAIEERFKPPAKDEVEIEARPRSRTASQTVARRLAALVKANPNWTPDQLAVEILAGIFRAAD
KLQKHLAATEYTDHRATIKLANLFNAAVVFAREPFRASGDYVASYGADMARQKAFGDLRGALHQPSDESSAIQITGFSVNEDGS
PNYNGLLVCKPEADGKDSWAFLYCHQEGQTFQLADEKAKLRGKKLSEWTGFASRGGSRKKAEASAKQLVKGRVWVSEKKPPTVL
PLAFGTRMGREYLWHFDRNLRTKSAWVLGNGRLLRVMPPARPDLAEFYLTITMEREAPPVASVVANKLIGIDRGEAVPAAYAVV
DSEGRLLDSGRIAEKYREEQRAFNKSKRELQSERGGYTRTLRSKERNRARALGGEVTRRLLSLAAEHRAPLVLENLNSSLATRG
GKNTMMSQMQYERVFTALEQKLAEAGLYSLPSAPKYRKGDNAFIKLVGPAYTSQTCSACGHVHSSEFYDQLTDTLAPCGDKQWR
VTVEGKPRILPESYTYWLRGKGDQTRNTNERIVELLKDRPISKLTKTDRKSLVSLVKNRWMQYRPTQADFQCVVCGHKMNADEQ
GALNIARKFLFALGRGKKSGEMTEAERRKVRGEWEKWYKEKLGTVWRK (SEQ ID NO: 17)

TABLE 3
Nucleotide sequences of Representative
CLUST.121143 Direct Repeats

| | Direct Repeat |
|---|---|
| CLUST.121143 Effector Protein | Nucleotide |
| Accession | Sequence |
| 3300013006\|Ga0164294_0029128_3\|M | GGTTTGCTGACCTCG |
| (SEQ ID NO: 1) | ATTGTTGGAGGAGTG |
| | CAAAAG |
| | (SEQ ID NO: 101) |
| 3300013087\|Ga0163212_1009539_3\|M | GGTTTCGCACTCCCT |
| (SEQ ID NO: 2) | CATTGTTGGTGGTGT |
| | GCAAAAG |
| | (SEQ ID NO: 102) |
| 3300013131\|Ga0172373_10014249_3\|P | GGTTTGCTCCGGTCC |
| (SEQ ID NO: 3) | TTGTTGGTAGGTGTG |
| | CAAAAG |
| | (SEQ ID NO: 103) |
| 3300013131\|Ga0172373_10021309_4\|M | GGTTAGCTCCCATCC |
| (SEQ ID NO: 4) | TTGTTGGGTGGTTTG |
| | CAAAAG |
| | (SEQ ID NO: 104) |
| 3300013132\|Ga0172372_10046622_2\|M | GGTTAGCTCCCATCC |
| (SEQ ID NO: 4) | TTGTTGGGTGGTTTG |
| | CAAAAG |
| | (SEQ ID NO: 104) |
| 3300014720\|Ga0172376_10007396_6\|M | GGTTTGCTCCGGTCC |
| (SEQ ID NO: 3) | TTGTTGGTAGGTGTG |
| | CAAAAG |
| | (SEQ ID NO: 103) |
| 3300014720\|Ga0172376_10013958_4\|M | GGTTAGCTCCCATCC |
| (SEQ ID NO: 4) | TTGTTGGGTGGTTTG |
| | CAAAAG |
| | (SEQ ID NO: 104) |
| 3300018790\|Ga0187842_1006374_2\|M | GGTTTGCTGACCTCG |
| (SEQ ID NO: 5) | ATTGTTGGAGGAGTG |
| | CAAAAG |
| | (SEQ ID NO: 101) |
| 3300018815\|Ga0187845_1009203_1\|M | GGTTTGCTGACCTCG |
| (SEQ ID NO: 6) | ATTGTTGGAGGAGTG |
| | CAAAAG |
| | (SEQ ID NO: 101) |
| 3300018868\|Ga0187844_10013314_2\|M | GGTTTGCTGACCTCG |
| (SEQ ID NO: 7) | ATTGTTGGAGGAGTG |
| | CAAAAG |
| | (SEQ ID NO: 101) |
| 3300019093\|Ga0187843_10013171_2\|M | GGTTTGCTGACCTCG |
| (SEQ ID NO: 8) | ATTGTTGGAGGAGTG |
| | CAAAAG |
| | (SEQ ID NO: 101) |
| 3300023179\|Ga0214923_10008431_2\|P | TTGCCTCCTGTGGGG |
| (SEQ ID NO: 9) | TTGTATGTGAAG |
| | (SEQ ID NO: 105) |
| 3300027730\|Ga0247833_1032983_1\|M | GGTTTGCTGACCTCG |
| (SEQ ID NO: 10) | ATTGTTGGAGGTGTG |
| | CAAAAG |
| | (SEQ ID NO: 106) |
| 3300028114\|Ga0247835_1021903_1lP | GGTTTGCTGACCTCG |
| (SEQ ID NO: 11) | ATTGTTGGAGGTGTG |
| | CAAAAG |
| | (SEQ ID NO: 106) |
| 3300028553\|Ga0247839_1034275_1\|M | GGTTTGCTGACCTCG |
| (SEQ ID NO: 10) | ATTGTTGGAGGTGTG |
| | CAAAAG |
| | (SEQ ID NO: 106) |
| 3300028569\|Ga0247843_1031164_2\|P | GGTTTGCTGACCTCG |
| (SEQ ID NO: 11) | ATTGTTGGAGGTGTG |
| | CAAAAG |
| | (SEQ ID NO: 106) |
| 3300014164\|Ga0181532_10001487_14\|M | GTGAGAACCGCTGCG |
| (SEQ ID NO: 12) | GTTCGCGGGGATGGT |
| | TTCAAAG |
| | (SEQ ID NO: 107) |
| 3300014164\|Ga0181532_10001487_14\|P | GTGAGAACCGCTGCG |
| (SEQ ID NO: 13) | GTTCGCGGGGATGGT |
| | TTCAAAG |
| | (SEQ ID NO: 107) |
| 3300014165\|Ga0181523_10000360_35\|M | GTGAGAACCGCTGCG |
| (SEQ ID NO: 12) | GTTCGCGGGGATGGT |
| | TTCAAAG |
| | (SEQ ID NO: 107) |
| 3300014165\|Ga0181523_10000360_33\|P | GTGAGAACCGCTGCG |
| (SEQ ID NO: 13) | GTTCGCGGGGATGGT |
| | TTCAAAG |
| | (SEQ ID NO: 107) |
| 3300014200\|Ga0181526_10000193_31\|M | GTGAGAACCGCTGCG |
| (SEQ ID NO: 12) | GTTCGCGGGGATGGT |
| | TTCAAAG |
| | (SEQ ID NO: 107) |
| 3300014200\|Ga0181526_10000193_30\|P | GTGAGAACCGCTGCG |
| (SEQ ID NO: 13) | GTTCGCGGGGATGGT |
| | TTCAAAG |
| | (SEQ ID NO: 107) |
| 3300027720\|Ga0209617_10001817_3\|M | GGTTTGCTGACCTCG |
| (SEQ ID NO: 14) | ATTGTTGGAGGAGTG |
| | CAAAAG |
| | (SEQ ID NO: 101) |
| 3300009764\|Ga0116134_1001531_13\|P | TTCGGAATCGCTGAC |
| (SEQ ID NO: 15) | GCTCGCGGGGCTGGT |
| | TTCAAAG |
| | (SEQ ID NO: 108) |
| 3300018009\|Ga0187884_10009678_2\|M | TTCGGAATCGCTGAC |
| (SEQ ID NO: 15) | GCTCGCGGGGCTGGT |
| | TTCAAAG |
| | (SEQ ID NO: 108) |
| 3300018014\|Ga0187860_1004616_12\|M | TTCGGAATCGCTGAC |
| (SEQ ID NO: 15) | GCTCGCGGGGCTGGT |
| | TTCAAAG |
| | (SEQ ID NO: 108) |
| 3300018016\|Ga0187880_1008327_2\|M | TTCGGAATCGCTGAC |
| (SEQ ID NO: 15) | GCTCGCGGGGCTGGT |
| | TTCAAAG |
| | (SEQ ID NO: 108) |
| 3300018017\|Ga0187872_10010132_5\|M | TTCGGAATCGCTGAC |
| (SEQ ID NO: 15) | GCTCGCGGGGCTGGT |
| | TTCAAAG |
| | (SEQ ID NO: 108) |
| 3300018022\|Ga0187864_10004559_12\|P | TTCGGAATCGCTGAC |
| (SEQ ID NO: 15) | GCTCGCGGGGCTGGT |
| | TTCAAAG |
| | (SEQ ID NO: 108) |
| 3300018040\|Ga0187862_10006434_12\|P | TTCGGAATCGCTGAC |
| (SEQ ID NO: 15) | GCTCGCGGGGCTGGT |
| | TTCAAAG |
| | (SEQ ID NO: 108) |
| OCZT010053476_2\|M (SEQ ID NO: 16) | GTTTGCATGATGACG |
| | TTTGCGGGGTTGGTT |
| | TGAAAG |
| | (SEQ ID NO: 109) |
| 3300014839\|Ga0182027_10014967_5\|M | CTCTGACGCGCTGAC |
| (SEQ ID NO: 17) | GTTCGCGGGGTTGGT |
| | TTCAAAG |
| | (SEQ ID NO: 110) |

TABLE 4
Direct Repeat Homology-containing
Regions of Representative
CLUST.121143 Systems

| Effector accession | Homologous Region |
|---|---|
| 3300018040\|Ga0187862_ | CCGCGAG |
| 10006434_12\|P | (SEQ ID NO: 200) |

TABLE 5
Direct Repeat Homology-containing Loci Sequences
of Representative CLUST.121143 Systems

>3300018040|Ga0187862_10006434_12|P
TGTTCCGTTCCGAACGAGGCAAAAAGACTGACGACGCAGGC
GAAGAAGGTGAGGCAAAGCGTGGTAAATACAAAGACGACTG
GCAGACTTGGTATCGCCAAAAGTTGGAAAAGGTCTGGTGCA
AGTAATCTGACATCGCCCATTCGAGACCCGCGAGCATGACA
TGGAGACCGACGATTTCCTTCCTATCTACTCGCTCGCGGAG
TGGCATTATTGCCCGCGCTCGTCTTTTCTGTCGTGGTTTGG
TGCAGAGCGTGAGGATCGCGTCACCCCGGCTTACCAGAAAA
TGCGCGAAGTCCACGCGGTG
(SEQ ID NO: 201)

TABLE 6
Non-coding Sequences of Representative CLUST.121143 Systems

>3300028569|Ga0247843_1031164_2|P
CACAATATTTCTTAAATGTCGAGAAAATAATATTTTACACCTATCCATATGCCAAATTACAAAAATGAATGTTTTACCTCATAA
AGAGTAAAAAGTGCTCCCCCGTCACAAAACATTAGTTTGGTGACTGGTTGTTTTTAACCACCAACTTCTCTGTCCAATTTATTG
GGGTTTTGTGAATCCCTATTCCAAGGACCGTTTTTGTACTACCCCCTAATTTACAGTTATCCTTATTATGAACTGGAATAGCTC
CCTTAAAAGTCCGATTATAACTGCTTGCTTAATACGATGAATCACAAAAATACCGTTAAAGCTCCTGTTCGCGAACTTACCCGG
CATGCATTGCAGAAACCCGGGGAGGAGAGTGAACCGGGAGAAACAACAAAGACTCTTAAGTTCCGTGTGGATGCCAAGGGACAT
ACCCGAACCCGACTCGACCAGGCAGCACTGAACATTGCTCGGAAAAGCATCTGGGTCAGTTCCCTTCCACCCCGTCAGGCAAAG
CAATCGGAAGGTGAAGACCGGCGTTCCACCGAGGAACCATGGCGCGTTTGGTACAAGAAGCAGATCATGGGAAAGTGGTCTTAA
AAAATCCCTTGCCACCCGGCTATCATAGGATCAAATTAACCCCTTAGTATTTTCTTTGCACAACGTCGACTGCCCTGCCAAGGA
GCGGCTGATAACCAACAAGTTCTTTTACATTTGAGGTGCAAACCGTTCTTGTGATTTTTTTATAAAAGGATGCACCCGAAAGGG
TTGGCTATACCCACATACTGCCCGCTTGCATTATGT (SEQ ID NO: 202)
>3300013006|Ga0164294_10029128_3|M
GTCTACTTTCACGGCAACGTGAAAGACTTGGAACCCAAACCCGGTTCGGTCACACCGATCCAGAAAACGGCCGCGGGGCTGATG
TTTCTTGTCGATGCGATCATGCTGGGACTCGCTCTTTATTTGATCCTTCGCTAACGCGAACCGATCGATAAGGATTAAGTCGCA
CTACCCCTGCATCGATTCCCGTTTTTTCGATTCCCTTCCAGGTATTTAATTTTTACTTCTCAACGAGGTATTTGGCTTGGCATA
GTCTCCTCTGCGCCCCCTGCCCCCTGTCCCCTGCCCCCTTTCACTTCCAGCCTACCAATTCCCAGTGTTCAAAACGTCGGTGTT
TTGGCGTTTATCCTTTCCTGAGATAACGCCCAGGACTGTTTTTGTCCCACCTCCTAATTTACACTTATCCTTATTATGAACTGG
AATAGCTCCGTTAAAAGTCCGATTATGACTGCTTGCTTAATACGATGAATCACAAAAGCACTGTCAAAGCCTCTGTTCGCCATC
TTACTCGGCATGCATTGCAGAAACCCGGGGAGGAGAGTGATCCGGGAGAAACGACAAAGACTTTAAAGTTCCGGGTAGATGCCA
AAGGACATACCCGAACCCGACTCGACCAGGCAGCACTGAACATTGCCCGCAAAAGCATCTGGGTCAGCTCCCTTCCACCCCGTC
AGGCAAAGCAATCGGAAGGTGAAGACCGGCGTTCCACCGAGGAACCATGGCGCGTTTGGTACAAAAAGCAGATCATGGAAAAGT
GGTCTTAAAAAATCCCTTGCCACCCGGCTATCATAGGATCATTTTAGTCTCTTAGTATTTTCTTTGCACAACGTCGACTGCCCT
GCCAAGGAGCGGCTGACAACCAACAAGTTCTTTTACATTTGAGGTGCAAACCGTTCTTGTGATTTTTTTACAAAAGGATGCACC
CGAAAGGGTTGGCCATACCC (SEQ ID NO: 203)
>3300018868|Ga0187844_10013314_2|M
ACGGCAACGTGAAAGACTTGGAACCCAAACCCGGTTCGGTCACACCGATCCAGAAAACGGCCGCGGGGCTGATGTTTCTTGTCG
ATGCGATCATGCTGGGACTCGCTCTTTATTTGATCCTTCGCTAACGCGAACCGATCGATAAGGATTAAGTCGCACTACCCCTGC
ATCGATTCCCGTTTTTTCGATTCCCTTCCAGGTATTTAATTTTTACTTCTCAACGAGGTATTTGGCTTGGCATGGTCTTCTCTG
CGCCCCCTGCCCCCTGTCCCCTGCCCCCTTTCACTTCCAGCCTACCAATTCCCAGTGTTCAAAACGTCGGTGTTTTGGCGTTTA
TCCTTTCCTGAGATAACGCCCAGGACTGTTTTTGTCCCACCTCCTAATTTACACTTATCCTTATTATGAACTGGAATAGCTCCG
TTAAAAGTCCGATTATGACTGCTTGCTTAATACGATGAATCACAAAAGCACTGTCAAAGCCTCTGTTCGCCATCTTACTCGGCA
TGCATTGCAGAAACCCGGGGAGGAGAGTGATCCGGGAGAAACGACAAAGACTTTAAAGTTCCGGGTAGATGCCAAAGGACATAC
CCGAACCCGACTCGACCAGGCAGCACTGAACATTGCCCGCAAAAGCATCTGGGTCAGCTCCCTTCCACCCCGTCAGGCAAAGCA
ATCGGAAGGTGAAGACCGGCGTTCCACCGAGGAACCATGGCGCGTTTGGTACAAAAAGCAGATCATGGAAAAGTGGTCTTAAAA
AATCCCTTGCCACCCGGCTATCATAGGATCATTTTAGTCTCTTAGTATTTTCTTTGCACAACGTCGACTGCCCTGCCAAGGAGC
GGCTGACAACCAACAAGTTCTTTTACATTTGAGGTGCAAACCGTTCTTGTGATTTTTTTACAAAAGGATGCACCCGAAAGGGTT
GGCCATACCC (SEQ ID NO: 204)
>3300023179|Ga0214923_10008431_2|P
TCAAAACCAGCCGTAGCTGGCGGAAAATATGCTTGGGATTGTCATTGGTATGTGGCCAATTCCTTTCCCACACGGCGTGCTTGT
CCACAGGATTTTAACCCTGCCTGTTTTTTTGATATCAAACCTGATATCGGTCATTTCTCCTCGATACTCTGAAACCAATCAGAA
ACACCCTTCCCAGGATGCTGAGTTAAGAACCACACGCTAACTTATCGTTTGGCTGAAAGGGATTTGAACCCTTTTCTTATTATA
TTGCTGAATACAGAGAAATTCTAAGAAATCTTAAAATATATTTTACCTGCAAACAGCACAAAGCACTAATAGCCAGTTATATGC
ACACCTAGTAGGATTGAATCTTATTAATACAACCTAGGACAGCAAATGACCCACCATATGAGTTATTATTGTGGAATGAAGAAT
TACCAAACTTGTGGAAAGTCGATATCTGGGGATACGATCAATGCTTAATCCAGCTGAAATGCAAATGCCCTCAAATAGTATAAA
ATCGATCAAAACTTCAGTACGAGAGCTGTACAACCACGCCTTGAACAAACAGGGCGATGGTGAGCCAGGAGAGACAACCAAGAC
ACTTAAGTTCAAGATCGATCCACAAGCGGCGCTGAACATTGCCAGGAAGACGATCTGGGTCAGTACCCTACCTCCTAAAAAAGC
CAAGAGCGGTGATCAGGACTTTCGTGGTGTAGGACAAGAGCTGTGGGCGAAATGGTACAAGGATAGGGTTCACAAAAACTGGGC
ATAAAATAGCACTTGTCTGAATTGGGATGGGTTAATACATTGAAATCTTAGTATTTACTTTGCATAGCGTTGCCAACCTTGCCC
AAGGATGAGTCAACGCCCCATAGAAGGCAGTTCATTAACACGCAAAAGGCACACCGCCGTAGTAAGTAGGGGGCAGCAACCACA
GATGGTGAGATGCCTCCTATGGGGTCATATGTGAAGTTCATGTTGTCTCCCATCTTGGACCCATAGCCGTTGGTGAGTTGCCTC
CTGTGGGGCCATATGTGAAGGAGGCCAATTCTGCTCGATTGTGATCCAATGTGATGGTGAGTTGCCTCCTGTGGGGTTGTATGT
GAAGGTCAATGTGGAGCGATTGCTGGGGGTATTCAGTTGGTGATTTGCCTCCTATGGGGCTATGTGTGAAGGGATGCGATCCTG
CAACATTCAAGCCTAGTCAGTTGCAAGCGTTTTCTGCAAATGATAGAGATCTGTTGGTGGTTTGCCTTCTGTGGGGCTATGTGT
GAAGGATGTCGAAGCGCCATTGCTCGGCATTGATGCGTGTGTGATCTGGTTTCTGTGGAGTTATGTGTGAAGGCTCGCTCTCAT
AGTTCTTTGGCTTGGCACTCATAGCACTTTTCCGTTTAAACCAGCAAAAAATCGCCGCACTAGATAATGCAACATAATTTGTAT
AAATTATCCAAATAAATGCCTGTTTTCTCAACACAAGAAAAACAGCCTATCCTTTCATACTTATCTGTTTCTCTCTGTGCATCA
ATTAAATATATAATAGGATCTTAATCGTATAATAGCTTAATCCAGAGTAACCTCCTATTAGAAATCCGCTGCTCTAGTCCCCTG
AGCTACGGGCGCAGCACGTAGTTTTGTAGCAGAAAATTGTCAGAGAGGCAGGGATTATGGTGGAGTAAGAAGGGGGTTAAGGGA
TGATGGGATTAAGGGGAAATCTGAAAAAGGGGTTAAGGGATTCAGGGGTTAAGGAATTAAGGAAGAGACAAATCGATGACTGAC
AAGACGTTTAAGGTTTTTTAGGACGGAGGAGGAGGCCGAGGTCGGGGAGCTTGAGCAGTTTGGCGACAAGGAAGTAGGCGAGTG
ACCCGAGAAGAACGAGCGCAAAGGTTCCACCGATACGAACAGGGATGGGTTGGCCCATGGGTTGAATGAATTGGAGGCCTATAA
GTAGGGCTCCAATTAAGGTGACGCAGCAGAGAAGTATCTTTCCGACTCCTTGGAGGAAGTGAGGTTCGAAGTAGCTGCCTACGT
GCCGTTGGGAGGCCCACACCAGCTGCAGAATATTCAGCGTCGCCACAGAGGCTGTGGTGAGAGTGAGTCCTAAAATGCCGAGGT
TGTAGACCCGAATAAGAATGTAGTTGAAGGTGAGGTTGAGAAAAATTCCCGTAAGGCTAACGCGAAGCGGAATCCCCGGGCGAT
CGATAGCCGAGAAGGCGGGGGCG (SEQ ID NO: 205)
>3300028553|Ga0247839_1034275_1|M
CCTCATAAAGAGTAAAAAGTGCTCCCCCGTCACAAAACATTAGTTTGGTGACTGGTTGTTTTTAACCACCAACTTCTCTGTCCA
ATTTATTGGGGTTTTGTGAATCCCTATTCCAAGGACCGTTTTTGTACTACCCCCTAATTTACAGTTATCCTTATTATGAACTGG
AATAGCTCCCTTAAAAGTCCGATTATAACTGCTTGCTTAATACGATGAATCACAAAAATACCGTTAGAGCTTCTGTTCGCGAAC
TTACCCGGCATGCATTGCAGAAACCCGGGGAGGAGAGTGAACCGGGAGAAACAACAAAGACTCTTAAGTTCCGTGTGGATGCCA
AGGGACATACCCGAACCCGACTCGACCAGGCAGCACTGAACATTGCTCGGAAAAGCATCTGGGTCAGTTCCCTTCCACCCCGTC
AGGCAAAGCAATCGGAAGGTGAAGACCGGCGTTCCACCGAGGAACCATGGCGCGTTTGGTACAAGAAGCAGATCATGGGAAAGT
GGTCTTAAAAAATCCCTTGCCACCCGGCTATCATAGGATCAAATTAACCCCTTAGTATTTTCTTTGCACAACGTCGACTGCCCT
GCCAAGGAGCGGCTGATAACCAACAAGTTCTTTTACATTTGAGGTGCAAACCGTTCTTGTGATTTTTTTATAAAAGGATGCACC
CGAAAGGGTTGGCTATACCCAAGGATTCCCAGCCACCCATCTGACGGACTGGTTTGCTGACCTCG (SEQ ID NO: 206)
>3300014839|Ga0182027_10014967_5|M
TGCCGGGCGGCGGGGAAAGCGTATGATTTGCGCCGTCACCAGAAAACAAACGAAGGCACAAAATGGGGCGGAAAAGAAAAGGCA
TCGGGCAGAAAGAGCAAGCCCAGCCGGATTTGGGTCCGGCCGGGCTTGTCCAAGAAACGCTTTTGAACAGGTGTATCCGCATGG
AGGATAAGTCCATATTCAGCTTCCCTTGCTTGACCACTTTACTTTACAACACTTCGAATTTATGAGCAAAGAATACCGACCGGA
TTCGATTCACGCCTCGCTTCGCCATCTGCTTGAACTTGGCTTGAGCAAAACCGGTTCGAGTGAACCGCAAAAGATCACGCGCAC
AGTTAAGTTCAAGGTACACACGCTTGCCCGACCGGAACTGGTGCAAGGCGCATTGAACATCGCCCGCAAGTTCCTTTTCGCCTT
GGGACGCGGGAAGAAATCCGGCGAGATGACCGAAGCGGAGCGGCGCAAAGTCAGGGGCGAGTGGGAAAAATGGTATAAGGAAAA
ACTCGGAACCGTTTGGAGAAAGTGATAAGTCTGGATTTTCGCCCATTGCGGGCCTGATATGGAAAACGATGATTTTCTGCCCAT
CTACTCGCTGGCGGAATGGCATTATTGCCCGCGCTCGGCGTTTCTCTCCTGGTTCGGCGCTGAACGCGAAGACCGCGTGACGCC
GGCTTACCAGAAGATGCGTGAAGTCCATGCCGTGAGCAACCATGTGGCCTTCCGCGGCGAACTCGAACGGGGCGGTTGGCTGGG
CGGAAAAAACGCGCTGCTCATTTACCCGCTTCACGATGACGACCTTGAAAATGTGGAGCGGTTCGGGGCGGTGCGCGTCTGGCA
GGCCACATTTGAACTTCTTTAAGAAAACGCTTGCGTCTGTCCTGCTAATGGCGTAAAAAGGCCCCGTTAGTATTTACTTTGATA
CGATCTGGCGTGCCTTTCCGCCCAACGGATTCGACACGACCCACGAACTTCAGCGTGTCTGAGGAAACCTCAAGCGGTGGATGA
GCCGCTGCTTGTCGCGATCCTTGATAAGTCAAACGGAGCGAGTCGGTTAGGTTTGGAAAAAGCCTTGAACCGTTCTCAGCGGTT
TCCTATGCGTAACTTGCCTAAAATCAACACGAGTTTGCGCGTTAGCCGCTGCGACAGCAGCGGCTCACGCGCAAACTCGTGTGA
TCCAAGGGCCGAAGA (SEQ ID NO: 207)
>3300018040|Ga0187862_10006434_12|P
GCGCATCGCCGAGTTCGGCTTCTTGGGCGTGCGCGTCATGACCTGAATGCACACCCCGCGTCGAAACGGCGAGCCTTCCAAGGC
TGGCGACTTGGACTTGTATTTAACTTTGCGTCGGCCCTGGCGGACCAGTTGATTGATAGTCGGCATTGCGTCTTAAAAATGGAA
CGGGGAATATAACAAACCAAATCCGGCTTGCAACAACTATTTCGCCCGATTTGCGGTCTTAACTCATCGTAGCGGCGGTCGGCA
GACCGCCGCATTTCTTCTGGAAATTGCGCTGTCCGGCACTTTGCCGGGAGCCGCTGCGCCCGTTTTGCAGAAGTCTCGTTTTAA
TCCTTTTGGAAAGCACCGCGAAGCCAGCTTCAACTACCGCTTGCCGCCGCCGGGACACGGGCGTAGCATCACTCCACAATCAGC
CATCAGGAAAATGGGTATGAAAATCTCTTTGGGGATTTCATCATGAAAGCAAAGTATCGGTCTAACTCAATTCATGCCTCGCTC
CGGCAGCTTTTGGAGCTTGGCTTAAGCAAGAGCAGTTCGGCAGAACCACAGAAAATCACGCGCACTGCCAAGTTCAAGATTAAT
ACCGACATCCGGCCGGATTTGGTTTTAAACATTGCCCGAAAGTTTTTGTTCCGTTCCGAACGAGGCAAAAAGACTGACGACGCA
GGCGAAGAAGGTGAGGCAAAGCGTGGTAAATACAAAGACGACTGGCAGACTTGGTATCGCCAAAAGTTGGAAAAGGTCTGGTGC
AAGTAATCTGACATCGCCCATTCGGGACCCGCGAGCATGACATGGAGACTGACGATTTCCTTCCTATCTACTCGCTCGCGGAGT
GGCATTATTGCCCGCGCTCGTCTTTTCTGTCGTGGTTTGGTGCAGAGCGTGAGGATCGCGTCACCCCGGCTTACCAGAAAATGC
GCGAAGTCCACGCGGTGAGCAAT (SEQ ID NO: 208)
>3300027720|Ga0209617_10001817_3|M
AAACCCAACGGCATAAAGAGGATCAGGAGTTTTGGGTGCCCTTATTATGAGATAAAGAATAAGAATACCCCATACGATATTCAT
CCATTTAAGCAGACGTTGCACTTGGGATATTCTGTCACCTGCGTTGACCATTTGGCAACGTGCCATTATCGATGACAAGGGAGA
GGTATACCCCCAAATGGATATGCCTGTTATAAAACATTAGTTTGGTGACTTGGCATTTTTAGCCACTTTTTCCCTGTCCAATTT
TAGGGGGTTTTGTGAATCCCTATTCCAAGGACTGTTTTTGTCCCACCTCCTAATTTACAGTTATCCTTATTATGAACTGGAATA
GCTCCGTTAAAGGTCCGATTATGACTTCTTGCTTAATACGATGAATCACAAAAGCACCGTCAAGGCTTCTGTTCGCCATCTTAC
CCGGCATGCATTGCAGAAACCCGGGGAGGAGAGTGATCCGGGAGAAACGACAAAGACTTTAAAGTTCCGGGTAGATGCCAAAGC
ACATACCCGAACCCGACTCGACCAGGCAGCACTGAACATTGCCCGCAAAAGCATCTGGGTCAGCTCCCTTCCACCCCGTCAGGC
AAAGCAATCGGAAGGTGAAGACCGGCGTTCCACCGAGGAACCATGGCGCGTTTGGTACAAAAAGCAGATCATGGAAAAGTGGTC
TTAAAAAATCCCTTGCCACCCGGCTATCATAGGATCATTTTAGTCTCTTAGTATTTTCTTTGCACAACGTCGACTGCCCTGCCA
AGGAGCGGCTGACAACCAACAAGTTCTTTTACATTTGAGGTGCAAACCGTTCTTGTGATTTTTTTACAAAAGGATGCACCCGAA
AGGGTTGGCCATACCC (SEQ ID NO: 209)
>OCZT010053476_2|M
CTGAAATTCCCGGCCATCAATCCCGCCAACGCCGCCGAGCGCGATGAATTTCGTCTGAATACCTTCCCACCGGACAACCACACC
GCGCCCGCGCCGCCGACTCCGCCCGCGACACCGAAGCCGTAAAATCAAATTTGGAAAACGCAAAGCGGCCTTTGGGAATGGAGG
CCGCTTTTTGTTTTGGATTGTTCCTGTTCACGAAATCCGCTATTCAGGCTACAAATCTAAAAGGGTAAATATGAGCAAAGAGCC
TCGGAAAAATTCGACTCACGCCTCGCTTCGCCAGTTGCTTGAACTTGGTTTGAGCAAAAGCAGTTCGGCAGAGCCACAGAAGAT
CACGCGCACGGTGAAGTTCAAGATTCACACGCTTGCACGACCGGAACTCGTTGGCGCGTTGAACATCGCCCGCAAATTCCTGTT
CGCCTCGGGACGCGGAAAAAAGTCCGGTGAAATGACCGAAGCCGAGCGGCGCAAAGTGCGGGGCGAGTGGGAAAAGTGGTATAA
GGAAAAACTTGCGACCGTCTGGAAAAAACAATGAAAGGCTTGCGTCAGTTCTGCGAAAGGCGTAAACAGGCGGCGTTAGTATTT
ACTTTGATACGATCTAGCGTGCCTTTCCGTCCAACGGATTCGACACGACCCGTGAACTTCAGCGTGCAAAAGGAAATCACAAGT
GGTGGTTGACCGCTGCCTGTAGCGATCCTTGAAAACTTAAACCGAGCGAATCAGTTAGGTTCGGAAAGAAGCCCGAACTGGTTT
CAAAGTCAATCGCGTCAATGATTTCCTGCAAACCATAAACGGGTCTAAACGGGTCAGTCCTAACTTT
(SEQ ID NO: 210)
>3300014720|Ga0172376_10007396_6|M
TGCCCCAGCCCCTAACTCAAGTTCTGTAAAAACATTATTCGAAAGAATAAATCCCCGATCAGAGGATTGCCCAACAATAGTGCC
TTGGTTGTTTATATCGTAACAGGCGCTCCACGTATGGGCGGAACTAAGCACCCCCACGGAACTCATGCTTTTATTTCTGTACAC
GAACCCCCTGTCACCAAGCGAACTTGAGGAGGCACCAACCACAACACCCGAGTCGTTAATGCTGTAGCTTCGACTGGAATTATT
CGTGTAGGTGTACTCGCCTCCGAACCACGATGTCCAAGTAACAACCTGAGGACCAAGAGTCCTAAGATTTACCAGCTTGTAAGT
CGGGGCTTGCGCATTTCCGTTTTCGCAAATTACGAGCGAAGCAATTAAAACGCAGAGCCACCGAACCATATGATTATTCAACAC
CTATTTTACTTTTTTTCAACTAGGGAAAACCGGCTTTTTAACTACTATCCCTTTTGTCCTAAGCATTAACTTTTGCCTCAGAAA
CTGTCCTGATCACTAAACTAATGGTTTGTGACCGGCTGTGTTTTGGGCGTTCCGACTCTGTGGGTTTCATTGGGGTTTTGTGAC
TGCCCCCTCCTAATACCGTATTTGTACCACCCCCTACCTTAGGGTTGGCCCTGTTTTGAACTGGAATACGTACCTTAATCGTCT
GATTATAACCCTTCGGTTAAAACGATGATTCATAAGAGTACCGTTAAAGCATCGGTTCGCCACCTGACCCGCCATGCGTTGGAC
AAACCCGGCGAGGGCGACCCGGGTGAAACAACAAAAACCCTAAAGTTCCGGATTGATGCCAAAGATCACACCCGCACCCGGCTC
GACCAGCAGGCAGCCCTAAACATTGCCCGAAAAAGCATCTGGGTCAGTTCCTTGCCGCACCGGCAGGCAAAACAGTCGGAGGGA
GAAGACCGGCGTTCCACTGAGGAGCCGTGGCGGGAGTGGTACAAGAAGCGGATCTGGGAGAAGTGGGCTTAAAAAAAAGCCCTT
GCCACCCGGTCCCCGCAGGATCATTTTAGTCTCTTAGTATTTTCTTTGCACAGCGTTGACTCCCCTGCCAAGGGTGCGGCCAAC
AATCCAACAAGTTCTTTCGCAACAGGGGGCAAACCCCGTTCAATTTTGCTTTAAGGAACTAAAGCCCATGGCGTTAGGTATAGC
T (SEQ ID NO: 211)
>3300014720|Ga0172376_10013958_4|M
AAATACGGTTCCGTCAAATAAGTTGCCTGAAGAGTCACTGGCGTTCCCTGAAAATTCGTATTTTCCAACCAAGCCATAATTGAG
GTTAACCACGGCAGACTTCGCTGTTGAACCCATCAACAAGATGGCTAATGCGACCACCCGATTCATATATTAATTAAACCATGT
TCTTTTGTTTTTTCAATGTCCCACAGGCTCTTTTTTTGACTAAATAAAATATATCCCTTTTGGTATATCCTTGGTTGGTTTCGT
ATTATCGCATAAAACAAATGGTTTGTGACCGGTCATGTTTTGAGCGTTCCATCTCTGTAGGTTTCATTGGGGTTTTGTGATTAC
CCCCTCCTAATACCGTATTTGTACCACCCCCTACCTTAGGGTTAGCCCTGTTTTGAACTGGAATACGTACCTTAATCGTCTGAT
TATAACCCTTCGGTTAAAACGATGATTCATAAGAGTACTGTTAAAGCATCCGTTCGCCACCTGACCCGCCATGCTCTGGACAAA
CCCGGCGAGGGCGATCCGGGTAAACCAACAACAAAAACCCTGAAGTTCCGGATTGATGCCAAGGGGCACACCCGCACCCGGCTC
GACCAAGCCGCTTTAAATATAGCTCGCAAGAGTATCTGGCTGAGTACTTTGCCCAAAAGATCCAAAAAGGAAACACTGGCTGAA
GCACGTGGGTCCACAAAACCAGCGTGGAAAAAGTGGTATCAAAATCGTATCAGCGAGAATTGGTGTTAAAAATACTTGTCTTAC
ACTCGAAAATATTATAAAAAGCAGTCTTAGTATTTTCTTAGCAACACGTTGTCTGTCCTCGCCAAGGAACGGTCAACAATCCAA
CAAGTTCTTTCGCAACGGGGGGGCAAACCAGAAAATTTTCCCTTAAATTTGAATCTTAGGACTTTAGATACTGCCATGCGCGGA
TACTACAAAACCAAATGCATATTTTTCGTA (SEQ ID NO: 212)
>3300013087|Ga0163212_1009539_3|M
GGCAGGGTCAAAGTCATCATGGCTTGGAAGTTGGACAGAATCGGCCGCTCGACCCAGCACTTGGCCGGAATCATCAATCGACTC
CTTAAGGCGGATGTTGGCCTCGTGGTTCCCTCTCAGGGTATTGATACCTCGGCCGGCAGCATGAATCCTGCCAGTAAGCTCCAG
TTGAACGTTCTGGCCGCAGTCGCTGAGTTTGAGAGGGACCTTATCCGCGAGAGGACGAAGGCCGGCATGAAGGCAGCTAAGGCC
AGAGGCGTTAAATTCGGCCGTGAAACGGTGGTAACCGAAGCCCAGCGAATCCTAGCCAAAGAGATTCTTCAGCTTAATCCGGAT
ACTTCGGTGACCAGCCTGTCAAAGCGGTTGGGCTTGTCTTTGGGAACGGCGAGCAGACTTAAGACGATTTTGGGAGGTAGCTGC
TAGTACCGCCAATGTCCTATCCACCCGCTTACGATTGACCCAGTTTTGAACTGGAATACGCACCTTAATCGTCTGATTATAACC
CTTCGGTTAACACGATGATTCATAAGAGTACCGTTAAAGCATCGGTTCGCCACCTGACCCGCCATGCTCTGGACAAACCCGGCG
AGGGCGATCCGGGTGAAACAACAAAAACCCTGAAGTTCCGCATTGATGCCAAAGGTCACACCCGCACCCGGCTCGATCAGCAGG
CCGCCCTGAACATCGCTCGTAAAAGCATCTGGGTCAGATCATTGCCGCCCCGACAGGCAAAGCAGTCGGAGGGCGAGGATCGGC
GTTCCACTGAGGAGCCATGGAGGGAGTGGTATAAGAAGCAGATCCGGGAGAAGTGGGCTTAAAAAAACCCTTGCCACCCGGTCC
CCCTAGGATCATCTTGGTCCCTTAGTATTTTCTTTGTACAGCGTTGACTCCCCTGCCAAGGGTGCGGCCAACAACCAACAAGTT
TTTTCTCAACGAGGGGTGTTATAACCCTGAAATTTCGACAGGGTGCTGAACTGTAGGGTTTTGGCGATAAC
(SEQ ID NO: 213)
>3300014200|Ga0181526_10000193_31|M
CACCACGCCGTCTTCGATGGCATTGAACAACGTCTCCAGAAACTTCCGTTCCCGGGCCAATCGTTGGACAACCGTTTGCAACCC
TTCGGCATCCAGGCGTCCGATCCTGCCCAGGACTTTATCGAGAAAAGTCGATATTACCGCAGCCATAATGATGATACCCGCCAA
TGCGCAACCACCGCGCCAATTTGACGCACCCAAAGTATTCAAGCAATTCTGATTAGGCAAGCATTGCTTTCAATTTGAAAGGCC
GGAAAAAACCGTGAAAAATTCGGGGTTTTTCAATTAAACAAGAAAGTTGTCAAGGGGAAAATTGCGCAAAAAAAACAGTCAAAA
TTTCGGCATTTTTGACCTAAGCAATTGCAAACCAAACACTTACGAATTTCCACCTGCTTGCTGCCCATGACTGCAAAATGGTAG
CTTGGGACTGATGGACGAATGCGACTGATGGTCTGGCAACCAGCCAGTGGTCAAGGCGGGTTTCTGCCAGATGGACAGATAGGA
TTGTAATCATAAGACGATTCGGGCAAAACCAGGGCGGACAGGGAAAGCAGGAATTGGACGGTGGGGCGAGATTCCATCGAGCCG
TAAAATCGAGTCGGAGCACAGCCCGTCACGTTGCAGGATTAATGGTGAAAATGGGTCTTGGCAGGACGTTCTGTTTGGGCCATA
ATCTTGCCCGCTCCACAAAACGAAGGGAAAAAGAAGATGAGTGCGGAAACTTCAGAGGCTGGCAAAACCATGTGGGCTTTCGAC
TTGGGCACAGACTGCGAATTGCGCGTCGAATGCCGCAAACCATCGAATTGAAACTATTTATGGATTCAATCCACAAATCCGTTC
GTTCGCTGCTATCGCTTGGCCTGTCGCGTAGCGCTTCGGCAAGTCCGCAAACAATTACGCGCACAGTAAAATTCAAGATCAATA
CCGACATTCGGCAAGCCGCGCTCAATATCGCCAGAAAGCATCTGTTTCGCATTGGCCGCGAAAAAAAATCAGATGAGGAGAATG
AAGCCGCACGACGCAAAGTTATGGGTGAGTGGGAAACTTGGTATCGGCAGAAACTCGCCAGCGTTTGGAATAAATAATTCACCC
TTAGCCATTTTGACTGTGAGCGAGATATGGAGATCGACGATTTCCTTCCAATCTATTCGCTCGCGGAGTGGCATTATTGCCCAC
GCTCGTCTTTTCTGTCGTGGTTCGGCGCGGAACGTGAGGATCGCGTCACCCCGGCTTACCAGAAGATGCGTGAAGTCCATGCCG
TGAGCAATCATGTGGCGTTTCGGGGCGCATTGGAACGCGGCGGCTGGCTGGATGGCAAACACGCGCTGCTGGTTTATCCCTTGC
ATGATGACGACCTTGAAAATGTGGAACGTTACGGCGCGGTACGGGTGTGGCAGGCAACGTTTGAATTTCTTTGAATTTAGGCTT
GCAGTTGGATGGGAGCAGGCATAATACTTCTCCCGTTAGCTGTCTCCTCGGAGGCAGATTTACTTTGATTCTGTGCGCCTTTAC
GCCCCACGTATTTGACGCACTTCGCGAACTTCAGCGGTTAACGGAAACCGCCAGCGGTGGATGGACGATTCACCCGGCGTTCCT
TGACAACACAGTCCGTAGCAGTTGGTTATATACGAAACGGACTAAAAGCGGTTTGCCATGCGTAACTTGTTGCCAGACAATGGA
GTCATCATCTCGGCAGAAATTTTGCATTTAGGTCTTGATGCAAATATTTGGACGGCAGGGCGTGAAGACAGATCAGCATCGCGC
TTGGCCAGGCCC (SEQ ID NO: 214)
>3300019093|Ga0187843_10013171_2|M
CTCTCTGGATTGGGTTGGTCTTCGCCACGATGGTCTACTTTCACGGCAACGTGAAAGACTTGGAACCCAAACCCGGTTCGGTCA
CACCGATCCAGAAAACGGCCGCGGGGCTGATGTTTCTTGTCGATGCGATCATGCTGGGACTCGCTCTTTATTTGATCCTTCGCT
AACGCGAACCGATCGATAAGGATTAAGTCGCACTACCCCTGCATCGATTCCCGTTTTTTCGATTCCCTTCCAGGTATTTAATTT
TTACTTCTCAACGAGGTATTTGGCTTGGCATGGTCTTCTCTGCGCCCCCTGCCCCCTGTCCCCTGCCCCCTTTCACTTCCAGCC
TACCAATTCCCAGTGTTCAAAACGTCGGTGTTTTGGCGTTTATCCTTTCCTGAGATAACGCCCAGGACTGTTTTTGTCCCACCT
CCTAATTTACACTTATCCTTATTATGAACTGGAATAGCTCCGTTAAAAGTCCGATTATGACTGCTTGCTTAATACGATGAATCA
CAAAAGCACTGTCAAAGCCTCTGTTCGCCATCTTACTCGGCATGCATTGCAGAAACCCGGGGAGGAGAGTGATCCGGGAGAAAC
GACAAAGACTTTAAAGTTCCGGGTAGATGCCAAAGGACATACCCGAACCCGACTCGACACCCGGGGTGGTCGGGGAACATTGAT
GGGCAACATGCACTATGAGCGAATTTTGTCTGGGTTGAGGGCAAGTTGGCCGAGGCAGGTTGCTACAAGGCACCCAGTGCCGCC
AAGTACCGCAAAGGAATCACGCCGTTCTTGCGATTTGTAAATCCAGCATATACCAGTGCCACCTGTTCAGCTTGTGGTGAGGTG
TTCAGTTCTAGTTGGTATAAGAAAACCGCAGCCACAATCAGTAAGGAATCACCCGGTTCATGGTCAGTGACGCTTCCCAACGGA
AAAAAACTTCAGTTGCCATGCTCCTACACCTCATACATGCGTGGGCGCGGTGAAAAGACATGGAACACCGCTGAACGGCTGGAG
GAACTTTTCAAGGATCGAACATGGTCAGATCTTCCCCCCAGCACCCGAAAAACAGTAGAGGGATTGGTTGCTCGCCTGACTCAT
CATCGCCCCAAACGGGGTCAGGCAGCACTGAACATTGCCCGCAAAAGCATCTGGGTCAGCTCCCTTCCACCCCGTCAGGCAAAG
CAATCGGAAGGTGAAGACCGGCGTTCCACCGAGGAACCATGGCGCGTTTGGTACAAAAAGCAGATCATGGAAAAGTGGTCTTAA
AAAATCCCTTGCCACCCGGCTATCATAGGATCATTTTAGTCTCTTAGTATTTTCTTTGCACAACGTCGACTGCCCTGCCAAGGA
GCGGCTGACAACCAACAAGTTCTTTTACATTTGAGGTGCAAACCGTTCTTGTGATTTTTTTACAAAAGGATGCACCCGAAAGGG
TTGGCCATACCC (SEQ ID NO: 215)

### Example 2—Identification of Transactivating RNA Elements

In addition to an effector protein and a crRNA, some CRISPR systems described herein can also include an additional small RNA that activates robust enzymatic activity referred to as a transactivating RNA (tracrRNA). Such tracrRNAs typically include a complementary region that hybridizes to the crRNA. The crRNA-tracrRNA hybrid forms a complex with an effector resulting in the activation of programmable enzymatic activity.

TracrRNA sequences can be identified by searching genomic sequences flanking CRISPR arrays for short sequence motifs that are homologous to the direct repeat portion of the crRNA. Search methods include exact or degenerate sequence matching for the complete direct repeat (DR) or DR subsequences. For example, a DR of length n nucleotides can be decomposed into a set of overlapping 6-10 nt kmers. These kmers can be aligned to sequences flanking a CRISPR locus, and regions of homology with 1 or more kmer alignments can be identified as DR homology regions for experimental validation as tracrRNAs. Alternatively, RNA cofold free energy can be calculated for the complete DR or DR subsequences and short kmer sequences from the genomic sequence flanking the elements of a CRISPR system. Flanking sequence elements with low minimum free energy structures can be identified as DR homology regions for experimental validation as tracrRNAs.

TracrRNA elements frequently occur within close proximity to CRISPR associated genes or a CRISPR array. As an alternative to searching for DR homology regions to identify tracrRNA elements, non-coding sequences flanking CRISPR associated proteins or the CRISPR array can be isolated by cloning or gene synthesis for direct experimental validation of tracrRNAs.

Experimental validation of tracrRNA elements can be performed using small RNA sequencing of the host organism for a CRISPR system or synthetic sequences expressed heterologously in non-native species. Alignment of small RNA sequences from the originating genomic locus can be used to identify expressed RNA products containing DR homology regions and sterotyped processing typical of complete tracrRNA elements.

Complete tracrRNA candidates identified by RNA sequencing can be validated in vitro or in vivo by expressing the crRNA and effector in combination with or without the tracrRNA candidate, and monitoring the activation of effector enzymatic activity.

In engineered constructs, the expression of tracrRNAs can be driven bypromoters including, but not limited to U6, U1, and H1 promoters for expression in mammalian cells or J23119 promoter for expression in bacteria.

In some instances, a tracrRNA can be fused with a crRNA and expressed as a single guide RNA.

### Example 3—Identification of Novel RNA Modulators of Enzymatic Activity

In addition to the effector protein and the crRNA, some CRISPR systems described herein can also include an additional small RNA to activate or modulate the effector activity, referred to herein as an RNA modulator.

RNA modulators are expected to occur within close proximity to CRISPR-associated genes or a CRISPR array. To identify and validate RNA modulators, non-coding sequences flanking CRISPR-associated proteins or the CRISPR array can be isolated by cloning or gene synthesis for direct experimental validation.

Experimental validation of RNA modulators can be performed using small RNA sequencing of the host organism for a CRISPR system or synthetic sequences expressed heterologously in non-native species. Alignment of small RNA sequences to the originating genomic locus can be used to identify expressed RNA products containing DR homology regions and sterotyped processing.

Candidate RNA modulators identified by RNA sequencing can be validated in vitro or in vivo by expressing a crRNA and an effector in combination with or without the candidate RNA modulator and monitoring alterations in effector enzymatic activity.

In engineered constructs, RNA modulators can be driven by promoters including U6, U1, and H1 promoters for expression in mammalian cells, or J23119 promoter for expression in bacteria.

In some instances, the RNA modulators can be artificially fused with either a crRNA, a tracrRNA, or both and expressed as a single RNA element.

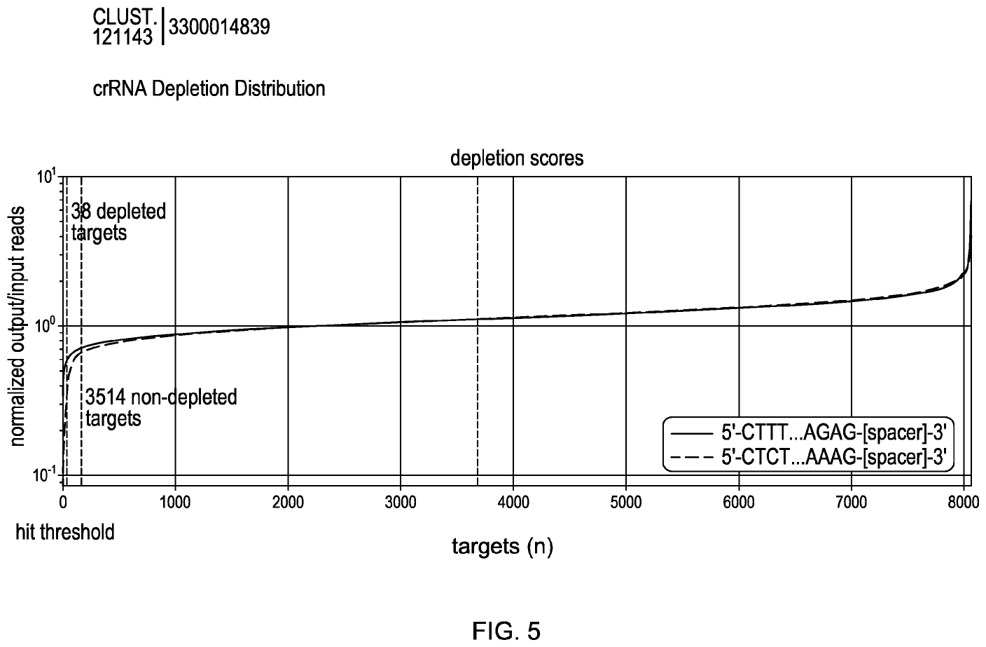

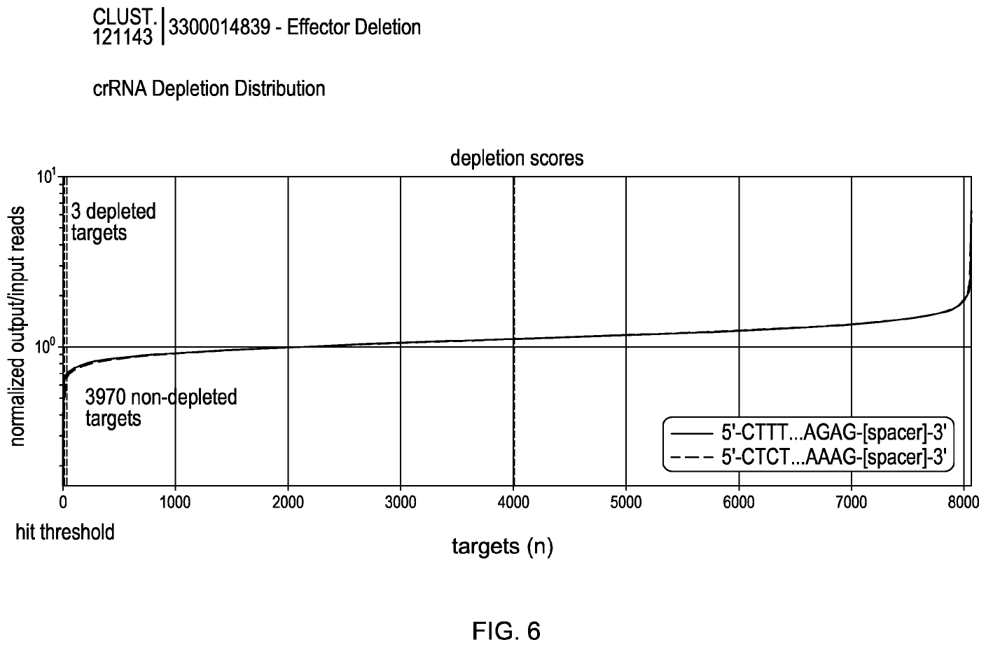

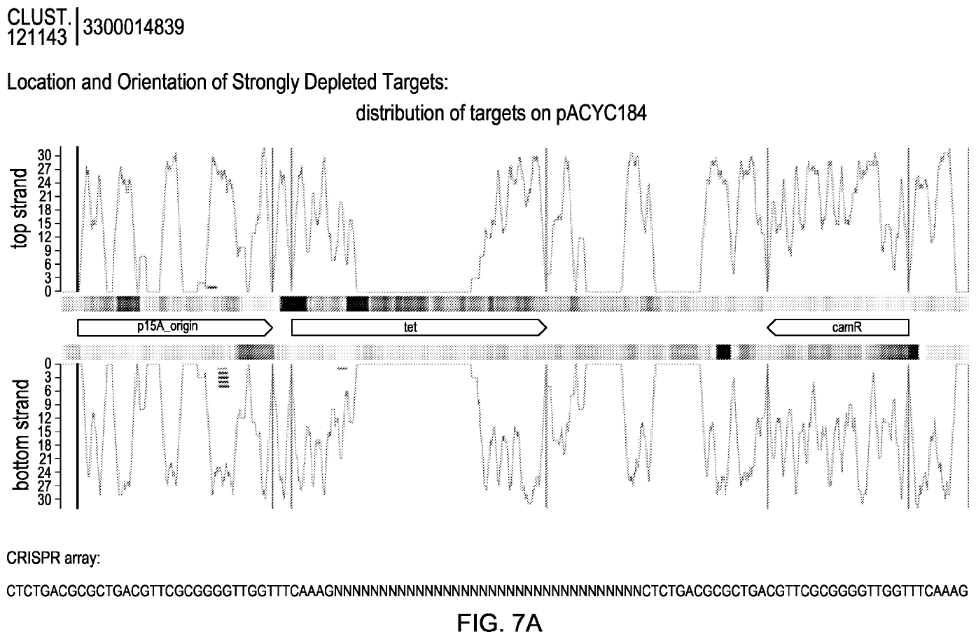

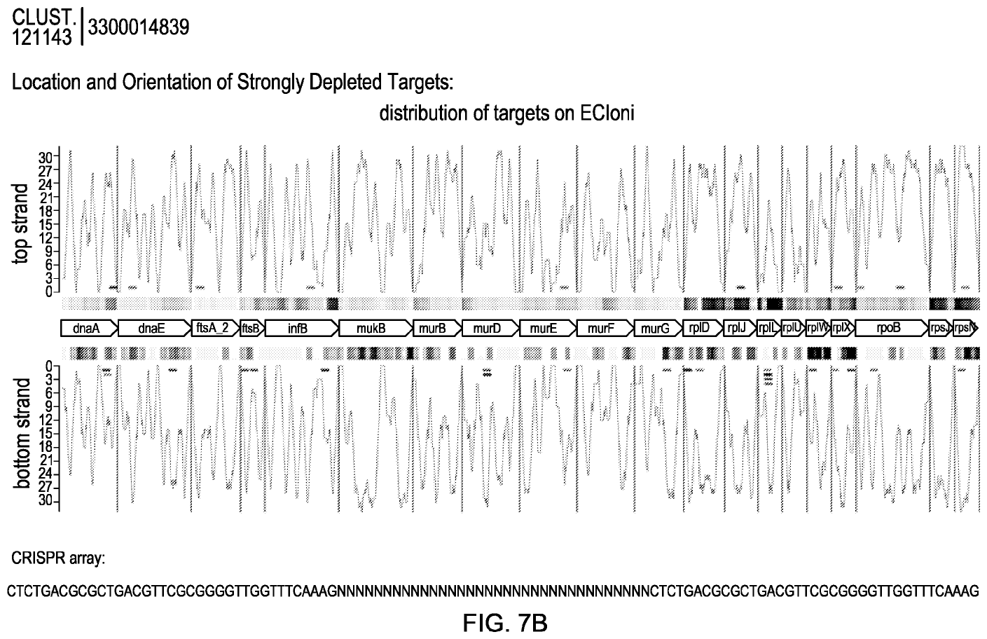

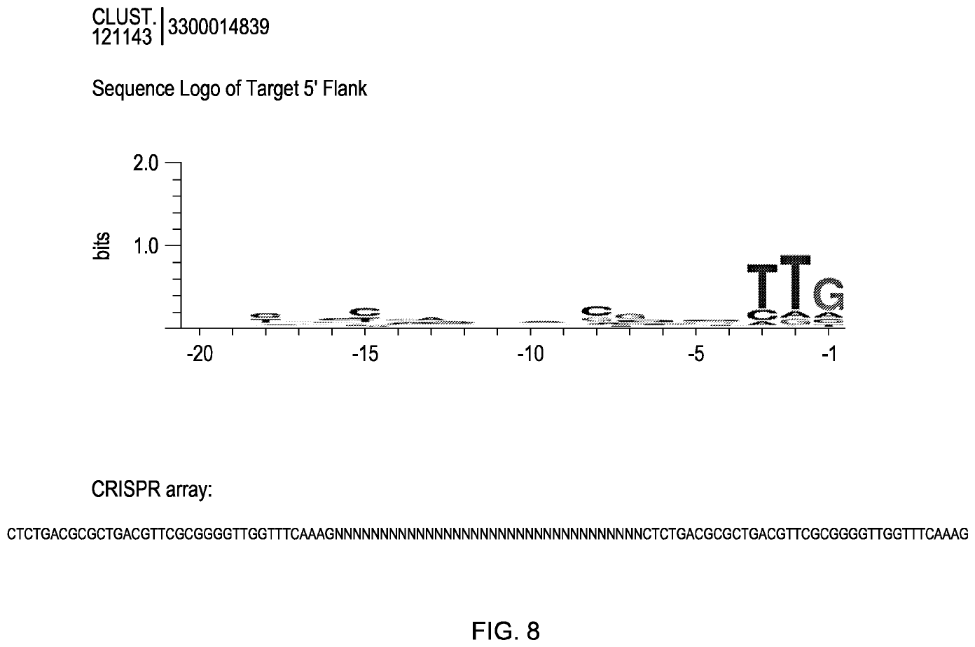

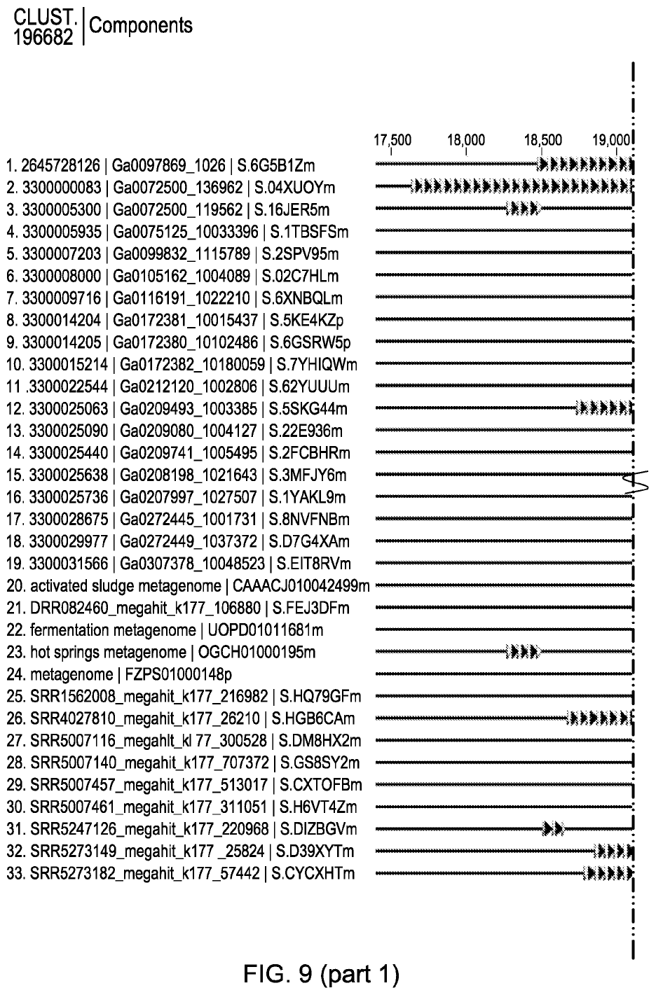

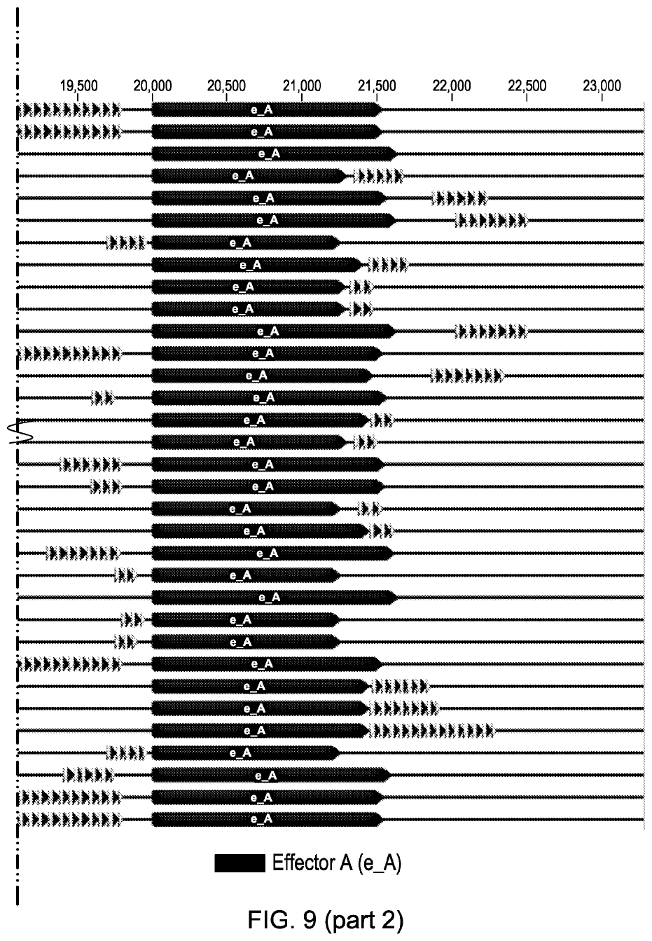

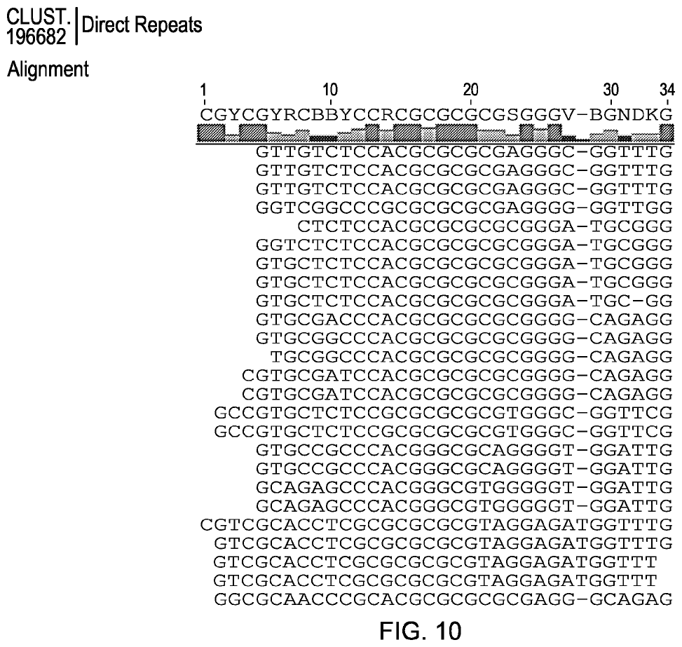

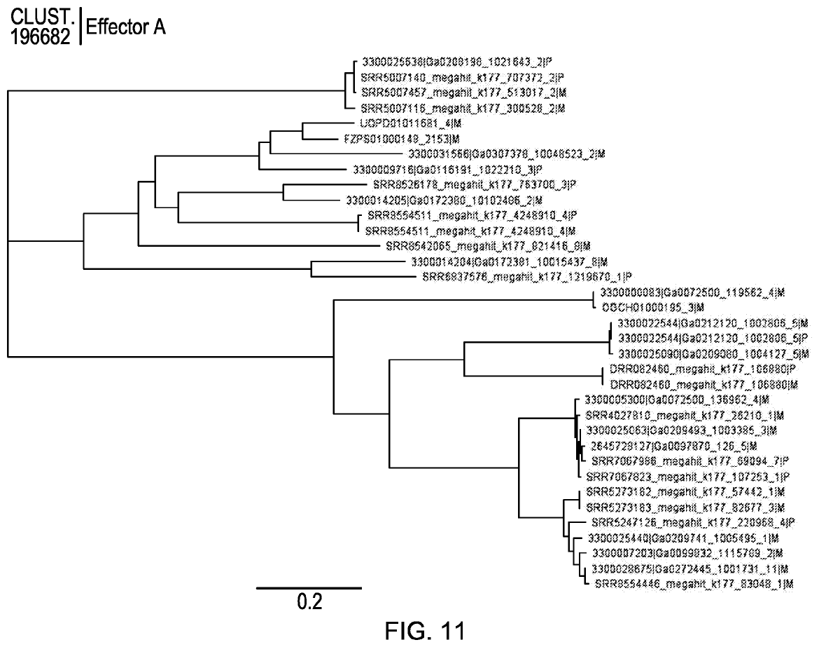

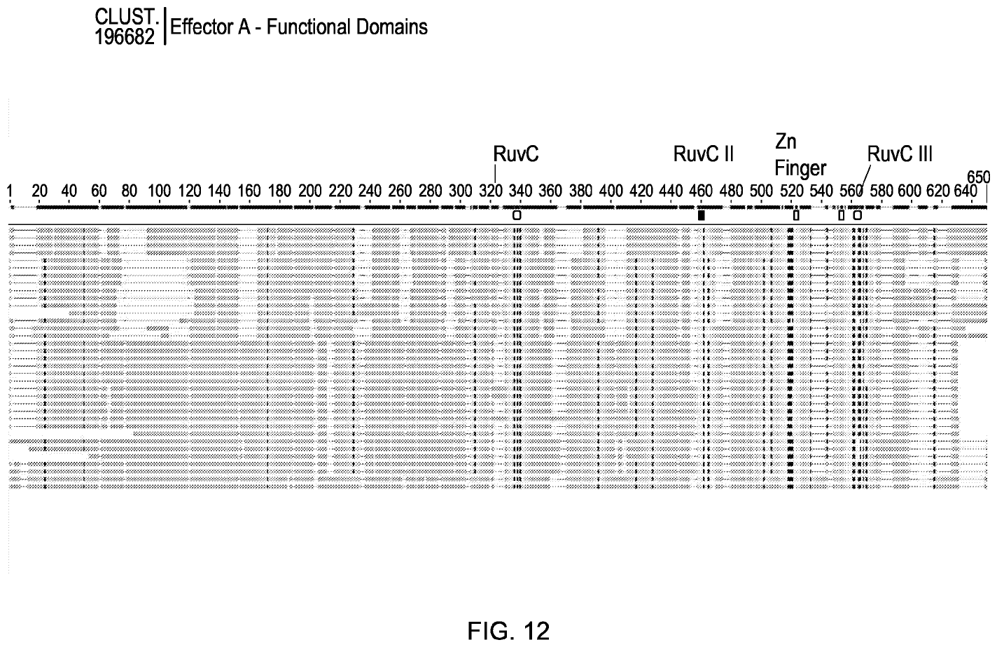

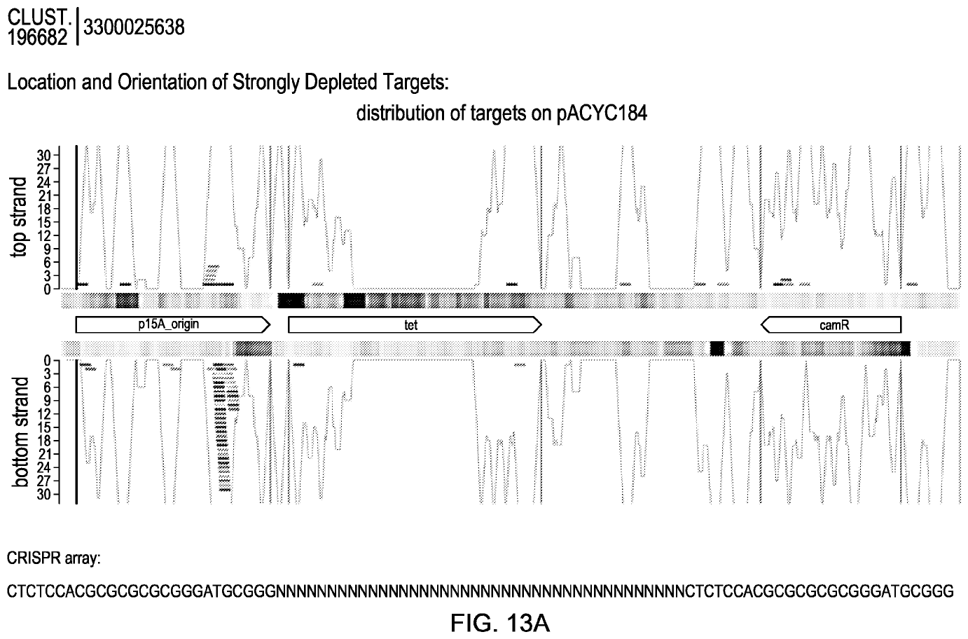

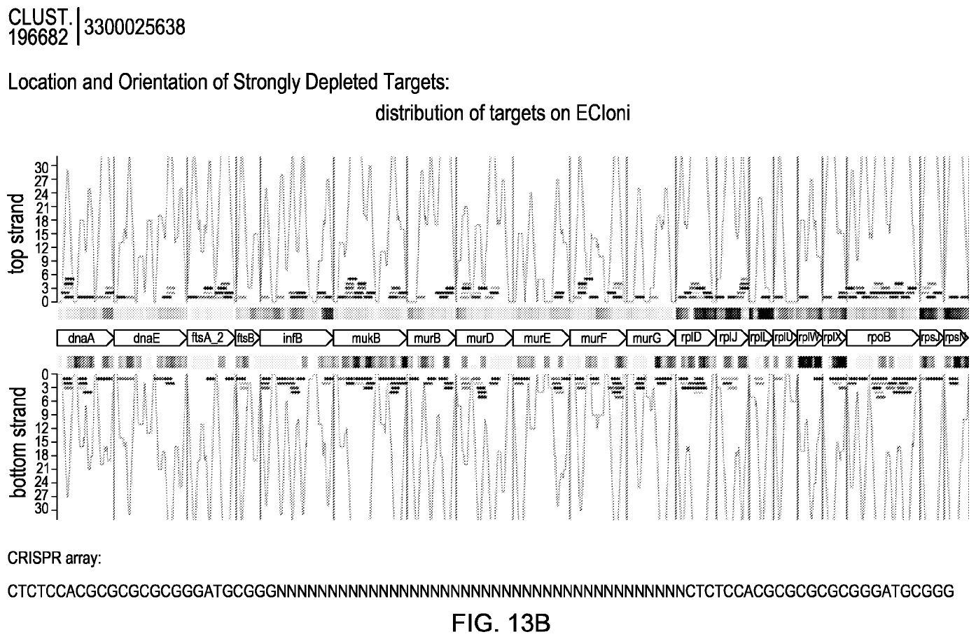

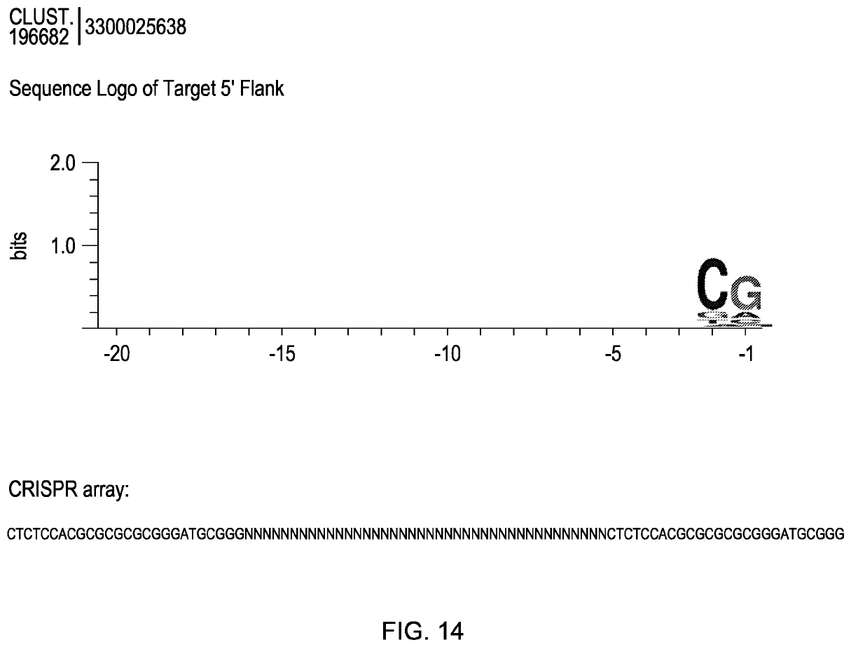

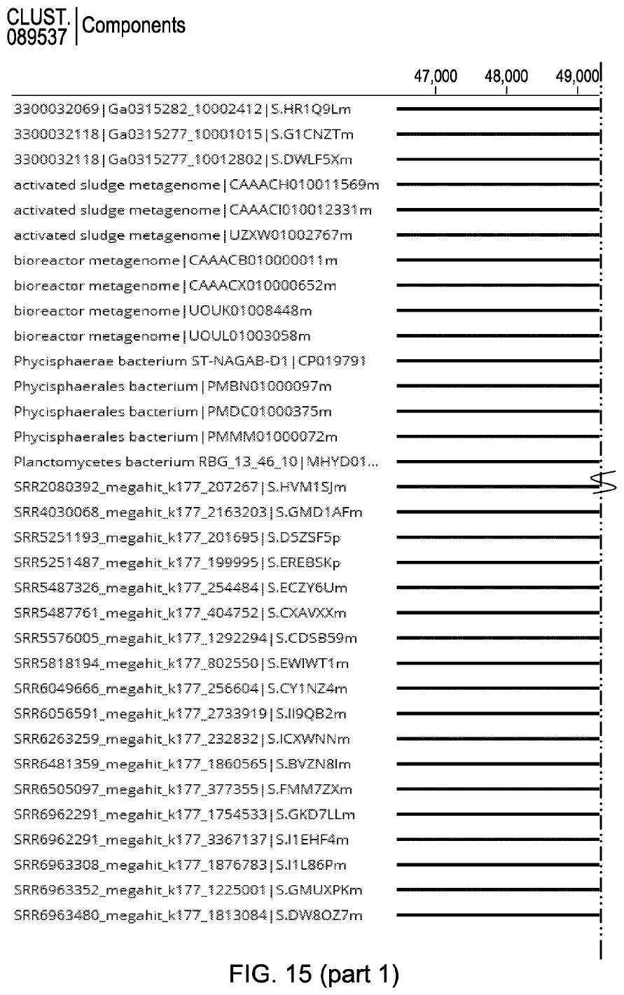

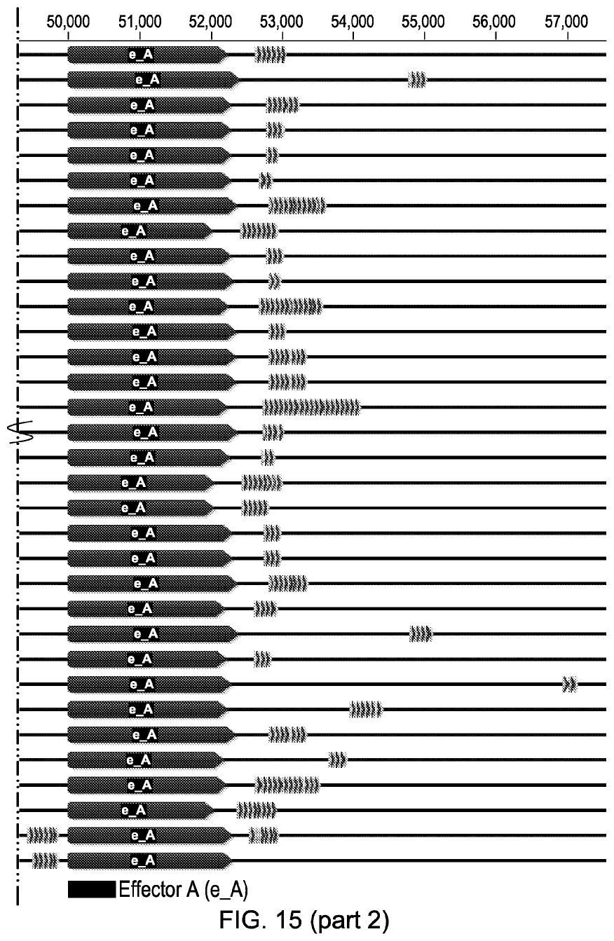

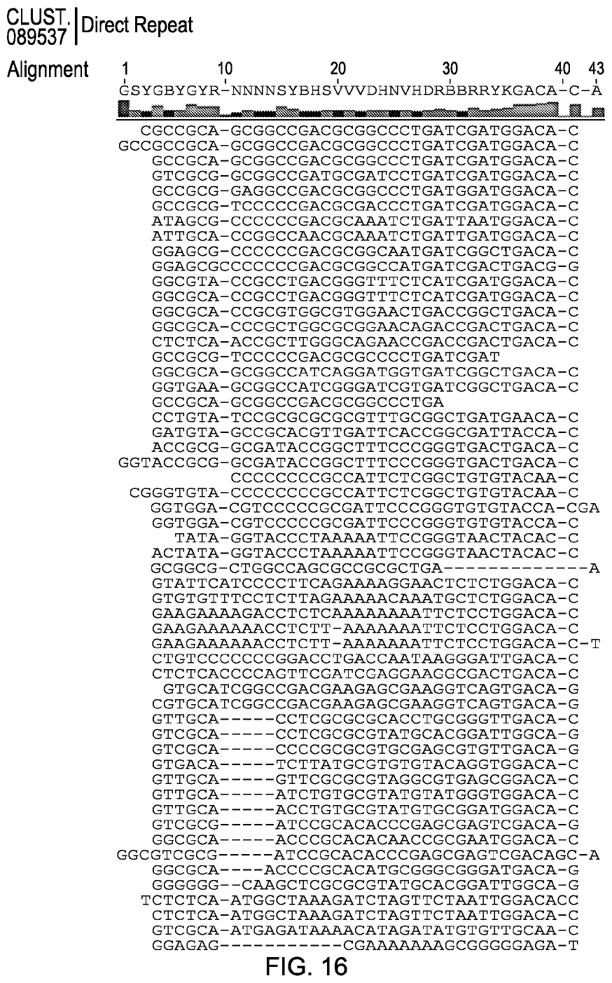

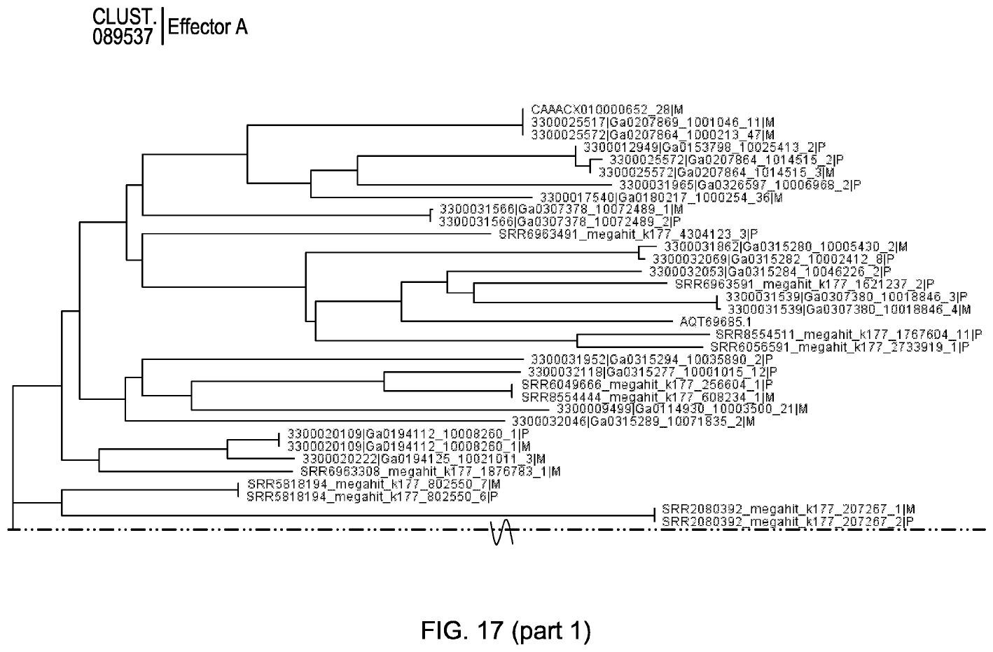

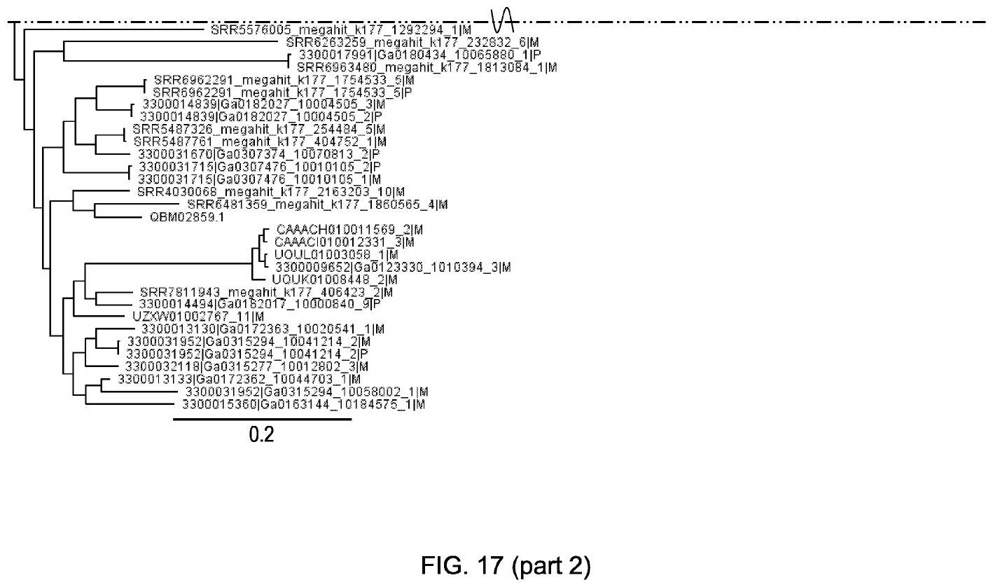

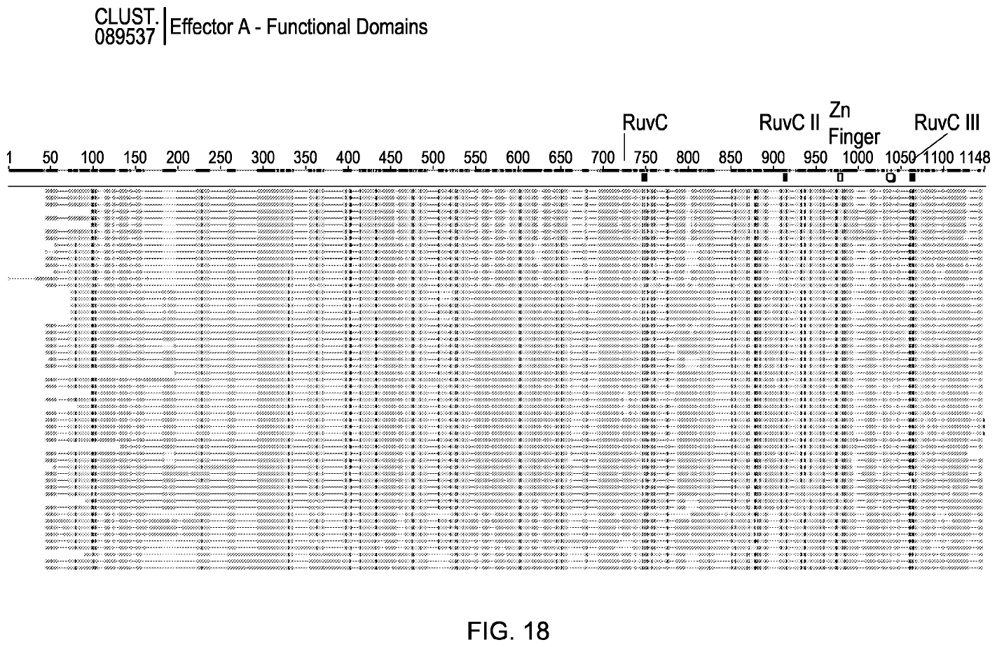

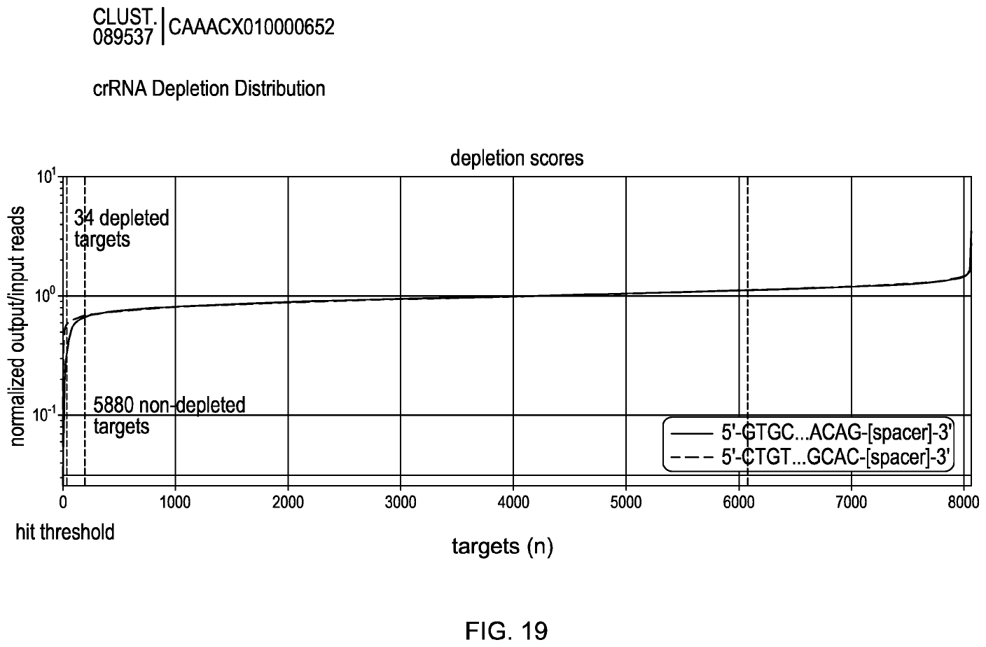

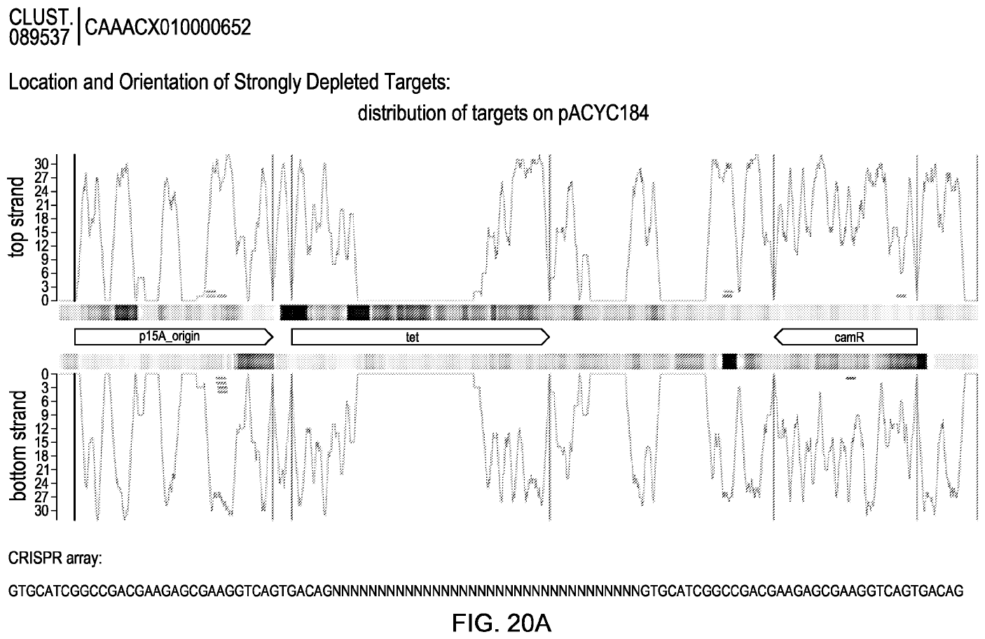

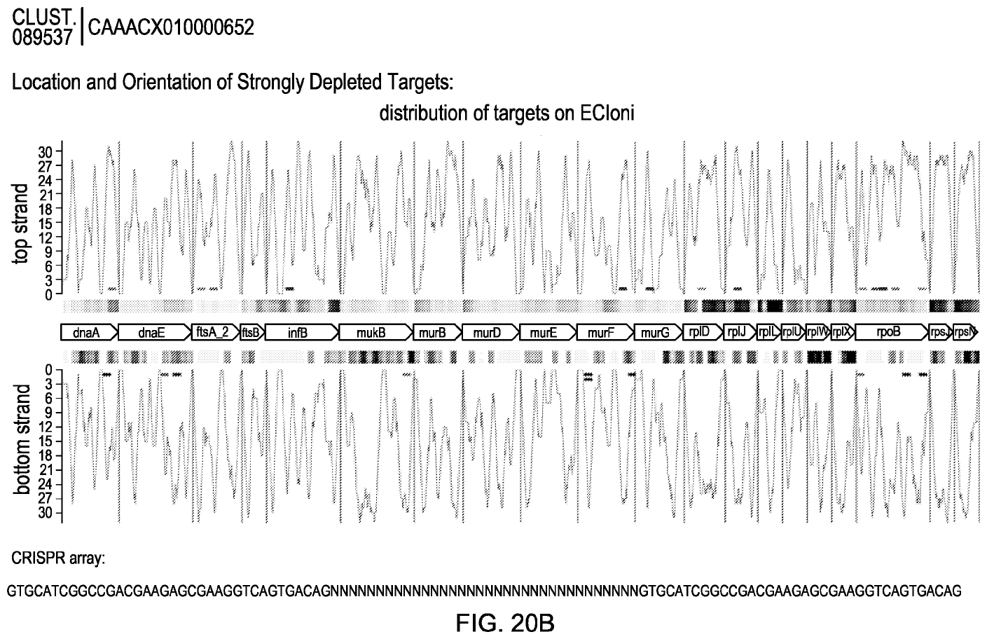

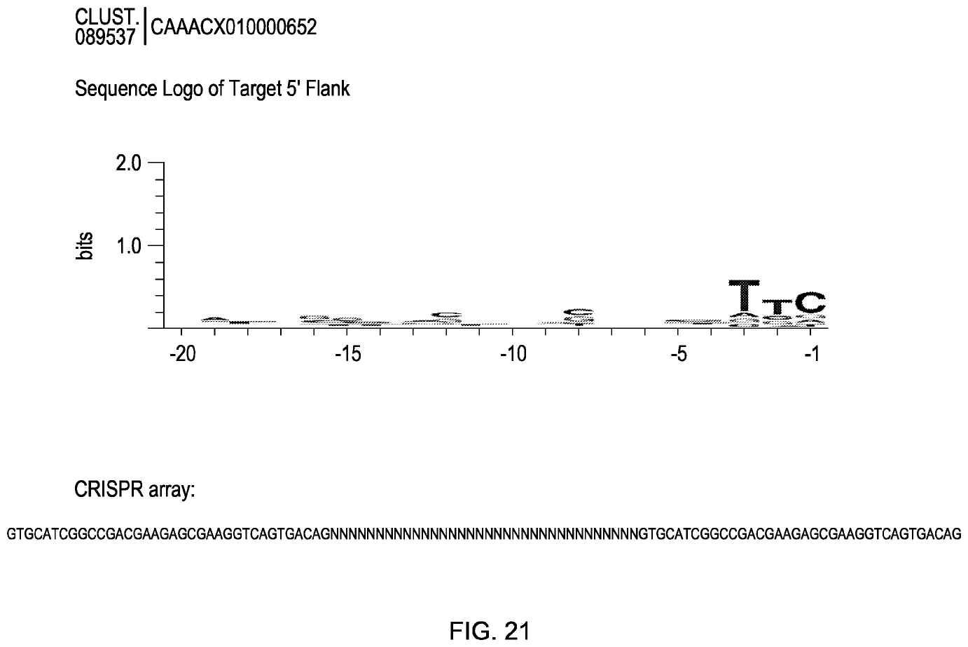

### Example 4—Functional Validation of Engineered CLUST.121143 CRISPR-Cas Systems (FIGS. 5-8)

Having identified the minimal components of CLUST.121143 CRISPR-Cas systems, we selected one loci for functional validation, from the metagenomic source designated 3300014839.

### DNA Synthesis and Effector Library Cloning

To test the activity of an exemplary CLUST.121143 CRISPR-Cas system, we designed and synthesized systems containing the pET28a(+) vector. Briefly, E. coli codon-optimized nucleic acid sequences encoding the CLUST.121143 3300014839 effector (amino acid sequence provided in Table 2) were synthesized (Genscript) and cloned into a custom expression system derived from the pET-28a(+) (EMD-Millipore). The vector included a nucleic acid encoding CLUST.121143 3300014839 effector protein under the control of a lac promoter and an E. coli ribosome binding sequence. The vector also included an acceptor site for a CRISPR array library driven by a J23119 promoter following the open reading frame for the CLUST.121143 3300014839 effector.

We computationally designed an oligonucleotide library synthesis (OLS) pool containing “repeat-spacer-repeat” sequences, where “repeat” represents the consensus direct repeat sequence found in the CRISPR array associated with the effector, and “spacer” represents sequences tiling the pACYC184 plasmid as well as E. coli essential genes. The spacer length was determined by the mode of the spacer lengths found in the endogenous CRISPR array. The repeat-spacer-repeat sequence was appended with restriction sites enabling the bi-directional cloning of the fragment into the aforementioned CRISPR array library acceptor site, as well as unique PCR priming sites to enable specific amplification of a specific repeat-spacer-repeat library from a larger pool.

We next cloned the repeat-spacer-repeat library into the plasmid using the Golden Gate assembly method. Briefly, we first amplified each repeat-spacer-repeat from the OLS pool (Agilent Genomics) using unique PCR primers, and pre-linearized the plasmid backbone using BsaI to reduce potential background. Both DNA fragments were purified with Ampure XP (Beckman Coulter) prior to addition to Golden Gate Assembly Master Mix (New England Biolabs) and incubated per the manufacturer's instructions. We further purified and concentrated the Golden Gate reaction to enable maximum transformation efficiency in the subsequent steps of the bacterial screen.

The plasmid library containing the distinct repeat-spacer-repeat elements and Cas proteins was electroporated into E. Cloni electrocompetent E. coli (Lucigen) using a Gene Pulser Xcell® (Bio-rad) following the protocol recommended by Lucigen. The library was either co-transformed with purified pACYC184 plasmid, or directly transformed into pACYC184-containing E. Cloni electrocompetent E. coli (Lucigen), plated onto agar containing chloramphenicol (Fisher), tetracycline (Alfa Aesar), and kanamycin (Alfa Aesar) in BioAssay® dishes (Thermo Fisher), and incubated for 10-12 hours at 37° C. After estimation of approximate colony count to ensure sufficient library representation on the bacterial plate, the bacteria were harvested and plasmid DNA extracted using a QIAprep Spin Miniprep® Kit (Qiagen) to create an “output library.” By performing a PCR using custom primers containing barcodes and sites compatible with Illumina sequencing chemistry, we generated a barcoded next generation sequencing library from both the pre-transformation “input library” and the post-harvest “output library,” which were then pooled and loaded onto a Nextseq 550 (Illumina) to evaluate the effectors. At least two independent biological replicates were performed for each screen to ensure consistency.

### Bacterial Screen Sequencing Analysis

Next generation sequencing data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source (pACYC184 or E. Cloni) or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

To identify specific parameters resulting in enzymatic activity and bacterial cell death, we used next generation sequencing (NGS) to quantify and compare the representation of individual CRISPR arrays (i.e., repeat-spacer-repeat) in the PCR product of the input and output plasmid libraries. We defined the array depletion ratio as the normalized output read count divided by the normalized input read count. An array was considered to be “strongly depleted” if the depletion ratio was less than 0.1 (more than 10-fold depletion). When calculating the array depletion ratio across biological replicates, we took the maximum depletion ratio value for a given CRISPR array across all experiments (i.e. a strongly depleted array must be strongly depleted in all biological replicates). We generated a matrix including array depletion ratios and the following features for each spacer target: target strand, transcript targeting, ORI targeting, target sequence motifs, flanking sequence motifs, and target secondary structure. We investigated the degree to which different features in this matrix explained target depletion for CLUST.121143 systems, thereby yielding a broad survey of functional parameters within a single screen.

FIG. 5 shows the degree of interference activity of the engineered compositions by plotting for a given target the normalized ratio of sequencing reads in the screen output versus the screen input. The results are plotted for each DR transcriptional orientation. In the functional screen for each composition, an active effector complexed with an active RNA guide will interfere with the ability of the pACYC184 to confer E. coli resistance to chloramphenicol and tetracycline, resulting in cell death and depletion of the spacer element within the pool. Comparing the results of deep sequencing the initial DNA library (screen input) versus the surviving transformed E. coli (screen output) suggest specific target sequences and DR transcriptional orientation that enable an active, programmable CRISPR system. The screen also indicates that the effector complex is only active with one orientation of the DR.

FIG. 6 shows the lack of interference activity of the negative control, where the coding region for the CLUST.121143 effector protein has been deleted from the pET-28a(+)-derived plasmid prior to electroporation into E. coli.

FIGS. 7A-B depict the location of strongly depleted targets for CLUST.121143 3300014839 effector targeting pACYC184 and E. coli E. Cloni essential genes. Notably, the location of strongly depleted targets appears dispersed throughout the potential target space.

FIG. 8 depicts a weblogo of the sequences flanking depleted target, indicating a prominent 5′ PAM of TTG.

### Example 5—Identification of Minimal Components for CLUST.196682 CRISPR-Cas System (FIGS. 9-12)

This protein family describes a large single effector associated with CRISPR systems found in Fervidibacteria, Haloarcula, Halohasta, Halorubrum, Natronoccus species and uncultured metagenomic sequences collected from freshwater, hot springs, salt lake, sediment, soil, and wastewater environments (TABLE 7). CLUST.196682 effectors include the examples of proteins detailed in TABLES 7 and 8, below. Examples of direct repeat sequences for these systems are shown in TABLE 9.
Examples of naturally occurring loci containing this effector complex are depicted in FIG. 9, indicating that for CLUST.196682 loci, the effector protein co-occurs with a CRISPR array. No other families of large proteins were identified within a bi-directional 15 kb window that co-occur with the effector protein or a CRISPR array.
The direct repeat sequences of CLUST.196682 exhibit multiple conserved motifs, including 5′-GYYYGNKRKNNAC-3′ (SEQ ID NO: 832) proximal to the 5′ end, or a central 5′-YCCRCGCGCGCGNGGG-3′ (SEQ ID NO: 833), where Y refers to C or T or U, K refers to G or T or U, R refers to A or G, and N refers to any nucleobase (FIG. 10).
FIG. 11 is a schematic representation of a phylogenetic tree of example CLUST.196682 effectors, showing that the family exhibits sequence diversity.
FIG. 12 is a schematic representation of a multiple sequence alignment of CLUST.196682 effector proteins revealing the location of the conserved catalytic residues of the RuvC domain, indicated by the colors Magenta, Blue, and Red.
Optionally, the system includes a tracrRNA that is a subset of a non-coding sequence listed in TABLE 10.

TABLE 7
Representative CLUST.196682 Effector Proteins

| | | # | | | effector |
|---|---|---|---|---|---|
| Species | effector accession | spacers | casl | cas2 | size |
| wastewater-anaerobic digestor- | 3300025638\|Ga0208198_1021643_2\|P | 2 | N | N | 481 |
| anaerobic digestor sludge | | | | | |
| (3300025638\|Ga0208198_1021643) | | | | | |
| aquatic-thermal springs-hot spring | 3300005300\|Ga0072500_136962_4\|M | 33 | N | N | 512 |
| (3300005300\|Ga0072500_136962) | | | | | |
| activated sludge metagenome | CAAACJ010042499_2\|P | 2 | N | N | 481 |
| (CAAACJ010042499) | | | | | |
| activated sludge metagenome | SRR5007116_megahit_k177_300528_2\|M | 6 | N | N | 481 |
| (SRR5007116) | | | | | |
| activated sludge metagenome | SRR5007140_megahit_k177_707372_2\|P | 7 | N | N | 481 |
| (SRR5007140) | | | | | |
| activated sludge metagenome | SRR5007457_megahit_k177_513017_2\|M | 13 | N | N | 481 |
| (SRR5007457) | | | | | |
| activated sludge metagenome | SRR5007461_megahit_k177_311051_7\|P | 4 | N | N | 419 |
| (SRR5007461) | | | | | |
| aquatic-freshwater-freshwater lake | 3300027896\|Ga0209777_10000273_30\|P | 2 | N | N | 341 |
| sediment | | | | | |
| (3300027896\|Ga0209777_10000273) | | | | | |
| aquatic-freshwater-freshwater lake | 3300027896\|Ga0209777_10000273_32\|M | 2 | N | N | 353 |
| sediment | | | | | |
| (3300027896\|Ga0209777_10000273) | | | | | |
| aquatic-freshwater-sediment | 3300013127\|Ga0172365_10030480_1\|M | 3 | N | N | 373 |
| (3300013127\|Ga0172365_10030480) | | | | | |
| aquatic-freshwater-sediment | 3300013128\|Ga0172366_10002984_1\|M | 3 | N | N | 373 |
| (3300013128\|Ga0172366_10002984) | | | | | |
| aquatic-freshwater-sediment | 3300013129\|Ga0172364_10030775_1\|P | 3 | N | N | 373 |
| (3300013129\|Ga0172364_10030775) | | | | | |
| aquatic-freshwater-sediment | 3300013130\|Ga0172363_10005240_1\|P | 3 | N | N | 373 |
| (3300013130\|Ga0172363_10005240) | | | | | |
| aquatic-freshwater-sediment | 3300013133\|Ga0172362_10039269_3\|M | 3 | N | N | 373 |
| (3300013133\|Ga0172362_10039269) | | | | | |
| aquatic-freshwater-sediment | 3300032070\|Ga0315279_10000130_51\|M | 8 | Y | N | 342 |
| (3300032070\|Ga0315279_10000130) | | | | | |
| aquatic-freshwater-sediment | 3300032118\|Ga0315277_10001250_38\|M | 2 | N | N | 368 |
| (3300032118\|Ga0315277_10001250) | | | | | |
| aquatic-marine-deep subsurface | 3300024429\|Ga0209991_10029076_1\|P | 2 | N | N | 343 |
| (3300024429\|Ga0209991_10029076) | | | | | |
| aquatic-marine-marine sediment | 3300014886\|Ga0180300_10007429_2\|M | 2 | Y | Y | 365 |
| (3300014886\|Ga0180300_10007429) | | | | | |
| aquatic-non marine saline and | SEJ25361.1 | 2 | N | N | 382 |
| alkaline-hypersaline | | | | | |
| (2140918017\|Ga0065697_1000127) | | | | | |
| aquatic-non marine saline and | 3300005935\|Ga0075125_10033396_1\|P | 5 | N | N | 432 |
| alkaline-saline lake | | | | | |
| (3300005935\|Ga0075125_10033396) | | | | | |
| aquatic-non marine saline and | 3300025736\|Ga0207997_1027507_2\|M | 2 | N | N | 432 |
| alkaline-saline lake | | | | | |
| (3300025736\|Ga0207997_1027507) | | | | | |
| aquatic-thermal springs | 3300007203\|Ga0099832_1115789_2\|M | 5 | N | N | 521 |
| (3300007203\|Ga0099832_1115789) | | | | | |
| aquatic-thermal springs-hot spring | 3300000083\|Ga0072500_119562_4\|M | 5 | N | N | 546 |
| (3300000083\|Ga0072500_119562) | | | | | |
| aquatic-thermal springs-hot spring | 3300008000\|Ga0105162_1004089_4\|M | 7 | N | N | 543 |
| (3300008000\|Ga0105162_1004089) | | | | | |
| aquatic-thermal springs-hot spring | 3300008000\|Ga0105162_1004089_4\|P | 7 | N | N | 530 |
| (3300008000\|Ga0105162_1004089) | | | | | |
| aquatic-thermal springs-hot spring | 3300022544\|Ga0212120_1002806_5\|M | 7 | N | N | 543 |
| (3300022544\|Ga0212120_1002806) | | | | | |
| aquatic-thermal springs-hot spring | 3300022544\|Ga0212120_1002806_5\|P | 7 | N | N | 530 |
| (3300022544\|Ga0212120_1002806) | | | | | |
| aquatic-thermal springs-hot spring | 3300025063\|Ga0209493_1003385_3\|M | 16 | N | N | 512 |
| (3300025063\|Ga0209493_1003385) | | | | | |
| aquatic-thermal springs-hot spring | 3300025090\|Ga0209080_1004127_5\|M | 7 | N | N | 490 |
| (3300025090\|Ga0209080_1004127) | | | | | |
| aquatic-thermal springs-hot spring | 3300025440\|Ga0209741_1005495_1\|M | 2 | N | N | 521 |
| and microbial mat streamer | | | | | |
| (3300025440\|Ga0209741_1005495) | | | | | |
| aquatic-thermal springs-sediment | 3300028675\|Ga0272445_1001731_11\|M | 6 | N | N | 516 |
| (3300028675\|Ga0272445_1001731) | | | | | |
| aquatic-thermal springs-sediment | 3300029977\|Ga0272449_1037372_1\|P | 3 | N | N | 516 |
| (3300029977\|Ga0272449_1037372) | | | | | |
| fermentation metagenome | SRR1562008_megahit_k177_216982_5\|P | 2 | N | N | 417 |
| (SRR1562008) | | | | | |
| fermentation metagenome | UOPD01011681_4\|M | 2 | N | N | 417 |
| (UOPD01011681) | | | | | |
| Fervidibacteria bacterium JGI | 2645728127\|Ga0097870_126_5\|M | 20 | N | N | 512 |
| MDM2 | | | | | |
| (2645728127\|Ga0097870_126) | | | | | |
| freshwater metagenome | SRR5272259_megahit_k177_4486292_121\|P | 2 | N | N | 341 |
| (SRR5272259) | | | | | |
| freshwater metagenome | SRR5272259_megahit_k177_4486292_118\|M | 2 | N | N | 353 |
| (SRR5272259) | | | | | |
| freshwater metagenome | SRR5273149_megahit_k177_25824_1\|M | 14 | N | N | 514 |
| (SRR5273149) | | | | | |
| freshwater metagenome | SRR5273182_megahit_k177_57442_1\|M | 15 | N | N | 514 |
| (SRR5273182) | | | | | |
| freshwater metagenome | SRR5273183_megahit_k177_82677_3\|M | 3 | N | N | 454 |
| (SRR5273183) | | | | | |
| freshwater sediment metagenome | SRR6481359_megahit_k177_932521_3\|M | 3 | N | N | 373 |
| (SRR6481359) | | | | | |
| fungi-mycelium-attine ant fungus | 3300012677\|Ga0153928_1011778_2\|M | 3 | N | N | 332 |
| gardens | | | | | |
| (3300012677\|Ga0153928_1011778) | | | | | |
| Haloarcula quadrata | WP_121304574.1 | 2 | Y | N | 384 |
| (NZ_RBWW01000003) | | | | | |
| Halohasta litchfieldiae tADL | SEJ25361.1 | 2 | N | N | 382 |
| (277550727412775622397) | | | | | |
| Halorubrum sp. Atlit-26R | WP_121597521.1 | 2 | N | N | 384 |
| (NZ_QPLT01000012) | | | | | |
| Halorubrum tebenquichense DSM | WP_006630662.1 | 4 | N | N | 384 |
| 14210 (AOJDO1000077) | | | | | |
| hot springs metagenome | DRR082460_megahit_k177_106880\|P | 7 | N | N | 535 |
| (DRR082460) | | | | | |
| hot springs metagenome | DRR082460_megahit_k177_106880\|M | 7 | N | N | 537 |
| (DRR082460) | | | | | |
| hot springs metagenome | OGCH01000195_3\|M | 3 | N | N | 546 |
| (OGCH01000195) | | | | | |
| hot springs metagenome | SRR4027810_megahit_k177_26210_1\|M | 17 | N | N | 512 |
| (SRR4027810) | | | | | |
| hot springs metagenome | SRR5247126_megahit_k177_220968_4\|P | 7 | N | N | 532 |
| (SRR5247126) | | | | | |
| lab enrichment-defined media- | 3300027941\|Ga0207719_100292_34\|M | 2 | N | N | 384 |
| aerobic enrichment media | | | | | |
| (3300027941\|Ga0207719_100292) | | | | | |
| lab enrichment-defined media- | 3300027941\|Ga0207719_100292_35\|P | 2 | N | N | 376 |
| aerobic enrichment media | | | | | |
| (3300027941\|Ga0207719_100292) | | | | | |
| landfill metagenome (SRR6484323) | SRR6484323_megahit_k177_106899_4\|P | 3 | N | N | 429 |
| landfill metagenome (SRR6484325) | SRR6484325_megahit_k177_1345066_6\|P | 2 | N | N | 429 |
| landfill metagenome (SRR6484787) | SRR6484787_megahit_k177_1514834_8\|M | 4 | N | N | 468 |
| landfill metagenome (SRR8542065) | SRR8542065_megahit_k177_1115558_2\|M | 3 | N | N | 429 |
| landfill metagenome (SRR8542065) | SRR8542065_megahit_k177_821416_8\|M | 4 | N | N | 440 |
| metagenome (FZPS01000148) | FZPS01000148_2153\|M | 2 | N | N | 417 |
| Natronococcus amylolyticus DSM | WP_005554257.1 | 2 | N | N | 389 |
| 10524 (AOIB01000014) | | | | | |
| peat metagenome (SRR7004341) | SRR7004341_megahit_k177_2665443_5\|P | 2 | N | N | 363 |
| salt lake metagenome (SRR8526178) | SRR8526178_megahit_k177_763700_3\|P | 5 | N | N | 432 |
| sediment metagenome | OVXO01016275_2\|M | 3 | N | N | 358 |
| (OVXO01016275) | | | | | |
| sediment metagenome | SRR7067823_megahit_k177_107253_1\|P | 7 | N | N | 512 |
| (SRR7067823) | | | | | |
| sediment metagenome | SRR7067986_megahit_k177_69094_7\|P | 45 | N | N | 512 |
| (SRR7067986) | | | | | |
| sediment metagenome | SRR8554444_megahit_k177_504614_2\|M | 2 | N | N | 351 |
| (SRR8554444) | | | | | |
| sediment metagenome | SRR8554446_megahit_k177_83048_1\|M | 8 | N | N | 516 |
| (SRR8554446) | | | | | |
| soil metagenome (SRR6201998) | SRR6201998_megahit_k177_1568738_2\|M | 4 | N | N | 359 |
| soil metagenome (SRR8554511) | SRR8554511_megahit_k177_4248910_4\|P | 3 | N | N | 457 |
| soil metagenome (SRR8554511) | SRR8554511_megahit_k177_4248910_4\|M | 3 | N | N | 437 |
| solid waste-landfill-landfill leachate | 3300014204\|Ga0172381_10015437_8\|M | 4 | N | N | 468 |
| (3300014204\|Ga0172381_10015437) | | | | | |
| solid waste-landfill-landfill leachate | 3300014205\|Ga0172380_10102486_2\|M | 2 | N | N | 429 |
| (3300014205\|Ga0172380_10102486) | | | | | |
| terrestrial-soil | 3300031566\|Ga0307378_10048523_2\|M | 2 | N | N | 419 |
| (3300031566\|Ga0307378_10048523) | | | | | |
| wastewater metagenome | SRR6837575_megahit_k177_854481_7\|M | 5 | N | N | 469 |
| (SRR6837575) | | | | | |
| wastewater metagenome | SRR6837576_megahit_k177_1219670_1\|P | 5 | N | N | 469 |
| (SRR6837576) | | | | | |
| wastewater-anaerobic digestor- | 3300009716\|Ga0116191_1022210_3\|P | 4 | N | N | 419 |
| anaerobic digestor sludge | | | | | |
| (3300009716\|Ga0116191_1022210) | | | | | |

TABLE 8
Amino acid sequences of Representative CLUST.196682 Effector Proteins

>3300025638|Ga0208198_1021643_2|P
[wastewater-anaerobic digestor-anaerobic digestor sludge]
MKATTITKSLRFWIDEPPERCRLLYGVSDELTDAYNAILEYWETDVRKVAVEATEAAIEAYHVWKDAPKETRGDKPKWWSADTA
TRAAFLRVGVAATSTIRTRLVDNMVQGEWLRDVKTYMSRRFKDHIAAGIRHRAKGVVISLDQSTMKPDVQLGDDGRWTIRCALW
AQGTEESARNNDRWLLSPYDRKRQTWTIANLLEADQHGEVRGVKLVPPKPGAPAGKRRWSAMITVTLPVESSVLDEERPNVAGV
DMGLTHFAVYSCPARNTFEFVSSRELQAAMEKARRRRRGVPRKRSTALGQKLGRRQDALCRLTARRLIDCCRRDRVGTLRVEDL
TGIRDQGSDDADRNFALGARFPYYKLQTYLEQAAASAGVRLEKVQPAGTSQTCSRCAVRDPESRDGKRFVCRHCGYKGDADLNA
ANNIASGRFRRSIRPSTQPAGRIPSAAPDCTGEAVKASDVDAELQPARTPAACGGDPYAGE
(SEQ ID NO: 601)
>3300005300|Ga0072500_136962_4|M
[aquatic-thermal springs-hot spring]
MEKVILTITSGVEFLQPQDEQLVIKAAKWYTEARKALLYNALQKGEEGVKQAASTLSTLNFPPAPHSRLPDLLTRDVGGTLQAL
KERREGYAQDFSRWWEEICRVANEKGSQAVSVLKPHVNKLSSLIHQLSWNSPEWDEEAQEVFKELKQIWAVVRQKDHERLKIKW
RAEVPPVPRPPRTPKFQRLVFNADGRVSGGSRSNALQIEEREDGDLYLRLQLRDPERVAKKGSYQDVWLRLVIEDEERKQMLKN
FAPKYLQLRQDGKRWIAHIVIERENEFEVPQYVYGVDIGARKYLLAAVAIPLNPALPKPPNIAVPSSKFWQIVERVEAEWRKWQ
QLRTGKRDKEGRVVLKKRKMAWRVLKRKRRMRREVTKSMIAEAVNTFIKSLERPCAIVIEQIKNLHLSPTLQDRKSRWRQRRWA
YRFLLEMLRYKAEWEGIRVIEVNPAYTSQTCPRCGKRDRNARQGNLFQCTKCRFQHNADFVAAYNITVRGLMKLRTRGKVDWKV
LTGKPRVL
(SEQ ID NO: 602)
>CAAACJ01004249 9_2|P
[activated sludge metagenome]
MKATTITKSLRFWIDEPPERCRLLYGVSDELTDAYNAILEYWETDVRKVAVEATEAAIEAYHVWKDAPKETRGDKPKWWSADTA
TRAAFLRVGVAATSTIRTRLVDNMVQGEWLRDVKTYMSRRFKDHIAAGIRHRAKGVVISLDQSTMKPDVQLGDDGRWTIRCALW
AQGTEESARNNDRWLLSPYDRKRQTWTIANLLEADQHGEVRGVKLVPPKPGAPAGKRRWSAMITVTLPVESSVLDEERPNVAGV
DMGLTHFAVYSCPARNTFEFVSSRELQAAMEKARRRRRGVPRKRSTALGQKLGRRQDALCRLTARRIIDCCRRDRVGTLRVEDL
TGIRDQGSDDADRNFALGARFPYYKLQTYLEQAAASAGVRLEKVQPAGTSQTCSRCAVRDPESRDGKRFECRHCGYKGDADLNA
ANNIASGRFRRSIRPSTQPAGRIPSAAPDCTGEAVKASDVDAELQPARTPAACGGDPYAGE
(SEQ ID NO: 603)
>SRR5007116_megahit_k177_300528_2|M
[activated sludge metagenome]
MKATTTTKSLRFWIDEPPERCRLLYGVSDELTAAYNAILEYWETDVRSVAVEVTEAAQAAYHVWKDAPKETRGDKPKWWSADTA
TRAAFLRVGVAATSTIRTRLVDNMVQGEWLRDVKTYMSRRFKDHIAAGIRHRAKGVVISLDQSTMKPDVQLGDDGRWTIRCALW
AQGTEESARNNDRWRLSPYDRRRQTWTIANLLEADQHGEVRGVKLVPPKPGAPAGKRRWSAMITVTLPVESSVLDEERPNVAGV
DMGLTHFAVYSCPARNTFEFVSSRELQAAMEKARRRRRGVPRKRSTALGQKLGRRQDALCRLTARRLIDCCLRDRVGTLRVEDL
TGIRDQGSDDPDRNFALGARFPYYKLQTYLEQAAASAGVRLEKVQPAGTSQTCSRCAVRDPESRDGKRFVCRHCGYKGDADLNA
ANNIASGRFRRSIRPSTQPAGRIPSAAPDCTGEAVEASNVGAELQPAREPVAGGGDPYAGE
(SEQ ID NO: 604)
>SRR5007140_megahit_k177_707372_2|P
[activated sludge metagenome]
MKATTITKSLRFWIDEPPERCRLLYGVSDELTDAYNAILEYWETDVRKVAVEATEAAIEAYHVWKDAPKETRGDKPKWWSADTA
TRAAFLRVGVAATSTIRTRLVDNMVQGEWLRDVKTYMSRRFKDHIAAGIRHRAKGVVISLDQSTMKPDVQLGDDGRWTIRCALW
AQGTEESARNNDRWLLSPYDRKRQTWTIANLLEADQHGEVRGVKLVPPKPGAPAGKRRWSAMITVTLPVESSVLDEERPNVAGV
DMGLTHFAVYSCPARNTFEFVSSRELQAAMEKARRRRRGVPRKRSTALGQKLGRRQDALCRLTARRIIDCCRRDRVGTLRVEDL
TGIRDQGSDDADRNFALGARFPYYKLQTYLEQAAASAGVRLEKVQPAGTSQTCSRCAVRDPESRDGKRFECRHCGYKGDADLNA
ANNIASGRFRRSIRPSTQPAGRIPSAAPDCTGEAVKASDVDAELQPARTPAACGGDPYAGE
(SEQ ID NO: 603)
>SRR5007457_megahit_kl77_513017_2|M
[activated sludge metagenome]
MKATTITKSLRFWIDEPPERCRLLYGVSDELTDAYNAILEYWETDVRKVAVEATEAAIEAYHVWKDAPKETRGDKPKWWSADTA
TRAAFLRVGVAATSTIRTRLVDNMVQGEWLRDVKTYMSRRFKDHIAAGIRHRAKGVVISLDQSTMKPDVQLGDDGRWTIRCALW
AQGTEESARNNDRWLLSPYDRKRQTWTIANLLEADQHGEVRGVKLVPPKPGAPAGKRRWSAMITVTLPVESSVLDEERPNVAGV
DMGITHFAVYSCPARNTFEFVSSRELQAAMEKARRRRRGVPRKRSTALGQKLGRRQDALCRLTARRIIDCCRRDRVGTLRVEDL
TGIRDQGSDDADRNFALGARFPYYKLQTYLEQAAASAGVRLEKVQPAGTSQTCSRCAVRDPESRDGKRFECRHCGYKGDADLNA
ANNIASGRFRRSIRPSTQPAGRIPSAAPDCTGEAVKASDVDAELQPARTPAACGGDPYAGE
(SEQ ID NO: 605)
>SRR5007461_megahit_k177_311051
[activated sludge metagenome]
MIKTVTFYIAEMPREDKRLCDRLCIDLGAAGARMVREWFRACDETPPGAAIDWPEVQRRGVAVGKAMVEAAGYRTELGNDLARA
EFFARFKMEFKKVLKGERASLPFPGRAPFIFLRMDPGHETAIIEQRDDAWYMRNPRFYPGGDSNEANRREWRLKPVDRKGWAEN
ILEAAEKFACVRIMPDRKKPGSWVVKITVHLPDALERKLVEEHECWAGIDLGINCPAVLSIPDRDKGGFVRFFGMDKYTALWAK
LNRYEERKRALNRVGQQHAARDLRAKITGLRQYINEQISREIIDLCLRMHVTGIRMEDLKGLRTEGTGKLKYWPRFHLTTRIEQ
KAREVGLAFEQIRAAGTSQTCSVCGHRHRDNRHGSDFLCLRCGFARHADVNAANNIARGMQDFDLPAGSAAPGRALRLELSAQ
(SEQ ID NO: 606)
>3300027896|Ga0209777_10000273_30|P
[aquatic-freshwater-freshwater lake sediment]
MTATKQQILQTEYDNLQHFLQTGEDLGVYSANKQQAKRYYKTIKPEHQYPLSIRNDLIQVQHKPNSIAEYWLKIPVKGRRGGLW
VAIRPHQQIPDNAEFLESKIRRKRKGWFANIVIQKQIEPIRTKNILAVDLGEHVIATVCGSFDNQRPRFYGRDVRGIRRHYAWL
RKRLGEKKLLGVIRRIKDTEKRKVNDTLHKISRSIVDLAQEHNATIVLGELQGIRNHAKGRRMNRIVSNMPYLKLSQMIEYKAE
WLGIPVIKMQEHDTSKTCSRCNEYGKRPTQGQFRCDTCHRTSFNADYNGALNILKRATSYMDIAGAVSEPARDWGNDDQTSEAT
QLFEW
(SEQ ID NO: 607)
>3300027896|Ga0209777_10000273_32|M
[aquatic-freshwater-freshwater lake sediment]
MAQKTITAKIVAMTATKQQILQTEYDNLQHFLQTGEDLGVYSANKQQAKRYYKTIKPEHQYPLSIRNDLIQVQHKPNSIAEYWL
KIPVKGRRGGLWVAIRPHQQIPDNAEFLESKIRRKRKGWFANIVIQKQIEPIRTKNILAVDLGEHVIATVCGSFDNQRPRFYGR
DVRGIRRHYAWLRKRLGEKKLLGVIRRIKDTEKRKVNDTLHKISRSIVDLAQEHNATIVLGELQGIRNHAKGRRMNRIVSNMPY
LKLSQMIEYKAEWLGIPVIKMQEHDTSKTCSRCNEYGKRPTQGQFRCDTCHRTSFNADYNGALNILKRATSYMDIAGAVSEPAR
DWGNDDQTSEATQLFEW
(SEQ ID NO: 608)
>3300013127|Ga0172365_10030480_1|M
[aquatic-freshwater-sediment]
MANRELVVVMQVQKVVKGKVVGLTHAKRQILDHEYEGLQRFLQGDSNVELYSANKQQAKRFYKTVKPEREYPLSIRKDLIRVER
RDTKIARYWARIPAKLRRGGVWVAVKPHCEITDDLEICESKLLRRNGEYFLHLAVKREVELNTSYSSVLAIDLGIRHVACSVDT
RSGETHFHGRELRRVRGHYFHLRRRLGEKKLLKVIKRIGARESRITNDQLHKIAKAIVDEAERLNALIAIGNPKGIRRNGKGRR
FNRKLNSWPFWKLRQYIEYKANWRGIAVVEVNEAYTSQRCWRCGTIGTRSGTHHGLFECPRCGLRENADRNGAFNIGRRALGQV
SKVGAVVSQPVTGAVPFSELGSVVQCPTPEATLFKGW
(SEQ ID NO: 609)
>3300013128|Ga0172366_10002984_1|M
[aquatic-freshwater-sediment]
MANRELVVVMQVQKVVKAKVVGLTHAKRQILDHEYEGLQRFLQGDSNVELYSANKQQAKRFYKTVKPEREYPLSIRKDLIRVER
RDTKIARYWARIPAKLRRGGVWVAVKPHCEITDDLEICESKLLRRNGEYFLHLAVKREVELNTSYSSVLAIDLGIRHVACSVDT
RSGETHFHGRELRRVRGHYFHLRRRLGEKKLLKVIKRIGARESRITNDQLHKIAKAIVDEAERLNALIAIGNPKGIRRNGKGRR
FNRKLNSWPFWKLRQYIEYKANWRGIAVVEVNEAYTSQRCWRCGTIGTRSGTHHGLFECPRCGLRENADRNGAFNIGRRALGQV
SKVGAVVSQPVTGAVPFSELGSVVQCPTPEATLFKGW
(SEQ ID NO: 610)
>3300013129|Ga0172364_10030775_1|P
[aquatic-freshwater-sediment]
MANRELVVVMQVQKVVKGKVVGLTHAKRQILDHEYEGLQRFLQGDSNVELYSANKQQAKRFYKTVKPEREYPLSIRKDLIRVER
RDTKIARYWARIPAKLRRGGVWVAVKPHCEITDDLEICESKLLRRNGEYFLHLAVKREVELNTSYSSVLAIDLGIRHVACSVDT
RSGETHFHGRELRRVRGHYFHLRRRLGEKKLLKVIKRIGARESRITNDQLHKIAKAIVDEAERLNALIAIGNPKGIRRNGKGRR
FNRKLNSWPFWKLRQYIEYKANWRGIAVVEVNEAYTSQRCWRCGTIGTRSGTHHGLFECPRCGLRENADRNGAFNIGRRALGQV
SKVGAVVSQPVTGAVPFSELGSVVQCPTPEATLFKGW
(SEQ ID NO: 609)
>3300013130|Ga0172363_10005240_1|P
[aquatic-freshwater-sediment]
MANRELVVVMQVQKVVKGKVVGLTHAKRQILDHEYEGLQRFLQGDSNVELYSANKQQAKRFYKTVKPEREYPLSIRKDLIRVER
RDTKIARYWARIPAKLRRGGVWVAVKPHCEITDDLEICESKLLRRNGEYFLHLAVKREVELNTSYSSVLAIDLGIRHVACSVDT
RSGETHFHGRELRRVRGHYFHLRRRLGEKKLLKVIKRIGARESRITNDQLHKIAKAIVDEAERLNALIAIGNPKGIRRNGKGRR
FNRKLNSWPFWKLRQYIEYKANWRGIAVVEVNEAYTSQRCWRCGTIGTRSGTHHGLFECPRCGLRENADRNGAFNIGRRALGQV
SKVGAVVSQPVTGAVPFSELGSVVQCPTPEATLFKGW
(SEQ ID NO: 609)
>3300013133|Ga0172362_10039269_3|M
[aquatic-freshwater-sediment]
MANRELVVVMQVQKVVKGKVVGLTHAKRQILDHEYEGLQRFLQGDSNVELYSANKQQAKRFYKTVKPEREYPLSIRKDLIRVER
RDTKIARYWARIPAKLRRGGVWVAVKPHCEITDDLEICESKLLRRNGEYFLHLAVKREVELNTSYSSVLAIDLGIRHVACSVDT
RSGETHFHGRELRRVRGHYFHLRRRLGEKKLLKVIKRIGARESRITNDQLHKIAKAIVDEAERLNALIAIGNPKGIRRNGKGRR
FNRKLNSWPFWKLRQYIEYKANWRGIAVVEVNEAYTSQRCWRCGTIGTRSGTHHGLFECPRCGLRENADRNGAFNIGRRALGQV
SKVGAVVSQPVTGAVPFSELGSVVQCPTPEATLFKGW
(SEQ ID NO: 609)
>3300032070|Ga0315279_10000130_51|M
[aquatic-freshwater-sediment]
MILQKTVNAKLFALTRTKEQKISREYWNFQKALHGEDAPLYSATKQQAKRKNAWNGDCEYPLVLRRDCFGVKELGLKFARWWAK
IPVFGGSIWCPVQLPYVQEPLLKGNIRETKLVRRKKGWFLRITVQQEIELDVPCASVVGVDFGERHIATSVEWQGRFQNPIFYG
AEVRGIRRHYAWLRKRLGNKKALRTIKKVGGKENRKVNAVLHNISRAIVDRAKELSAVILLGAPNGGSMRRKALSKGKRFTRIV
YSMPYYRLRQFIEYKAVQEGILVVNANEEWTSKTCSFCGEISDRPRQDLIVCPHGHRLNADINACRNLIKRFIDHWLIDGAILA
SPKLPH
(SEQ ID NO: 611)
>3300032118|Ga0315277_10001250_38|M
[aquatic-freshwater-sediment]
MKSQKIIKCKITYLTNIKAQQLETEHHNLQNLLQLEREGLDFLPIYQNIPLHSANKQQALRFYKRISTDKIYAISIRNDIIKVE
LKPNNKISKYWCKIPVKQRYGGIWVAIKPHKKDIDFSQYKLGESKLFKKKNEWWIYITIIREVQIKDSYSNILGIDLGSKVIAT
VCGSFYNQRPTFYGREVRGIRRHYQYIRTELGKKKLLKKIKQLSDKEHRIVNDLLHKISKQIVEKAEQTDSYIVLGDLKGIRNS
TKNKGRNFRRIVSNMPYLKLTQFIEYKAKEKGIKIIRISEKGTSKICSKCGYEDKGNRKSQGLFKCKQCGFELNADANGARNIL
KFSDGYTLPERAMSELALNSNQLEKPTCFSGG
(SEQ ID NO: 612)
>3300024429|Ga0209991_10029076_1|P
[aquatic-marine-deep subsurface]
MLVEKTVKAKVIAPTNRKQELLDREYQSFQYTLYGGEANLYSATKQQAERFLRKIKTPKHRHYPLILRRDVIRLERQETKVAPY
WFKFPVFGVRGGIWLGLKPHCDIPVDCSLREAKLIRRKNAWFIHLTIQKEVPESIIDEHHILAVDLGERYLATVCGNNGTNPQF
LGKEVRGIRRHYAWIRKRLGERKLLNTTRKVGHTEQKKVNDICHQISRRIVNEARETDSVIILGDLKGIRKNAKGKRMNRIVSN
MPYYKLTQFIKYKAAWDGVPVLVIPEAYTSKGCHRCGSLGSRPYRGLFLCHSCKLHYNADLNGARNILKRALDYMSSAGAALAQ
PVTQSLN
(SEQ ID NO: 613)
>3300014886|Ga0180300_10007429_2|M
[aquatic-marine-marine sediment]
MKVKKTIKAKILELRKGKEELLKREYENWQRYLRGDKTVPLYSATKQQADRLLKRLKGRLKPNKEYPMILRRDVYRANTKLTPY
WLKISIYGVRGGINVPIKTHEPITEDMVCREAKIIRKGDEWFVYITVEKEVEERNPKSILAIDLGIRWMATTVNSNNPKPNFYG
KELRKVKGHYFYLRRSLTLKKAYKTIKKIGLKERRVVNDILHKISRAIVNEALENDSMIVLGNLKGIRRNGKGRRFNRKLNNGF
PYYRLSQFIEYKAKWLGIKVIKISEKNTSKLCHKCGEEGVRVGSSFKCPNCGYSCHADYNGAMNILKRAMGLVSVAGAGLTQPR
TRYDEARRPKNRESPHLSGKSVKSYSFKF
(SEQ ID NO: 614)
>SEJ25361.1
[aquatic-non marine saline and alkaline-hypersaline]
MKQTAEKTVVAKIFTNDLTQSKLDAINHEYEAFQRYIRGDEEADLYSATKQCADAYVDTDNLRDDHDYPWFIRNDVFDVGVDVE
QHDTDLTDWWLKLPVSQVWGGVNVPIQPHDEIPDEADVKDSKIVRVNGDYYAHLTVQQEVEVSEEYGGVIGVDFGVRWVATSVA
LPSRNTEFYGREIRRIRRHHHDLRTRLQEKGAYGTLQSVKVKEYRQVNDRLHKISRAIVEEAKECDALIVVGDLEGIQNQDLGA
EMNRRLHSMPHHTLKTYIEYKAMFAGIAVKSVNEYMTSQTCWRCGEQEATTRKGQGQHLCHECGLDDNADKNGATNIAKKGLGK
DIACPLSSLGAVCEPVLGPSGADQASATVCLRADLEAPASTKRTVR
(SEQ ID NO: 615)
>3300005935|Ga0075125_10033396_1|P
[aquatic-non marine saline and alkaline-saline lake]
MSLKTLTFWPIGMSAKYRRIFDNIRKQLGAAQREMVKAWLRLALAEDAYGIDPGKRAGREVFGDNEYSNDLLNNLAYADAHENF
KQQWKRVRSGNATSVSFSDAAANVLFLMPPSGGHHATVEHIDGNWWITRLKVFGGCTKADRERNDQFRWQLKGVSRFGGKYEKD
LMDGAQRICQVRVITSRKDPSKYVVQVTVEVPDLQTVGARTPVCAGIDLGINCPVVLSIPDKGFVQFMGRERDEYPRLWEKLRE
YEDRKRRLNRAGKRRAARDLRPRITNVRKYINEAISKDVADTCRALGVTHLRMEDLKGLSRHKSMTGKLRYWPRYHLQERIERK
CCEVGIEVERIKPAGTSQTCSVCGHREKANRNGAQFVCIECGYQQHADLNAANNIARGMQTFGPCAKSSASDDAIGLRFDSEGQ
ESALGQAKRLFA
(SEQ ID NO: 616)
>3300025736|Ga0207997_1027507_2|M
[aquatic-non marine saline and alkaline-saline lake]
MSLKTLTFWPIGMSAKYRRIFDNIRKQLGAAQREMVKAWLRLALAEDAYGIDPGKRAGREVFGDNEYSNDLLNNLAYADAHENF
KQQWKRVRSGNATSVSFSDAAANVLFLMPPSGGHHATVEHIDGNWWITRLKVFGGCTKADRERNDQFRWQLKGVSRFGGKYEKD
LMDGAQRICQVRVITSRKDPSKYVVQVTVEVPDLQTVGARTPVCAGIDLGINCPVVLSIPDKGFVQFMGRERDEYPRLWEKLRE
YEDRKRRLNRAGKRRAARDLRPRITNVRKYINEAISKDVADTCRALGVTHLRMEDLKGLSRHKSMTGKLRYWPRYHLQERIERK
CCEVGIEVERIKPAGTSQTCSVCGHREKANRNGAQFVCIECGYQQHADLNAANNIARGMQTFGPCAKSSASDDAIGLRFDSEGQ
ESALGQAKRLFA
(SEQ ID NO: 616)
>3300007203|Ga0099832_1115789_2|M
[aquatic-thermal springs]
MKKVVITVTTGVEFLQPQDEQIVRKAAEWYTKAREALFAHALQQGKVKEAASTLRTLNFPMAPHSRLPDLLTRDVGGILKALKE
KREGYLGEFKRWWEEDCRVANEKGSQAVADLKTHVGKLLNLVHQLPWNSPVWDGEAQEAFKNLKEAWAVVRQKDREQLKIRWRA
EVSPLRRPPRITKFQRLVFNADGRVSGGSKSEALQIEEREDGDLYLRLQLRDPEGVVGGGAYQEVWLRLVVEDEERKQMLRDFA
PKYLQLRQDGKRWIAHIVIEREIKFGLKGIMWTAGDYLYGVDIGARKYLLAAVAVPLKPGLPKLPNIAIPSGKFWQAVERVEAE
WRKWQRLRTGKKDKEGKVVLKKRTAWRVLKRKRRMRREVAKNMIAEAVNTFINFIKGDNVDIRLWLAIEQIKNLNLSPAFQDRK
SRWRQRRWAYRLLLEMLRYKAEWEGIRVIEVNPAHTSQICPRCGKKDGKARQGNRFCCTKCKFQHNADFVAAYNIAVRALKKLG
MREEIGWKVLTGKPRVL
(SEQ ID NO: 617)
>3300000083|Ga0072500_119562_4|M
[aquatic-thermal springs-hot spring]
MKHLVSNAQVNEKNEARLTLKCGLEFENAEGEELVKRAAEWYCKARIFLLSYALKNNKTRVQKIASLLKSEGFPSPPHPRLIDL
LQRTITPAIKNMRRLRRRYPKTLRAWFDRVKKFAEEKGSDAAERLQPYVEDLLQLVKNLPINSPAWDTDAQEKWKELKKLWGKV
RESDWKKGIVWKFQIPNPKKPPSTPAFRCADSSNIFVTADYRMSGKNARNSYDMKTDSEENLWVLVRLDDVDGDVKVRKDVWIK
LCVDKSRYDLLRIGKITEIKILRNRDGRWEGRIVVKMNAGYNSPQVVVGVDVGASEHLIAAVGIPLDGSNNNFLLSVPASYFWK
RVEQAERLWRRWQRLYTGRTSEKKKAPILPRRRKAYQILKRNRNWRREVTKHCIFHAVKKFVQLLPPNSVVVVENLKDIPPNVW
KEDKDKRFRRWVYAFLLFTLKYKCQQRKIAVIAVSPHNTSQTCPRCGVVDPKSRGLWKDGELISRKHFCCTQCKFTHNADFVAA
HNIAVLGAKALGITLPESWRALRGEVQYVIPYQNMPKVRKSW
(SEQ ID NO: 618)
>3300008000|Ga0105162_1004089_4|M
[aquatic-thermal springs-hot spring]
MNNKQVTFGGLFLMALQKKRVRRTIVAGVEIESAEEENLVTEAAKWYLQAKQKLLQYAIEKRKERITAPARILKDELKLPEPPH
SRLPDELAQVINGVLKALKKRRTEYLRRFQDWWNKVVEVGNQHGLESYRAMKPKVEALISSVPNNPFDPTWDSDIQKRFAEVKE
IWSKIRLKDSNERNFRWRYHVPPLVKPPKTPEFKLLGFDAGARVSGGGKAKTLTIKSQDGEIFLRLLLRQPGVSNHGRGSYREI
WLHIILEDESRKALLLSDKVQAKFIRLRRRTDKWEAHITVEKEVSFEKPRVIYGIDIGMKDALLFCVPIFIDSQETTETASEGK
KLIVVEAGQFWHRLEVEDAQWRKWQFRRSGKVKEGIKGKRMVWRILKRRRRRRREVTKHLIHQIVNALISQLQRPCAVAIERLK
GLRNKPHLTDKRSQWKFHRWAYRFLLETLRYKLEWEGIMIIEVPPYGTSQTCPRCSHRDKRSRDGRRFRCTKCKFQHNADAAGA
FNIAMRAVEQLQSSRDLQEVSIAESAWKILTGKPMVLKY
(SEQ ID NO: 619)
>3300008000|Ga0105162_1004089_4|P
[aquatic-thermal springs-hot spring]
MALQKKRVRRTIVAGVEIESAEEENLVTEAAKWYLQAKQKLLQYAIEKRKERITAPARILKDELKLPEPPHSRLPDELAQVING
VLKALKKRRTEYLRRFQDWWNKVVEVGNQHGLESYRAMKPKVEALISSVPNNPFDPTWDSDIQKRFAEVKEIWSKIRLKDSNER
NFRWRYHVPPLVKPPKTPEFKLLGFDAGARVSGGGKAKTLTIKSQDGEIFLRLLLRQPGVSNHGRGSYREIWLHIILEDESRKA
LLLSDKVQAKFIRLRRRTDKWEAHITVEKEVSFEKPRVIYGIDIGMKDALLFCVPIFIDSQETTETASEGKKLIVVEAGQFWHR
LEVEDAQWRKWQFRRSGKVKEGIKGKRMVWRILKRRRRRRREVTKHLIHQIVNALISQLQRPCAVAIERLKGLRNKPHLTDKRS
QWKFHRWAYRFLLETLRYKLEWEGIMIIEVPPYGTSQTCPRCSHRDKRSRDGRRFRCTKCKFQHNADAAGAFNIAMRAVEQLQS
SRDLQEVSIAESAWKILTGKPMVLKY
(SEQ ID NO: 620)
>3300022544|Ga0212120_1002806_5|M
[aquatic-thermal springs-hot spring]
MNNKQVTFGGLFLMALQKKRVRRTIVAGVEIESAEEENLVTEAAKWYLQAKQKLLQYAIEKRKERITAPARILKDELKLPEPPH
SRLPDELAQVINGVLKALKKRRTEYLRRFQDWWNKVVEVGNQHGLESYRAMKPKVEALISSVPNNPFDPTWDSDIQKRFAEVKE
IWSKIRLKDSNERNFRWRYHVPPLVKPPKTPEFKLLGFDAGARVSGGGKAKTLTIKSQDGEIFLRLLLRQPGVSNHGRGSYREI
WLHIILEDESRKALLLSDKVQAKFIRLRRRTDKWEAHITVEKEVSFEKPRVIYGIDIGMKDALLFCVPIFIDSQETTETASEGK
KLIVVEAGQFWHRLEVEDAQWRKWQFRRSGKVKEGIKGKRMVWRILKRRRRRRREVTKHLIHQIVNALISQLQRPCAVAIERLK
GLRNKPHLTDKRSQWKFHRWAYRFLLETLRYKLEWEGIMIIEVPPYGTSQTCPRCSHRDKRSRDGRRFRCTKCKFQHNADAAGA
FNIAMRAVEQLQSSRDLQEVSIAESAWKILTGKPMVLKY
(SEQ ID NO: 619)
>3300022544|Ga0212120_1002806_5|P
[aquatic-thermal springs-hot spring]
MALQKKRVRRTIVAGVEIESAEEENLVTEAAKWYLQAKQKLLQYAIEKRKERITAPARILKDELKLPEPPHSRLPDELAQVING
VLKALKKRRTEYLRRFQDWWNKVVEVGNQHGLESYRAMKPKVEALISSVPNNPFDPTWDSDIQKRFAEVKEIWSKIRLKDSNER
NFRWRYHVPPLVKPPKTPEFKLLGFDAGARVSGGGKAKTLTIKSQDGEIFLRLLLRQPGVSNHGRGSYREIWLHIILEDESRKA
LLLSDKVQAKFIRLRRRTDKWEAHITVEKEVSFEKPRVIYGIDIGMKDALLFCVPIFIDSQETTETASEGKKLIVVEAGQFWHR
LEVEDAQWRKWQFRRSGKVKEGIKGKRMVWRILKRRRRRRREVTKHLIHQIVNALISQLQRPCAVAIERLKGLRNKPHLTDKRS
QWKFHRWAYRFLLETLRYKLEWEGIMIIEVPPYGTSQTCPRCSHRDKRSRDGRRFRCTKCKFQHNADAAGAFNIAMRAVEQLQS
SRDLQEVSIAESAWKILTGKPMVLKY
(SEQ ID NO: 620)
>3300025063|Ga0209493_1003385_3|M
[aquatic-thermal springs-hot spring]
MEKVILTITSGVEFLQPQDEQLVIKAAKWYTEARKALLYNALQKGEEGVKQAASTLSTLNFPPAPHSRLPDLLTRDVGGTLQAL
KERREGYAQDFSRWWEEICRVANEKGSQAVSVLKPHVNKLSSLIHQLSWNSPEWDEEAQEVFKELKQIWAVVRQKDHERLKIKW
RAEVPPVPRPPRTPKFQRLVFNADGRVSGGSRSNALQIEEREDGDLYLRLQLRDPERVAKKGSYQDVWLRLVIEDEERKQMLKN
FAPKYLQLRQDGKRWIAHIVIERENEFEVPQYVYGVDIGARKYLLAAVAIPLNPALPKPPNIAVPSSKFWQIVERVEAEWRKWQ
QLRTGKRDKEGRVVLKKRKMAWRVLKRKRRMRREVTKSMIAEAVNTFIKSLERPCAIVIEQIKNLHLSPTLQDRKSRWRQRRWA
YRFLLEMLRYKAEWEGMRVIEVNPAYTSQTCPRCGERDRNARQGNLFRCTKCRFQHNADFVAAYNITVRGLMKLRTRGRIDWKI
LTGKPRVL
(SEQ ID NO: 621)
>3300025090|Ga0209080_1004127_5|M
[aquatic-thermal springs-hot spring]
MLQYAIEKRKERITAPARILKDELKLPEPPHSRLPDELAQVINGVLKALKKRRTEYLRRFQDWWNKVVEVGNQHGLESYRAMKP
KVEALISSVPNNPFDPTWDSDIQKRFAEVKEIWSKIRLKDSNERNFRWRYHVPPLVKPPKTPEFKLLGFDAGARVSRGGKAKTL
TIKSQDGEIFLRLLLRQPGVSNHGRGSYREIWLHIILEDESRKALLLSDKVQAKFIRLRRRTDKWEAHITVEKEVSFEKPRVIY
GIDIGMKDALLFCVPIFIDSQETTETASEGKKLIVVEAGQFWHRLEVEDAQWRKWQFRRSGKVKEGIKGKRMVWRILKRRRRRR
REVTKHLIHQIVNALISQLQRPCAVAIERLKGLRNKPHLTDKRSQWKFHRWAYRFLLETLRYKLEWEGIMIIEVPPYGTSQTCP
RCSHRDKRSRDGRRFRCTKCKFQHNADAAGAFNIAMRAVEQLQSSRDLQEVSIAESAWKILTGKPMVLKY
(SEQ ID NO: 622)
>3300025440|Ga0209741_1005495_1|M
[aquatic-thermal springs-hot spring and microbial mat streamer]
MEKVVITITTGVEFLQPQDEQIVRKAAEWYTKAREALFDHALQQGKVKKAASTLRTLNLPMAPHSRLPDLLTRDVGGILKALKE
KRKGYLREFKRWWKEVCKVANEKGSQAVAVLKPHVDKLLNLVRQLPWNSPVWDGEAQEAFKNLKEAWAVVRQKDREQLKIKWRA
EVSPLRRPPRTPKFQRLVFNADGRVSGGSKSEALQIEEREDGDLYLRLQLRDPEGVVGGGAYQEVWLRLVVEDEERKQMLRDFA
PKYLQLRQDGKRWIAHIVIEREIKFGLKGIMWTAGDYLYGVDIGARKYLLAAVAVPLKPGLPKLPNIAIPSGKFWQAVERVEAE
WRKWQRLRTGKKDKEGKVVLKKRTAWRVLKRKRHMRREVAKNMIAEAVNTFINFIKGDNVDIRLWLAIEQIKNLNLSPAFQDRK
SRWRQRRWAYRLLLEMLRYKAEWEGIRVIEVNPAHTSQICPRCGKKDGKARQGNRFCCTECKFQHNADFVAAYNIAVRALKKLG
MREEIGWKVLTGKPRVL
(SEQ ID NO: 623)
>3300028675|Ga0272445_1001731_11|M
[aquatic-thermal springs-sediment]
MKKVVITITTGVEFLQPQDEQIVRKAAEWYTKAREALFAHALQQQQGKVEKAASTLRTLNFPMAPHSRLPDLLTRDVGGILKAL
KEKREGYLGEFKRWWEEVCRVANEKGSQAVAALKTHVDKLLNLVHQLPWNSPVWDGEAQEAFKNLKEAWAVVRQKDREQLKIKW
RAEVSPLRRPPRTPKFQRLVFNADGRVSGGSKSEALQIEEREDGDLYLRLQLRDPEGVVGGGAYQEVWLRLVVEDEERKQMLRD
FAPKYLQLRQDGKRWIAHIVIEREIKFGKPDYLYGVDIGARKYLLAAVAVPLKPDLPKLPNIAIPSGKFWQAVERVEAEWRKWQ
RLRTGKKDKEGRVVLKKRTAWRVLKRKRRMRREVAKNMIAEAVNTFINFIKGDNVDIRLWLAIEQIKNLNLSPAFQDRKSRWRQ
RRWAYRLLLEMLRYKAEWEGIRVIEVNPAHTSQICPRCGKKDGKARQGNRFCCTKCKFQHNADFVAAYNIAVRALKKLGMREEI
GWKVLTGKPKVL
(SEQ ID NO: 624)
>3300029977|Ga0272449_1037372_1|p
[aquatic-thermal springs-sediment]
MKKVVITITTGVEFLQPQDEQIVRKAAEWYTKAREALFAHALQQQQGKVEKAASTLRTLNFPMAPHSRLPDLLTRDVGGILKAL
KEKREGYLGEFKRWWEEVCRVANEKGSQAVAALKTHVDKLLNLVHQLPWNSPVWDGEAQEAFKNLKEAWAVVRQKDREQLKIRW
RAEVSPLRRPPRTPKFQRLVFNADGRVSGGSKSEALQIEEKEDGDLYLRLQLRDPEGIVGGGAYQEVWLRLVVEDEERKQMLRD
FAPKYLQLRQDGERWIAHIVIEREIKFGKPDYLYGVDIGARKYLLAAVAVPLKPDLPKLPNIAIPSGKFWQAVERVEAEWRKWQ
RLRTGKKDKEGRVVLKKRTAWRVLKRKRRMRREVAKNMIAEAVNTFINFIKGDNVDIRLWLAIEQIKNLNLSPAFQDRKSRWRQ
RRWAYRLLLEMLRYKAEWEGIRVIEVNPAHTSQICPRCGKKDGKARQGNRFCCTKCKFQHNADFVAAYNIAVRALKKLGMREEI
GWKVLTGKPKVL
(SEQ ID NO: 625)
>SRR1562008_megahit_k177_216982_5|P
[fermentation metagenome]
MIKTATFYLADMPQEDKRLCQLLCDELGAAGSAMVREWYRACDEVDGERINWGDVQARGVQVGKQAFADRGYRGELVNDLQRAE
FFARFKTEFKKIAAGERASLPFPGSAPFIFLRMDPGREVASVVNEGGTWYLCNPHFYPGGSRSEDNRREWRLKDVGRNGWGYAI
LAEAEKFACIRIMPDRKKRGSWVVKITVHLPDAIARREVEKQEVWAGIDLGLNCPAVLSIPDHPDGGFVRFLGNDQYPRLWKRL
DDYEERKRVLNRAGKREAARDLRAKITGVRQHINELISREIVDLCLAMRVTGLRMEDLKGLGVEGSGRLKFWPRFHLVTRIEQK
CGEVGLTFEKIKPAGTSQTCSSCGHRDKDNRNGPDFVCLRCGFARHADVNAANNIARGMDDFDLPTGASAPTKARRLEIAS
(SEQ ID NO: 626)
>UOPD01011681_4|M
[fermentation metagenome]
MIKTATFYLADMPQEDKRLCQLLCDELGAAGSAMVREWYRACDEVDGERINWGDVQARGVQVGKQAFADRGYRGELVNDLQRAE
FFARFKTEFKKIAAGERASLPFPGSAPFIFLRMDPGREVASVVNEGGTWYLCNPHFYPGGSRSEDNRREWRLKDVGRNGWGYAI
LAEAEKFACIRIMPDRKKRGSWVVKITVHLPDAIARREVEKQEVWAGIDLGLNCPAVLSIPDHPDGGFVRFLGNDQYPRLWKRL
DDYEERKRVLNRAGKREAARDLRAKITGVRQHINELISREIVDLCLAMRVTGLRMEDLKGLGVEGSGRLKFWPRFHLVTRIEQK
CGEVGLTFEKIKPAGTSQTCSSCGHRDKDNRNGPDFVCLRCGFARHADVNAANNIARGMDDFDLPTGASAPTKARRLEIAS
(SEQ ID NO: 626)
>2645728127|Ga0097870_126_5|M
[Fervidibacteria bacterium JGI MDM2]
MEKVILTITSGVEFLQPQDEQLVIKAAKWYTEARKALLYNALQKGEEGVKQAASTLSTLNFPPAPHSRLPDLLTRDVGGTLQAL
KERREGYAQDFSRWWEEICRVANEKGSQAVSVLKPHVNKLSSLIHQLSWNSPEWDEEAQEVFKELKQIWGVVKQKDHERLKIKW
CAEVPPVPRPPRTPKFQRLVFNADGRVSGGSRSNALQIEEREDGDLYLRLQLRDPERVAKKGSYQDVWLRLVIEDEERKQMLKN
FAPKYLQLRQDGKRWIAHIVIERENEFEVPQYVYGVDIGARKYLLAAVAIPLNPALPKPPNIAVPSSKFWQIVERVEAEWRKWQ
QLRTGKRDKEGRVVLKKRKMAWRVLKRKRRMRREVTKSMIAEAVNTFIKSLERPCAIVIEQIKNLHLSPTLQDRKSRWRQRRWA
YRFLLEMLRYKAEWEGMRVIEVNPAYTSQTCPRCGERDRNARQGNLFRCTKCRFQHNADFVAAYNITVRGLMKLRTRGRIDWKI
LTGKPRVL
(SEQ ID NO: 627)
>SRR5272259_megahit_k177_4486292_121|P
[freshwater metagenome]
MTATKQQILQTEYDNLQHFLQTGEDLGVYSANKQQAKRYYKTIKPEHQYPLSIRNDLIQVQHKPNSIAEYWLKIPVKGRRGGLW
VAIRPHQQIPDNAEFLESKIRRKRKGWFANIVIQKQIEPIRTKNILAVDLGEHVIATVCGSFDNQRPRFYGRDVRGIRRHYAWL
RKRLGEKKLLGVIRRIKDTEKRKVNDTLHKISRSIVDLAQEHNATIVLGELQGIRNHAKGRRMNRIVSNMPYLKLSQMIEYKAE
WLGIPVIKMQEHDTSKTCSRCNEYGKRPTQGQFRCDTCHRTSFNADYNGALNILKRATSYMDIAGAVSEPARDWGNDDQTSEAT
QLFEW
(SEQ ID NO: 607)
>SRR5272259_megahit_k177_4486292_1|M
[freshwater metagenome]
MAQKTITAKIVAMTATKQQILQTEYDNLQHFLQTGEDLGVYSANKQQAKRYYKTIKPEHQYPLSIRNDLIQVQHKPNSIAEYWL
KIPVKGRRGGLWVAIRPHQQIPDNAEFLESKIRRKRKGWFANIVIQKQIEPIRTKNILAVDLGEHVIATVCGSFDNQRPRFYGR
DVRGIRRHYAWLRKRLGEKKLLGVIRRIKDTEKRKVNDTLHKISRSIVDLAQEHNATIVLGELQGIRNHAKGRRMNRIVSNMPY
LKLSQMIEYKAEWLGIPVIKMQEHDTSKTCSRCNEYGKRPTQGQFRCDTCHRTSFNADYNGALNILKRATSYMDIAGAVSEPAR
DWGNDDQTSEATQLFEW
(SEQ ID NO: 608)
>SRR5273149_megahit_k177_25824_1|M
[freshwater metagenome]
MEKVVITITTGVEFLQPQDEQIVRKAAEWYTKAREALFAHALQQGKVKKAASTLRTLNLPMAPHSRLPDLLTRDVGGILKALKE
KREGYLREFKRWWEEVCRVANEKGSQAVAALKTHVDKLLNLVHQIPWNSPVWDGEAQEAFKNLKEAWAVVRQKDREQLKIKWRA
EVSPLRRPPRKPKFQRLVFNADGRVSGGSRAEALQIEEREEGDLYLRLRLRDPEGVVGGGAYQEVWLRLVIEDEGRKQMLRDFA
PKYLQLRQDGKRWIAHIVIEREIKFGEPDYLYGVDIGARKYLLAAVAVPLKPDLPKLPNIAIPSGEFWQAVERVEAEWRKWQRL
RTGKKDKEGRVVLKKKRMAWRVLKRKRRMRREVAKNMIAEAVNTFINFIKRGNVNSGLAIEQIKNLNLSPTLQDRKSRWRQRRW
AYRLLLEMLRYKAEWEGIRVIEVNPTHTSQICPRCRKKDGKARQGNRFCCTKCHFQHNADFVAAYNIAMRGLRKLGMRGEIGWK
VLTGKPRVLQ
(SEQ ID NO: 628)
>SRR5273182_megahit_k177_57442_1|M
[freshwater metagenome]
MEKVVITITTGVEFLQPQDEQIVRKAAEWYTKAREALFAHALQQGKVKKAASTLRTLNLPMAPHSRLPDLLTRDVGGILKALKE
KREGYLREFKRWWEEVCRVANEKGSQAVAALKTHVDKLLNLVHQIPWNSPVWDGEAQEAFKNLKEAWAVVRQKDREQLKIKWRA
EVSPLRRPPRKPKFQRLVFNADGRVSGGSRAEALQIEEREEGDLYLRLRLRDPEGVVGGGAYQEVWLRLVIEDEGRKQMLRDFA
PKYLQLRQDGKRWIAHIVIEREIKFGEPDYLYGVDIGARKYLLAAVAVPLKPDLPKLPNIAIPSGEFWQAVERVEAEWRKWQRL
RTGKKDKEGRVVLKKKRMAWRVLKRKRRMRREVAKNMIAEAVNTFINFIKRGNVNSGLAIEQIKNLNLSPTLQDRKSRWRQRRW
AYRLLLEMLRYKAEWEGIRVIEVNPTHTSQICPRCRKKDGKARQGNRFCCTKCHFQHNADFVAAYNIAMRGLRKLGMRGEIGWK
VLTGKPRVLQ
(SEQ ID NO: 628)
>SRR5273183_megahit_k177_82677_3|M
[freshwater metagenome]
MAPHSRLPDLLTRDVGGILKALKEKREGYLREFKRWWEEVCRVANEKGSQAVAALKTHVDKLLNLVHQIPWNSPVWDGEAQEAF
KNLKEAWAVVRQKDREQLKIKWRAEVSPLRRPPRKPKFQRLVFNADGRVSGGSRAEALQIEEREEGDLYLRLRLRDPEGVVGGG
AYQEVWLRLVIEDEGRKQMLRDFAPKYLQLRQDGKRWIAHIVIEREIKFGEPDYLYGVDIGARKYLLAAVAVPLKPDLPKLPNI
AIPSGEFWQAVERVEAEWRKWQRLRTGKKDKEGRVVLKKKRMAWRVLKRKRRMRREVAKNMIAEAVNTFINFIKRGNVNSGLAI
EQIKNLNLSPTLQDRKSRWRQRRWAYRLLLEMLRYKAEWEGIRVIEVNPTHTSQICPRCRKKDGKARQGNRFCCTKCHFQHNAD
FVAAYNIAMRGLRKLGMRGEIGWKVLTGKPRVLQ
(SEQ ID NO: 629)
>SRR6481359_megahit_k177_932521_3|M
[freshwater sediment metagenome]
MANRELVVVMQVQKVVKGKVVGLTHAKRQILDHEYEGLQRFLQGDSNVELYSANKQQAKRFYKTVKPEREYPLSIRKDLIRVER
RDTKIARYWARIPAKLRRGGVWVAVKPHCEITDDLEICESKLLRRNGEYFLHLAVKREVELNTSYSSVLAIDLGIRHVACSVDT
RSGETHFHGRELRRVRGHYFHLRRRLGEKKLLKVIKRIGARESRITNDQLHKIAKAIVDEAERLNALIAIGNPKGIRRNGKGRR
FNRKLNSWPFWKLRQYIEYKANWRGIAVVEVNEAYTSQRCWRCGTIGTRSGTHHGLFECPRCGLRENADRNGAFNIGRRALGQV
SKVGAVVSQPVTGAVPFSELGSVVQCPTPEATLFKGW
(SEQ ID NO: 609)
>3300012677|Ga0153928_1011778_2|M
[fungi-mycelium-attine ant fungus gardens]
MKITVKAKVTDDSKNKLGKIESEFQNYQVYLQSGCTTRVNLYSATKQQAQRALKKIKQPNPKKQYPVILRRDLVDIQQDIKFEG
VYWIKIPVYPNSINIRIKTSCKYDLTQHSIRESKILKIQNVWYIFITIEKQDERIIRADNVLAVDLGCKNIAVTVNTINTKPNF
YGAAIRGIRGFYFNLRRKLGEKKASDKIKLLKNIEFLQVNHELHKISKAIVEEAKRTNAVIVLGKLKGIRKNIKGSRRIRRLIN
NFPYYRLVQYIKYKAAWHSIKTLEISEADTSNTCFNCRTKDKKARKTQGLFQCGNCSIKTNADYNGAMNILQRGVGYFQL
(SEQ ID NO: 630)
>WP_121304574.1
[Haloarcula quadrata]
MKRNVSTTVRAKLHSLTNRKTDLLAREYEAFQTEVHGGDADLYSATDQQASKVQRQKYPNPGTEQPVVLRNDVFDVAYDEDTVL
SSWWVKVPVYDPEKGQGNSIWCPAHVPHKDEQLVREGNLRDSELVRSDGDWYVHLVVKRSVTVQDEYDDVLAIDMGARWVATCA
FLSDRKTTFYGEEVRRIREHYKQLRKSIGKAKPRQGQQVVERIGDAEARKVDDRLHKIARSIVEDAEERNAVIVVGDLGGIRKD
NDKGRYVNDKTHKMPFARLLNYIEYKAHGAGIDVQLVEEYDTSKTCNRCACEGVRTTQGRFECPECGLDDNADKNGALNIGKRA
LGKFLKPLSEAGAVLAQSETQVIVQRDDEPANLSVSVDSTPSGGTPRL
(SEQ ID NO: 631)
>SEJ25361.1
[Halohasta litchfieldiae tADL]
MKQTAEKTVVAKIFTNDLTQSKLDAINHEYEAFQRYIRGDEEADLYSATKQCADAYVDTDNLRDDHDYPWFIRNDVFDVGVDVE
QHDTDLTDWWLKLPVSQVWGGVNVPIQPHDEIPDEADVKDSKIVRVNGDYYAHLTVQQEVEVSEEYGGVIGVDFGVRWVATSVA
LPSRNTEFYGREIRRIRRHHHDLRTRLQEKGAYGTLQSVKVKEYRQVNDRLHKISRAIVEEAKECDALIVVGDLEGIQNQDLGA
EMNRRLHSMPHHTLKTYIEYKAMFAGIAVKSVNEYMTSQTCWRCGEQEATTRKGQGQHLCHECGLDDNADKNGATNIAKKGLGK
DIACPLSSLGAVCEPVLGPSGADQASATVCLRADLEAPASTKRTVR
(SEQ ID NO: 615)
>WP_121597521.1
[Halorubrum sp. Atlit-26R]
MQRNVSTTVRVKLHSLTNRKADLLAREYEAFQTAVHGGDADLYSATDQQASKVQRQKDPNPDTEQPVVLRNDVFDVAYDEDTVL
SSWWIKVPVYDPEQGRGNSIWCPAHVPYKDEQLVREGDVRDSELVRROGNWYVHLVVRRSVTVQDEYDDVLAIDMGARWVATCA
FLSDRKTAFYGEEVRRIREHYKQLRKSIGKAKPRQGQQVVKRIGDAEARKVDERLHKIARQIVEDAEERNAVIVVGDLGGIRKD
NDKGRYVNDKTHKMPFARLLNYIEYKAHDAGIDVQLTNEYDTSQTCNRCACKGVRETQGRFECPECGLDDNADKNGALNIGKRA
LGKFSKPLSEAGAVLAQPETQVIVPRDDEPANLSVSVGSTPSGGTPRL
(SEQ ID NO: 632)
>WP_006630662.1
[Halorubrum tebenquichense DSM 14210]
MQRNVSTTVRVKLHSLTNRKSDMLAREYEAFQREVHGGDADLYSATKQQASKVQQQKDPNPDTEQPVVLRNDVLDIAHDKDTVL
SSWWMKVPVYDPERGQGNSIWCPAHVPHKDETLVQEGDIRDSELVRRDGDWYVHLVVKRSVTIPDEYDDVLAIDMGARWVATCA
FLSDRDTTFYGEEVRRLREHYKQLRKSIGKAKPRQGQQVVERIGDAEARKVDDRLHKIARHIVEDAEERNTVIVVGDLGGICRD
NDKGRYINDKIHKMPFARLLNYIEYKAHGAGIDVVLVEEYDTSKTCNRCACEGVRETQGRFECPECGLDDNADKNGALNIGKRA
SGKFSKPLSEAGAVLAQPKTQVIVERDEEPANLSISVGSTPSGGTPRL
(SEQ ID NO: 633)
>DRR082460_megahit_k177_106880|P
[hot springs metagenome]
MRRRKPQQEGKEVQRTIICGVKFVAHEDEQLITESAKWYLEAKQKLLRYALQQEKVSISNPATVLKGLSVPAPPHSRLPDELSQ
AVSGILKALKKRRSEYLEKFQNWWQEVVRVSNKHGLQSFIPLKPKVEAFLNALANVKSWADPAWDGELQRRFEELKDMWAVVRQ
RDSNERRFRWRLHVPPLPKPPKTPELRLLGFDAGVRVSGGSRAQTLQIEQRNNDLFLRLLLARPGEKSKAGQYREVWLQLVLED
DLRKNWLLSPDIEAKFVRLRQIEDGKWQAHITVRKKVAKSEDFNIVYGIDIGLKRALLYCVPVATANAKPENWKELRLNKFIVP
AGEFWHRVIVEDERYRKWQRRFKGKKTEGIKGKRMAWRILKRLRKRRRKVTELLIHRVVNSLLENLMKASETAGFAIAIERLKD
LRNKPHLTSGKAQWKFHRWAYRFLLNTLRYKAEWEGVTVIEVNPAGTSQTCPRCGYKSRESRHDRLFRCRRCKFQHNADAVAAF
NIAKRVIEMKNGQVNQDAWKVLTGEWNVLKY
(SEQ ID NO: 634)
>DRR082460_megahit_k177_106880|M
[hot springs metagenome]
MQVRRRKPQQEGKEVQRTIICGVKFVAHEDEQLITESAKWYLEAKQKLLRYALQQEKVSISNPATVLKGLSVPAPPHSRLPDEL
SQAVSGILKALKKRRSEYLEKFQNWWQEVVRVSNKHGLQSFIPLKPKVEAFLNALANVKSWADPAWDGELQRRFEELKDMWAVV
RQRDSNERRFRWRLHVPPLPKPPKTPELRLLGFDAGVRVSGGSRAQTLQIEQRNNDLFLRLLLARPGEKSKAGQYREVWLQLVL
EDDLRKNWLLSPDIEAKFVRLRQIEDGKWQAHITVRKKVAKSEDFNIVYGIDIGLKRALLYCVPVATANAKPENWKELRLNKFI
VPAGEFWHRVIVEDERYRKWQRRFKGKKTEGIKGKRMAWRILKRLRKRRRKVTELLIHRVVNSLLENLMKASETAGFAIAIERL
KDLRNKPHLTSGKAQWKFHRWAYRFLLNTLRYKAEWEGVTVIEVNPAGTSQTCPRCGYKSRESRHDRLFRCRRCKFQHNADAVA
AFNIAKRVIEMKNGQVNQDAWKVLTGEWNVLKY
(SEQ ID NO: 635)
>OGCH01000195_3|M
[hot springs metagenome]
MKHLVSNAQVNEKNEARLTLKCGLEFENAEGEELVKRAAEWYCKARIFLLSYALKNNKTRVQKIASLLKSEGFPSPPHPRLIDL
LQRTITPAIKNMRRLRRRYPKTLRAWFDRVKKFAEEKGSDAAERLQPYVEDLLQLVKNLPINSPAWDTDAQEKWKELKKLWGKV
RESDWKKGIVWKFQIPNPKKPPSTPAFRCADSSNIFVTADYRMSGKNARNSYDMKTDSEENLWVLVRLDDVDGDVKVRKDVWIK
LCVDKSRYDLLRIGKITEIKILRNRDGRWEGRIVVKMNAGYNSPQVVVGVDVGASEHLIAAVGIPLDGSNNNFLLSVPASYFWK
RVEQAERLWRRWQRLYTGRTSEKKKAPILPRRRKAYQILKRNRNWRREVTKHCIFHAVKKFVQLLPPNSVVVVENLKDIPPNVW
KEDKDKRFRRWVYAFLLFTLKYKCQQRKIAVIAVSPHNTSQTCPRCGVVDPKSRGLWKDGELISRKHFCCTQCEFTHNADFVAA
HNIAVLGAKALGITLPESWRALRGEVQYVIPYQNMPKVRKSW
(SEQ ID NO: 636)
>SRR4027810_megahit_k177_26210_1|M
[hot springs metagenome]
MEKVILTITSGVEFLQPQDEQLVIKAAKWYTEARKALLYNALHKGEEGVKQAASTLSTLNFPPAPHSRLPDLLTRDVGGTLQAL
KERREGYAQDFSRWWEEICRVANEKGSQAVSVLKPHVNKLSSLIHQLSWNSPEWDEEAQEVFKELKQIWAVVRQKDHERLKIKW
RAEVPPVPRPPRTPKFQRLVFNADGRVSGGSRSNALQIEEREDGDLYLRLQLRDPERVAKKGSYQDVWLRLVIEDEERKQMLKN
FAPKYLQLRQDGKRWIAHIVIERENEFEVPQYVYGVDIGARKYLLAAVAIPLNPALPKPPNIAVPSSKFWQIVERVEAEWRKWQ
QLRTGKRDKEGRVVLKKRKMAWRVLKRKRRMRREVTKSMIAEAVNTFIKSLERPCAIVIEQIKNLHLSPTLQDRKSRWRQRRWA
YRFLLEMLRYKAEWEGIRVIEVNPAYTSQTCPRCGKRDRNARQGNLFQCTKCRFQHNADFVAAYNITVRGLMKLRTRGRIDWKI
LTGKPRVL
(SEQ ID NO: 637)
>SRR5247126_megahit_k177_220968_4|P
[hot springs metagenome]
MEKVVITITTGVEFLQPQDEQIVRKAAEWYTKAREALFDHALQQGKVKKAASTLRTLNLPMAPHSRLPDLLTRDVGGILKALKE
KRKGYLREFKRWWKEVCKVANEKGSQAVAVLKPHVDKLLNLVRQLPWNSPVWDGEAQEAFKNLKEAWAVVRQKDREQLKIKWRA
EVSPLRRPPRTPKFQRLVFNADGRVSGGSKSEALQIEEREDGDLYLRLQLRDPEGVVGGGAYQEVWLRLVVEDEERKQMLRDFA
PKYLQLRQDGKRWIAHIVIEREIKFGLKGIMWTAGDYLYGVDIGARKYLLAAVAVPLKPGLPKLLESQGDNVGIRHIAIPSGKF
WQAVERVEAEWRKWQRLRTGKKDKEGRVVLKKKRMAWRVLKRKRRMRREVAKNMLAKAVNTFILDFIKRNNVDIWLAIEQIKNL
NLSPTLQDRKSRWRQRRWAYRLLLEMLRYKAEWEGIRVIEVNPAHTSQICPRCGKKDGKARQGNRFCCTKCHFQHNADFVAADN
IAMRGLRKVGMRGEIGWKVLTGKPRVLQ
(SEQ ID NO: 638)
>3300027941|Ga0207719_100292_34|M
[lab enrichment-defined media-aerobic enrichment media]
MQQTVSTTVRVKLPSLTNRKADLLAREYEAFQTEVHGGDADLYSATKQQASKVRRQKDPNPDTEQPVVLRNDVFDVSYDEDTVL
SSWWVKVPVYDPERGRGNSIWCPAHVPHKDEQLVQEGDVRDSELVRRDGDWYIHLVVKRSVTVQDEYDDVLAIDMGARWVATCA
FLSDHKTTFYGEEVRRIREHYKQLRKSIGKAKPRQGQQVVERIGDAEARKVDDRLHKIARQIVGDAEERNAVIVVGDLGGIRKD
NDKGRYVNDKTHKMPFARLLNYIEYKAHDAGIDLQLVDEYDTSQTCNRCACEGVRATQGRFECPGCGLDDNADKNGALNIGKRA
LGKFSRPLSEAGAVLAQSETQVIVHRDEEPANLSVSVDSTPSGGTPRL
(SEQ ID NO: 639)
>3300027941|Ga0207719_100292_35|P
[lab enrichment-defined media-aerobic enrichment media]
MRVKLPSLTNRKADLLAREYEAFQTEVHGGDADLYSATKQQASKVRRQKDPNPDTEQPVVLRNDVFDVSYDEDTVLSSWWVKVP
VYDPERGRGNSIWCPAHVPHKDEQLVQEGDVRDSELVRRDGDWYIHLVVKRSVTVQDEYDDVLAIDMGARWVATCAFLSDHKTT
FYGEEVRRIREHYKQLRKSIGKAKPRQGQQVVERIGDAEARKVDDRLHKIARQIVGDAEERNAVIVVGDLGGIRKDNDKGRYVN
DKTHKMPFARLLNYIEYKAHDAGIDLQLVDEYDTSQTCNRCACEGVRATQGRFECPGCGLDDNADKNGALNIGKRALGKFSRPL
SEAGAVLAQSETQVIVHRDEEPANLSVSVDSTPSGGTPRL
(SEQ ID NO: 640)
>SRR6484323_megahit_k177_106899_4|P
[landfill metagenome]
MALKTLTFWPLEIPPEHKRIFDDIRKQLGNAQREMVKAWLKLALAAEPYGITPGEQAGRVLFGETDYSNGLLKSLAYTDARDSF
KEQWKGVLKGDATSVSFPDEASGVVLLVGSGQATVEKIDGEWWVTRLLVFGGRTAEDRSRNDQFRWQLKGVSRFGGHYEKSVMD
RAERICQVRVLPARKDRGKYVVQVTVEVPDEQIERTAIPVCAGIDLGINCPVVLSIPDKGFVRFMGRERDEYPRLWEKLREYED
RKRTLNRAGKRRAARDLRPRLTNIRKYINEAISKDVADTCRAMRVTTLRMENLKGLSFHKSGTGKLRHWPRYHLQERIEQKCGE
VGIEVERIKPAGTSQTCSMCGHRDKGNRNGSDFICLACGYRQHADLNAANNIARGMQEFGPCVQSSASDNTIELRFDGAGQEPA
LGRAMRLFA
(SEQ ID NO: 641)
>SRR6484325_megahit_k177_1345066_6|P
[landfill metagenome]
MALKTLTFWPLEIPPEHKRIFDDIRKQLGNAQREMVKAWLKLALAAEPYGITPGEQAGRVLFGETDYSNGLLKSLAYTDARDSF
KEQWKGVLKGDATSVSFPDEASGVVLLVGSGQATVEKIDGEWWVTRLLVFGGRTAEDRSRNDQFRWQLKGVSRFGGHYEKSVMD
RAERICQVRVLPARKDRGKYVVQVTVEVPDEQIERTAIPVCAGIDLGINCPVVLSIPDKGFVRFMGRERDEYPRLWEKLREYED
RKRTLNRAGKRRAARDLRPRLTNIRKYINEAISKDVADTCRAMRVTTLRMENLKGLSFHKSGTGKLRHWPRYHLQERIEQKCGE
VGIEVERIKPAGTSQTCSMCGHRDKGNRNGSDFICLACGYRQHADLNAANNIARGMQEFGPCVQSSASDNTIELRFDGAGQEPA
LGRAMRLFA
(SEQ ID NO: 641)
>SRR6484787_megahit_k177_1514834_8|M
[landfill metagenome]
MDTAANETTPKALCFWLGALSDAEFKLLHELSRQLARCHATFVQAALEYQFAADRGSAPEVIAQTGPNKGKPSSDPRETYAKRA
FQAEFAKAPFMRKFGDTYCRQVSGKDFKRQWGLIRTGQKSSVSFGPGSHANIDILTGRQAAIEHDADGWWLCGLGITLEDRKER
PGRRWALIPQTNGGAAVKAKQAARMLRQLEEAVRTPQVRLVWRDGKRGGSKGWEAQVVCHMPVKVAKPAEPVVCGIDVGMRCLL
VASIPERRKARFIGGNTFGKLWHEIERHGKRRSVLNKAHKRHAAAAKSDKVSRIRRHACELASRQLIDWCVYHHVSTIRMEDLS
GIRDRCSEDGATEQQRTWNKRLAHWPWYTLQQRIEQKAAAVNIAVEKINPHLTSQTCSACGHVDKGNREGAQFRCLSCGFECHA
DLNAANNIAGRDPATDFDTPDRAAVRSDGPKSRPQAGRASRLVAASTP
(SEQ ID NO: 642)
>SRR8542065_megahit_k177_1115558_2|M
[landfill metagenome]
MALKTLTFWPLEIPPEHKRIFDDIRKQLGNAQREMVKAWLKLALAAEPYGITPGEQAGRVLFGETDYSNGLLKSLAYTDARDSF
KEQWKGVLKGDATSVSFPDEASGVVLLVGSGQATVEKIDGEWWVTRLLVFGGRTAEDRSRNDQFRWQLKGVSRFGGHYEKSVMD
RAERICQVRVLPARKDRGKYVVQVTVEVPDEQIERTAIPVCAGIDLGINCPVVLSIPDKGFVRFMGRERDEYPRLWEKLREYED
RKRTLNRAGKRRAARDLRPRLTNIRKYINEAISKDVADTCRAMRVTTLRMENLKGLSFHKSGTGKLRHWPRYHLQERIEQKCGE
VGIEVERIKPAGTSQTCSMCGHRDKGNRNGSDFICLACGYRQHADLNAANNIARGMQEFGPCVQSSASDNTIELRFDGAGQEPA
LGRAMRLFA
(SEQ ID NO: 641)
>SRR8542065_megahit_kl77_821416_8|M
[landfill metagenome]
MLGQVKTVKFHITGVTRDQGEALTGSLRALGAAARTAVKTALMEVADKDLHGVKDAYWQGVQVRAEQSAREAFKETGYSPAILN
EYWNAFGFTGFKIRWSQVRKGERTSIPFPGDAPFLQFTNGATIQVAETEDGYCIRRIRIYGLVGNKPDTRNEVEWALKPVGRGR
HAAWGIEQLRRANKICAVRIHKAERGWIAAVSVRIQAEAVAASRPERWAGIDLGLNSPAVLSVPDANFSRFYGRDLYSGLWRSL
DEYEARKRELVRAGKKQAARDLDDRISAKRKHINELISRQIVDDCRRLRVTGIRLEDLKGIQQFATGRLKHWPRHNLALRIEQK
AAEVGIETEMVRPAYTSLTCSHCGHMEKDNRSRENVSLFLCRRCGFSRHADLNGANNIARKLERFGIEEPNLSVFERAEQPRLH
RLGATTGPEHLEGRLTEAAT
(SEQ ID NO: 643)
>FZPS01000148_2153|M
[metagenome]
MIKTATFYIADMPQEDKRLCERLCAELGAAGATMVREWYRACDEVTGERINWPEVQKRGVQVGRQAFAERGYRAELVNDLQRAE
FFARFKMEFKKIQAGERASLPFPGSAPFIFLRMDPGKEKATLVQEDGTWYLRDPHFYPGGGRNEDNKREWRLKDVGRNGWGYAI
LAEAEKAACIRIMPDRKKRGSWVVKITVHLPDAALRKEVERQEVWAGIDLGLNCPAVLSIPDHPDGGFVRFFGNDEYARLWQRL
KEYEDRKRRLNRAGKRRAARDLRAKITGVRQHINELISREIIDLCIAMRVTGLRMEDLKGLGADGSGKLKFWPRFHLVTRIEQK
AHEAGLEFVKIKPAGTSQTCSACGYRDKDNRNGSDFTCKRCGFSRHADVNAANNIARGMEEFSLPTGATAPNKAQRLVIAS
(SEQ ID NO: 644)
>WP_005554257.1
[Natronococcus amylolyticus DSM 10524]
MHREVTTTVRVKLHSLTERKARLIEREYTAFQDAVHGGDASLYSATKQQAGKVRSNKNPREDTDQPVVLRNDCITIEHDEDTVL
SSWWFKLPIYNPKRERGDSIWVPVHVPEKDTHLLTDEYIRDSELVHRDGEWYVHLVCKRSVAVADEYDDVLAVDMGAKWIAVST
FLSDRDTQFHGAEVRRVREHYKQLRKSIGKAKVRQGAQVIERIGDKESWTVEHELHQVANELIARARERNAVIVFGDMTGLRFD
NDEGRYVNDKTHKMPYAKLANILTYKSHLDGRECIPVSEYDTSVTCWRCGSQNTSRGAQGRVECCDCGLDDNADKNGASNIGKR
AVGKDIQSPLSTVGAAVAQPETQVVLDGTSGEMEPANSPKDVGLTLSEGSPRL
(SEQ ID NO: 645)
>SRR7004341_megahit_k177_2665443_5|P
[peat metagenome]
MSLVKVKKCYYCKVIEDLTNTKLVALQQEYDNLQHYLQTKEDLGLYSANKQQANRFYKVIKRGKKYPLSIRNDLIRIEHKPSTI
AEYWIRIPVKSVKGGLWIGLIKPYEPIPTDAKICESKLYKKONSWFLDLVVEKDIPEKLQYQNVIGIDMGIRHIATSVELANGL
TKFYGKDLNRVRGHYFWLRRKLGIKKAIDTIKKIGDHEKRIANDIIHNISRDIVNQAIESNAMIVLGDIKHLRRRKKNTRGKKF
NRKLAGFQYCKLTQYITYKAALAGIKVIQVSQARTSQYCRKCYQKGVRKIQGLFTCAKCGEENADRNAAFNIIYRALGYISKVG
VTVNIPNARTFASVDRNAMMTREATCF
(SEQ ID NO: 646)
>SRR8526178_megahit_k177_763700_3|P
[salt lake metagenome]
MSLKTLTFWPIGMSAKYRRIFDNIRKQLGAAQREMVKAWLRLALAEDAYGIDPGKRAGREVFGDNEYSNDLLNNLAYADAHENF
KQQWKRVRSGNATSVSFSDAAANVLFLMPPSGGHHATVEHIDGNWWITRLKVFGGCTKADRERNDQFRWQLKGVSRFGGKYEKD
LMDGAQRICQVRVTPSRKDPSKYVVQVTVEVPDLQTVGARTPVCAGIDLGINCPVVLSIPDKGFVQFMGRERDEYPRLWEKLRE
YEDRKRRLNRAGKRRAARDLRPRITNVRKYINEAISKDVADTCRALGVTHLRMEDLKGLSRHKSMTGKLRYWPRYHLQERIERK
CCEVGIEVERIKPAGTSQTCSVCGHREKANRNGAQFVCIECGYQQHADLNAANNIARGMQTFGPCAKSSASDDAIGLRFDSEGQ
ESALGQAKRLFA
(SEQ ID NO: 616)
>OVXO01016275_2|M
[sediment metagenome]
MLVRKVIKAKIMDEANSRKLKALEKEYNNFQRALRGEDVKLYSATRQQAERLLRKIRKQRGKLKQKEYPLILRNDVYDVRITNN
KLSKYWIKIPVYGVRGGIKLPIKPHCEIDASWKFKEAKVIRKENGWFVHITVEKEIEIKKEYENVIAIDLGIRNIATVVSTADG
KPRFYGKELRRIRGHYFHLRRRLAKLKKFKAIKKIGNKERRVVDDLLHKISREIVNRAKEENAVIVLGDLKRIRKQNRGRKFNR
KLNTFPYYRLSKFIEYKALWEGIAVIKVNEANTSKICSRCGSQGLRRNGKFVCSKCGVELNADYNGALNILKRALGYMSKAGAD
LTQPFQSHFGLILTMKKRYGRD
(SEQ ID NO: 647)
>SRR7067823_megahit_k177_107253_1|P
[sediment metagenome]
MEKVILTITSGVEFLQPQDEQLVIKAAKWYTEARKALLYNALQKGEEGVKQAASTLSTLNFPPAPHSRLPDLLTRDVGGTLQAL
KERREGYAQDFSRWWEEICRVANEKGSQAVSVLKPHVNKLSSLIHQLSWNSPEWDEEAQEVFKELKQIWAVVRQKDHERLKIKW
RAEVPPVPRPPRTPKFQRLVFNADGRVSGGSRSNALQIEEREDGDLYLRLQLRDPERVAKKGSYQDVWLRLVIEDEERKQMLKN
FAPKYLQLRQDGKRWIAHIVIERENEFEVPQYVYGVDIGARKYLLAAVAIPLNPALPKPPNIAVPSSKFWQIVERVEAEWRKWQ
QLRTGKRDKEGRVVLKKRKMAWRVLKRKRRMRREVTKSMIAEAVNTFIKSLERPCAIVIEQIKNLHLSPTLQDRKSRWRQRRWA
YRFLLEMLRYKAEWEGIRVIEVNPAYTSQTCPRCGERDRNARQWNLFRCTKCRFQHNADFVAAYNITVRGLMKLRTRGRIDWKI
LTGKPRVL
(SEQ ID NO: 648)
>SRR7067986_megahit_k177_69094_7|P
[sediment metagenome]
MEKVILTVTSGVEFLQPQDEQLVIKAAKWYTEARKALLYNALQKGEEGVKQAASTLSTLNFPPAPHSRLPDLLTRDVGGTLQAL
KERREGYAQDFSRWWEEICRVANEKGSQAVSVLKPHVNKLSSLIHRLSWNSPEWDEEAQEVFKELKQIWGVVKQKDHERLKIKW
CAEVPPVPRPPRTPKFQRLVFNADGRVSGGSRSNALQIEEREDGDLYLRLQLRDPERVAKKGSYQDVWLRLVIEDEERKQMLKN
FAPKYLQLRQDGKRWIAHIVIERENEFEVPQYVYGVDIGARKYLLAAVAIPLNPALPKPPNIAVPSSKFWQIVERVEAEWRKWQ
QLRTGKRDKEGRVVLKKRKMAWRVLKRKRRMRREVTKSMIAEAVNTFIKSLERPCAIVIEQIKNLHLSPTLQDRKSRWRQRRWA
YRFLLEMLRYKAEWEGIRVIEVNPAYTSQTCPRCGERDRNARQGNLFRCTKCRFQHNADFVAAYNITVRGLMKLRTRGRIDWKI
LTGKPRVL
(SEQ ID NO: 649)
>SRR8554444_megahit_k177_504614_2|M
[sediment metagenome]
MVAVKTIKAKIIAPTRRKAEILEKEYQGFQDILHGQQAEIYSRTRDQALRVLRRIKAPKDRHYPLILHRASIRLERRDTKIAQY
WFRFPCHGVRGGIWLAIKPHCQIPEGCSLRETKLIKRNNAWFLYIIIKKEVPEPVINPDRIVAVDLGERYLATVCGGEGIRPQF
LGKEARGIRRHYAWLRRRLGELKLLRTIKKVGNTERRKINTICHQISRKIVDLAKQTGSTIVLGDLKGIRQRARGRRMNRIVSG
MPYYKLTQFIKYKAAWESVPVLIVSERDTSKTCHRCGAIGTRPYQGLFLCQACGLRYSADLNGAKNIMKRAREYISLAGAAFSQ
PMTRALAGNPLMETG
(SEQ ID NO: 650)
>SRR8554446_megahit_k177_83048_1|M
[sediment metagenome]
MKKVVITITTGVEFLQPQDEQIVRKAAEWYTKAREALFAHALQQQQGKVEKAASTLRTLNFPMAPHSRLPDLLTRDVGGILKAL
KEKREGYLGEFKRWWEEVCRVANEKGSQAVAALKTHVDKLLNLVHQLPWNSPVWDGEAQEAFKNLKEAWAVVRQKDREQLKIRW
RAEVSPLRRPPRTPKFQRLVFNADGRVSGGSKSEALQIEEKEDGDLYLRLQLRDPEGIVGGGAYQEVWLRLVVEDEERKQMLRD
FAPKYLQLRQDGERWIAHIVIEREIKFGKPDYLYGVDIGARKYLLAAVAVPLKPDLPKLPNIAIPSGKFWQAVERVEAEWRKWQ
RLRTGKKDKEGRVVLKKRTAWRVLKRKRRMRREVAKNMIAEAVNTFINFIKGDNVDIRLWLAIEQIKNLNLSPAFQDRKSRWRQ
RRWAYRLLLEMLRYKAEWEGIRVIEVNPAHTSQICPRCGKKDGKARQGNRFCCTKCKFQHNADFVAAYNIAVRALKKLGMREEI
GWKVLTGKPKVL
(SEQ ID NO: 625)
>SRR6201998_megahit_k177_1568738_2|M
[soil metagenome]
MKITVKAKVTDDSKNKLEKIESEFQNYQVYLQSGCTAIVNLYSATKQQTQRALKKIKQPNQKKQYPVILRRDLIDIQKDIKFKG
VYWMRIPVYPNSINIRIKTSCKYDLTEYSIRECKILKIRNEWYIFITIEKQDEPIIPPDNVLAVDLGCKNIAVTVNTINTKPNF
YGAALRGIRGFYFNLRRKLGEKKASNKIKSLKNIEFLQVNHELHKISKAIVEEAKRTNAVIVLGKLKGIRKNIKGSRRMRRLIN
NFPYYRLVQYIKYKAAWRSIRALEISEADTSNTCFNCRTKDKKARKTQGLFQCSNCSIRTNADYNGAMNILQRGVGILSTLGGF
LTYPEPSVIIDRNKMITKEPQML
(SEQ ID NO: 651)
>SRR8554511_megahit_k177_4248910_4|P
[soil metagenome]
MIKTATFWLCEVTSDQRAVLVDISKQLGAAHRAMVCEWLRLAMAGEPYGENAGKQKAREVFADTPYSNGLVNNYSHADAHPRFK
KQWEAVLKGERSSVAFPGRAPFIYLGAPEQVSVELLDGGWHIVDPRLYIGDSKDNGRSITWQEGRSRNRSIAWRLDPVDNRRTG
WCLGLLRDAECVSCVRLMKSDRRPGRWLAKISVELAEAASARDKEEVELCAGIDLGLNCPVVLSVPDKGLLRFFGREGDMYPRL
WQKLRQYEDRKRAFNRAGHKREARDLRMNLTNVRQYINECVSKAVVDYCLANRVTKLRLENLKGLGVGATGKLKHWPRYHLQQR
IEQKGAAAGLEVEYVKAAGTSQTCSVCGHRHRDNRQRSQFECLACGYTAHADVNAANNIARNLETFGPVTGSRSAQEAPVGLRF
DWEGREPGLRGKSSRLQLDLGTQVSRSAEGGVINAAT
(SEQ ID NO: 652)
>SRR8554511_megahit_k177_4248910_4|P
[soil metagenome]
MDISKQLGAAHRAMVCEWLRLAMAGEPYGENAGKQKAREVFADTPYSNGLVNNYSHADAHPRFKKQWEAVLKGERSSVAFPGRA
PFIYLGAPEQVSVELLDGGWHIVDPRLYIGDSKDNGRSITWQEGRSRNRSIAWRLDPVDNRRTGWCLGLLRDAECVSCVRLMKS
DRRPGRWLAKISVELAEAASARDKEEVELCAGIDLGLNCPVVLSVPDKGLLRFFGREGDMYPRLWQKLRQYEDRKRAFNRAGHK
REARDLRMNLTNVRQYINECVSKAVVDYCLANRVTKLRLENLKGLGVGATGKLKHWPRYHLQQRIEQKGAAAGLEVEYVKAAGT
SQTCSVCGHRHRDNRQRSQFECLACGYTAHADVNAANNIARNLETFGPVTGSRSAQEAPVGLRFDWEGREPGLRGKSSRLQLDL
GTQVSRSAEGGVINAAT
(SEQ ID NO: 653)
>3300014204|Ga0172381_10015437_8|M
[solid waste-landfill-landfill leachate]
MDTAANETTPKALCFWLGALSDAEFKLLHELSRQLARCHATFVQAALEYQFAADRGSAPEVIAQTGPNKGKPSSDPRETYAKRA
FQAEFAKAPFMRKFGDTYCRQVSGKDFKRQWGLIRTGQKSSVSFGPGSHANIDILTGRQAAIEHDADGWWLCGLGITLEDRKER
PGRRWALIPQTNGGAAVKAKQAARMLRQLEEAVRTPQVRLVWRDGKRGGSKGWEAQVVCHMPVKVAKPAEPVVCGIDVGMRCLL
VASIPERRKARFIGGNTFGKLWHEIERHGKRRSVLNKAHKRHAAAAKSDKVSRIRRHACELASRQLIDWCVYHHVSTIRMEDLS
GIRDRCSEDGATEQQRTWNKRLAHWPWYTLQQRIEQKAAAVNIAVEKINPHLTSQTCSACGHVDKGNREGAQFRCLSCGFECHA
DLNAANNIAGRDPATDFDTPDRAAVRSDGPKSRPQAGRASRLVAASTP
(SEQ ID NO: 642)
>3300014205|Ga0172380_10102486_2|M
[solid waste-landfill-landfill leachate]
MALKTLTFWPLEIPPEHKRIFDDIRKQLGNAQREMVKAWLKLALAAEPYGITPGEQAGRVLFGETDYSNGLLKSLAYTDARDSF
KEQWKGVLKGDATSVSFPDEASGVVLLVGSGQATVEKIDGEWWVTRLLVFGGRTAEDRSRNDQFRWQLKGVSRFGGHYEKSVMD
RAERICQVRVLPARKDRGKYVVQVTVEVPDEQIERTAIPVCAGIDLGINCPVVLSIPDKGFVRFMGRERDEYPRLWEKLREYED
RKRTLNRAGKRRAARDLRPRLTNIRKYINEAISKDVADTCRAMRVTTLRMENLKGLSFHKSGTGKLRHWPRYHLQERIEQKCGE
VGIEVERIKPAGTSQTCSMCGHRDKGNRNGSDFICLACGYRQHADLNAANNIARGMQEFGPCVQSSASDNTIELRFDGAGQEPA
LGRAMRLFA
(SEQ ID NO: 641)
>3300031566|Ga0307378_10048523_2|M
[terrestrial-soil]
MSLKTVGLYLAEMPQEDKVVCKAICRDLGRAGARMVREWYRACDEVAPGEKIPWGEVQTRGVVVGKATVQEAGLRPELANEMQR
TEFFARFKTEFGKILRGEKASLPFPGDAPFIYLRLDPGNAAATIEKREGVWYLRDPHFYPGGGRNQDNKREWRLKGRSRDGWEY
RWLEAAEKLACVRIKPDHKKRGAWVALITLHVPQVDDRPGSPEAVWAGVDLGLNSPAVLSIPDHPEGGFVRFFGRDLYLRMWDR
LDQYEERKARLNRAGKRRAARDLRAKITGIRQHINELISREIVDLCRRLRVTGIRMEDLRGIQDAATGKLKHWPRYHLMMRIQQ
KAGEAGLAFELVRAAGTSQTCSACGYREKGNREGALFHCLRCGFERHADVNAANNIARGMEEFDLPTGAQAMRPARRLELVAG
(SEQ ID NO: 654)
>SRR6837575_megahit_k177_854481_7|M
[wastewater metagenome]
MTTDKQTVTKTLRFWLTDLSKEDFDLLHELSRELARCHRLFVQASLEYAFAGGTNTTERLRLARRVYQEEFGKSRFVKRFGNTY
TGEIADKDFKRQMGLIQRGQKSSVSFGPGSHANIDLLTEGLQCTLVKEGDGWYITGAALTLDEDKATRAARKWGIIPQTSGGAA
VKAKQHARTMRQIEQAERTSRVRLVWRDGKKGGRKGWEAQVVCRMPVTASKPSEPTVCGIDVGMHCLLVASIPTKDKATFIGGN
TFGELWHEIERHCERRTILNKAHKRHAAAAQSDKISRIRKHGCELASRQLIDWCMYHHVTTIRLEDLSGIRDRCSEDENSEKAR
TWNRRLSHWPWYQLQQRIEQKADAYGITVEKVNPHLTSQTCSSCGYANPANREGTQFKCQRCGFERHADLNAANNIARREPGVD
FDAPNTPGRAAVLSDGPKSRPKAGRSGRLVLSDNPVTPRPDTLQLDMVL
(SEQ ID NO: 655)
>SRR6837576_megahit_k177_1219670_1|P
[wastewater metagenome]
MTTDKQTVTKTLRFWLTDLSKEDFDLLHELSRELARCHRLFVQASLEYAFAGGTNTTERLRLARRVYQEEFGKSRFVKRFGNTY
TGEIADKDFKRQMGLIQRGQKSSVSFGPGSHANIDLLTEGLQCTLVKEGDGWYITGAALTLDEDKATRAARKWGIIPQTSGGAA
VKAKQHARTMRQIEQAERTSRVRLVWRDGKKGGRKGWEAQVVCRMPVTASKPSEPTVCGIDVGMHCLLVASIPTKDKATFIGGN
TFGELWHEIERHCERRTILNKAHKRHAAAAQSDKISRIRKHGCELASRQLIDWCMYHHVTTIRLEDLSGIRDRCSEDENSEKAR
TWNRRLSHWPWYQLQQRIEQKADAYGITVEKVNPHLTSQTCSSCGYANPANREGTQFKCQRCGFERHADLNAANNIARREPGVD
FDAPNTPGRAAVLSDGPKSRPKAGRSGRLVLSDNPVTPRPDTLQLDMVL
(SEQ ID NO: 655)
>3300009716|Ga0116191_1022210_3|P
[wastewater-anaerobic digestor-anaerobic digestor sludge]
MIKTVTFYIAEMPREDKRLCDRLCIDLGAAGARMVREWFRACDETPPGAAIDWPEVQRRGVAVGKAMVEAAGYRTELGNDLARA
EFFARFKMEFKKVLKGERASLPFPGRAPFIFLRMDPGHETAIIEQRDDAWYMRNPRFYPGGDSNEANRREWRLKPVDRKGWAEN
ILEAAEKFACVRIMPDRKKPGSWVVKITVHLPDALERKLVEEHECWAGIDLGINCPAVLSIPDRDKGGFVRFFGMDKYTALWAK
LNRYEERKRALNRVGQQHAARDLRAKITGLRQYINEQISREIIDLCLRMHVTGIRMEDLKGLRTEGTGKLKYWPRFHLTTRIEQ
KAREVGLAFEQIRAAGTSQTCSVCGHRHRDNRHGSDFLCLRCGFARHADVNAANNIARGMQDFDLPAGSAAPGRALRLELSAQ
(SEQ ID NO: 606)

TABLE 9
Nucleotide sequences o f Representative
CLUST. 196682 Direct Repeats

| CLUST. 196682 all | |
|---|---|
| Effector Protein | Direct Repeat |
| Accession | Nucleotide Sequence |
| 3300025638\|Ga0208198_ | CTCTCCACGCGCGCGCGG |
| 1021643_2\|P | GATGCGGG |
| (SEQ ID NO: 601) | (SEQ ID NO: 701) |
| 330005300\|Ga0072500_ | GCCTGCTGTCCACACTATC |
| 136962_4\|M | CCCCAAAAAC |
| (SEQ ID NO: 602) | (SEQ ID NO: 702) |
| CAAACJ010042499_2\|P | GTGCTCTCCACGCGCGCGC |
| (SEQ ID NO: 603) | GGGATGCGG |
| | (SEQ ID NO: 703) |
| SRR5007116_megahit_ | GGTCTCTCCACGCGCGCGC |
| k177_300528_2\|M | GGGATGCGGG |
| (SEQ ID NO: 604) | (SEQ ID NO: 704) |
| SRR5007140_megahit_ | GTGCTCTCCACGCGCGCGC |
| k177_707372_2\|P | GGGATGCGGG |
| (SEQ ID NO: 603) | (SEQ ID NO: 705) |
| SRR5007457_megahit_ | GTGCTCTCCACGCGCGCGC |
| k177_513017_2\|M | GGGATGCGGG |
| (SEQ ID NO: 605) | (SEQ ID NO: 705) |
| SRR5007461_megahit_ | GTGCGGCCCACGCGCGCGC |
| k177_311051_7\|P | GGGGCAGAGG |
| (SEQ ID NO: 606) | (SEQ ID NO: 706) |
| 3300027896\|Ga0209777_ | TGGTGACCATGTTTTTGTTGAA |
| 10000273_30\|P | (SEQ ID NO: 707) |
| (SEQ ID NO: 607) | |
| 3300027896\|Ga0209777_ | TGGTGACCATGTTTTTGTTGAA |
| 10000273_32\|M | (SEQ ID NO: 707) |
| (SEQ ID NO: 608) | |
| 3300013127\|Ga0172365_ | CCAGTCCAAGTTTCGATGAG |
| 10030480_ | (SEQ ID NO: 708) |
| 1\|M | |
| (SEQ ID NO: 609) | |
| 3300013128\|Ga0172366_ | CCAGTCCAAGTTTCGATGAG |
| 10002984_ | (SEQ ID NO: 708) |
| 1\|M | |
| (SEQ ID NO: 610) | |
| 3300013129\|Ga0172364_ | CCAGTCCAAGTTTCGATGAG |
| 10030775_ | (SEQ ID NO: 708) |
| 1\|P | |
| (SEQ ID NO: 609) | |
| 3300013130\|Ga0172363_ | CCAGTCCAAGTTTCGATGAG |
| 10005240_ | (SEQ ID NO: 708) |
| 1\|P | |
| (SEQ ID NO: 609) | |
| 3300013133\|Ga0172362_ | CCAGTCCAAGTTTCGATGAG |
| 10039269_3\|M | (SEQ ID NO: 708) |
| (SEQ ID NO: 609) | |
| 3300032070\|Ga0315279_ | GGAAACAAAATAACAAACAA |
| 10000130_51\|M | (SEQ ID NO: 709) |
| (SEQ ID NO: 611) | |
| 3300032118\|Ga0315277_ | TATAAGGTGTTTTGAAAATGA |
| 10001250_38\|M | (SEQ ID NO: 710) |
| (SEQ ID NO: 612) | |
| 3300024429\|Ga0209991_ | GAGAAGGCAAAAAGAGAAGC |
| 10029076_1\|P | (SEQ ID NO: 711) |
| (SEQ ID NO: 613) | |
| 3300014886\|Ga0180300_ | TTTCAATTCCCATTATATGG |
| 10007429_2\|M | GATTTCTCTTTGAAACC |
| (SEQ ID NO: 614) | (SEQ ID NO: 712) |
| 330005935\|Ga0075125_ | GTTGTCTCCACGCGCGCGAG |
| 10033396_1\|P | GGCGGTTTG |
| | (SEQ ID NO: 714) |
| (SEQ ID NO: 616) | |
| 3300025736\|Ga0207997_ | GTTGTCTCCACGCGCGCGAG |
| 1027507_2\|M | GGCGGTTTG |
| (SEQ ID NO: 616) | (SEQ ID NO: 714) |
| 3300007203\|Ga0099832_ | CTTTCAATCCCACAATGGTG |
| 1115789_2\|M | CGATTGCTGCG |
| (SEQ ID NO: 617) | (SEQ ID NO: 715) |
| 3300000083\|Ga0072500_ | AGTGCGGGGGGAACCTAGGA |
| 119562_4\|M | GGGCAGTGGGC |
| (SEQ ID NO: 618) | (SEQ ID NO: 716) |
| 3300008000\|Ga0105162_ | GTTCGCGAGAGACTTTGTGA |
| 1004089_4\|M | ATAACAAGC |
| (SEQ ID NO: 619) | (SEQ ID NO: 717) |
| 3300008000\|Ga0105162_ | GTTCGCGAGAGACTTTGTGA |
| 1004089_4\|P | ATAACAAGC |
| (SEQ ID NO: 620) | (SEQ ID NO: 717) |
| 3300022544\|Ga0212120_ | GTTCGCGAGAGACTTTGTGA |
| 1002806_5\|M | ATAACAAGC |
| (SEQ ID NO: 619) | (SEQ ID NO: 717) |
| 3300022544\|Ga0212120_ | GTTCGCGAGAGACTTTGTGA |
| 1002806_5\|P | ATAACAAGC |
| (SEQ ID NO: 620) | (SEQ ID NO: 717) |
| 3300025063\|Ga0209493_ | GCCTGCTGTCCACACTATCC |
| 1003385_3\|M | CCCAAAAAC |
| (SEQ ID NO: 621) | (SEQ ID NO: 702) |
| 3300025090\|Ga0209080_ | GTTCGCGAGAGACTTTGTGA |
| 1004127_5\|M | ATAACAAGC |
| (SEQ ID NO: 622) | (SEQ ID NO: 717) |
| 3300025440\|Ga0209741_ | GCCTGATGTCCACATTATCC |
| 1005495_ | CCTTAAGAC |
| 1\|M | (SEQ ID NO: 718) |
| (SEQ ID NO: 623) | |
| 3300028675\|Ga0272445_ | GCCTGATGTCCACATTATCC |
| 1001731_ | CCTTAAGAC |
| 11\|M | (SEQ ID NO: 718) |
| (SEQ ID NO: 624) | |
| 3300029977\|Ga0272449_ | GCCTGATGTCCACATTATCC |
| 1037372_ | CCTTAAGACC |
| 1\|P | (SEQ ID NO: 719) |
| (SEQ ID NO: 625) | |
| SRR1562008_megahit_ | CGTGCGATCCACGCGCGCGC |
| k177_216982_5\|P | GGGGCAGAGG |
| (SEQ ID NO: 626) | (SEQ ID NO: 720) |
| UOPD01011681_4\|M | CGTGCGATCCACGCGCGCGC |
| (SEQ ID NO: 626) | GGGGCAGAGG |
| | (SEQ ID NO: 720) |
| 2645728127\|Ga0097870_ | GCCTGCTGTCCACACTATCC |
| 126_5\|M | CCCAAAAAC |
| (SEQ ID NO: 627) | (SEQ ID NO: 702) |
| SRR5272259_megahit_ | TGGTGACCATGTTTTTGTTG |
| k177_4486292_ | AA |
| 121\|P | (SEQ ID NO: 707) |
| (SEQ ID NO: 607) | |
| SRR5272259_megahit_ | TGGTGACCATGTTTTTGTTG |
| k177_4486292_ | AA |
| 118\|M | (SEQ ID NO: 707) |
| (SEQ ID NO: 608) | |
| SRR5273149_megahit_ | GCCTGCTGTCCACATTATCC |
| k177_25824_ | CCTTAAGAC |
| 1\|M | (SEQ ID NO: 721) |
| (SEQ ID NO: 628) | |
| SRR5273182_megahit_ | GCCTGCTGTCCACATTATCC |
| kl77_57442_ | CCTTAAGAC |
| 1\|M | (SEQ ID NO: 721) |
| (SEQ ID NO: 628) | |
| SRR5273183_megahit_ | GCCTGCTGTCCACATTATCC |
| k177_82677_3\|M | CCTTAAGAC |
| (SEQ ID NO: 629) | (SEQ ID NO: 721) |
| SRR6481359_megahit_ | CCAGTCCAAGTTTCGATGAG |
| k177_932521_3\|M | |
| (SEQ ID NO: 609) | (SEQ ID NO: 708) |
| 3300012677\|Ga0153928_ | ATATTATGATAAAGCATTGG |
| 1011778_2\|M | CTATAGATCCAA |
| (SEQ ID NO: 630) | (SEQ ID NO: 722) |
| WP_121304574_1 | CAACAGAGTCACCAACAGC |
| (SEQ ID NO: 631) | (SEQ ID NO: 723) |
| SEJ25361_1 | AGTAATTACTGAGTCATTAC |
| (SEQ ID NO: 615) | TC |
| | (SEQ ID NO: 724) |
| WP_121597521_1 | GCAGCTGGGGCTTTGGCGGT |
| (SEQ ID NO: 632) | GTT |
| | (SEQ ID NO: 725) |
| WP_006630662_1 | ACACTTCGATAGAGGGGTGT |
| (SEQ ID NO: 633) | |
| | (SEQ ID NO: 726) |
| DRR082460_megahit_ | AGCTTACTATCCACAAAGTC |
| k177_106880\|P | TTCCGCGAAC |
| (SEQ ID NO: 634) | (SEQ ID NO: 727) |
| DRR082460_megahit_ | AGCTTACTATCCACAAAGTC |
| k177_106880\|M | TTCCGCGAAC |
| (SEQ ID NO: 635) | (SEQ ID NO: 727) |
| OGCHO1000195_3\|M | AGTGCGGGGGGAACCTAGGA |
| (SEQ ID NO: 636) | GGGCAGTGGGC |
| | (SEQ ID NO: 716) |
| SRR4027810_megahit_ | GCCTGCTGTCCACACTATCC |
| k177_26210_1\|M | CCCAAAAAC |
| (SEQ ID NO: 637) | (SEQ ID NO: 702) |
| SRR5247126_megahit_ | GCCTGATGTCCACATTATCC |
| k177_220968_4\|P | CCTTAAGAC |
| (SEQ ID NO: 638) | (SEQ ID NO: 718) |
| 3300027941\|Ga0207719_ | GACCCTGAGCGGTGAAAGCC |
| 100292_34\|M | CTCGACCCGCTCGC |
| (SEQ ID NO: 639) | (SEQ ID NO: 728) |
| 3300027941\|Ga0207719_ | GACCCTGAGCGGTGAAAGCC |
| 100292_35\|P | CTCGACCCGCTCGC |
| (SEQ ID NO: 640) | (SEQ ID NO: 728) |
| SRR6484323_megahit_ | CGTCGCACCTCGCGCGCGCG |
| k177_106899_4\|P | TAGGAGATGGTTTGG |
| (SEQ ID NO: 641) | (SEQ ID NO: 729) |
| SRR6484325_megahit_ | GTCGCACCTCGCGCGCGCGT |
| k177_1345066_6\|P | AGGAGATGGTTT |
| (SEQ ID NO: 641) | (SEQ ID NO: 730) |
| SRR6484787_megahit_ | GTGCCGCCCACGGGCGCAGG |
| k177_1514834_8\|M | GGTGGATTG |
| (SEQ ID NO: 642) | (SEQ ID NO: 731) |
| SRR8542065_megahit_ | GTCGCACCTCGCGCGCGCGT |
| k177_1115558_2\|M | AGGAGATGGTTTG |
| (SEQ ID NO: 641) | (SEQ ID NO: 732) |
| SRR8542065_megahit_ | GGTCGGCCCGCGCGCGCGAG |
| k177_821416_8\|M | GGGGGTTGG |
| (SEQ ID NO: 643) | (SEQ ID NO: 733) |
| FZPSO1000148_2153\|M | GTGCGACCCACGCGCGCGCG |
| (SEQ ID NO: 644) | GGGCAGAGG |
| | (SEQ ID NO: 734) |
| WP_005554257_1 | GTCGTCTTCGTCGCCGATAT |
| (SEQ ID NO: 645) | CG |
| | (SEQ ID NO: 735) |
| SRR7004341_megahit_ | TAACCAGAATAGCGGTAAT |
| k177_2665443_5\|P | (SEQ ID NO: 736) |
| (SEQ ID NO: 646) | |
| SRR8526178_megahit_ | GTTGTCTCCACGCGCGCGAG |
| k177_763700_3\|P | GGCGGTTTG |
| (SEQ ID NO: 616) | (SEQ ID NO: 714) |
| OVXO01016275_2\|M | CTTTCAATCCCATTTTGGTC |
| (SEQ ID NO: 647) | TGATTTTAAC |
| | (SEQ ID NO: 737) |
| SRR7067823_megahit_ | GCCTGCTGTCCACACTATCC |
| k177_107253_1\|P | CCCAAAAAC |
| (SEQ ID NO: 648) | (SEQ ID NO: 702) |
| SRR7067986_megahit_ | GCCTGCTGTCCACACTATCC |
| k177_69094_7\|P | CCCAAAAAC |
| (SEQ ID NO: 649) | (SEQ ID NO: 702) |
| SRR8554444_megahit_ | GTTGAGTGGCCACATAGTGG |
| k177_504614_2\|M | GAGCTGAAC |
| (SEQ ID NO: 650) | (SEQ ID NO: 738) |
| SRR8554446_megahit_ | GCCTGATGTCCACATTATCC |
| k177_83048_1\|M | CCTTAAGAC |
| (SEQ ID NO: 625) | (SEQ ID NO: 718) |
| SRR6201998_megahit_ | GTATTATGATAAAGCATTGG |
| k177_1568738_2\|M | CTAT |
| (SEQ ID NO: 651) | (SEQ ID NO: 739) |
| SRR8554511_megahit_ | GCCGTGCTCTCCGCGCGCGC |
| k177_4248910_4\|P | GTGGGCGGTTCG |
| (SEQ ID NO: 652) | (SEQ ID NO: 740) |
| SRR8554511_megahit_ | GCCGTGCTCTCCGCGCGCGC |
| k177_4248910_4\|M | GTGGGCGGTTCG |
| (SEQ ID NO: 653) | (SEQ ID NO: 740) |
| 3300014204\|Ga0172381_ | GTGCCGCCCACGGGCGCAGG |
| 10015437_8\|M | GGTGGATTG |
| (SEQ ID NO: 642) | (SEQ ID NO: 731) |
| 3300014205\|Ga0172380_ | GTCGCACCTCGCGCGCGCGT |
| 10102486_2\|M | AGGAGATGGTTT |
| (SEQ ID NO: 641) | (SEQ ID NO: 730) |
| 3300031566\|Ga0307378_ | GGCGCAACCCGCACGCGCGC |
| 10048523_2\|M | GCGAGGGCAGAG |
| (SEQ ID NO: 654) | (SEQ ID NO: 741) |
| SRR6837575_megahit_ | GCAGAGCCCACGGGCGTGGG |
| k177_854481_7\|M | GGTGGATTG |
| (SEQ ID NO: 655) | (SEQ ID NO: 742) |
| SRR6837576_megahit_ | GCAGAGCCCACGGGCGTGGG |
| k177_1219670_1\|P | GGTGGATTG |
| (SEQ ID NO: 655) | (SEQ ID NO: 742) |
| 3300009716\|Ga0116191_ | TGCGGCCCACGCGCGCGCGG |
| 1022210_3\|P | GGCAGAGG |
| (SEQ ID NO: 606) | (SEQ ID NO: 743) |

TABLE 10
Non-coding Sequences of Representative CLUST.196682 Systems

```
>3300009716|Ga0116191_1022210_3|P
GCCTTCCGCGCGTGGCTGTCCTACGTGGGCGCGGAGGTGACGCCGGAGGAGCGGCGCAAGGCGGAGAGCGCGGCGGTGAACGGG
GAGTGCCCGCCACAACGCAGTTACGGGACTTCCCAAGTCTTTGATGGCTTTGAGAACCCAGCGGGCGCAGGGATGACCAACCCA
GAACACGCCGCGCAACGTCACCGCGACGGCGCGGGTATCCTCACCCGCCTGCTGACCGCGCACTGGCCGTCGTCGGCGCAGGAG
CAGCGCGCGCACTGGGAGGCGGTCATCGGCGAGGTGCCGGAGGGCTGGGATTGCACGAGGCCGCCGGGGGTGAGGGTTTCGCAG
GGGCGATCCCATTGGGGGCGCGAGAGAGGCCGCAATGGTTCTCATGCTGGCGTGTTGGACAAGTCCTCCACTTTCGCGAACGAG
GCATCGCACGACACCGCCCCATCGACTCCCGACGCCGCGCGCTTCGACGGCCTGCGCGGCTCCGTCGCCCCGCTCAAGCCCTTC
GCCTACCCCATCCTGCACGCGCAGAAGCCGCGCGAGGGCAGCTACCTCGCGAACCTGCGCGCGCATGGCGTCGGCGCGTGGAAT
GTGGAGGAGGCGAGGGTGCCGTTCAGTGAGGATGGTGTCGAGGGCTTCTGGCCGGGCGCCCGAAGTGGACGCGGCGACCTGCAG
CCCCCGTCGTGGGGAGCCACAGAAGGGGGGCAGCGCGATGACGCAGGCCGCACCCCCTCCAACCTCCTGTCCTACGGCGACCTG
CTCGGCGACCTGCAACGCTACTGCGACCTCGACGCCTGGTGCGAGCGCCTCGGCCTGCCCGACGCCGCCGCCGACCTGCTCGAA
GCGGGGTTGGTCTACGCGCCGAAGCCGAGCAGGGCGGAGAAAGAGGCCGGGCTGGAGGCAGACAAGCCGCCCGCGTTCGGCAAC
GATAAGGGCGATGGCCTTGGGCGGGGCATCAGCAACACGAGACAGGATTGGGCGCGGGGCAACACGCATCCCACCGCGAAGCCC
GCCGCGCTCTGCGCCTACCTCGTCGCCCTCGCCACCCGCGAGGGGCAGACCGTGCTTGACCCGTTCTGCGGGTCGGGCAGCACG
GGCGTCGCCTGCGCGCGCATCGGCAGGAACTTCGTCGGCGTCGAACTCGACGCCGACTTCGCCGCCATCGCACGAGCGCGGATC
GCACACGCCAAGACCACACGACCGGCTCAGGAGGCCCTCCCGCTATGACCGCGGACCTCCGCGACCGGAAGTTGACTCATTCCC
TCCATTCTGGTATACTGTCAGCGTTCCAGGCAAGGGACTCAGAGGAGGTCGTTTCAGTGTACGCTCGACAGTTTTGCCCCCGTG
TCTCCAGCGACCTCCTGTTCCCTTGCCTGGGCAGAGACGCTGTCACGGGGGCTGTTCGTGTACGCGCTTCGAGACAAGCCGACC
CGGCTGCGGTTGGGTTGGTTGCGGCTACTCTGGGCCGCGAGGCCGGGCGATCAGACCCGGCAAACCCGCGAGTACAAGTGTGGG
CAGCATGGGGCGGGCAACTCTGCTCATCGTAGTGGGAGCTGGCAAGCATGATCACAGGAGGCAAGACTGATGATCAAGACCGTC
ACCTTCTATATCGCTGAAATGCCGAGGGAGGACAAGCGCCTCTGCGACCGCCTCTGCATCGACCTCGGGGCCGCCGGGGCGCGC
ATGGTGCGCGAGTGGTTCCGCGCCTGTGACGAAACCCCGCCCGGTGCGGCCTCAGACTTCCTCTGCTTGCGGTGCGGCTTCGCG
CGCCATGCCGACGTGAACGCCGCGAACAACATCGCGCGGGGGATGCAGGACTTCGATTTGCCAGCCGGGTCCGCCGCCCCTGGG
CGAGCGCTTCGGCTGGAGTTGAGCGCGCAATGAGACAATGCGGGCGGGCTGCGGTTCGCGCGTGTGCGGCGACTGTGCGCCGCG
AGGACGACGGTCAAGTTGGCGAGTCATTGTTATGCAATGAACACAGGCCTGCCCACGGGACGGATGAAGGGCGCGGTGCGCCTC
ACCGCCTGCGCGACCGTCAGCGACCTGGTGCGCGCGGGGCTGGTCGTCGTCGCGTGGGCCTACGAGAACGGCGACCTCGGGCTG
CGGGGGACGGAGGCGGCGGCGACTTTCTTTCGCGAGGGTTGA (SEQ ID NO: 800)
>3300012677|Ga0153928_1011778_2|M
AGAATGAAGATGCATTAATTTGGAAAGGTATAGCTTTCTTTACAATGAAGAAAACGTTATTTCTGACAATCGCAATAACAATCG
CTGTGGTGGCGGTAGCGTTACCTTTCGTAGCACCAATAATCGCAAAAGTAGCTAATGCTGGCGGTAGCGTTACCTTTCGTAGCA
CCAATAATCGCAAAAGTAGCTAATGCTGACGGTAATTAGTTTTGATAAATTGTCGGCCTATAAGGTCATGGGCATTAGCAATTT
ACAATATATTTGAAGCCTGAAAGCCCGCCTCCTTTAGGAGCGGGATGAAATGCTGACCGATATCTTAATAGCTCCAAAGCTCTA
GTATTTGTATGAGTCGAGGACTCTTCGAATGAAAATCACAGTTAAAGCAAAAGTCACAGATGACAGCAAAAACAAACTAGGAAA
AATAGAATCAGAGTTTCAAAACTATCAGGTATATTTGCAATCAGGATGTACCACAAGAGTTAATCTATATTCTGCGACGAAACA
ACAAGCACAAAACACATGCTTTAACTGTCGCACTAAAGATAAGAAAGCAAGAAAAACACAAGGATTGTTCCAATGTGGTAATTG
CAGCATTAAAACGAATGCTGATTATAATGGAGCAATGAACATCTTGCAACGAGGGGTTGGATACTTTCAACTCTAGTGAGCTTT
CTGACATACCCCGAACCGTCGGTGATTATAGACAGGAATAAAATGATCACGAAGGAACCCCACATGCTTTATCTCTGGGAGGAT
GTCAGAGGATCAAACGGCTAACAATACCATAGATAGGTTCTCCTCCGAAATGATGTCCTGTGTCTTTATCGTTTAGGCTAAGAG
TCATTGTGTTTCCCTTGGAAATCAATACTGATATATGCGCATTATTCCACATAGTTACACCATTTATTTTTACATCAGCAGTAC
CATTCAAAGATAAGCTATTGTCTGATGTCATTTGAACTAGTTGTGTTTTTCCTCCAATATTATCAATTTTGAAGTTTGTTATTT
GGTGAATATGTGGTCTTCTACCATCCATCGGTGATGCAAGGAAGTCTACACCAAAATTCGTAACGTTCCCTTTATGCACATTCA
AGTTCCATTCTCCAGATAAGATAACCTTAAAAGCATCAGTTATATTAAATCCAGATTCGGGAATTGTAATAATTAAAGAATTTA
TTGTTCCTATTGCAGAGAATGTATCCTTTGCTGGGATTGGAAAAAATACTGGTAGTGAAGTCACCGCTATTTTTTTAGCTGCAT
ATGCCTCATAGTAATAACTATGAGTATTGTTATTACTAGCGTTGATATCTACTACAACGACGATCCCTATTATTATCCA
(SEQ ID NO: 801)
>SRR6837576_megahit_k177_1219670_1|P
CTGCGGCCACCCTGCCCCGTGGACTCCCCCGAGGGGCAGCAGGTGCTGCGGGGCCTCTTTCGGCAGGCGTTCGCGGCGTTGCCG
CCGGAGGCGCTGGAGCGGTGCTGCGAGGCGATCACGGCGGAGGATGACGAGGACGCTGGTGGGTGATGCCCACACCCGCCACGC
GGCAGGCCCCCTGTCCGGCACATCCTCGACTGGGCAGGGGGCCAGGCACACACAGGACGCACCCGCCGCCGGCCTCCTTCCCCC
GCGGCGGTAGACGGACCCGGTCTTGCTCCCGCGCGGACCGGCGACGACAGGGGCCGGGGCTCAACTCTCACTCCCGGCCCCGGG
GCCCGAGCAACAGACTACAGACAGGAGTAACGGCAGTGACCACCGACAAGCAGACGGTAACCAAGACGCTCCGCTTCTGGCTCA
CCGACCTGAGCAAGGAGGACTTCGACCTCCTGCACAAGCTCAGCCGCGAACTGGCACGTTGCCACCGTCTGTTTGTCCAGGCGT
CCCTTGAGTACGCGTTCTTTGACGCACCCAACACACCCGGCAGGGCTGCGGTTCTGTCGGACGGGCCGAAATCACGGCCCAAGG
CGGGGCGGTCTGGGAGGTTGGTTCTGTCAGACAACCCCGTAACCCCGCGCCCAGACACACTGCAACTTGACATGGTGCTCTGAC
CTGTACAGGTTTCGCGAGATTCCTTGTAGGAGCGTGGTAGACATGCCGACATGCAGCAACTGCCTGGCCCTCACTGAGCGCATC
CGTCGACTCAAGGCTGACGAGGACGCGCACCTGGAACAGATCGCCGCCCTGGGAGCCCGGCTTGCGCGCAGCGAGGCCAAACTT
GCGCGGTCGCAGGCGCTGTGCCGGCGGCTGGCGAGCAACGTCGCCGCGCTGGGTGGC(SEQ ID NO: 802)
>OVXO01016275_2|M
CTCGTTGTGCCTATGACGGCCTGAATGACTTCCTC (SEQ ID NO: 803)
>3300013128|Ga0172366_10002984_1|M
TCGCTTAACCAAAGATGGCGAATAGAGAACTGGTGGTTGTGATGCAGGTGCAGAAAGTCGTCAAGGCAAAAGTCGTGGGGCTGA
CTCACGCTAAACGCCAGATACTCGACCACGAATACGAAGGGCTCCAGCGATTCTTGCAGGGGGATTCCAACGTTGAACTCGCCT
TCAACATAGGCAGACGAGCCTTGGGGCAAGTCTCCAAGGTAGGGGCTGTCGTGAGCCAGCCCGTAACTGGGGCAGTGCCGTTCA
GCGAACTCGGTAGCGTCGTTCAATGTCCGACCCCAGAAGCCACCCTTTTTAAAGGGTGGTAGTTCACGTGCGCGAGCGCCTGCG
GCGTTTAGTCCAATCTACTAATAGGTGCTCTCCCATCTCCCTAAATTTCCTCGCCGCTGGAGAACGCGGGGCATAGCGAAGAAC
TGAGCTGCCAGCGGTCAAGCTCTCACGAACTTTATCGTCATCGGGTTGCCACTCAATTGGTGCTTGGACAGCCCCAGTCCATGT
TTCAATCAGATTCCACCCAATTGGAGCTTGCACCGTGCCAATCCAAGTTTCAATCAGTTGCCATGCGGCAGTGACCGTGACTGT
CCCAGTCCAGGTTTCGATGAGTTTCCATTCACCAGTGGCAGTGACTGTTCCAGTCCAAGTTTCAATCTCGTACCACTGAGCAGC
TGCTGGTGCTTGGACCGCCCCGGTCCATGACTCGGCGAGTTGCCATTCAGCCGGAGCACTTACTGTCCCAGTCCAAGAATCGAT
CTGCTGCCACTGTCCCGTGGTTGCATTAACAGTCCCGCTCCAAGACTCGACCTGACGCCACTGACCAGTGGTCGCGTTGACCGT
TCCAGTCCAACTCTCAATCTGCCGCCACTGTCCCGTGGTCGCGTTCACAGTCCCAGTCCAGCTCTCAATCTCGTACCACTGAAC
AGCTGCTGGTGCTTGGACCGTCCCGGCCCATGACTCGATGAGTTGCCATGCAGCAGGGGCGGTCACCGTGCCCGTCCAGCTCTC
GACGAGCTGCCACGCGGCCGGGGCGCTCACGGTGCCAGTCCATGTTTCGATGATCTGCCAGACTGCGGAAGCATTGACCGTCCC
CATCCACGACTCCACCAATTGCCATTCAGCTGGCGCCTGAACGGTTCCAGTCCACGTCTCTATGAGCTGCCACGCTGCAGGTGC
ATTAATAGTACCCGTCCATGTCTCGATCACTTGCCAGACGGCAGGCGCTGTCACGGTCCCAGTCCAGGTTTCGATTGTTTGCCA
AGCCGCGGGTGCGCTGACCGTTCCAGTCCAGGCCTCGATTAGCTGCCATTGGGCAGGAGGTGGAACCACCGTGAACTGCCAAGT
GTTTTCAGAAACATTCTCACCCGCAGCGTTGACCGCCCACACTCTCCAGTAGTAGGTCCCCGGGGTGGAGAAGTTGCGCTGGTA
GATGTTGTCGGTGTTGTCTTGGTAGTTGTCCACGAGCGGCGTGGAGAAGGTGGGCTCGTTGTCAATCTCGATCCTATGGTTCTC
CCCAAACCACCCGTTGTCCCACTCGAAGGTCACAGTTTCCCCGACCCCGAAGACAGAGTTGTTCTCTGGACTGTAGAGCCTGGG
CCTCTTCGGAAAGAATGTGTACTCGTATGTGAAAAAGATAGTACCGGTAAGAGCGGCGCCTGTTCCCATGTCGTATTCAAAGTT
GTAGGTCAGGTCGGCAAGGGTCCTATCTACAACAACGTCGATGTTGTCTTTGTTTACAGAGATGGATGTCAAGGCCTCCCCAGA
GCTGCCTGTGCTCATACTGACGGCTGAGCCCCAGTTTACCCCATCAGCTTAGATCCTGGTCTGTCGCGTTTCCGAAGCGGTTGC
ACTTCCTATCCAATATGCCGTGAGGCCAACGCTCTTCACAGTCTTTTTGTTTTCCGGGGCATAGAACGTCAGCTCCCCGCTGGT
CCAAGGGTCACCGGCAGCCCCGCTGGGTTTTTGGCCCGCGGTGAACATCGTGGTTTTCAGCTTTGTCGCGGCCGATGGGCGGAA
GTGATAGGTGATGTGCAAAAGGCCGCTTACCTCATAGAGGTCGGAGGCCGCAGCGCTCTGGATGTTGAGGTTCACATTGTCCCC
GTTCGACCAGTTCTCCCTCATGAAGGCGCTGATGTCGTGGAAGTAGTCGAACCCCTCTACATGCTCGCTGCCTATGTTGGTCGT
GTAAGCTCCCGAGGTCTGGGTTTTCGCCCCGACGCTCGCGTACACGCTCGTGCTGCCGTCACTAGTCAAGATGGTGTGGAAAAT
TTCAAAGTACGCGTTCTCGATGTTGTCCACGCGAGAGCTGTCCAGGTCCGTCAGATAAACGGGCGTCCCGAAGGCGAACAGGTT
GGCCGAGCCGTTGACGTTGGCGGCCCCCATGGCAAACGTGAGTGTTTTTATCTGGTCCGCGCTGTTGTCTGAATACTCATATGT
GACGTAAGTTTCACAGCTGAGCGCATCGTTTGTTTGTCCTCCGGTAGTAATTGTAATATCCTGATCAACGTTGTAGTTCCAGGA
AGCCGTCGGCCGGGAAATATACCAGAAATTGAGATCGTTGCCGGTACGGGCTTGCTCTGTCCAAGCATTTGTGGTGGCTGTATC
GCTTCCGATCTTGATGTTAAGAGAAAAGTCGGTTGAAGAGGCTTGGGAAGAGTGGGCATGGATTTCGTACCAGTTGTCCCTCAG
CGTGACCCCGTCTTCTGGGATTTCGATTCTGTGTGTGTAGGTCGCCGTGCCGCCAGCCCCTATCTCGGTGTTCGTGCTCCCCAC
AAAATACCTGACAGTCTTGATCTTCGTGTCCGCCAGCTCGTTCTCCTCATATGTGATGTACATCTTGGCCGTGTGGCTCTGGAT
ACCCGCGTCAGTGACGGTTAATTTTGGGTAGACGGCGGCCCCGTTTGTCCAGTATGTTGTAAAAGCAGAGGTAAAATCTGCGAC
CACCCATGCCGCCACGCTTTCCCCGCTGTTGGCGGTGTACTGGCCGGTCACTGGGTCTGCGTCTGGGTTCCGGTCGGTCCCCGC
GATGTTCAGGGTGACGGTCATATCTGTCACGTTGGCAGCAGTCGATGCCCCCGTCTGGACGTGATACATCACGAACACCTTCCT
GAACGGCCCAGCCTTGTCCGGGATGTAGATGGTTTTTGTCGGGGTGAACGAGTAGGCCGTGCCAGTGCCCAAAGCGGACGATGA
GGTTCCTTGCCTCGTATCCCATGAATATTGGATTGTGTTTATGGTCACTATATTTTCGTTGGGTTTGGTGGGGGAGGGAGTAGC
CACGGCTAGGGGCTGCATCGAGATAACGAAAACGCTGGCTAACATCATCGCTATCAATACGAATACAAAGATTTGTTTTGCCTC
CTTCCGCCCGAACTCTTCCCGCCCCAACCTAAACAAGCTACAACCCATCTCCCTCACCTGCTTGCCTCCACTTGGACTATGGAA
TGGGCGCGGCTCACGGCAGAGCCATCTCTCATCTCTCTCAGCAGGTTCGTGAAGTACTCAATCCCGCTCGCGGTGAGCTTGCAG
AGCTTTCGAGGCCTCCCCCGGGTTAAACGGGGCTC (SEQ ID NO: 804)
>UOPD01011681_4|M
CGCGTTCGTGCAGGGCTTCCTTGCGATCCGCGCAGACGTGAAGCACCCGCAGGCGGAGGATGGCGAGGACTCGTTCGGCGATCA
GTAGCGAGTGGTATTGACAAGCCGCCCCATATCATGTAAACTGAAAGTGTCCCGGCAAGGACTTGAGAGGAGGTCGCGTTCACA
ATGCACATCAATAGGATTGCCCCCGCGAAAGCGCGACCTCCGTTTCCTTGCCGGGACGAGAGCGTTGCTTTTCGCGGGGGTTTT
GCATGTACGCTTACAAGACGCCTCGAACCGGCTGCGGTTGGTTCGACTGCGGCTACTCTGAGCCGCGAGGCCGGGCAACGAGTC
GTCCGGCAAACCTGCGCGCGCCCCAAGCGTGCGATCCACGCGCGCGGGGCAGAGGACAACGGCACGTGACGACCGGTTCGACGT
GCGATCCACGCGCGCGCGGGGCAGAGGTAACCGGGTCAGAGTGTCGTACATAGGGCACCGTGCGATCCACGCGCGCGCGGGGCA
GAGGCAAGGCGGCCTGGGCGGCGCTGGAGAAGGCGGAGAGGCAGGCCTCGCAGCGTGCGATCCACGCGCGCGCGGGGCAGAGGA
GGAGAGATAGCTAAAGCAATGATAAAGACCGCGACATTCTATCTGGCCGACATGCCGCAGGAGGACAAGCGCCTGTGTCAACTC
CTCTGCGATGAACTCGGCGCGGCGGGCTCCGCGATGGTTCGCGAGTGGTATCGCGCCTGTGACGAAGTAGATGGCGAGCGTATC
GGCCCCGACTTTGTTTGCCTGCGGTGCGGTTTCGCCCGCCATGCCGACGTGAACGCAGCGAACAACATTGCCCGTGGCATGGAT
GACTTCGACTTGCCGACGGGCGCAAGCGCGCCGACGAAGGCGCGGCGATTGGAGATTGCATCATGAGAAGTGGCTGTCGGGCTG
AGGTTTGACGGCTTGCGGTGATTGCGCACCGCGAGGATAGCCGATGGTTATCAAGTGCTGATACAGCATCGCAAGCGCAGATGA
TGGTGCAGAGATCGTGCTTGACGAAGACGAAGTGCAGAAGCTCAGGGAGGCGCTGGAGGAGCACGCGAAGAGCAGTCCGCAGGA
CTGACGCACACGCCGCACCTGCTGACGCGCATGGGGACGCACGGGGATGCAGAGCGCGCAGCGAGAGGTTGCCGGGATGCGCTT
GCAATCGACGAACGTTCGTGCTATAATGGAGGTGTCTGGAGAGACACGGCGCGGCTTTGCGTTCTATGGCCGCCCACCACAATC
TGACCGTGACCATATTGCCCTCGTGCAAACCTCCGCGCGTGTCTCTCCAGACTGCGTAGGCGGCCAGCGCGAGGGCTTTTCTTT
GCTCCGGGGTGATCCAGTTCGTGGCTAACCCAAACCACTACGTCAAACTCTTCCTCGCAACGTTCGACGGGCAGTCCGCCGATA
TGCAGGCGCTCACTATCGGCCAGAAGTACGTGTGGCTGGCGCTCCTCGCATTTGCCCGAGCCTACAGACCGTATGGCATTCTGA
ACGCAGGAGAGCACGAGGGGCGTCCTATGCCCGCGAGTGTCCGCCGCCTGGCTGACTGGGCTTCCGTTGGAGTCGGCACTGTGA
GTGAGGCGCTGGCAATCTATGAGAAGAGAGGAATGATAAAGCAGGATAACCGGCGCAGTGATCCGTGTTGGGAGATCGTCAACT
GGGAGAAGTGGCAGGTGTCTCACGGCCTCGAAGAGTGTTCGGGAGCCGAACAAGATGGTGTTCGGGAGGCGAACACACAGACTG
TAGCGCGTTCGGGAGATGAACAGCACTGTTCGCCATCTGAACGCCAGTGTTCGGCAAATGAACAGCAGTGTTCAGATGGCGAAC
ACCTATCTCCGGACGCAGAGTGTTCAGGAGGCGAACACAAACAGGTAGAAAGCCTAGGGGCGCAAGGGCTATTGGCCTTAGTAT
CTGACGAGCAGGGGTGTTCGCCTACCGAACACGATTCCCTAGACGTTAAGAGAACAACATTAACAGCAGAAGAAGGCACGGCTA
CTTCTGCTTCTCAAGTAGATCAGGCACGACAAATCCTCTGCACCCTTCTTCGCTTGCCCGATGGATCAATCGCGATTGATGCCG
CGCTTATGGCGGCCTCTCGTGTTCATCCGCGCGCCGCCGATGTCCTTGTCCAAGAGTGTGGAGCACTTGCGGCGCGGGAAGCTC
AAGGCGGCAAGCCGGTTGATCTACAATACCTCGCCTCGAATGTTCGCCAAGCGGCGGAGCGACTTGAGCAG
  (SEQ ID NO: 805)
>3300025638|Ga0208198_1021643_2|P
GTGCGGGTGTGGCGCTCGCACCGGGCTGAATATCAGGAGGCGAGCGGCATGCAGGAGGCGCTGATGGTCGTGGCTGGGATCGTG
ATCGCGCTCGCGGCGACCGGATGGGTCTGGTTCGCGCTGTGTATGCGGGAGGGCTAGATTGAAGGCAACGACCATCACAAAGAG
CCTCCGCTTCTGGATCGACGAACCGCCTGAGCGGTGCCGTCTCCTCTATGGCGTCAGCGATGAACTCACCGATGCATACAACGC
TATCCCCGAGTACTGGAAGACCGATGTCCGCAAGGTAGCCATCCGACCGTCTACACAGCCGGCGGGACGTATCCCGTCGGCCGC
GCCCGACTGCACGGGCGAGGCAGTGAAGGCGTCAGACGTTGACGCTGAGCTGCAACCTGCGCGCACGCCGGCAGCTTGTGGAGG
TGATCCATATGCCGGCGAGTGAGCGAGTGGAGAGGTGTGGGGCCCTGGTCTGTACGGGGTAG (SEQ ID NO: 806)
>3300014204|Ga0172381_10015437_8|M
GACCTTAATGCTGCGAATAACATCGCGCGGGGGATGCAGGAGTTTGGGCCTTGTGTCCAGTCCTCCGCGTCAGACAACACTATC
GAGCTGCGGTTCGATGGTGCGGGGCAAGAACCTGCCCTGGGGCGGGCAATGCGATTATTCGCATGAGTCCGCAAGTCTCTACAA
GCGAGTGCGACCCCGAGATGGTTTGGAGGCAACCAGTGACCATACAGGAGCGAGTCTACCAGACATTAGCCGCACGCAACATCC
GTCCATCAGCGCACCTGCTGCACGATATGGGCCACTACGGCGAGGCTCAGGCGCTCAGTCAGAACCCTGCCGAGAACCCGTTGC
AGCAGGCGCTCAGA (SEQ ID NO: 807)
>3300014204|Ga0172381_10015437_8|M
ATCTACCGTATGTGCCAGATAGTCCGCATAAAGCGCCTGCAGATGGAGCAGTTTCGGAGAGCGCAGGCCAGGGCGGAAGCGGAG
ATGGACTGGCGCCGTCAGGCGCATCGCAAATGGCAGCGGTTGCGCGAAGCGATGGACAGCCGGTAGTCGTCTTGCCGTGGACCT
CTCCCGCCGCGACTGACATGCCCGGAGGAAACTCTGGCCCCCCCCACACCCGGGGCTCCGATCACAGCCGGGGCCCCGGCCCAC
AGGCCGCACCCCCGCGTTGGCGGGGACAACGACATAAGAGGTAGGCCGTAGACGCAGAGGCACAGAGGACGCGTGACGAGGGGC
GCTGCGACAACTGCACGGGAAGCAGTGACGTAGCGCCCAACCCACCATAATCGAGGAGCAAGCGGACCGTGGATACTGCAGCGA
ACGAGACGACACCCAAGGCTCTGTGCTTCTGGCTCGGCGCTCTGAGTGATGCCGGATTCAAGCTCCTACATGAGCTGAGTCGCC
AACTGGCACGGTGCCATGCGACGTTCGTGCAGGCGGCCCTGGAGTATCAGGCAGACCTGAACGCTGCGAACAACATCGCCGGGC
GTGACCCGGCGACAGACTTTGACACACCCGATCGGGCTGCGGTTCGGTCGGACGGGCCGAAATCACGGCCCCAGGCGGGGCGGG
CGTCGCGACTGGTCGCGGCGTCAACCCCGTAACCCTGCGCACCTAGACATCGGTGTTGGCACATTTCTGGTCTGCGCAGCTTGC
ACGGAAAGCAGTTGACGTGGCGCCCCACCCAACACACAGGCCATTCCCCGCGTTAGCGGGTTGGCAGGCAAGGCGTGCAGGGGC
CGGGCTGGACACCCGGCCCCACTATTCCACAGGAGGCGGACGATGCCAGGCACGGAGACGGTGACGGTGACAATCCCTGTGGAC
TACGTGACGGCCCGCCGGATAGCCGAGCTGGAGAGGAAACTCTCCCAGGCCAGGGGCGAGGCCAACGACCAGGAGTGCGACGCC
ACCTTCTGGCGGGAGAGGTGCGAG (SEQ ID NO: 808)
>WP_005554257.1
GCGGAACCGTGCCGTTTCATCGTCGACGCGAAACACGACTAACCCGCGGCCGCCTGCGTGAAGCGCGGTTCGCTCGGGTAATCC
CGACACCGGCTCCGTCGGCTCGATCGCCGCCGGGTCAACTACCTGAATCTCGTCGCTCATGTTCATTGTACCGATGGGGTCGGG
TTACAGAGGTTCGAGGAGAAAGCCCACGACTTCAGTCGTGGGATGAATCCGATCAAATACGACACAATCCATCCTCAATAGCCG
ACCGAAGGTTGATAGAATCATAACATCTAATATAGATTGACGTCTATATAACAGTATGCCACGGGGGTACAGCCGAGAGCGAAC
GTCGGTTCACAACCTCCACTACCACTTCGTGTGGTGTCCGAAGTATCGTAAACCGGTACTGACAGACGAGGTTGCCGACCGCCT
CGAACACCTCATCGAGGAGAAAGCCGACGAACTCGGACAGGTCAAGGGCTACACCTCGCGGAACCTTCGCGACGAGTTTGATTT
CGGCCTCCCGTCGCTGTGGACACGCTCGTACTTCGTCTCCTCGGCGGGCGAAGTGTCGAGTCAGACCATCGAGGAGTACATCGA
AGCACAGACTGGGCAATAGCGATGCACCGCGAAGTCACTACCACAGTACGTGTCAAACTCCACTCGCTCACCGAGCGGAAAGCC
CGCCTCATCGAACGCGAGTATACGGCGTTCCAAGACGCCGTTCACGGTGGCGACGCGAGCCTCTACTCCGCCACCAAACAACAG
GCGGACATTCAGAGCCCGCTATCCACGGTGGGGGCTGCCGTGGCTCAGCCCGAAACGCAGGTCGTACTCGACGGAACCAGCGGT
GAGATGGAACCTGCGAACTCCCCGAAAGACGTGGGCCTAACCCTCAGTGAGGGAAGTCCACGACTTTAGTCGTGGGAGAAGTCA
CGTCTGTCGGATGACCCACGGGCTCGCGGTTCGAGCCGTCGCTCCGCCGCTATACCTCCCAGGGTTCGCCGGTCGTCTCGCCGA
ACAGCTCCCGCATGAGCGTGACGATGCTTTCCGGCGGGAACTGGCCCCGTTCGGTGACGATCGCGTCGACGTGACGCGGCGGCG
TGACGTCGAACGCCGGGTTCTCCACCGTGAGTCCTCCTCGGAGTCGTCTATCTCGTCGTCGCCGTCGTCATCCGAGTCGTACTC
CTCTCCGTCGTACTGCTCGTCGTCTCCGTTTTCGCTCTCGTCTTCGTCGTCGGCCACCCCTTCGTTGTCATCCGCTTCGCCCTC
CTCATCTCCGTCGTACTGCTCGTCGTGGTCGTCCCTCTGGTCTTCGCTCTCCTCCTCACCGTCGTCGTCATCCTCGTCGTCCCC
ATCTCCGCGATCATCGTCCGCCGTTTCCTCACTTTCGTCCTCCTCTCCATCGTCTTCGGCATCGTCGCCGTTTTCTCCATCACC
TTCGTCACCCTCGTCGTCCTCGCCAGTCTCTTCGTCCCCATCATCCTCCTCGCGAAGCGCGACGAGACTGTCCTCGTGTTCGAC
GTAGAGCGTTTCGTCGCTGATACCCAGGACGTTGCCGACTGGCAGCTCCCCGGCTTGGATCGCCCAGCGCTCGCTTCCGTCGTC
CTTGTCGAGGGCGACGAGTTCGCCGTCGTATTCTCCGTACGTCGCATCCGTCCCCCTGAAGTAGACGTAGACGGTGTCGCCGGC
GATGATCGCGCGGCCGACGTTGCGAGACACGTCGACACCCCAGTCGTCCTCGTCGCCGTGATAGGCGCTTCCCGTGAGCTGGTC
GGCCTCGTACCCGCTGAGGTAGCGCTCCTTGCCGAGCGTCACCGAGATCCCCTCCGCCGACACCTTCGCAATATGCTCGCCGGT
CTGTGCGTCGAGGAGCACCCGGGCGTAGAAATCGACCCGGCTCACGACGACGGCGGTCGACGTCGCCCGTGCCTGGGCGCCTCC
GGTGTAAAAATTCGAATCCCCTCGCCAGACCTCATCACCCGTATCGGGGTCGAGTGCAACCGTTGCACCGCCTTCCAGATCCCC
CGCGCCGGCGTAGACGACGCCGTTCGCCGCGGCGGTCGTATCGAGGAAGTCGTACTCCTGTTCGCCCTCGTCATCGTGTTGGGT
TTCAGCGGTGAGCGTGTCGATCTCCCATCGCACCGACCCGTCGTCGGTCCCGAGGGCGTACAGCGTGCCGTTGACGACGACGAA
GACGCCTCCGTACGCGACGGTCTGACTCTGAATCGAATCCTCCGGCTCGAACTCGGTCTGCCACCGGACGCTTCCGTCGGCGAT
GTCGAGCGCGAACAGTTCGTCGCCGGTAACGTAGACGGTGTCGTCGACAACCGACGGCGCGTTCGCGTCGACGTCGGTGTTCTC
CCAGATAATCGAACCGTCGGCGACGTCGAGCGCGACGACTCCGTTCGCATTCGTCGCGTAGACGACGTCGTCGGCGACGGCCAC
GCCGGCGTCTCGTCCGAAGCCGAAGTCGACCGACCAGGCTGCCTCGGGCGCGTCGCCGTCGAGCGCGGTCCCGTCGGGGACGTA
CCTCGCGTGCCCGGCGTCGCCGTCGAACGACGGCCAGTCCTCGTCCATCCCTGGGTTCGGATCGCGGTCCGAATCCGGTAGATC
GTCGGTCGACTCCTCCCCGGCACCGATCGACGCGAGGGCTCCCCCTGCAGTGAGCGTTGCACCGGTCGCCAGCACCGAACGCCG
TCGCCACTTCGTCATACGGGTGTCGACGACAGAATATCGGATTACTATCCAGTTGCTACGGCTGACTTGTCCTTCCTGCAACTG
TTTCAGACGGACGAGCACCGAGCTTACTCGACGGAACGGAACCGACGGCATCGACCCTCGAAGACGGACTCGCGGGCTCGGGCG
AAGCGGCTTCCGGCCGTTACCACTCCTGGCCGGCGGGTTCGGCCTGCCGTGGTTCCCCCTCGTCCTCGGACGTCTTCTCCCCGC
GGACGACGAAGTACGCGATTGCGGCGACCACGCCCAGCGGGAAGAACAGGAACAACAGCCCGGCGGTGCCGAGCGCCCAGAG
(SEQ ID NO: 809)
>SEJ25361.1
GCAGTCGTTGCATCGATCTCCCGACTCGCAGCTGGCGAGATTTCGTATTCGGCCCGTCCATCCTCGGCAGTGAGGTTCTGTTTG
ATTAGGTCAGCCCGCTCGTTGAACTCGACGGCGGTGCTGGTGTCGGCGTCGTATTCCGGATCGCGGAAGTATTCCCGTGGCGTT
TCGGGTTTGCGGTAGGCGGCGTTGGGGTTGTCGCGGTTCCACTCTCTGTTTTCAGGGATCTCGGCGTCAAGCTGTTGGTGGAAG
AACTCGATAGCCGCTGCCATTGCCGCCTTGATCCGGTGTTCTTCGGGTGGGTCGACGGCGAGTTCGGCTTCGAGTTCCGAGTAG
TCTTCATCAAGATAGTCCTGTTTGGTCTTGGTGGGAGTCGTGTCTGTCTCGGTCCCTTCCCCCGTCTCTGATTCAGTCTCCGTC
GACTCGTCGGTGGGTGTTGCGGTGTCGTCTTCGTCGGCGTCGGCGTCTTGATCGTCGCTAGTTGCTGTGGTTTCGGCAGTCCCG
TCTGTGTTGGCCGCTGTCTGCGGTTCGTCTGTGTCGGTGGTAGGATGTATCGGCTTCGGCGTCCGATTGCTCACCAGCCGATTC
TGCGGCGTCTGTGTCAGCGGCAGTCTGTGCGGGGGCTTCGTCTGACGTGTTCGAGGTGTCGGCCCCATCACTTCCATTTTCACT
CTCAGCACTTTCCCCACCTTCCTCACCCGTGGTTGCTGGGCTGTCGTCTGCAGTCTCGGTGTCGATTGATTGCTCACCGTCTCC
GTTGTCGCCTCCATCATCGTCATCGTCGTTGTCGTCGGTGGTTTCACCGCCGAGTTTCCGGGTGATCTCTGGGCCCTCGTCTGT
GGCGGGGTCGTTGATCACATACTGGCCGTCTGACTCAAACGCCGGAGAGTAGATCTCGCCTCGTGAGACTGCATCCTGGAGTAT
CTCTTCGGCTTTGCCGAGTGGCTTCGGGTCTGCTGAATAAGATTATCTCATGTATGGTGGTTGCGACTTTGTATGGCTTCGAAC
GCCTGAGTATATTATTTTTATTTGTTAATATTGCAGTTGTAATATAAATAAGACCATGAAGAATACGATACTAGAGATAATTAC
GACGCGTCGAAACCAACCAACACGGAAGCACCAGTATTGCTCTCGATCCGACGGATCAGGATACTGGCACCTCATCTATGAGTG
GGTAGACGGTTCGTGGCAGATCGTAAGCCGCGACCCACTCGACGAGATCGAGATCGACATCTCCAACTCGCGTAGTGATTGCAC
ACCAACCCCACTCGTTGAGTGTCCTGTGTGTGGTCGGACTGGACTTCCCGAACGGATCGCCCAGCACAACTGTACACCAAGGTC
GACACAGTAAGCTAGGAGTCAAAGATACAACCAATATAAACACACCAAGCCATTACCAACAAAAATATATGGTTACGGGTGTTG
TATAACTAGTACTAGTTGTTATTCACGCATCTCCGCTCGCTCTCGTAGGCTGTTTTCGGTGCTATCATAGAGTATAAATTGGCT
ATCGACTCATCTGGTGAGTTCCTCAGTCGACCGCCCGACGCGGCGATCAGTGGTGAGAGGAACAGCAAGACAGCAGAAGAGTGA
GATTGAGAACTAAGATAGAGCACCTCGCACCTCCAAACAGATGGACGACCAGTTCCGAACACGCTCGTGTTTCGTCGGAATCGG
CGCCACCGGTGCCGAGCGCATCGGTGACGGCATCGAACGTCGTGACGCAAGTGCCACGATGGTTCCGGACGAAGTTCCGGACCC
AGCCGACGCCGTCGTCGACTGCGTTGCCGCACCGAGGGCAGGTTGTACTCTCCCAGAACTGCTTGAGTCGCCGCTCGATGGCCG
CATCGAGCGGCTCCGTGTTGATTGTGATCACGCCCAGATCGACAAGCTGGCGAAGCATCCGGCCGAAGGCTGGATCTGAGGAGG
ACATACTCCCTCATTTGACACCGTAGACCGTAGCTATATGGACTTTCCACGTTTCTACATAAGAGCGCAACAGGTTAACCAACA
AATCTATCTTATACTCGTTTTGTTGGTTAACACAAGGCGCTCTTATGTGAACCACCTCGCGGTCAAGCCCCGAGGCACTCGCCT
TGCTTATCTGTAGACGTGGAAATGTTCAAAAAGTGGTTCGATACCCTCAAGAGATCACCAGCCGGCCAACATTCGACGTCCCAT
ACAGGTACTAAGTGACGCTAGCACTGATTGTCAGAAAAAAATTTATGAAACGGTGGTGTGGCCCTCCGATTTAACTCTTGCAAG
GCTAAGTCCCCGTCCTCAAGGAGCAACGCAGGGAGCGTAGCGACCGAGTAGCGCAGTAGGGCGGGGATACAGCCGACACGAAAT
TAGGCACCCCACGAAATCGCACACCCTAACCAAAGAATTACATGATTGTCACCCGATAGTGGAAGTGAACCCACAATGGAATAC
GACCTCGATACGGGAGCGCACTCAACGTACTCCCTGCACTACCACCTCGTCCTCGTCGTGAAATATCGACGCGAGGTGTTCACC
GAAGAACTTCATGTTCACCGAAGAACTTCAAGGGTTCTTCCGCGAGGTCGTGAGCGGGTTCGCTGACAACTACGGCGTCGAAAT
CAAAAACCTCGAAGCCGACCGAGGCCACGTTCACCTCCTGTTCAAAGCCACGCCCACAACCGACCTCACCAAATTCATCAACGT
CCTCAAAGGCGCATCGGCTCGTCGCATCCGCAATGAGTACGGCGAGCAAATCGGGGACAAACTGTGGGGTGACTCGTTCTGGGC
AGATTCGTACTGTCTCATCTCGACGGGACAGGTATCGCTGGACATGCTGAAAGAATACGTCGAGAACCAGCGGGAGCGTGACGA
CGAATGAAACAGACTGCCGAGAAAACGGTCGTGGCAAAAATCTTCACCAACGACCTAACGCAGTCGAAACTTGACGCCATCAAC
CACGAGTATGAGGCGTTCCAGCGATACATTCGTGGTGACGAGGAAGCAGATCTCTACTCGGCCACGAAATTGGGTAAGGACATC
GCGTGCCCACTATCCAGTCTCGGCGCAGTCTGTGAACCTGTGCTCGGACCGAGCGGTGCTGACCAAGCCTCTGCCACGGTATGC
TTGCGAGCCGACTTGGAAGCCCCTGCCTCAACGAAGCGAACCGTTAGGTGAGCGAAGTAGGCAGGGGTAGTTCACTTGAAATTG
CTGAAATGGTGGGGGGTGTTGAGGAGCCTAATATTGGTTCAAGACCCGCTCACCAACAAGACTAGGCGTTCGGTCGATCAGCAC
TGATGCGCTCAAAATCGATATCTGCGAATCGAGTCGTATCCTTGATAACTTCGACTGCGATCTCGGTATCAACTTGCAGCTGGT
ACTCCCATGCTGGCCCACCGATCGAACCTGCCGTCACCTTTTC (SEQ ID NO: 810)
>3300014886|Ga0180300_1000742_9_2|M
GGCAGGTTGTATTAGACGGATTTTATTTAAAATTGTTTCTGGTAGGTTGTTTTCTCTGGGTCGTTGTGAAGGTTGGAAAATCTG
TTAGGGGTTTATAAAGATCTTCACGGTTTGGTTGGTGGTTTAGAAAGTGTTAAAAATCTTATAGGTTTAGATTTCTTTACTGGG
TGGTGTGTGTTGAAGACTTTTGTGATCTATGATATCTCCGATGATCGGATGCGGGAGGGGTTGAGGGAGCATATTAGGAACTAT
GGTCTTCGGCGGGTTCAGTATAGTGGTTTCCTGGGGGAGATGAGTCCCCATGATCGGTTTGTCCTTTCTCGTGAGGTTGATCGT
GAGGACCTGAGGGCACTTTACAGCGATGTGATGAAAACCTTGCATGATGAGGCCTTTTATGAGGGCGTTTATAACCCAGAGGAG
GTCGAGTATGCTATAAAACAGGTTAAGAAGATGATTGAAGAATTTAAAGGAAGTTAAAACCGCATATTGTTAGGATTCTTCTTT
TTACCTATTCTCGGCGAGAAATCCTCCCACTTTAGTGGGGGGATGAATCGCCGTTTAAGTTTAAATATCAGAAATCTTATATTT
GTTTCGGGCGAGTCGCCCATGAAGGTTAAGAAGACAATTAAGGCTAAGATTCTTGAGCTTCGGAAGGGCAAGGAAGAGCTTTTG
AAACGTGAGTATGAGAATTGGCAACGCTACCTAAGAGGCGATAAAACGGTTCCGCTTTATTCTGCGACCAAGCAACAGGCAGAT
ATACTCAAGCGGGCTATGGGGCTAGTCTCCGTAGCAGGGGCTGGATTGACACAGCCCAGAACTCGGTATGATGAAGCCAGAAGG
CCGAAAAACCGAGAATCTCCCCACTTAAGTGGGAAGAGTGTCAAGAGTTATTCCTTCAAGTTCTAAGATGAGTTTCTCGCATAG
GTTGTAGGTCTTCTTGGCTCTGTTGGTGGCGTCCTCTGCATCGTTCTTGTCCATGACCTCGTTGGGTGGTGTGAGGGTCTTTTC
ATCCCCGTATAGGCTTGGCTCCCTTTTCTTGAAGAGGAGTTTGGAGGATTCGCGTAGGAAGTCTATCTCGGTTTTGAACCAGTC
TGGGAATCAGTCTCTGATGTCGGAGAGAAGGTCTGATACCTCGTGGATTTTGGGGTACTCGATT (SEQ ID NO: 811)
>WP_006630662.1
CCCTTCCGGTCAGTTGAACCGTAGATCGAAGCGGAAAATTAAGTCAAGTCCAAATGTTGGTCACCGTCGATACTGGCAGGTAGA
ACAGGTTTATAGCCGGATATTGATGTCTACACACCCCGTTCTAGTTATGGAAGCGACAGCACTGAACGCTACTTTCCTTCGAAG
AAATCATCTATTTGTGCGTTGACAACAGATTTGATTAGATCGCCTTCATTGTCTGCCTCTTCAATCCGGATGTCAGCTCGAAGA
GTTTCAGCAACCACACCCGGATCATCAACAAACGTGTACTCCTTGTGAACGACCTCGTCACGGATCTCGGCGAGTGCTTCGTCG
TCGACGCCGTGGTCGTGTAATCTCGATTCGAGGCGATCGATCGAATCCCGCGCGAGTGCCGCCTCCTCGTCCTCATCAGGCCGA
TACGTCTGTGGATCGCCCATGAAGTGGCCCATCCGTCGGTGGACTTGAACTTCGAGGAGTGTGGGTCCGTTCCTGTCCCGGGCT
CTGCCGATTGCGTCAGCGGCAGTTTCGTAGATGGCAACGGCGTCGTCGCGGTCGACGCGAACACCTGGCATATCAAACCCGGCT
GCTCTGTCTGACCCGTCTTCTACGTCCGTTACAGCGGTAGCAAGGCGAAAGCCCACCCGTTCACGGGTGGGATGAAGCCGACAA
CTGGGAATGTATCCACGTTCGTAGCCACGGCTTGGGGTTGATATTTGCGTAACATCTTTCGATGAGTTACTTCTATATACCGAC
GATGCCTCGCGGGTTCGACCGAGAACGTACCAACATCCACAAACTCCAGTACCACTTCGTCTGGTGTCCGAAGTACCGCAAGTC
GGTACTCAAAGGCGAGGTACGCCATCGACTCGAAGAACTCATCGAGGACAAAGCTAACGAACTCGACCAGGTCAAGGGCTACTC
CTCGCATCACCTCCGTGAGAAGTACGACTTTGGCTTGCCCTCGCTGTGGACGCGCTCGTACTTCGTCTCCAGTGGGGGTGACGT
CTCCAGCGAGGTCATCGAGGAGTATATCGACGCACAAGCGGGTGAATAACGATGCAACGGAACGTCTCAACCACGGTGCGGGTC
AAGCTCCACTCGCTGACCAACAGAAAATCCGATATGCTGGCCCGTGAGTACGAGGCGTTCCAACGCGAGGTTCACGGCGGGGAT
GCCGACCTCTACTCCGCGACGAAACAGCAAGCGGCCTCGGGCAAGTTTTCGAAACCGCTGTCCGAGGCGGGGGCTGTACTGGCA
CAGCCCAAAACGCAGGTCATCGTCGAACGTGACGAGGAACCTGCGAACCTCTCAATTTCTGTGGGCTCAACCCCCAGTGGAGGA
ACCCCACGGCTTTAGCCGTGGGAGGATGTCAGACGGTCTTTGGGCATGCTGATTGCCCAGTCATTATCCTCGACGACGAATACA
ACTGGTAGGTTGTGGACACTCGCGAAATTCAGCGATTCGAGGAACGCACCCTGATCGATCGCTCCCTCACCGAGGAACGCGACT
GCCACTGCGTCAGTATTGCGCTTTTTGGCCGCAAGCCCTGCCCCGGCTGCTGGTGGACATCCCTCAGCGATGATGCC (SEQ
ID NO: 812)
>3300024429|Ga0209991_10029076_1|P
CGAGAAAGCTAAGGAAGCCAGAGAAGCAGAGAAGCTAGCTCAGAAACAAGCCGAACTGGAGGCTAAAGAAAAGGCAAAGAGAGA
GGCCGAGGAGGCAAAAAGAGAGACTGAAGAAGCTAAGAAGACCAAAGCTGAAAGGATGCTAGCCGAGAAGGAAGCCGAGATAAG
CCGTCTGGAAGCGGCATTATCACGCGGAGATATACGACTGGGTAAAGTTATCTATCATTCCTGGAAGCTGGGCTCAGCACGAGC
CACTAAGGAAAAGGCCAGACGAGACGCCGAGAAAGCCAAGCAGGCCAAGGCGGCTCAGCAGTAGGCCGAGCGGGAAGCGGAGCG
GGGCGCTAAGGAGAAGGCGAAGCGAGAGGCTGAAGAGGCTAAGCAGGCACTACAATCGCTGATAGATTTTGACCAAGAGAAACA
AATTGAAGAATGTCTAAAAGAGGTGAAGAGCATTTGTGAAGAACTAAGGGCAGGGAAAGCTGAGACAAAGGAAGCAATACAAAG
GCTTTTAGATATTTCAGAAAGGATTCCCGAGTAGATATTCCCCAGCTAATATCAGTAGGCTGATGTGACTGGGCTTCATCCGTG
GCAACCTATTTGAGTTTGATAATTCACTACCTCAGTGTACATTCAGGCGGACTCCTAAAATTACAGAATCAGGCTGAGAAAGCC
CCCTGCTTCAGCGGGGGGATGAATCAGCCCTTCCCTCCTTTCCCAAAGTATACCAATATATGTTTATGGGACAAGAATATAGAC
ATAACAGACATTCGATCTCTTTGGTCAACTATCATCTCATCTGGATTCCGAGGCGTCGGAGAAAAGTATTGGTGAATCAGGTTA
AGGATAGACTTGATAAACTGCTTAGGCAAAAAGCTGAAGAGCTTGGTATAGGAGTGATAGCTTTGGAGATAAACCCAGACCATG
TTCATCTTTTTGTGAATGCTATGCCGGATTTGGCCATTCATCAAGTGGCCTTTCCTTATCGCGGTCTTTTTCTTTGCCATTCTT
GCAAACTCCACTATAATGCCGACCTTAATGGTGCCCGTAATATTCTAAAGAGGGCTTTGGACTATATGTCCTCAGCCGGGGCTG
CTTTGGCACAGCCCGTAACTCAGTCTCTGAACTGAGAATCCTCCGCCTTCAGACGGGGGAGTGTCAACTGCAGGAACATTACTG
GCAA (SEQ ID NO: 813)
>FZPS01000148_2153|M
GGGCAGAGAAGGTAGATGCAGTAGAGGCAGGAGAGGATTGGCCTACTCATGATTAAGACAGCGACATTCTACATCGCCGATATG
CCGCAGGAGGACAAGCGCCTCTGCGAACGCCTTTGCGCAGAACTCGGAGCGGCGGGCGCGACGATGGTTCGCGAGTGGTATCGC
GCCTGTGACGAAGTGACCGGCGAACGTATC (SEQ ID NO: 814)
>3300013133|Ga0172362_10039269_3|M
AAGCTGTTTCACATGGCAAGAGATTTTTGATTCGAGTCGTGCGGCCAGGTCGGGCTGATTGAAAAGAAGGAGATGGGTTTTTAT
CGCTAACTTTTACCTTAAAACTGCTTCACAACCGAAAATCGCTTAACCAAAGATGGCGAATAGAGAACTGGTGGTCGTGATGCA
GGTGCAGAAAGTCGTCAAGGGGAAGGTCGTGGGGCTGACTCACGCTAAACGCCAGATACTCGACCACGAATACGAAGGGCTCCA
GCGATTCTTGCAGGGGGATTCCAACGTTGAACTCGCCTTCAACATAGGCAGACGAGCCTTGGGGCAAGTCTCCAAGGTAGGGGC
TGTCGTGAGCCAGCCCGTAACTGGGGCAGTGCCGTTCAGCGAACTCGGTAGCGTCGTTCAATGTCCGACCCCAGAAGCCACCCT
TTTTAAAGGGTGGTAGTTCACGTGCGCGAGCGCCTGCGGCGTTTAGTCCAATCTACTAATAGGTGCTCTCCCATCTCCCTAAAT
TTCCTCGCCGCTGGAGAACGCGGGGCATAGCGAAGAACTGGGCTGCCAGCGGTCAAGCTCTCACGAACTTTATCGTCATCGGGT
TGCCACTCAATTGGTGCTTGGACAGCCCCAGTCCATGTTTCAATCAGATTCCACCCAATTGGAGCTTGCACCGTGCCAATCCAA
GTTTCAATCAGTTGCCATGCGGCAGTGACCGTGACTGTCCCAGTCCAGGTTTCGATGAGTTTCCATTCACCAGTGGCAGTGACT
GTTCCAGTCCAAGTTTCAATCTCGTACCACTGAGCAGCTGCTGGTGCTTGGACCGCCCCGGTCCATGACTCGGCGAGTTGCCAT
TCAGCCGGAGCACTTACTGTCCCAGTCCAAGAATCGATCTGCTGCCACTGTCCCGTGGTTGCATTAACAGTCCCGCTCCAAGAC
TCGACCTGACGCCACTGACCAGTGGTCGCGTTGACCGTTCCAGTCCAACTCTCAATCTGCCGCCACTGTCCCGTGGTCGCGTTC
ACAGTCCCAGTCCAGCTCTCAATCTCGTACCACTGAACAGCTGCTGGTGCTTGGACCGTCCCGGCCCATGACTCGATGAGTTGC
CATGCAGCAGGGGCGGTCACCGTGCCCGTCCAGCTCTCGACGAGCTGCCACGCGGCCGGGGCGCTCACGGTGCCAGTCCATGTT
TCGATGATCTGCCAGACTGCGGAAGCATTGACCGTCCCCATCCACGACTCCACCAATTGCCATTCAGCTGGCGCCTGAACGGTT
CCAGTCCACGTCTCTATGAGCTGCCACGCTGCAGGTGCATTAATAGTACCCGTCCATGTCTCGATCACTTGCCAGACGGCAGGC
GCTGTCACGGTCCCAGTCCAGGTTTCGATTGTTTGCCAAGCCGCGGGTGCGCTGACCGTTCCAGTCCAAGTCTCGATTAGCTGC
CATTGGGCAGGAGGTGGAACCACCGTGAACTGCCAAGTGTTTTCAGAAACATTCTCACCCGCAGCGTTGACCGCCCACACTCTC
CAGTAGTAGGTCCCCGGGGTGGAGAAGTTGCGCTGGTAGATGTTGTCGGTGTTGTCTTGGTAGTTGTCCACGAGCGGCGTGGAG
AAGGTGGGCTCGTTGTCAATCTCGATCCTATGGTTCTCCCCAAACCACCCGTTGTCCCACTCGAAGGTCACAGTTTCCCCGACC
CCGAAGACAGAGTTGTTCTCTGGACTGTAGAGCCTGGGCCTCTTCGGAAAGAATGTGTACTCGTATGTGAAAAAGATGGCACCG
GTAAGAGCGGCGCCTGTTCCCATGTCGTATTCAAAGTTGTAGGTCAGGTCGGCAAGGGTCCTATCTACAACAACGTCGATGTTG
TCTTTGTTTACAGAGATGGATGTCAAGGCCTCCCCAGAGCTGCCTGTGCTCATACTGACGGCTGAGCCCCAGTTTACCCCATCA
GCTTGAATCCTGGTCTGTCGCGTTTCCGAAGCGGTTGCACTTCCTATCCAATATGCCGTGAGGCCAACGCTCTTCACAGTCTTT
TTGTTTTCCGGGGCATAGAACGTCAGCTCCCCGCTGGTCCAAGGGTCACCGGCAGCCCCGCTGGGTTTTTGGCCCGCGGTGAAC
ATCGTGGTTTTCAGCTTTGTCGCGGCCGATGGGCGGAAGTGATAGGTGATGTGCAAAAGGCCGCTTACCTCATAGAGGTCGGAG
GCCGCAGCGCTCTGGATGTTGAGGTTCACATTGTCCCCGTTCGACCAGTTCTCCCTCATGAAGGCGCTGATGTCGTGGAAGTAG
TCGAACCCCTCTACATGCTCGCTGCCTATGTTGGTCGTGTAAGCTCTCAAGGTCTGGGTTTTCGCCCCGACGCTCGCGTACACG
CTCGTGCTGCCGTCACTAGTCAAGATGGTGTGGAAAATTTCAAAGTACGCGTTCTCGATGTTGTCCACGCGAGAGCTGTCCAGG
TCCGTCAGATAAACGGGCGTCCCGAAGGCGAACAGGTTGGCCGAGCCGTTGACGTTGGCGGCCCCCATGGCAAACGTGACTGTT
TTTATCTGGTCCGCGCTGTTGTCTGAATACTCATATGTGACGTAAGTTTCACAGCTGAGCGCATCGTTTGTTTGTCCTCCGGTA
GTAATTGTAATATCCTGATCAACGTTGTAGTTCCAGGAAGCCGTCGGCCGGGAAATATACCAGAAATTGAGATCGTTGCCGGTA
CGGGCTTGCTCTGTCCAAGCATTTGTGGTGGCTGTATCGCTTCCGATCTTGATGTTAAGAGAAAAGTCGGTTGAAGAGGCTTGG
GAAGAGTGGGCATGGATTTCGTACCAGTTGTCCCTCAGCGTGACCCCGTCTTCTGGGATTTCGATTCTGTGTGTGTAGGTCGCC
GTGCCGCCAGCCCCTATCTCGGTGTTCGTGCTCCCCACAAAATACCTGACAGTCTTGATCTTCGTGTCCGCCAGCTCGTTCTCC
TCATATGTGATGTACATCTTGGCCGTGTGGCTCTGGGTATCCGCGTCAGTGACGGTTAATTTTGGGTAGACGGCGGCCCCGTTT
GTCCAGTATGTTGTAAAAGCAGAGGTAAAATCTGCGACCACCCATGCCGCCACGCTTTCCCCGCTGTTGGCGGTGTACTGGCCG
GTCACTGGGTCTGCGTCTGGGTTCCGGTCGGTCCCCGCGATGTTCAGGGTGACGGTCATATCTGTCACGTTGGCAGCAGTCGAT
GCCCCCGTCTGGACGTGATACATCACGAACACCTTCCTGAACGGCCCAGCCTTGTCCGGGATGTAGATGGTTTTTGTCGGGGTG
AACGAGTAGGCCGTGCCAGTGCCCAAAGCGGACGATGAGGTTCCTTGCCTCGTATCCCATGAATATTGGATTGTGTTTATGGTC
ACTATATTTTCGTTGGGTTTGGTGGGGGAGGGAGTAGCCACGGCTAGGGGCTGCATCGAGATAACGAAAACGCTGGCTAACATC
ATCGCTATCAATACGAATACAAAGATTTGTTTTGCCTCCTTCCGCCCGAACTCTTCCCGCCCCAACCTAAACAAGCTACAACCC
ATCTCCCTCACCTGCTTGCCTCCACTTGGACTATGGAATGGGCGCGGCTCACGGCAGAGCCATCTCTCATCTCTCTCAGCAGGT
TCGTGAAGTACTCAATCCCGCTCGCGGTGAGCTTGCAGAGCTTTCGAGGCCTCCCCCGGGTTAAACGGGGCTC (SEQ ID
NO: 815)
>WP_121597521.1
GCGAAGCGAACGAACGAGGAGCGCAGCGAGTGTGCGCCGCCCTCGCGGCTGGGGCTTTGGCGGTGTTTTCTGTCGATCCGTTGG
CGGCTATTTATAAATGAGCAGCTGGGGCTTTGGCGGTGTTTTCTGTCGATCCGTTGGCGGCTATTTATAAATGAGCAGCTGGGG
CTTTGGCGGTGTTCCCCGCCGATCCAGAATCAGCAATTTATAAGCGAGCAGATAGGGATATAGCGGTGCTCTCTGCCATTCCGC
AGTCAGCCATTTATAAGCAGTCGGCGGTAGTTCAGTACGTCTCTTTCCCCCACCGCAGGTCGTCGTAGGTGGCCTGCTGGTCGC
TGCCGAGCCGCTCCTCGAACTTCCGGCGGAGCGCGGCCTTCTCGGTCGCGGAGATCCGCGCCAGCTCGTCCGCCTTCGAAACCG
GCTCCGGCGGGCCCAGCCCGCGACCGACGGCGACGTCGACGAGGCGCTTCGCCTGGTCGGGCGAGAACCACTCCCGCTCTAACG
ACAGCGCGACGACGAACTCCCCCTCCCCGAGCCGGTCCTGGGCCCGGGTCTTGAACGGGGCGGCGACGGCGACCTCCAGACTCA
TGTGCGAATCGGGGGGCGCCGACGGCGGTAAACCTACCGGACCGCATCCCGCTTCAGGGCGGCCGCGACGTCCGGCTTATAGCG
GTAGCGAGGCGAAAGCCCACCCGTTTACGGGTGGGATGAAGTCGACAACTGGGAATCTATCCACGCTCGTAGCCACGGCTGGGG
TTGGATGCTTGTGCAACATATTTAGGTGAAGATACTCTATATACTGACGATGCCTCGTGGGTTCGACAGGGAACGTACCAACGT
CCACAAACTCCAGTACCACTTCATCTGGTGTTCGAAATACCGTAAATCGGTGCTTGAGGGCGAGGTACGCGACCGTCTCGAAGA
ACTCATCGAGGAGAAAGCCGACGAACTCGACCAGGTCAAGGGCTACTCCTCGCGCCGACTTCGGGACGAGTTCGACTTCGGCTT
GCCGTCGCTGTGGACGCGCTCGTACTTCGTCTCAAGCGCGGGCGACGTGTCCAGCGAAGTCATCGAGGAGTACATCGACACCCA
AGCAGGTGAATAGTCATGCAACGGAACGTCTCGACCACGGTGCGAGTCAAACTCCACTCGCTGACCAACAGGAAAGCCGACCTA
CTCGCCCGCGAGTATGAGGCGTTCCAGACCGCAGTTCACGGCGGAGACGCTGACCTCTACTCCGCGACCGACCAGCAGGCGGCC
TTGGGCAAGTTCTCGAAACCGCTGTCCGAGGCGGGGGCTGTACTGGCACAGCCCGAAACGCAGGTCATCGTCCCACGTGACGAC
GAACCTGCGAACCTCTCCGTGTCCGTGGGTTCAACCCCCAGTGGGGGAACCCCACGGCTTTAGCCGTGGGAGGATGTCAGGCCC
GGCAACCGTTATGTCGGGCCAGCGAGATGGCGCACGTATGAAGACCCGCCCGCTCGGCGACACCGGCCACGACAGCACCGTCAT
GACGTTCGGCGCGATCGCGCTCAACTGGCTCGAACAGGAGGGCGCGAACCAGCTGGTCGAACACGTCCTCGACTACGGGGTCAA
TCACTTCGACGTGGCCCCG (SEQ ID NO: 816)
>3300027896|Ga0209777_10000273_32|M
TCCGGGGTGTATCTTGCTTCTGGTTTGTATGTGAATGCAGGGACCATGTCTGCGAATGTGTCTGGGAATTGGATTGAGGCGCAT
GTGATGTCTGGTGCTTTGACCATCATCTCTGGTGGCGTGTTTGTCTCTGGTTCTATCACAGTGATTTCTGGTTTGATTGGTGCG
TATGTTTCTGGTTTTGTCACTGTGCAGAGTGGTGTGATTCAGGTTCTATCTGGTGTAGGTGTTCTTACTTCTGTTTCTGGGAAT
ATTGTTTCTATTGCTTCTGGGTTGTATGTGACTATTCAGTCTGGTGGTGCTGTTTCTGTTCCTATCACTTCTGGTTCTGTGATT
GTGTCTGGGAATATTGGTGTCTCTGGCTGGGTGAGTGTGCAGAGTGGTGTGATTCAGGTTCTATCTGGTGTAGGTGTTCTTACT
TCTGTGTCTGGGAATATTGTTTCTGTCGCTTCTGGGTTGTATCTTGAATCTGGTCAAGAGATTATCTGGCGTGGTGTGCAAGTC
TCTGGCACTGTCGCTGTCTCTGGTTTGATTGGTGTGCAATCTGGTGAGGTTCATGTGATGTCTGGTGCTTTGACCATCATCTCT
GGTGGCGTGTTTGTCTCTGGTTCTATCACAGTGATTTCTGGTTTGATTGGTGCGTATGTTTCTGGTTTTGTCACTGTGCAGAGT
GGTGTGATTCAGGTTCTGTCTGGTGCGGGTGTGGTTGCTTCTGTTTCTGGGAATATTGTTTCTGTTGCTTCTGGGTTGTATCTT
GCTTCTGGGTTGTATGTGAATGCGGGGACCATGTCTGCGAATGTGTCTGGGAACTGGGTTGAGACGCATGTGATGTCTGGGAAT
CTTCAGGTTTTACAGACATCTGGTTTTGTTGCTCTTCAATCTGGTGAAATTCATATAATGAGTGGTTCAGTGACTATACCCGCA
TTGACAACAGTTGTTGCCACCCCAGCAACAAACCCCATGCTGATTACCGGTCTTTCAGGAGGACAAATTTTGACTTCAAGTGTA
GTAATAAGCGCTATTGTGAAAGCAGTATCACTAAACAGTAGTGATATGTATATTGGTGGAGCATCGGGGGTAAATAGCCCTCCG
TATTCTGGTGGAGGGTTTGTGCTTGCTCCTGGTGAAGCTATTAATCTTGATGTCGCAAATATCGGGTATGTGCGGGCCTGTGCA
GTGATGAGTGGTGATAAAGTATCATGGATTGGGATAGTATAAGAGGCAAATATGGTTAAAGAATTTCTCTTTGAACAGTTTTAC
ACAGGGACAACAGGTGTCGATACAGCGTATGGAGTAAATTGGAGGGCAACGACATTCACGATAGGGACTGTTGGCGCGAAAGCG
TCTCATTATGCGACAAGCATAGGATGGCGAGGGCAAGATTTTCCAACCATTCGTTTCAGCGATAATGTTGAAACAGTCCTTGAG
GAAAATTTCAATATCTCCGACCAAGACTTGTTTTCTATATTTCGGGCAGAAAACAACATGGTAATTTAGATTGTATACTGCGGA
ACTTGACCTGCCCATAAATCTTAAATACCTATAGGTGTATATAAGATTTGTGGCACAGAAAACCATCACCGCAAAAATCGTAGC
AATGACCGCTACGAAACAACAAATACTCCAAACCGAGTATGACAACCTCCAGCATTTTCTACAAACTGGAGAAGACCTTGGAGT
ATACTCAGCGAACAAACAACAAGCCAAAAGAACAAGTTTCAATGCCGACTACAATGGTGCATTAAATATCTTGAAGAGGGCAAC
ATCCTACATGGATATTGCTGGGGCTGTGAGTGAACCAGCCCGTGACTGGGGCAACGACGACCAGACCTCAGAAGCCACCCAACT
TTTTGAGTGGTAGTTCACTAATGTAACTGGGAATAAGAAAATCATTGAAGTATTTGGTGAACGATGGCACACTGGGGAAAAGAT
TCCATATAGACGAACTGAGATTGGGAGGAAAAAAATATTCGAAGAAAATGGGTATGCTCTTTTGATAATATGGGATTATGAATT
AATTGATGAACAAAAAGTTATAGAAAAAATAAAAGATTTTGTCTAAATTAGACAAAATTAAATCCTTCCTGTTTCGAGTACTAT
TGTTGTTGTGGTTGTGATTCCCGAGTGAGTATATGTTTTAGTAACAGTTCCATTCACAGTGACGCTTTGACTCCAATATGAACC
AGCGAGTTCGATTCCACCCACGAGCAGATGAGCGTAGAG (SEQ ID NO: 817)
>WP_121304574.1
TACCCCAACACCGGCGCCAACAGAAGCACCAACTGCGACCCCGACGCCGACTCCAACTCCAGATCCAGTAATCGTTGAAGTCGG
CGAATGGGGGAAGATAGAGCAGAACGGTACAGTGGTTCGGTTTCGAGTGGTTAGCTATGACATCACGGAATCTGTTGAAGAAGC
CGATGGAGAGACAGAAGAGCCATCTGAAGGGTATAAATTTGTAGTTGCAGACGTACATATCTCTACTGATCCCGGCAATGAAGT
TACGATTCGAAAAGACCAGTGGTCGTTTACTGAACTTACCGATAAGAAGCGCGGCCCTGATGGCGTGACCAACGGAATTAGAGA
GCCGTTCCCAAACAAGACTGCATTACAGGGTGATTCGGTTCGAGGAACTATTGTTTGGGAAGTAAAATATCCGTCTGATGGGCA
GTTCGAGGTGGAGATACATAAAAACAAAACAGGCAGGGCTGGTAAAGTGTTAATATAACAGAGTAGCTTGATAAGAGTTTTACA
TATTATCGTGGATAATTTATGCTCGAAAGGGGTTCAAAAACAGTTCTGCTCGGAGATAATTTTTGAGGGCTGACTGTAGTTGTG
GAAGTGGTCCATCGTGCCGGGTGATGCTCTGTATAATTATACCATGTAGCATCGCTAGGAGAAATTCAGCGGTTTCTTCGGGGG
AGAGAGTCACCGCCGAAGCGACCGGAGTTCCCGGCAGAAGGCCGGTGACGATGACTCGGCTATGGATGGGGCCTCTTTGATCGG
GTCACAAGACAGCCACGCTGATGCTGAACCCCAGCAGACGACGCGTGGAACGTATGCGTCTTGAAACCAACAGGCCGCTACACT
CGGCCTGAAATCCCGTGGTGGGACTCCTCCACGTTCACGCGGAGGAGGAGGTCAATTTGCCGATCTTGTTTGCACGCATTCAGT
GAGACAATGCGAGAAACCGCCCGGTTGCAGTCCAAACTACGAGAAGGTCGATACAATAGCGAGCGTATCCGGCGGTTGTACCGG
AAACAGACTCGTCGTCACGACACACCCGAGGAGCACTGTGTCATGACCTGCTGGAACAACCTGTACGCCGAGGGTATCAACATC
GTGTATATCGGTGACTTGACCGACGTGCTGGATACGCACTGGTCGGTCGAAAGCAACGCCAAGACCCAGAACTTTTGGGCGCTC
AAGAAGTTTCAGTCGATCACATCGAAAGCTCACAAACCCTTCCAGTAGAAGTTGAATCTGTGTCTACGGTGCCTGGCCAGTTCT
CGAAGTGAGACGGACAGTGAGTTTTGGCGGTGAGGAGTCGCTGTCGGCGGCGGATCGCCGCCGACGCGACAGTGTCGCCACTCA
CGCCCGCTGTCCTAGCTGGGACAACTACCTGAAGAACCGGTCGTCAAGTGGTTGGTTCGCTGGAACAGACCTGCGGACACCAAG
TGCCGTCCAAGCTGCGCGAAGCGTCATTTCGCAGAACTCGGTGAACGACCACTGTAAGAGGCGGCGCCCCCCGCGGCGGGGCGC
CGCCAGACTGCACATCCTCTCCAAGCAGACGCTTACTCTAACCCGATGTGATCGACTGAGTTTACAGCGGTAGCGAGGCGAAAG
CCCATCGGCTTTAGCCGTGGGATGAAGCCGACAACTGGGAATCTATCCACGCTTGTAGCCACGACTGGGGTTAGATGTTTGCGT
AACATCTTTAGCTGAGTGATATCTATATACTGACGATGCCTCGTGGGTTCGACAGGGAACGTACCAGCGTCCACAAACTCCAGT
ACCACTTCATCTGGTGTCCGAAATACCGTAAATCGGTGCTTGAGGGCGAGGTACGTACCCGCCTCGGGGAACTCATCGAAGAGA
AAGCCGACGAGTTCGGGCAGGTCAAGGGCTACTCCTCACGCCGACTTCGGGACGAGTTCGACTTCGGTTTGCCGTCGCTGTGGA
CGCGCTCGTACTTCGTCTCAAGTGCGGGCGACGTATCCAGCGAAGTCATCGAGGAGTACATCGACGCCCAAGCAGGTGAGTAGC
CATGAAACGGAACGTCTCAACCACGGTTCGGGCCAAACTCCACTCACTGACAAACAGAAAGACTGATCTGCTCGCCCGTGAGTA
TGAGGCGTTCCAGACTGAGGTACACGGTGGAGACGCTGACCTGTACTCTGCAACCGACCAGCAGGCGGCCTTGGGCAAGTTCTT
GAAACCGCTGTCCGAGGCGGGGGCTGTACTGGCACAGTCCGAAACGCAGGTCATCGTCCAACGCGACGACGAACCTGCGAACCT
CTCTGTTTCCGTGGATTCAACCCCCAGCGGGGGAACCCCACGGCTTTAGCCGTGTGGAGGATGTCAGAAGCGGTCTAGGAGAAT
GCCCCGGGGCTTGACCCCGGGGTTGAATCCGATAAGCAGGAGTACAAATCATTAAGTGAACATATCCCGAATCAAGAGACAGCG
ATGGAGTACAGTCACCGCTACCCCGCGTACCCAACACAAGAGGTGGCGGCTGAACTGGAACGACACATCGACGCTCATCGCCAA
GCGTACAACTACACCCTGTACAAGTACGAGAACGTGGACGCCGACAATATCGGTTCCGCGTACAAA
(SEQ ID NO: 818)
>330002794|Ga0207719_100292_34|M
ACCCTGAGCCTAGGCGTCGTCGGCGTGATCGCCGGCCCGCTCGCCATCGCCCTGCTCGTCGAAGTGGTCGAACTGATCACCGAC
GAACGGGTCAGCATCCAGCAGACCTTCCAGGAGAGCGCCACCGAGGCCGCCGGCGACGACGACTGAGCGGCGGCCGTACCGACG
CGAGTGCGGGCGGCGTTACAGCGGTAGCGAGGCGAAAGCTCACCCGTTTACGGGTGGGATGAAGCCGACAATTGGGAATCTATC
CACGTTCGTAGCCGCGGCTGGGGTTGGATACTTGCATAACATCTTTAGGTGAATAGTATCTATATACTGACGATGCCTCGTGGG
TTCGACAGGGAACGTACCAGCGTCCACAAACTCCAGTATCACTTCATTTGGTGTCCGAAGTACCGCAAATCGGTGCTTGAGGGT
GAGGTACGTGACCGTCTCGAAGAACTCATTGAGGAGAAAGGAGACGAACTCGGCCAGGTCAAGGGCTACTCCTCGCGCCACCTC
CGCGACGAGTTCGACTTCGGTCTTCCCTCGCTGTGGACGCGCTCGTACTTCGTCTCAACTGCGGGCGACGTATCCAGCGAAGTC
ATCGAGGAGTACATCGACGCCCAAGCAGGTGAGTAGTCTTGCAACAAACCGTCTCGACCACGGTGCGGGTCAAACTCCCCTCGC
TGACCAACAGGAAGGCAGACCTGCTCGCCCGTGAGTACGAGGCGTTCCAGACCGAAGTTCACGGCGGCGATGCTGACCTCTACT
CCGCGACTAAGCAACAGGCGGCCTTGGGCAAGTTCTCGAAACCGCTGTCCGAGGCGGGGGCTGTACTGGCACAGTCCGAAACGC
AGGTCATCGTCCACCGTGACGAGGAACCTGCGAACCTCTCCGTATCCGTGGATTCAACCCCCAGTGGGGGAACCCCACGGCTTT
AGCCGTGGGAGGATGTCAGTCGCCCGTCTCGGGTTCGCGCCCCGCGGCCTTCCACTCGGTCAGACTCCCCTCGTAGAAGGCGAG
GTTCTCGTACCCGAGGTGTCGGAGGACGACGTAGGTGTGACTGATCCGGCGAGCGGTGTTGCAGTACAGCAGAACGCGCCGGGT
CGCTGAGCGGGATATTCTCACTCTCCGCACCGCCCGCGAGAATAGGGCCGCGCTCAGTCGACCACGCAAGCGCGAGCGCGCCTC
GCCCTTTCAGTCCACCAGGACCGCACCGCTCCGCCCCGCACCGCCACCGCAGACAGCCTCACACCTCCCCAGCCTCCTCGCGTC
TCGTCGCTCGCGTGACTCGCTCCTCGGTGCTCGTCCCTCGCACGGTGCGTCGCGGCACGGAGGCCGCGACGGCGCGCGCCACCG
CAATCGATCGGTGTCGGAACGGCCGCGAACGCACCTCTCAAGAGCCTGCGGGCCGTGGCGATCGGTAATGAGCGACACCGGCGG
CCCACTCTCACCGGACAGACCGGACGCCGAGAGCGATTTCCGGGTGGACGCCCCCTTCGACCCCGCGGGCGACCAGCCCGAGGC
CATCGAGCAACTGGTCGACGGCTTCGAACGCCAGGGCCACGACAAACAG
  (SEQ ID NO: 819)
>SRR8526178_megahit_k177_763700_3|P
ATGGTCACGGTTCGGATCAATGAAGTCACCAAGTCAAAGCTCGACAAGTTGTGCGAGGACAACGGCCTCAAGATGCGGTGGGTT
GTTGAACAGGCGATCCTTCAATATTGTGAGATGCACGAGTCGAAACATCGTCCAGAGGATGGGTAACTTATGTCACTCAAAACA
CTCACATTCTGGCCGATTGGTATGTCTGCAAAATACAGGCGGATATTTGACAACATCCGTAAGCAACTTGGGGCGGCGCAGCGC
GAAATGGTGAAAGCATGGCTACGGCTGGCGTTGGCAGAGGATGCTTATGGTGACCTTAATGCTGCGAATAACATCGCGCGGGGG
ATGCAGACGTTTGGGCCTTGTGCCAAGTCCTCTGCGTCAGACGACGCTATCGGGCTGCGGTTCGATAGCGAGGGGCAAGAATCT
GCCCTGGGGCAGGCGAAGCGATTGTTTGCTTGAGCTTGCAAGTTCTTGTGTGTGTGTGGGTATGAACTGCTGAGCGTTTGTGTA
GT (SEQ ID NO: 820)
>3300028675|Ga0272445_1001731_11|M
GTCGTAACGATGGCTGATGTGTTAGAGGGAGGGGCTGTGGGGATCAATGACAATAAAATGAACACTCTCGTATTAATGCATGCT
AACCCACTATGGGGCGAGATTGAGATAAAAACTCCCGCAGGGCGAACGGTTTGGCGTGCTCCTTAACCGTTGGTGACCTGAATG
TGGGAAACAATCTTGATTGGCGTTTGTGGGGCGCACTTTCCCGCTACCGTAGGGGCGACAAAAAGCAGCACTATTCTGGCTTTC
CGTCGCCATCATCGCTATCATCGCTCTCATAGCCATCAACACTTTGACCGTCCGCTGGCTCGGTCATCCGCCACAAAACTCATC
CACAACAATAGCACTGTCAGAGTGAAAGGACTTAGCAGCGTTCGTTAAAGCGCCGACCTGCAGCGCCGAAAGACACTTGCTAAG
CCTTACCGTCGGCTTGGTCACTTCCCGTTTCCCGTAGCGCGTGAAGCCAGAATGGTAAAGCCTAAAATGCCGTTTTTGCGTTGC
GACAACGCGCAAGTTAGCGTCAGCAGGCGCACATAGGCACAGGCGCACGGGCGTAGGCACGCAGGCGCACGCACGCGGGCACGG
GCGCGCTTTCAACGGGGATACCCGAGCGCGCAGGCGGGTCAGGCTGCCCGCCGACCTTAAAGTCAACCTTAAAGACTACAACCC
CCAAACCGACGCTATGTGCGCTATGCTTTGGGACGCTTATAAAGTTCAGGTGCTTAAGGAACTTGTTGGCGAGGATGAGGGCGA
CTACATTAAGTAAAGCCGTGAGGAGTTATTAGAGGCTTTGCGGGAACGGGAGCACAAGTATGGTTATAAAGCGCCCCTTAATGC
GAACTTAACAAAACCGTCCCGACCAGTTATTTCGGGTAAAATTTTAACCCATTTGCCCAAATTCCGCAACTTCGTTGCAGCAAA
CCTCCAGTCACCCCTTTCCCCTAAGGGGTTTGGCGCAAGCAATTCTTGAAATTCCTGCCAAAATCGGCAAAATTCAAAGGTCCC
GTTCTTTGATGCCTATCACGGCAGAATAAAGGGGGAGGTTGACGGCAAATGAAAAAAGTGGTCATAACCATCACTACTGGAGTA
GAGTTTCTACAACCTCAAGATGAGCAAATCGTGAGGAAGGCAGCGGAATGGTATACAAAGGCACGAGAAGCCCTTTTTGCCCAT
GCCCTCCAACAACAACAAGGTAAAGTTGAGGGAAACCGATTTTGTTGCACAAAGTGTAAATTTCAGCACAATGCGGACTTTGTG
GCGGCGTATAACATTGCCGTAAGGGCATTGAAGAAGTTAGGGATGAGGGAAGAGATAGGTTGGAAGGTATTGACAGGCAAGCCG
AAGGTGTTATAATGACGATGGCTCATGGGGAGCAGGCTCCCCATGGGCAGCGTGCCCGCCGAAAGGTGGGTGGAGGGCAGAGCC
TGTCTGCCTGTAAGCAGCACCTTCCCACATGGGGAGGTTGTCTGCAAGTTCACATTATCCCCTGACCCCAGCGGTGAAGAGCCG
CTGGGGTTTCAATTGTCTTAGCACCCTGATGTCCACATTAACCCCTTAAGACAGAAAACTTGCCCAAGCGAGGATTAGCACGAA
CTATTGCCTTGTAACGCTCCCCTTAGCAGTTGCACATTGCAAAGCAAGTTTCTTGGCATTAAGAGACTCTTTCAGTCCCACAAT
GGTTTGATTGCTACATTGCCTGCCAAGATAAAGCCCTTCCTTTCCATTATCACTTGTAAAGGACCAGTGTCATAATTCCTCAGG
GGTGAGGGAGATGAAGGTCATTATTGGGCGTGTAATGGAAGACACTTACACGGTCAAAAGTCAAGAACCCTTGCCACCAGGACA
GGAAGTCTTCATTGTAACTAAGATGGACGCTGAGGATGTCTATTACCTTGCAAAAACTTTGGCGTGG
(SEQ ID NO: 821)
```

### Example 6—Functional Validation of Engineered CLUST.196682 CRISPR-Cas Systems (FIGS. 13A, 13B, and 14)

Having identified the minimal components of CLUST.196682 CRISPR-Cas systems, we selected one loci for functional validation, from the metagenomic source designated 3300025638.

### DNA Synthesis and Effector Library Cloning

To test the activity of an exemplary CLUST.196682 CRISPR-Cas system, we designed and synthesized systems containing the pET28a(+) vector. Briefly, E. coli codon-optimized nucleic acid sequences encoding the CLUST.196682 3300025638 effector (amino acid sequence provided as SEQ ID NO: 601 in TABLE 8) were synthesized (Genscript) and cloned into a custom expression system derived from the pET-28a(+) (EMD-Millipore). The vector included a nucleic acid encoding CLUST.196682 3300025638 effector protein under the control of a lac promoter and an E. coli ribosome binding sequence. The vector also included an acceptor site for a CRISPR array library driven by a J23119 promoter following the open reading frame for the CLUST.196682 3300025638 effector.

We computationally designed an oligonucleotide library synthesis (OLS) pool containing “repeat-spacer-repeat” sequences, where “repeat” represents the consensus direct repeat sequence found in the CRISPR array associated with the effector, and “spacer” represents sequences tiling the pACYC184 plasmid as well as E. coli essential genes. The spacer length was determined by the mode of the spacer lengths found in the endogenous CRISPR array. The repeat-spacer-repeat sequence was appended with restriction sites enabling the bi-directional cloning of the fragment into the aforementioned CRISPR array library acceptor site, as well as unique PCR priming sites to enable specific amplification of a specific repeat-spacer-repeat library from a larger pool.

We next cloned the repeat-spacer-repeat library into the plasmid using the Golden Gate assembly method. Briefly, we first amplified each repeat-spacer-repeat from the OLS pool (Agilent Genomics) using unique PCR primers, and pre-linearized the plasmid backbone using BsaI to reduce potential background. Both DNA fragments were purified with Ampure XP (Beckman Coulter) prior to addition to Golden Gate Assembly Master Mix (New England Biolabs) and incubated per the manufacturer's instructions. We further purified and concentrated the Golden Gate reaction to enable maximum transformation efficiency in the subsequent steps of the bacterial screen.

The plasmid library containing the distinct repeat-spacer-repeat elements and Cas proteins was electroporated into E. Cloni electrocompetent E. coli (Lucigen) using a Gene Pulser Xcell® (Bio-rad) following the protocol recommended by Lucigen. The library was either co-transformed with purified pACYC184 plasmid, or directly transformed into pACYC184-containing E. Cloni electrocompetent E. coli (Lucigen), plated onto agar containing chloramphenicol (Fisher), tetracycline (Alfa Aesar), and kanamycin (Alfa Aesar) in BioAssay® dishes (Thermo Fisher), and incubated for 10-12 hours at 37° C. After estimation of approximate colony count to ensure sufficient library representation on the bacterial plate, the bacteria were harvested and plasmid DNA extracted using a QIAprep Spin Miniprep® Kit (Qiagen) to create an “output library.” By performing a PCR using custom primers containing barcodes and sites compatible with Illumina sequencing chemistry, we generated a barcoded next generation sequencing library from both the pre-transformation “input library” and the post-harvest “output library,” which were then pooled and loaded onto a Nextseq 550 (Illumina) to evaluate the effectors. At least two independent biological replicates were performed for each screen to ensure consistency.

### Bacterial Screen Sequencing Analysis

Next generation sequencing data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source (pACYC184 or E. Cloni) or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

To identify specific parameters resulting in enzymatic activity and bacterial cell death, we used next generation sequencing (NGS) to quantify and compare the representation of individual CRISPR arrays (i.e., repeat-spacer-repeat) in the PCR product of the input and output plasmid libraries. We defined the array depletion ratio as the normalized output read count divided by the normalized input read count. An array was considered to be “strongly depleted” if the depletion ratio was less than 0.1 (more than 10-fold depletion). When calculating the array depletion ratio across biological replicates, we took the maximum depletion ratio value for a given CRISPR array across all experiments (i.e. a strongly depleted array must be strongly depleted in all biological replicates). We generated a matrix including array depletion ratios and the following features for each spacer target: target strand, transcript targeting, ORI targeting, target sequence motifs, flanking sequence motifs, and target secondary structure. We investigated the degree to which different features in this matrix explained target depletion for CLUST.196682 systems, thereby yielding a broad survey of functional parameters within a single screen.

FIGS. 13A-B depict the location of strongly depleted targets for CLUST.196682 3300025638 effector targeting pACYC184 and E. coli E. Cloni essential genes. Notably, the location of strongly depleted targets appears dispersed throughout the potential target space for E. Cloni essential genes.

FIG. 14 depicts a weblogo of the sequences flanking depleted target, indicating a prominent 5′ PAM of CG.

### Example 7—Identification of Minimal Components for CLUST.089537 CRISPR-Cas System (FIGS. 15-18)

This protein family describes a large single effector associated with CRISPR systems found in Phycisphaerales and Planctomycetes organisms, and uncultured metagenomic sequences collected from freshwater, permafrost, sediment, soil, anaerobic, and hot springs environments (TABLE 11). CLUST.089537 effectors include the examples of proteins detailed in TABLES 11 and 12, below. Examples of direct repeat sequences for these systems are shown in TABLE 13.
Examples of naturally occurring loci containing this effector complex are depicted in FIG. 15, indicating that for CLUST.089537 loci, the effector protein co-occurs with a CRISPR array. No other families of large proteins were identified within a bi-directional 15 kb window that co-occur with the effector protein or a CRISPR array.
The direct repeat sequence of CLUST.089537 exhibit multiple conserved motifs, including 5′-RBBNRBKGACAS-3′ (SEQ ID NO: 834) proximal to the 3′ end (FIG. 16), where R refers to A or G, B refers to C or G or T or U, K refers to G or T or U, S refers to C or G, and N refers to any nucleobase.
FIG. 17 is a schematic representation of a phylogenetic tree of CLUST.089537 effectors, showing that the family exhibits sequence diversity.
FIG. 18 is a schematic representation of a multiple sequence alignment of CLUST.089537 effector proteins revealing the location of the conserved catalytic residues of the RuvC domain, indicated by the colors Magenta, Blue, and Red.
Optionally, the system includes a tracrRNA that is a subset of a non-coding sequence listed in TABLE 14.

TABLE 11
Representative CLUST.089537 Effector Proteins

| | | # | | | effector |
|---|---|---|---|---|---|
| species | effector accession | spacers | cas1 | cas2 | size |
| bioreactor metagenome | CAAACX010000652_2\|M | 7 | Y | N | 674 |
| (CAAACX010000652) | | | | | |
| activated sludge metagenome | CAAACH010011569_2\|M | 3 | Y | Y | 759 |
| (CAAACH010011569) | | | | | |
| activated sludge metagenome | CAAACI010012331_3\|M | 2 | Y | Y | 759 |
| (CAAACI010012331) | | | | | |
| activated sludge metagenome | UZXW01002767_11\|M | 2 | Y | N | 758 |
| (UZXW01002767) | | | | | |
| aquatic-freshwater-freshwater lake | 3300009161\|Ga0114966_10008307_8\|M | 4 | Y | N | 728 |
| (3300009161\|Ga0114966_10008307) | | | | | |
| aquatic-freshwater-freshwater lake | 3300009161\|Ga0114966_10008307_7\|P | 4 | Y | N | 762 |
| (3300009161\|Ga0114966_10008307) | | | | | |
| aquatic-freshwater-freshwater lake | 3300010885\|Ga0133913_10083255_2\|P | 4 | Y | N | 762 |
| (3300010885\|Ga0133913_10083255) | | | | | |
| aquatic-freshwater-freshwater lake | 3300020109\|Ga0194112_10008260_1\|P | 2 | Y | N | 676 |
| (3300020109\|Ga0194112_10008260) | | | | | |
| aquatic-freshwater-freshwater lake | 3300020109\|Ga0194112_10008260_1\|M | 2 | Y | N | 678 |
| (3300020109\|Ga0194112_10008260) | | | | | |
| aquatic-freshwater-freshwater lake | 3300020222\|Ga0194125_10021011_3\|M | 4 | Y | N | 687 |
| (3300020222\|Ga0194125_10021011) | | | | | |
| aquatic-freshwater-freshwater lake | 3300027770\|Ga0209086_10006474_1\|P | 3 | Y | N | 762 |
| (3300027770\|Ga0209086_10006474) | | | | | |
| aquatic-freshwater-freshwater | 3300015360\|Ga0163144_10184575_1\|M | 2 | Y | N | 779 |
| microbial mat | | | | | |
| (3300015360\|Ga0163144_10184575) | | | | | |
| aquatic-freshwater-sediment | 3300013130\|Ga0172363_10020541_1\|M | 27 | Y | N | 761 |
| (3300013130\|Ga0172363_10020541) | | | | | |
| aquatic-freshwater-sediment | 3300013133\|Ga0172362_10044703_1\|M | 8 | Y | N | 654 |
| (3300013133\|Ga0172362_10044703) | | | | | |
| aquatic-freshwater-sediment | 3300031862\|Ga0315280_10005430_2\|M | 4 | Y | N | 710 |
| (3300031862\|Ga0315280_10005430) | | | | | |
| aquatic-freshwater-sediment | 3300031952\|Ga0315294_10035890_2\|P | 12 | Y | N | 691 |
| (3300031952\|Ga0315294_10035890) | | | | | |
| aquatic-freshwater-sediment | 3300031952\|Ga0315294_10041214_2\|M | 2 | Y | N | 741 |
| (3300031952\|Ga0315294_10041214) | | | | | |
| aquatic-freshwater-sediment | 3300031952\|Ga0315294_10041214_2\|P | 2 | Y | N | 745 |
| (3300031952\|Ga0315294_10041214) | | | | | |
| aquatic-freshwater-sediment | 3300031952\|Ga0315294_10058002_1\|M | 7 | Y | N | 794 |
| (3300031952\|Ga0315294_10058002) | | | | | |
| aquatic-freshwater-sediment | 3300032046\|Ga0315289_10071835_2\|M | 3 | Y | N | 727 |
| (3300032046\|Ga0315289_10071835) | | | | | |
| aquatic-freshwater-sediment | 3300032053\|Ga0315284_10046226_2\|P | 7 | Y | N | 740 |
| (3300032053\|Ga0315284_10046226) | | | | | |
| aquatic-freshwater-sediment | 3300032069\|Ga0315282_10002412_8\|P | 6 | Y | N | 744 |
| (3300032069\|Ga0315282_10002412) | | | | | |
| aquatic-freshwater-sediment | 3300032118\|Ga0315277_10001015_12\|P | 3 | Y | Y | 796 |
| (3300032118\|Ga0315277_10001015) | | | | | |
| aquatic-freshwater-sediment | 3300032118\|Ga0315277_10012802_3\|M | 6 | Y | N | 759 |
| (3300032118\|Ga0315277_10012802) | | | | | |
| aquatic-marine-deep subsurface | 3300009499\|Ga0114930_10003500_21\|M | 2 | Y | N | 702 |
| (3300009499\|Ga0114930_10003500) | | | | | |
| aquatic-marine-deep subsurface | 3300011118\|Ga0114922_10000510_27\|M | 3 | Y | N | 736 |
| (3300011118\|Ga0114922_10000510) | | | | | |
| aquatic-non marine saline and | 3300017960\|Ga0180429_10052609_1\|M | 3 | Y | N | 679 |
| alkaline-hypersaline lake sediment | | | | | |
| (3300017960\|Ga0180429_10052609) | | | | | |
| aquatic-non marine saline and | 3300017963\|Ga0180437_10045654_1\|M | 13 | Y | N | 741 |
| alkaline-hypersaline lake sediment | | | | | |
| (3300017963\|Ga0180437_10045654) | | | | | |
| aquatic-non marine saline and | 3300017971\|Ga0180438_10003700_2\|P | 16 | Y | N | 741 |
| alkaline-hypersaline lake sediment | | | | | |
| (3300017971\|Ga0180438_10003700) | | | | | |
| aquatic-non marine saline and | 3300017991\|Ga0180434_10065880_1\|P | 4 | Y | N | 762 |
| alkaline-hypersaline lake sediment | | | | | |
| (3300017991\|Ga0180434_10065880) | | | | | |
| aquatic-non marine saline and | 3300017992\|Ga0180435_10008274_7\|P | 14 | Y | N | 670 |
| alkaline-hypersaline lake sediment | | | | | |
| (3300017992\|Ga0180435_10008274) | | | | | |
| aquatic-non marine saline and | 3300018080\|Ga0180433_10080815_1\|M | 4 | Y | N | 762 |
| alkaline-hypersaline lake sediment | | | | | |
| (3300018080\|Ga0180433_10080815) | | | | | |
| aquatic-thermal springs-hot spring | 3300009503\|Ga0123519_10002165_1\|M | 4 | Y | Y | 792 |
| (3300009503\|Ga0123519_10002165) | | | | | |
| bioreactor metagenome | CAAACB010000011_94\|M | 12 | Y | N | 789 |
| (CAAACB010000011) | | | | | |
| bioreactor metagenome | SRR5251193_megahit_k177_201695_120\|P | 8 | Y | N | 679 |
| (SRR5251193) | | | | | |
| bioreactor metagenome | SRR5251487_megahit_k177_199995_1\|P | 5 | Y | N | 679 |
| (SRR5251487) | | | | | |
| bioreactor metagenome | SRR5576005_megahit_k177_1292294_1\|M | 8 | Y | N | 789 |
| (SRR5576005) | | | | | |
| bioreactor metagenome | U0UK01008448_2\|M | 3 | Y | Y | 757 |
| (U0UK01008448) | | | | | |
| bioreactor metagenome | U0UL01003058_1\|M | 2 | Y | Y | 769 |
| (U0UL01003058) | | | | | |
| bioreactor-anaerobic-anaerobic | 3300009652\|Ga0123330_1010394_3\|M | 2 | Y | Y | 769 |
| biogas reactor | | | | | |
| (3300009652\|Ga0123330_1010394) | | | | | |
| bioreactor-switchgrass degrading | 3300012949\|Ga0153798_10025413_2\|P | 13 | Y | N | 638 |
| (3300012949\|Ga0153798_10025413) | | | | | |
| cave metagenome (SRR7811943) | SRR7811943_megahit_k177_406423_2\|M | 3 | Y | N | 778 |
| freshwater metagenome | SRR5818194_megahit_k177_802550_7\|M | 4 | Y | N | 728 |
| (SRR5818194) | | | | | |
| freshwater metagenome | SRR5818194_megahit_k177_802550_6\|P | 4 | Y | N | 762 |
| (SRR5818194) | | | | | |
| freshwater sediment metagenome | SRR6481359_megahit_k177_1860565_4\|M | 6 | Y | Y | 743 |
| (SRR6481359) | | | | | |
| hot springs metagenome | SRR6049666_megahit_k177_256604_1\|P | 4 | Y | N | 792 |
| (SRR6049666) | | | | | |
| hydrothermal vent metagenome | SRR2080392_megahit_k177_207267_1\|M | 4 | Y | N | 785 |
| (SRR2080392) | | | | | |
| hydrothermal vent metagenome | SRR2080392_megahit_k177_207267_2\|P | 4 | Y | N | 742 |
| (SRR2080392) | | | | | |
| lab enrichment-defined media-ionic | 3300025517\|Ga0207869_1001046_11\|M | 6 | Y | N | 674 |
| liquid and high solid enriched | | | | | |
| (3300025517\|Ga0207869_1001046) | | | | | |
| lab enrichment-defined media-ionic | 33000255721Ga0207864_1000213_47\|M | 7 | Y | N | 674 |
| liquid and high solid enriched | | | | | |
| (3300025572\|Ga0207864_1000213) | | | | | |
| lab enrichment-defined media-ionic | 33000255721Ga0207864_1014515_2\|P | 3 | Y | N | 679 |
| liquid and high solid enriched | | | | | |
| (3300025572\|Ga0207864_1014515) | | | | | |
| lab enrichment-defined media-ionic | 33000255721Ga0207864_1014515_3\|M | 3 | Y | N | 638 |
| liquid and high solid enriched | | | | | |
| (3300025572\|Ga0207864_1014515) | | | | | |
| permafrost metagenome | SRR6505097_megahit_k177_377355_35\|M | 7 | Y | N | 784 |
| (SRR6505097) | | | | | |
| permafrost metagenome | SRR6962291_megahit_k177_1754533_5\|M | 3 | Y | N | 722 |
| (SRR6962291) | | | | | |
| permafrost metagenome | SRR6962291_megahit_k177_1754533_5\|P | 3 | Y | N | 762 |
| (SRR6962291) | | | | | |
| permafrost metagenome | SRR6962291_megahit_k177_3367137_15\|M | 12 | Y | N | 735 |
| (SRR6962291) | | | | | |
| permafrost metagenome | SRR6962291_megahit_k177_3367137_14\|P | 12 | Y | N | 760 |
| (SRR6962291) | | | | | |
| Phycisphaerae bacterium ST- | AQT69685.1 | 13 | Y | N | 747 |
| NAGAB-D1 (CP019791) | | | | | |
| Phycisphaerales bacterium | PMBN01000097_4\|M | 3 | Y | N | 784 |
| (PMBN01000097) | | | | | |
| Phycisphaerales bacterium | PMDC01000375_4\|M | 7 | Y | N | 784 |
| (PMDC01000375) | | | | | |
| Phycisphaerales bacterium | PMMM01000072_9\|M | 7 | Y | N | 784 |
| (PMMM01000072) | | | | | |
| Planctomycetes bacterium | QBM02859.1 | 19 | Y | Y | 743 |
| RBG_13_46_10 (MHYD01000012) | | | | | |
| sediment metagenome | SRR6963308_megahit_k177_1876783_1\|M | 8 | Y | N | 679 |
| (SRR6963308) | | | | | |
| sediment metagenome | SRR6963352_megahit_k177_1225001_5\|P | 12 | Y | N | 762 |
| (SRR6963352) | | | | | |
| sediment metagenome | SRR6963480_megahit_k177_1813084_1\|M | 5 | Y | N | 762 |
| (SRR6963480) | | | | | |
| sediment metagenome | SRR6963491_megahit_k177_4304123_3\|P | 17 | Y | N | 670 |
| (SRR6963491) | | | | | |
| sediment metagenome | SRR6963587_megahit_k177_2272343_29\|M | 16 | Y | N | 741 |
| (SRR6963587) | | | | | |
| sediment metagenome | SRR6963591_megahit_k177_1621237_2\|P | 16 | Y | N | 741 |
| (SRR6963591) | | | | | |
| sediment metagenome | SRR8554444_megahit_k177_608234_1\|M | 6 | Y | Y | 792 |
| (SRR8554444) | | | | | |
| soil metagenome (SRR4030068) | SRR4030068_megahit_k177_2163203_10\|M | 2 | Y | N | 754 |
| soil metagenome (SRR5487326) | SRR5487326_megahit_k177_254484_5\|M | 3 | Y | Y | 767 |
| soil metagenome (SRR5487761) | SRR5487761_megahit_k177_404752_1\|M | 3 | Y | N | 767 |
| soil metagenome (SRR8554485) | SRR8554485_megahit_k177_439309_3\|P | 8 | Y | N | 767 |
| soil metagenome (SRR8554511) | SRR8554511_megahit_k177_4512460_1\|M | 2 | Y | N | 669 |
| soil metagenome (SRR8554511) | SRR8554511_megahit_k177_1767604_11\|P | 8 | Y | N | 744 |
| soil metagenome (SRR8554643) | SRR8554643_megahit_k177_4328113_1\|P | 3 | Y | N | 669 |
| soil metagenome (SRR8554643) | SRR8554643_megahit_k177_1281129_3\|P | 5 | Y | N | 740 |
| soil metagenome (SRR8554643) | SRR8554643_megahit_k177_1281129_3\|M | 5 | Y | N | 706 |
| subsurface metagenome | SRR6056591_megahit_k177_2733919_1\|P | 3 | Y | N | 736 |
| (SRR6056591) | | | | | |
| terrestrial metagenome | SRR6263259_megahit_k177_232832_6\|M | 2 | Y | N | 758 |
| (SRR6263259) | | | | | |
| terrestrial-soil | 3300031539\|Ga0307380_10018846_3\|P | 5 | Y | N | 740 |
| (3300031539\|Ga0307380_10018846) | | | | | |
| terrestrial-soil | 3300031539\|Ga0307380_10018846_4\|M | 5 | Y | N | 706 |
| (3300031539\|Ga0307380_10018846) | | | | | |
| terrestrial-soil | 3300031539\|Ga0307380_10072822_2\|M | 3 | Y | N | 669 |
| (3300031539\|Ga0307380_10072822) | | | | | |
| terrestrial-soil | 3300031539\|Ga0307380_10072822_2\|P | 3 | Y | N | 691 |
| (3300031539\|Ga0307380_10072822) | | | | | |
| terrestrial-soil | 3300031566\|Ga0307378_10072489_1\|M | 4 | Y | N | 669 |
| (3300031566\|Ga0307378_10072489) | | | | | |
| terrestrial-soil | 3300031566\|Ga0307378_10072489_2\|P | 4 | Y | N | 691 |
| (3300031566\|Ga0307378_10072489) | | | | | |
| terrestrial-soil | 3300031670\|Ga0307374_10070813_2\|P | 6 | Y | N | 767 |
| (3300031670\|Ga0307374_10070813) | | | | | |
| terrestrial-soil | 3300031965\|Ga0326597_10006968_2\|P | 12 | Y | N | 698 |
| (3300031965\|Ga0326597_10006968) | | | | | |
| terrestrial-soil-compost | 3300017540\|Ga0180217_1000254_36\|M | 8 | Y | N | 687 |
| (3300017540\|Ga0180217_1000254) | | | | | |
| terrestrial-soil-fen | 3300014494\|Ga0182017_10000840_9\|P | 7 | Y | N | 784 |
| (3300014494\|Ga0182017_10000840) | | | | | |
| terrestrial-soil-fen | 3300014839\|Ga0182027_10004505_3\|M | 21 | Y | N | 735 |
| (3300014839\|Ga0182027_10004505) | | | | | |
| terrestrial-soil-fen | 3300014839\|Ga0182027_10004505_2\|P | 21 | Y | N | 760 |
| (3300014839\|Ga0182027_10004505) | | | | | |
| terrestrial-soil-hardwood forest soil | 3300031715\|Ga0307476_10010105_2\|P | 3 | Y | Y | 768 |
| (3300031715\|Ga0307476_10010105) | | | | | |
| terrestrial-soil-hardwood forest soil | 3300031715\|Ga0307476_10010105_1\|M | 3 | Y | Y | 699 |
| (3300031715\|Ga0307476_10010105) | | | | | |

TABLE 12
Amino acid sequences of Representative CLUST.089537 Effector Proteins

>CAAACX010000652_28|M
[bioreactor metagenome]
MGVKAWKAKIIVADPDLAAALDHTVRQWNLWAEQACIAIMRLRRGDYGDEGQAVWRFLSEKGTFGTSILDKITRVTGRGIKNDE
ERTIASNFLSRHGPFEPLRAARDALGPNFWYQFQRAVRERIESHDAKLRQWREDHAEWIETRRTWEEEHRAYMNVRPVIEAFEA
AEGQSGRRRGRWRRWLDFLSSHPELAAWRGGPATIVPLSKEELAACRRRRRRAVAEQARLFFEKNPELRALNREHDEYQEKFAR
PWAKRRNPDGFRHRPTFTLPHPDSHPDWPRFKGNAGWRDLSVPDRTVSLELLCPDGSRRWFNVRFVPDPRLASLRPATQPVRQG
NTTYHYVSDALGVAEQPVQPQGIKLILRPDGAYLSVSVSVLAPPCGFDVRQSAIEKYGSIWACRKYKQDHPDRPLVTCAVDLAV
RHNGALTVARDGEVFARRLLHNRFFLPGDDRERAMNIPSLLEIAAVKRQLRRARRQTGRIAPGKPSCARLQAHYRALCEDRYKK
LVAAIFAYARHHGAEVVIFEDLKNLLPNAVNERGVNRALQSWNRGAIVRFARQSAEDVGLRVATVPAFHTSIVCARCGALGCRF
EQTREGVRIAKLGPWFGCRACGRRIHADLNASENLHKVLIGTFPQVRRISRDPPVLSIADVRVDQREADRLAAAWLSELSVGPT
PF (SEQ ID NO: 301)
>CAAACH010011569_2|M
[activated sludge metagenome]
MQLWRTHCVFNQRLPALLKRLFAMRRGEVGGNEAQRQVYQRVAQFVLARDAKDSVDLLNAVSLRKRSANSAFKKKATISCNGQA
REVTGEEVFAEAVALASKGVFAYDKDDMRAGLPDSLFQPLTRDAVACMRSHEELVKTWEKEYQEWRDRKSEWEAEPEHARYLNL
RPKFEEGEAARGGRFRKRAERDHAYLDWLEANPQLAAWRGKAPPAVVPIDEAGKRRIARAKAWKQASVRAEEFWKRNPELHALH
KIHVQYLREFVRFKQRPTFTMPDPVHHPRWCLFNAPQTSPQGYRLLRLPQSRRTVGSVELRLLTGPSDGAGFPDAWVNVRFKAD
PRLAQLRPVKVPRTVTRGKNKGAKVEADGFRYYDDQLLIERDAQVSGLKLLFRDIRMAPFADKPIEDRLLSATPYLVFAVKIKD
EARTERAKAIRFDETSELTKSGKKQKSKTLPAGLVSVAVDLDTRGVGFLTRAVIGVPEIQQTHHGVRLLQSRYVAVGQVEARAS
GEAEWSPGPDLAHIARHKREIRRLRQLRGKPVKGERSHVRLQAHIDRMGEDRFKKAARKIVNEALRGSNPAAGDPYTRADVLLY
ESLETLLPDAERERGINRALLRWNRAKLIEHLKRMCDDAGIRHFPVSPFGTSQVCSKCGALGRRYSLARENGRAVIRFGWVERL
FACPNPECPGRRPDRPDRPFTCNSDHNASVNLHRVFALGDQAVAAFRALPPRDSTARTLAVKRVEDTLRPQLMRLHKLADAGVD
SPF (SEQ ID NO: 302)
>CAAACI010012331_3|M
[activated sludge metagenome]
MQLWRTHCVFNQRLPALLKRLFAMRRGEVGGNEAQRQVYQRVAQFVLARDAKDSVDLLNAVSLRKRSANSAFKKKATISCNGQA
REVTGEEVFAEAVALASKGVFAYDKDDMRAGLPDSLFQPLTRDAVACMRSHEELVATWKKEYQEWLDRKSEWEAEPEHALYLNL
RPKFEEGEAARGGRFRKRAERDHAYLDWLEANPQLAAWRGKAPPAVVPIDEAGKRRIARAKAWKQASVRAEEFWKRNPELHALH
KIHVQYLREFVRFKQRPTFTMPDPVHHPRWCLFNAPQTSPQGYRLLRLPQSRRTVGSVELRLLTGPSDGAGFPDAWVNVRFKAD
PRLAQLRPVKVPRTVTRGKNKGAKVEADGFRYYDDQLLIERDAQVSGLKLLFRDIRMAPFADKPIEDRLLSATPYLVFAVKIKD
EARTERAKAIRFDETSELTKSGKKQKPKTLPAGLVSVAVDLDTRGVGFLTRAVIGVPEIQQTHHGVRLLQSRYVAVGQVEARAS
GEAEWSPGPDLAHIARHKREIRRLRQLRGKPVKGERSHVRLQAHIDRMGEDRFKKAARKIVNEALRGSNPAAGDPYTRADVLLY
ESLETLLPDAERERGINRALLRWNRAKLIEHLKRMCDDAGIRHFPVSPFGTSQVCSKCGALGRRYSLARENGRAVIRFGWVERL
FACPNPECPGRRPDRPDRPFTCNSDHNASVNLHRVFALGDQAVAAFRALPPRDSTARTLAGKRVEDTLRPQLMRLHKLADAGVD
SPF (SEQ ID NO: 303)
>UZXW01002767_11|M
[activated sludge metagenome]
MSVRAIQAKVMCDRATLENLWRTHRVFNERVPRILSLLFRMRRGECGKTPAERQLYQRIGQFITNCSAQNADYLMNAVTMRGWK
PGSAKKYKIRVKDENGETVEISGESWADEAAALSAKGSILCDKEDLAGDLPGCMRQMLNREAVAIISGHDELVANWSTDHVQWL
AEKAKWEREDEHKQYLALRSKFDEFEKSIGGKVGKRRGRWHLYIDWLRANPQLAAWRGGQATVNDLPPDALARIKKAKPWKTRS
VEAEEFWKVNPELAALDKLHGYYEREFVRRRKTKRNPDGFDHRPTFTMPDPTNHPRWFVFNAPQTNPAGYADLNLPDNQGDSGS
IKLCLLTGEKGDATEYPSDWITAQFRADPRLSRFRRVKVATTVNRGKAKGQAKEKDGYEFFDAHLMQWRPAEISGVKLILRHIK
LSGDSSLKSATPYLVFTVSTDDLALTEKARKIEWSDTGHVTKTGKPRKKRTLPDGLVACAIDLGLRHAGFATLALYQDGRPHVL
RSRNIWLEPDGRGPELGHIGRHKRKIRKLRRLRGKPVKGEESHIGLQKHITHMGEDRFKKAARGIINFAWNADGTVATKGADPF
PRADVILIEKLEGFIPDAEKERGVNRALAAWNRGQLVNRLKELAIDAGYKARIFEILPHGTSQVCSRCGAMGRRYSIARDEESG
RPEIRFGWVEKLFACPKCHFSANSDHNASVNLHRKFLLGDAAVAAFFDWKNLPEEKRRGELDAIDEALRGYLRKMHKLQAEIET
PF (SEQ ID NO: 304)
>3300009161|Ga0114966_10008307_8|M
[aquatic-freshwater-freshwater lake]
MKLLFKLRRGKIGKNKQQRDDYKRWFEYVIYNGAKNADYNLNALSINNWTPNTAKKYITKLMAGKNKIHAKGLDLVVSRIKSLS
DGGILAFDKDKIFIEIPYSFSQLIIRDAVAGISGHDALVKIWKEDHKKWRDEKKSWEDKTENKQYLKLQHIFKQFSDTAGGVGS
KKRGRWFTYLCWLSKQSALANWRGNGSFIEPFGEENCGKPSCNKGFKSKLIKESKKFFDANPELFEIHKLHQFYEKNFIRPKAK
KHNIDGFEHPPTFTLPDPVRHPIWACYNAPQTNPIGYKDLVLPKKYSGKSAGSFALNLLTGDIIDGKYPKEWVPFKFIADKRLS
CFKGKIKVVNNKIRTEYIFEDPHLSKKRQATIGGIKIILKDIKIDNLGHLLSATPYVVFACDVDNEPISEQAKKLKSEDTGETG
NSGLPIRKRILPEGCLISCAVDLGIRNLGFATLAESGGIPSECTHGDIKLLRSYNLLLEDGPSLEKIAKQKKLLGKFRRLRGKP
IKGEKSHVFLQNHITKMGVDRFNKAARLIVNFALNTEKKKNVQGIEYPRADVIVLENLAGLIPDAAKRKGINKALMNWNRGHLV
KRIQEIARDSGYLYRVYIMSPWGSSQVCCKCKGLGRRYSIIKDENGRQIQFGNGEKLFACANCRYMANADHNASINLHHIFKDK
DAVGFYKDYANKNEEEKQKIIDNLEAILRPHLQKMHFTTKKERSLDVPEHPLDVPF (SEQ ID NO: 305)
>3300009161|Ga0114966_10008307_7|P
[aquatic-freshwater-freshwater lake]
MGTRAIKAKIICDKTTLGYLWRSHCMYNQKLSEYMKLLFKLRRGKIGKNKQQRDDYKRWFEYVIYNGAKNADYNLNALSINNWT
PNTAKKYITKLMAGKNKIHAKGLDLVVSRIKSLSDGGILAFDKDKIFIEIPYSFSQLIIRDAVAGISGHDALVKIWKEDHKKWR
DEKKSWEDKTENKQYLKLQHIFKQFSDTAGGVGSKKRGRWFTYLCWLSKQSALANWRGNGSFIEPFGEENCGKPSCNKGFKSKL
IKESKKFFDANPELFEIHKLHQFYEKNFIRPKAKKHNIDGFEHPPTFTLPDPVRHPIWACYNAPQTNPIGYKDLVLPKKYSGKS
AGSFALNLLTGDIIDGKYPKEWVPFKFIADKRLSCFKGKIKVVNNKIRTEYIFEDPHLSKKRQATIGGIKIILKDIKIDNLGHL
LSATPYVVFACDVDNEPISEQAKKLKSEDTGETGNSGLPIRKRILPEGCLISCAVDLGIRNLGFATLAESGGIPSECTHGDIKL
LRSYNLLLEDGPSLEKIAKQKKLLGKFRRLRGKPIKGEKSHVFLQNHITKMGVDRFNKAARLIVNFALNTEKKKNVQGIEYPRA
DVIVLENLAGLIPDAAKRKGINKALMNWNRGHLVKRIQEIARDSGYLYRVYIMSPWGSSQVCCKCKGLGRRYSIIKDENGRQIQ
FGNGEKLFACANCRYMANADHNASINLHHIFKDKDAVGFYKDYANKNEEEKQKIIDNLEAILRPHLQKMHFTTKKERSLDVPER
PLDVPF (SEQ ID NO: 306)
>330001008|Ga0133913_10083255_2|P
[aquatic-freshwater-freshwater lake]
MGTRAIKAKIICDKTTLGYLWRSHCMYNQKLSEYMKLLFKLRRGKIGKNKQQRDDYKRWFEYVIYNGAKNADYNLNALSINNWT
PNTAKKYITKLMAGKNKIHAKGLDLVVSRIKSLSDGGILAFDKDKIFIEIPYSFSQLIIRDAVAGISGHDALVKIWKEDHKKWR
DEKKSWEDKTENKQYLKLQHIFKQFSDTAGGVGSKKRGRWFTYLCWLSKQSALANWRGNGSFIEPFGEENCGKPSCNKGFKSKL
IKESKKFFDANPELFEIHKLHQFYEKNFIRPKAKKHNIDGFEHPPTFTLPDPVRHPIWACYNAPQTNPIGYKDLVLPKKYSGKS
AGSFALNLLTGDIIDGKYPKEWVPFKFIADKRLSCFKGKIKVVNNKIRTEYIFEDPHLSKKRQATIGGIKIILKDIKIDNLGHL
LSATPYVVFACDVDNEPISEQAKKLKSEDTGETGNSGLPIRKRILPEGCLISCAVDLGIRNLGFATLAESGGIPSECTHGDIKL
LRSYNLLLEDGPSLEKIAKQKKLLGKFRRLRGKPIKGEKSHVFLQNHITKMGVDRFNKAARLIVNFALNTEKKKNVQGIEYPRA
DVIVLENLAGLIPDAAKRKGINKALMNWNRGHLVKRIQEIARDSGYLYRVYIMSPWGSSQVCCKCKGLGRRYSIIKDENGRQIQ
FGNGEKLFACANCRYMANADHNASINLHHIFKDKDAVGFYKDYANKNEEEKQKIIDNLEAILRPHLQKMHFTTKKERSLDVPER
PLDVPF (SEQ ID NO: 306)
>3300020109|Ga0194112_10008260_1|P
[aquatic-freshwater-freshwater lake]
MVDPEARAALWRTHGVYNECLRFALRHMHKMKRGEADPRYAEIFGAIRNSQHATAVMEAVTDPRWKPKKDPKPWHAPAAALIAE
GKLLFDRWKELPGLPGRFHRKLWEGAYQAMDSHKELMAIWASAHEEWQKARAEWEAENPEYMKLRPALEAFEAEHGQAAKRRQR
WHKWLAFLQANPGLAAWRGGADVVNPISDDGKERLRRAKRSKRNKVESEEFFNANPELKELDKLHGYYQREFVRPWAKKRNPDG
FRHRPTFTEPSPEKHPFWYQFKKGETYKALDLQAGTISLQLLAPEGAKRAALWTTFAFRPDPRLRLIQKVNPPLNEKFTYAFAD
PAYRGQPARPAEIRGAKLIFRPAQPDGAVYLSFTCDMPDLHCRLSVGQDSIDRYTTKGTKWLVGRLREEPELAGHEPVTCAVDL
GLRHLGAATVRRDGKIVRARLLREQGDPGKGPGLPAIAGHKRALAKGRRKRGKPIAGEESFVELQRHTDKMGEDRFKKGARRIV
AFAHENGADLIILEKLAGLVPDAEKERGINRALVSWNRANLEKWVKQLANDAGMRVVEVFPHWTSQLCSRCGSMGARFSAKEGL
PAFEPVGKLFACPECGYIANADHNASVNLHKRFYGELPEVERPNKDKRVFRLTPRGQPTVEVDMADVEKRILARGRAEDLCRGK
PTPF (SEQ ID NO: 307)
>3300020109|Ga0194112_10008260_1|M
[aquatic-freshwater-freshwater lake]
MDMVDPEARAALWRTHGVYNECLRFALRHMHKMKRGEADPRYAEIFGAIRNSQHATAVMEAVTDPRWKPKKDPKPWHAPAAALI
AEGKLLFDRWKELPGLPGRFHRKLWEGAYQAMDSHKELMAIWASAHEEWQKARAEWEAENPEYMKLRPALEAFEAEHGQAAKRR
QRWHKWLAFLQANPGLAAWRGGADVVNPISDDGKERLRRAKRSKRNKVESEEFFNANPELKELDKLHGYYQREFVRPWAKKRNP
DGFRHRPTFTEPSPEKHPFWYQFKKGETYKALDLQAGTISLQLLAPEGAKRAALWTTFAFRPDPRLRLIQKVNPPLNEKFTYAF
ADPAYRGQPARPAEIRGAKLIFRPAQPDGAVYLSFTCDMPDLHCRLSVGQDSIDRYTTKGTKWLVGRLREEPELAGHEPVTCAV
DLGLRHLGAATVRRDGKIVRARLLREQGDPGKGPGLPAIAGHKRALAKGRRKRGKPIAGEESFVELQRHTDKMGEDRFKKGARR
IVAFAHENGADLIILEKLAGLVPDAEKERGINRALVSWNRANLEKWVKQLANDAGMRVVEVFPHWTSQLCSRCGSMGARFSAKE
GLPAFEPVGKLFACPECGYIANADHNASVNLHKRFYGELPEVERPNKDKRVFRLTPRGQPTVEVDMADVEKRILARGRAEDLCR
GKPTPF (SEQ ID NO: 308)
>3300020222|Ga0194125_10021011_3|M
[aquatic-freshwater-freshwater lake]
MSTKAFTARLIVESPEARDALWRTHRLYNECLRFVLRHMHKMKRGEADPRYGEIFGAIRNSQHATAVMEAVTDLRWKPKKDPKP
WHTPAAALIAEGKLLFDRWKELPGLPGRFHRKLWEGAYQAMDSHKELMALWVSAHQEWQKARAEWEAENPEYMKVRPALEAFER
EHGQAAKRRQRWHKWLAFMRADPRLAAWRGGKALANPISDQGQQRLRRARRNKRNKVESEEFFNANPELKELDKLHGYYQREYV
RPWAKKRNPDGFRHRPTFTEPSPEKHPFWYQFKKGETYKALDLQAGTISLQLLAPEGAKRAALWTTFAFRPDPRLRLIQKANPP
LKEKFTYAFADPAFPGQPARPAEIRGAKLIFRRAQPDGAIYLSFTCDVPDLDSRLSILQKSCDKYGAKWACKKAMSDLGGQQPV
TCAVDLGLRHLGAATVRRDGKIVRARLLREEDQPGKGPALPAIAGHKRALAKGRRKRGKPIAGEESFIELQRHTDKMGEDRFKK
GARRIVAFARENGADLIILEKLGGDRRGALIPDAERERGINRALVLWNRGNLAKWVKQLAADAGMRVVEVHPYMTSQLCSRCGA
MGARFSAKEGLPAFEPVGKLFACPDCGYLANADHNASVNLHKRFFGELPEVQRPNRDKRVFRVTPDDQPTVEVDMADVEKRILA
RGRADDLCRGKRTPF (SEQ ID NO: 309)
>3300027770|Ga0209086_10006474_1|P
[aquatic-freshwater-freshwater lake]
MGTRAIKAKIICDKTTLGYLWRSHCMYNQKLSEYMKLLFKLRRGKIGKNKQQRDDYKRWFEYVIYNGAKNADYNLNALSINNWT
PNTAKKYITKLMAGKNKIHAKGLDLVVSRIKSLSDGGILAFDKDKIFIEIPYSFSQLIIRDAVAGISGHDALVKIWKEDHKKWR
DEKKSWEDKTENKQYLKLQHIFKQFSDTAGGVGSKKRGRWFTYLCWLSKQSALANWRGNGSFIEPFGEENCGKPSCNKGFKSKL
IKESKKFFDANPELFEIHKLHQFYEKNFIRPKAKKHNIDGFEHPPTFTLPDPVRHPIWACYNAPQTNPIGYKDLVLPKKYSGKS
AGSFALNLLTGDIIDGKYPKEWVPFKFIADKRLSCFKGKIKVVNNKIRTEYIFEDPHLSKKRQATIGGIKIILKDIKIDNLGHL
LSATPYVVFACDVDNEPISEQAKKLKSEDTGETGNSGLPIRKRILPEGCLISCAVDLGIRNLGFATLAESGGIPSECTHGDIKL
LRSYNLLLEDGPSLEKIAKQKKLLGKFRRLRGKPIKGEKSHVFLQNHITKMGVDRFNKAARLIVNFALNTEKKKNVQGIEYPRA
DVIVLENLAGLIPDAAKRKGINKALMNWNRGHLVKRIQEIARDSGYLYRVYIMSPWGSSQVCCKCKGLGRRYSIIKDENGRQIQ
FGNGEKLFACANCRYMANADHNASINLHHIFKDKDAVGFYKDYANKNEEEKQKIIDNLEAILRPHLQKMHFTTKKERSLDVPER
PLDVPF (SEQ ID NO: 306)
>3300015360|Ga0163144_10184575_1|M
[aquatic-freshwater-freshwater microbial mat]
MECNREELQHLWRTHCAFNERLPSILSVLFKMRRGEWGDTEQIKKKMQAFAWGVLQLEAQNSYDPLIKAASGSARNSTARNALR
KIAAKASKAIDDETAKPSESQDAAKLAKARKTLLQCQEYESSFDLFAKGLTPLKDVKDALADLPNHIERKLFEESSACIRGHLA
LVENWENEHVEWLKKKKDWEADPEHRKYLDVRHRFDAFEEEAGGKAGKRRGRWHLYLDWLRKNPDLAAWRGGQSSIGEPDASAK
LRIQKAKPWKQRSVEAEEFWKVNPELSALNKLHGFYEREFIRRRKTKKHSDGFDHRPTFTLPHAVNHPRWFVFNAPQTSPKGYC
ELKLPGAPGAEGSLELLLLTGEKEMGVYPKKWTSLVFKADPRLSDFKPVTVKKNANKGKVKGQETEKPAYSFYDRQLKQHRAAQ
ISGVKLIFKQVKLNEDGTLKSAVPYLYFTCSVVDIAITQKAQDIEWSETGETTKTGKARKRKTIPNGLIACAVDLGLRNLGFAT
LGVLESGEVRVLRSRNLWIGVAEPSGAHAGRWAEGPDLAHIGAHKRNIKRLRRQRGKPVEGERSHVELQEHIDHMGEDRFKKSA
RAIINFALNVDQTIDKKTGKPFPRADVLVMEKLEGFIPDAEKERGINRALVSWNRGQLIERLKQLAEDSGFKKRLFEIHPAGTS
QVCSRCGALGRRYSIARDETGQPEIRFGWVEKLFACPNSKCGYRANADHNASVNLQRSFFLDGQAVQAFYDWKGKPEPERKTFF
AAMDGKLQPALRVEHKLDGPTPF (SEQ ID NO: 310)
>3300013130|Ga0172363_10020541_1|M
[aquatic-freshwater-sediment]
MSIRAIQARVECDRPTLERLWLTHRIFNERLPVILSFLFRMRRGECGDTVVERKMYHEIADFILTHSQEAPYLFNAVSIKGWKP
GTAVKQSPGSEWPAQAAQLSKEGKLLYDKSNVLGDLPGTLQQMVCRESAAIISGHHELVRMWEAEHAQWLEDKSAWEADAEHQK
YLALRPRFDAFEQEAGGPAGKRRGRWHLYLNWLKQNPDLAAWRGGKPEVIELSPAARRRVERAKPNRQRSVEAEEFWKGNPELS
ALDRLHGYYEREFVRRRKTKKNADGFKHRPTFTLPHPRIHPRWFVFNAPQTQPSGYADLVLPTKPGEEGSVRMLVINAEKANGK
YPSEWVRIRFHGDPRLCNFRAVSSTRTISRGKDKGKTKESPSYVFRDRHLGQDRPATISGIKLIFRTNTDRTPKAAYLYFACTI
DDLPQSDAAKSIQWTETGEITSKGKKRKNMKVPDGLVGCAVDLGIRNLGFATLARYADGQPQILRSRNIWVRRQEAKGARTGRW
SAGADLAHIGEHKRTIRRLRRQRGKPVKDEKSHVGLQLHIDHMGEDRFKQAARAIINFALNADGDCNRKTGEVHPRADVLVLEN
LGGLIPDAEKERGINRALVNWNRGQLVERIKEMAQDVGLKVLEVSPVGTSQVCCKCGSLGRRYSVVRNEDTGRLDIRFGQVEKL
FACPASDCPGREAGRPDRPFTCNADYNASANLHRCFLLRDQAVKTYRDWRSGGDSKKAKEERETKIRNVEDALLGPLRIMHGME
DAPPF (SEQ ID NO: 311)
>3300013133|Ga0172362_10044703_1|M
[aquatic-freshwater-sediment]
MDGDTWANEAAALSAQGVLLYDKHAVLGDLPAHLRQMVCRESVAILSGHDELVARWNKEHQEWLTAKAKWEDEEEHKKYLALRP
RFEAFEKEAGGKAGKRRGRWHLYFSWLKANPDLAAWRGKADSVALLSDKAKERIRKARPWKKRSVEAEEFWKVNPELQALDKLH
GYYEREFVRRRKTQRNPDGFHHRPTFTLPHFTRHPRWFVFNAPQTSPQGYDNLQLPAKPHAGGSVELLLLTGDKTNGEYPSQWI
SIGFQGDPRLSLFRKVKKQRTVNRGKDKGTQKESDAYEFRDLHLNRWRKAEISGIKLLFRLNPNGSPKAAYLCFTCSITDEPLS
SNARKIKWSETGETTKKGKKRKRKTLPDGLVTCAVDLGIRNTGFATLAVHDQGNVLIRRSRNLWAGWLGEDSKDRDRWMDGPVL
AHIAAHKRKIRFLRRKRGKPVKGETSHVELQTHIDHMGEDRFKRAARAIINFALNVGSECDKKTGEVYPRADVLVLENMDGLIP
DAEKEHGVNRALVAWNRGQLTKRIEEIAEDVGLKILFVSPMGTSQVCSKCGSLGRRYSIKHDDMIDRPDIQFGFVEKLFACPSC
QYMANADHNASVNLHRRMVLDDKAVVAYNSFREKNKTDQATEIERIENILRPRLRLKHSLEGSVPF (SEQ ID NO: 312)
>3300031862|Ga031580_10005430_2|M
[aquatic-freshwater-sediment]
MVELYLRMRKGKHRETARKIAEIILKSPKNTAAAIMEGLTAKEWTSTRTTEWANLVRERRNAEGLLFDRHESVGSADGRNACCQ
RQQGPRKKPTDPSRLPVSSKFWRWLCDDACEVLKSNRGQMDDWRSEYREWRDEKTQWEKDHPEFMAFWNGLYQQFRQDCERAQI
EAQQAKGQKPTFRKRGGRQWGKRFERWHLWPEWIEQHPEILAWRGQAAAADFKPVPQQEKDALRKRFKRQDKYIGALLDKLRES
NSELAELDRLRRAYERQYVRFRRFPTLTLPDPDKHPRWMSLERDCLYKGFDPEKGTIKLSLVGENADGVWYMDWFDVQVQVDRR
LKPSFRREVFGREGRLPPFIEGRVGRKLSQPAPSPAERQSGVAGAKLIFKQGAAHLTFTIIDQDRAPRVKPRKVADRRCPADNI
FDPQGDRIPLRVVAVDLGVRHVGGYAVAEGRFENGAWSVGYITKGIVSGPQVPERFSIDAHHRQLKWARRLRGKPVFDEESCAE
LQDHVTHMGEDRFKKAAHAIVELARRHDVHVILFENLKGFVPSACDERWANRTRSKMLRRKTVEMVKAQCHDFGFYCNEEISPK
LTSHICSKCFRPGWRFSMKAKQPWSEKQPRSKCRDFGYPAWDAGGHLFRCPHCGYRVNADINAAGNLVAKFFGLWPDEKLERKD
WVYRWRENGESRQFDAKAEFEKWSDDVRSRKAARSFPY (SEQ ID NO: 313)
>3300031952|Ga0315294_10035890_2|P
[aquatic-freshwater-sediment]
MSVKCFQAKVVCDTPEKREYLWLTHTLFHEALRALIPHLFKMRRGDYGADFKEIYEAITDNQNSFAKLEPITTPKAWRSKHADT
GDTKNRWADLCLAVNREGRILFDRTQTPFGFGSEFWRKVCEMAIQLIHSHDGLLADWRKERAEWLDNKKQWEAANPEYMAAKPT
FDAFEDEAGRLRGSRKRWLLYLEFLQKHPELAAWRGGEAKVAPLTPAERKQCRRPGDQFEIFWEKNPQLAELNRLDRDWRRNFA
RFKRRPTWTNPDPELHPAWFSFKRGQTYKDLDFARRTFNLNVLVGEDVKGRKGKWVTYEFRHDPRLLRLRPAEQPVKVGPNKFT
WLYSDPLLKKDRPAEVKGIKLVFRNRHPYLLFSVDIADEKSAPLTRPGFDDKRLKRKATAADIPDGTRILSIDFGQRSLAACSV
CEFIKGHPERPDAVFFMKLPGLKFGDIGRHEQTISRRSSRMFRAGPRSRESRHAPRGGTTFREFRRHVLKMKDDRYKKATRLIL
DAAVRYDAQVILVENLENYKPDLERSARENRARMRWNVQRIVEFLDKSAHPLGLRLWKVWPRWSSRFCSACGHPGARFTIPRKN
AWKRFYERRHGPIRKPVTEPGGQFFICSNPKCPRSTGIINADVNASLNLPRILADDLEKPTGQGKTRLWRGQEYNPKAIAEQCQ
QRLDEHFRSKADLAQQTPW (SEQ ID NO: 314)
>3300031952|Ga0315294_10041214_2|M
[aquatic-freshwater-sediment]
MFQARIECDRQTLDRLWLTHRVFNERLPAIMSVLFNMRRGESGRNPQERRLYQRMARFFLAMDSKNAEYLLHSVSIKGWTPNTA
LKMRAKVRDAHGAEVPVSGDSWAPAAAALSAKGRLLYEKAQVLGDLPATLRQMVCREAVAVISGHDELVARWEKAHADWLKDKA
RWEADAEHQQYLALRPRFEAFEAEAGGKAAKRRGRWHKYLSWLRANPDLAAWRGKAAPGVQDLSPQARRRVQRAGLKRRAAVEG
EEFWKANPELSALDRLHGYYERAFVRRRKTKKNPDGFDHRPTFTLPHATRHPRWFVFNAPQTSPSGYADLAFPEKRGATGSIRL
CLLTGDKAADGKYPHEWVPVRFLADPRLSAFRPVKRQRVIGRGKRKGEAAEAKSAYEYFDRHLGKTREARFSGVKLVFRLYPDG
KPKAAYLYFACELTDEPLSERAKAIQWTETGEVTRKGKKRKSKRLPDGLVSAAVDLGIRNLGFATLARKEDGAVQVLRSRNLWT
GPDLHHITRHKRRLRALRRDRGKPVAGESSHVRLQGHIDHMGEDRFKRAARAIVNFALNTDGARNGRGQAHPRADVLVLESMEG
LIPDAEKERGINRALVAWNRGHLVERIRDMALDAGLKVFEVSPFGTSQVCSRCGALGRRYSLVREAETGEQAVRFGFVEKLFAC
PACGYRANADHNASVNLHRRFILEDAAVKAYQDLYNRPESERKVALADMESRLLSRLGVEHGLAKPPPF (SEQ ID NO:
315)
>3300031952|Ga0315294_10041214_2|P
[aquatic-freshwater-sediment]
MSVRMFQARIECDRQTLDRLWLTHRVFNERLPAIMSVLFNMRRGESGRNPQERRLYQRMARFFLAMDSKNAEYLLHSVSIKGWT
PNTALKMRAKVRDAHGAEVPVSGDSWAPAAAALSAKGRLLYEKAQVLGDLPATLRQMVCREAVAVISGHDELVARWEKAHADWL
KDKARWEADAEHQQYLALRPRFEAFEAEAGGKAAKRRGRWHKYLSWLRANPDLAAWRGKAAPGVQDLSPQARRRVQRAGLKRRA
AVEGEEFWKANPELSALDRLHGYYERAFVRRRKTKKNPDGFDHRPTFTLPHATRHPRWFVFNAPQTSPSGYADLAFPEKRGATG
SIRLCLLTGDKAADGKYPHEWVPVRFLADPRLSAFRPVKRQRVIGRGKRKGEAAEAKSAYEYFDRHLGKTREARFSGVKLVFRL
YPDGKPKAAYLYFACELTDEPLSERAKAIQWTETGEVTRKGKKRKSKRLPDGLVSAAVDLGIRNLGFATLARKEDGAVQVLRSR
NLWTGPDLHHITRHKRRLRALRRDRGKPVAGESSHVRLQGHIDHMGEDRFKRAARAIVNFALNTDGARNGRGQAHPRADVLVLE
SMEGLIPDAEKERGINRALVAWNRGHLVERIRDMALDAGLKVFEVSPFGTSQVCSRCGALGRRYSLVREAETGEQAVRFGFVEK
LFACPACGYRANADHNASVNLHRRFILEDAAVKAYQDLYNRPESERKVALADMESRLLSRLGVEHGLAKPPPF (SEQ ID
NO: 316)
>3300031952|Ga0315294_10058002_1|M
[aquatic-freshwater-sediment]
MSVRSIQARLHCDAKTLDDLWRTHRVFNERLPGINAILFKMRRGEFGGTPKEREMMQAFAWCVLRLDAKNSYDPLNKAAIGSAR
SASAVKAVNALIAQAKKTIAKPSAPQAMQDRGTLEEARKTMLFGPVCLDAFSKFATGYLPLKDLKDSLGDLPNAIGRRVLEEAC
ACLRGHFALAAKWQTEHADWLTEKAKWEADLEHQKYLGLRPRFEAFEKEAGGKAGKRRGRWHLYLAWLKANPDLAAWRGKADGV
TDLSKQARERIRRAKPRKQLSVEFDEFWKVNPEFQALNKLHGFYERRFSPPGKQQCGAKSKSLRKKKRNPDGFKHRPTFTLPHP
VRHPRWFVFNAPQTSPKGYWNLCLPTNPEAEGSVELLLLKGEKGADGKYPSDWVKVQYRGDPRLCLFRKVKKRRTVNRGKDKGA
EKEADAYEFRDAHLGLWRPTDISGVKLIFRFNHDGTPKAAYLYFTCNIKDVPLTANAKAIQWTETGEVTKKGKKRKRKTLPDGI
LTCAVDLGIRNLAFATLAVHHEKGNLVIKRSRNLWVGWQEGGDAKGERWLDGPDLAHIAKHKRKIRWLRGKRGKPVKGESSHVE
LQTHIDHMGEDRFKRAARAIINFALNAQAACDRHGEVYPRADVLILENMDGLIPDAEKERGINRALIAWNRGQLTKRIEEMAKD
VGLKIIPVSPMGTSQVCSRCGSLGRRYSIGRDEKTGQAEIRFGFVEKLFACPNSDCGYLANADHNASVNLHRRFALDDKAVAGY
AEHRVKGDDERTKEIADIEARLRPRLRLKHSLDGPVPF (SEQ ID NO: 317)
>3300032046|Ga0315289_10071835_2|M
[aquatic-freshwater-sediment]
MSARAFQAKVVVERPEDLQALRDLHRRFNEFLPPVIEALFKMKRGALGPDAQLLFSSMTSNQNAVAKLEPLTTPKPWKPRKKQQ
LDETWVQAAVRLREGRGILFDRFEAMPDVSSEFRRKVFEMAFACVDGHLQLVKRWRKEHEDWLKQKERWERGLVVTEEGEDEAS
AETRTWRERYMRLRPLFEEFEQRGVVRRNRRRWHEYIEFLSTHPAFAEFRPNALTDQERADLRDPIARFERLLEKNADLERLDN
LHGQYQDLIRPWARKRNPDGFKHRPTFTMPSVASHPQWFSFKKSATYKNLDLEKGVLALALGKEGAKAQWVTVRLATDPRLARF
RPAQRVGKGKRARSWVYLDPISGEEFDAEVRGLKLMFPKGIQGPAYLMFTVQLSDRPRQVVPRKDWTPPEGLLTLACDFGQRHL
GAITMCRYENGSPVQVPFTPAYLDHGRGRRTPSKPVSAWLPVIPGLRFDDIREHELEISARLSATYGDPRSRRQGGVGASRGRH
AAAGSRPFAQLRRHIDEMREVHFKKAAHLIVATALKNGAQVVIMEFLANYRPDLERSHRENRARMIWAVRRIQEFVEQTAALYG
IAVRTPSPYLTSQVCSACGSPGARFTRPSAMDWTRKPPGRKESWYEKRFGPKAKAVIEPGGPWFCCSNPNCPRPCRVIQADINA
SLNLHRRFYERFDLSWLPDGKPARKQFWGRLKGAIQAYLDQFDSLPRRPGATSHE (SEQ ID NO: 318)
>3300032053|Ga0315284_10046226_2|P
[aquatic-freshwater-sediment]
MATKTYRARFVNLTGRLDAALARTHHVFVECLRQLIGRYIEMRKGKYGPECRQLAEVILSRFNTFAHGVMDKLSRPNWESRLTD
DWVLLANSVCEAQGPLFDQHDGFAVVDGVTIHTKPHSKSEPVKNRLPVPAKFWHQVCDSASAYLQSNRALMEQWRNERKDWFKN
KTEWEQKHQEFMTFWNGMFKQFEDACEKQRLLSQQAAGQLPTAQKKQSRERGKRLGRWHLWYEWLVAHPEIIEWRGKAKATDFV
FVPDELKKIMSKHSRQDKFIPKHLDWLKENNPELKVLDDLRRTYVRNYLRFKRPPTLTLPSPKKHPYWFTLELDQFYKSPDFDK
GTVKVLLIDENDNGAWFFNWFDAGLLCDKRLKPSYRAEMFRNKGRYPPYIEGKVGRTLNRPAGSSKDRKAGYAGAKLVINGKRK
ELLFTVIEQDCPPSIKWTKVKHRRCSADNALSPDGERIPIKVMAIDLGIRHAGAYAIAEGTADDDIWQVKWLKKGIIDNSAMPD
LYQIRGHDRQLRRGRSKTGKAPKGERSFIDLQSHRTNMAQDRFKKAANAIINIARSYKVDVIIFEKLESLAPTAFDERWMNRQL
RDMNRRKIVDMVKQQAAEFGIKADDNVSPYLTSHVCSRCFKGGWRFSMKAKQPYKEKLSRKNCCDYGYPTWDAGGHLFRCPHCG
YSVNADINAACNVGAKFFGLWPATMSKIKREWVYTWDAENSEPFNAKEAFDKWAEDVKKHKALADSPF (SEQ ID NO:
319)
>3300032069|Ga0315282_10002412_8|P
[aquatic-freshwater-sediment]
MVTRALRARVIHPDATLLAALKRTHRVYNVLLRQMVELYLRMRKGKHRETARKIAEIILKSPKNTAAAIMEGLTAKEWTSTRTT
EWANLVRERRNAEGLLFDRHESVGSADGRNVCCQRQQGPRKKPTDPSRLPVSSKFWRWLCDDACEVLKSNRGQMDDWRRDYKEW
RNEKARWEKDHPEFMAFWNGLYQQFRQDCERAQIEAQQAKGQKPTFRKRGGRQWGKRFERWHLWPEWIEQHPEILAWRGQAAAA
DFKPVPQQEKDALRKRFKRQDKYIGALLDKLRESNSELAELDRLRRAYERQYVRFRRFPTLTLPDPDKHPRWMSLERDCLYKDF
DPEKGTIKLSLVGENADGVWYMDWFDVQVQVDRRLKPSFRREVFGREGRLPPFIEGRVGRKLSQPAPSPAERQSGVAGAKLIFK
QGAAHLTFTIIDQDRAPRVKPRKVADRRCPADNIFDPQGDRIPLRVVAVDLGVRHVGGYAVAEGRFENGAWSVGYITKGIVSGP
QVPERFSIDAHHRQLKWARRLRGKPVFDEESCAELQDHVTHMGEDRFKKAAHAIVELARRHDVHVILFENLKGFVPSARDERWA
NRTRTKMLRRKTVEMVKAQAGEFGFLCNEKISPKLTSHICSKCFRPGWRFSMKAKQPWSEKQPRSKCRDFGYPAWDAGGHLFRC
PHCGYRVNADINAAGNLVAKFFGLWPDEKLERKDWVYRWRENGESRQFDAKAEFEKWSNDVRSRKAAKSFPY (SEQ ID NO:
320)
>3300032118|Ga0315277_10001015_12|P
[aquatic-freshwater-sediment]
MATKSFEARVLCRDDPPDKKGEKYSAEQKKQFLWYTHQVFNESIRKVLPYVFKMKRGELGDEFQAIYYAISSSQDAVGKLEAIT
SLDWSSKKIGKSEPEKWKEILRYRELEKEFRQKLKDEEKNTKKFRKELQKKKKALANEIGQKDIWADAASIIQQKNNLLFDREK
ILPNLPSEFRRKIFEMCIQLIHGHQELVANWQDEHAEWLQEKDKFEEEHPDYMKVRPILEEFEKEAGKVKGSRIRWLKYLDYMS
SHPELAGWRGGEAKVEPLTSAERAKFRKPREHFEAFFKKNPELSALDSIHKQYQEKFARTQTKRTPNPDGFKHRPTFTLPSANL
HPAWYSFKGATDPTKGATYKLNFREDRRVDSFMNLKVLTDMEGEGRNPKGMITYDFEPDDRIRDFRYIGTTEKGKKAKGFVYYD
KILEKERPAKIQGIKLVFRPPRSDGVPYLIFSCSIEDEKSKIKIWQDKESEEGPDEARKGKKAKKIYPPELVTLAIDFGQRHLG
AITICKNNNGKPEQIKFIPAYPKRKQGKQTKPVSAWLAKVPGLTFNAIGMHEKEIRAGMHQRFQDPKSIRQAGEKEGRASKPRH
IPETETSFAHLRDHIAGMKEDHYKKAANLIIKTALQNGAQVILLEDLKKYRPMLEKESRENRRRMQWAVRQTAEFLKKTAKPLG
LLIYDRINPAYTSSMCSVCGNPGARISLPSKRNWEKYYAEYYGKERRMIAVVGGQFFCCPVCKKIINADINASQNMHKVFYNTF
NWPPKMEKKEKKIFWESVREQAQNILDQKAEPKREEDISY (SEQ ID NO: 321)
>3300032118|Ga0315277_10012802_3|M
[aquatic-freshwater-sediment]
MSVRAIQARVECDRATLEHLWRTHRVFNERVPVVLSWLFKMRRGECGRTEEEKRLYRTIADFVLANSQNAEAILNAVSIPNWTP
TSARKLKAKTVLSDGTEKVSTGMDWADKAAELSAAGRLLFDKREMLFDLPGTLRQMVCRDAGAIISGHEELVAVWKKAHADWLK
AKADWESQEENKLYLNLRPEFEAFEQAAGGRAAKRRGRWHLYLKWLKDHPRLGGWRGGEPIVNELSEQARRRIQKAKPWKQRSV
EGEEFWRANPELAALDRLHGYYEREFIRRRKTKKNPDGFTHPPTFTVPHAVRHPRWFVFNAPQTSPIGYRNLVLPPHAGPEGTV
ELRLLTGDKTDGKHPGNWVKVRFRADPRLSDFRPAKRTRSFNRGRNKGQTTQSDAYEYFDHHLGLMRSAEIKGLKLIFRFNPDD
SLRAAYLYFTCDVGSIPFTERTRAIQWTETGEVTKKGKKRKTKKLPDGLIACAVDLGLRHLGFATRARYAGANPEILRSRNLWL
GREEQKGRHPGRLSPGADLRHIAHHKRNIRRLRRLRGKPVKSESSHVELQTHIDHMGEDRFKQGARAIINFALNVKKGPDTRTG
EVHPRADVLILEKLAGLLPDAEKERGINRALVAWNRGQLVKRVKEMAQDVGLKVFEVSPVGTSQVCSRCGALGRRYSIVPDDRT
GRPGFRFGWVEKLFACPNPDCRYRGNADHNASVNLHRRFMLEDAAVKAFLDWRAKTKKEQEEVLRRIEKALCADLLRMHGLEEP
TPF (SEQ ID NO: 322)
>3300009499|Ga0114930_10003500_21|M
[aquatic-marine-deep subsurface]
MSTKAIQAKVICDTWEHRYFLWITHKLFNDSVQFVIPKVFAMKRGELGDDFKLIYDSIRSNQDAIGKLEQITSLEAKSNRQEKW
VLAAKKIREEKNLLFDRKKELPGFSSEFRRKVFEMTIQKIHGHQELVDDWEKEHNAWLKEKEKFEKDHQDLMALWSEIEKFEKE
AGSLKGSKKRWYRYLEFLGTHPAFSTYRDGGPLSKEERAGLKRPGEHFELFFKKNPKLVPVDSAIKKYCDLLRPWAKKRNSDGF
KLKPTFTLPNPIVHPEWYSFKKNSTYRKLDLQKGEVELKIIPSEDIDKRDPKKYISYRFGADPRIKNLIPSNIEKIGREKYDHN
YFDPVLDKKRPAVIKGIKLIFKGLNRKRKLWAEYEREKNTDKKKQLFAQWIDKLRPYFVFTLTLADEAKPVKLEWNKKEKHSVI
PKELVTMAIDVGQRDLGAVTIAREHNGVPVQIAFLPQYWKPLKNGNSAKPVKAWLVDIPGISFEALKKHEKEARGRERGAKARR
VKGENYAIGLRQHILHMKEDRYKKAGSILLRTAIKNKVHVIILEDLKKYKMNLERSKQENRQLMVWAIRKIQDFLELQAKPLGV
RVRSVWPHYTSQVCSACGEPGKRASLPSKSSWEKFYQKKYGEKRKWIVENGGKMFVCSNPNCVRKSRMINADINASLNLHKKFY
GILTYPPKTKEFKQQLTLDIQNYLDTILKF (SEQ ID NO: 323)
>3300011118|Ga0114922_10000510_27|M
[aquatic-marine-deep subsurface]
MATKSYRSRIVCTKAELDDLVRTHRVFNAGVRAMVDLYISMKKGTYGETAKEIARYLLDHSGTKGHDLMNPLTDRGGKDTRDDE
GMKLVRRHRQEHGILFPRHERICEIDGRKVRAGSSAKKEKSRDTLTISSIFWHFICNQAIEYLRSNNELMAAWREERGKWLRDK
EKFFAENPGFREFVEGYLAAFDAHAEKARQEFQTKAGQRAHARKRKKSGSGKRFSRWHLWCEWICDHPEIIEWRGKAKGEGFAF
LTAEEVRAIEEEFPKREDRRAGRMLDMLTRKNPELAEVEKLRKKYNNDYARFRRPPTLTLPSAKKHPCWYRLEKGKLYRNEDFE
KGTIEILVICHDEKEDAYYMDWRPFSIKPEPRLVPSHRARAFAAEGRYPPYKEGKVGNTLNRPAESAEERLAGLKGAKLILKPK
GNAQLVFTVVEQNEPLKAKFRKKAGATSSADRLDILDGNKATRVMGIDLGARHVGAYVITEGKKKGDGWKLAPLRKGFIDSPDL
PRLRSITRHENQLKKGRRKRGKPVKNERSFISLQGHRTKMSTDRFKKAANLIVEKAREHDVHIIVFEDLRRLKPTAYDERWLNR
QVRSMNHRHIFDLAKEMAPEFGIQVTDINPYNTSRLCSKCGFPGYRFSMKRKNPYREQAPRRECADFGYPVWDSGGHLFRCPHC
GYRVQADLNAAGNIARKFFGHFMDWECKEYTYRNAVQPSFDARKDFDQWASGVLKRRTMPDAPF (SEQ ID NO: 324)
>3300017960|Ga0180429_10052609_1|M
[aquatic-non marine saline and alkaline-hypersaline lake sediment]
MSQRALAVRLLVDSEDDRAALWRTHRIFNERLGWVLRQMHRMKRGEPDARYAEIFNAITTSQRATAALEAVTSLAWTGTKEDRW
CQLARALIQEGTLLFDREARLAGLSKEFRRKLFEAAFQMISGHRELLAQWERDHNEWQQARETWEAENPEYMAIRPALQRFEAE
HGQAAKRRARWHKWRAFLQGSSQLAGWRGGPQVVNEISADARRRIERARRNKRNKIEAEEFFRVNPELKELDAKHGYYQREFLR
PWAKRRNADGFKHRPTFTEPSAENHPFWFQFKKHETYKDLNLKVRSLRLKLLASDSAVPGSQWRDFRFRVDPRLLDLSEAQSPA
KIGIARFSLLFNDRALAMEDRPAEIRGAKLIFRPAQRDGRVYLSFTCDVADLPCRVPLKQESWDKYSAEWLCKKSREALDGEDP
VTCAVDLGIRRLGAASVRRDGKIVRARIIREEQQRGCGPRLAAIKNHKRALKAGRRKRGKPVRGEQSLVELQEHVDKMGRDRFK
KGARRIVNFAHQNHCDVIVMEKLAGLIPDAERERGINSALVNWNRGHLVKWVKQLANDAGMRVVEVYPHWTSQLCSRCGAMGAR
FSADHGHVAFEYVGSLFACADCGYVANADHNASVNLHRRFYGELPKVKKLREGVYELAKPDHAPKEVKLAELRSAVSVAAAAMC
RGQASPF (SEQ ID NO: 325)
>3300017963|Ga0180437_10045654_1|M
[aquatic-non marine saline and alkaline-hypersaline lake sediment]
MATKTYRARIVQISPRLGAALERTHYLFAEALNQMVRRYIDMRKGKYGQECKKLADIMLTRSNTFAHGVMDQLTREISTSGLTD
EWVDLARAVHKSHGPLFLQHDGFAEVDGVMLHTKRHGKVSPMPGRLPVNAKFWHQVCDSASAFLKSNNELLENWRQGRKEWLQE
KADWESANADFMRFWNGSYQEFEETCEIKRKESQALEGQKVVEKKRHRRDRGKRMSRWHLWYNWVITHPEIIEWRNRAKAAEFR
PVPPTVHKKIKNKYPRQDKHITKFLDWLKENNPELKALDSRRRFYVRNYLRFRRPPTLTLPSANKHPYWFTFELNQFYKNVDFD
KGSIQLLLVDETEEGSWFFRWFDAKIQCDPRLRPCVRAGVFATKGRYPPYIGGKVGNKLNRPAERPENRKAGYAGAKLVLNKRR
KELFFTVIEQDCPRKVSWKKTKQRTCRGDNVFAKDGSQIPLKVMAIDLGIRHIGAYVIAEACRNRGVWDISWQHKGITGRHAVP
GLKEIRQHDRQLRKARSQRGKPVQGEESFIELQDHRTNMADDRFKKAANTIVELARQYDVHLILFERLGNLSPTAFDERWMNRQ
LRDMNRRQIVDTVKLQAREFGIECLEDISPWMTSHICSRCFKPGWRFSIKRKNPYGERFTRKNCRDYGYPVWDTGGHLFRCPHC
EYKCNADINAACNVAAKFFGLWSGLDGLKRDQWVYTWQENQPKTFDAKKTFLEWALDVRNRKLMGDSPF (SEQ ID NO:
326)
>3300017971|Ga0180438_10003700_2|P
[aquatic-non marine saline and alkaline-hypersaline lake sediment]
MATKTYRARIVQISPRLGAALERTHYLFAEALNQMVRRYIDMRKGKYGQECKKLADIMLTRSNTFAHGVMDQLTREISTSGLTD
EWVDLARAVHKSHGPLFLQHDGFAEVDGVMLHTKRHGKVSPMPGRLPVNAKFWHQVCDSASAFLKSNNELLENWRQGRKEWLQE
KADWESANADFMRFWNGSYQEFEETCEIKRKESQALEGQKVVEKKRHRRDRGKRMSRWHLWYNWVITHPEIIEWRNRAKAAEFR
PVPPTVHKKIKNKYPRQDKHITKFLDWLKENNPELKALDSRRRFYVRNYLRFRRPPTLTLPSANKHPYWFTFELNQFYKNVDFD
KGSIQLLLVDETEEGSWFFRWFDAKIQCDPRLRPCVRAGVFATKGRYPPYIGGKVGNKLNRPAERPENRKAGYAGAKLVLNKRR
KELFFTVIEQDCPRKVSWKKTKQRTCRGDNVFAKDGSQIPLKVMAIDLGIRHIGAYVIAEACRNRGVWDISWQHKGITGRHAVP
GLKEIRQHDRQLRKARSQRGKPVQGEESFIELQDHRTNMADDRFKKAANTIVELARQYDVHLILFERLGNLSPTAFDERWMNRQ
LRDMNRRQIVDTVKLQAREFGIECLEDISPWMTSHICSRCFKPGWRFSIKRKNPYGERFTRKNCRDYGYPVWDTGGHLFRCPHC
EYKCNADINAACNVAAKFFGLWSGLDGLKRDQWVYTWQENQPKTFDAKKTFLEWALDVRNRKLMGDSPF (SEQ ID NO:
326)
>3300017991|Ga0180434_10065880_1|P
[aquatic-non marine saline and alkaline-hypersaline lake sediment]
MSVKTIQAKLECDRETLETLWRTHTVFNEGLLPILALFFKMKRRETGSTEEEKDLYKRICEFILRTNASNPGYLLNSVCLENWK
PDSARKTKAEVVRDGKKEKISAEEWIDDLLNMKEESGLLFLKQELLNGLPIMFFEKLVAESKAILSAYEELQYQWEVDHNKWLK
EKEGWEKKHKEYLAVRSLFQKFEQEVKGKITKKRRRWHLYKDWMKRTPELVAWRGKETKVYGLTEEDREAIKKAPLKKKIGVEV
GLFFKRNPELSALDKLHSYYEEKFVRRGKKKRNCDGYEHRPTFTLPHSVKHPRWYVFSGPQTGNGYKNLNIPERKKDFGQLDLL
VLTDDKVDGEYPREYRTVKFRGDHRFLDIRGATRTRLVTKGKNKGQEVEEKFAYEMYDRYLKIWRPLKISGAKLAFRDIKINQD
GSLKSAVPYLWVTLNLEDLPISEKAKEIKWVETDEVKDNGDKRKKIQIPEGLITCAVNIGIRNLGFGTLAVGEKGKARILRSRN
IWIGEEEKTGKHKGRWQKGPTLRELRDHKQEIRRLRKLRGKVVKGERSHIKLQNHITNFADDRLKKAARKILNFALNVENHIDK
KTGKPYPRADVLLLEDLSTLVPTTKKRRDQNQLLIEFMHRGTLSKIEEMAKDAGLKVLRIKPGGISQVCPKCGGIGRRYEIRID
QETKKPDIHFTDVIDDIDCQKLFVCASCSYRGNGDHVASVNLHRKFYDWSVVDKYFTLLRSDNYKEQKKVIEEELREFLRKEHF
PQMALN (SEQ ID NO: 327)
>3300017992|Ga0180435_10008274_7|P
[aquatic-non marine saline and alkaline-hypersaline lake sediment]
MTIKSFEAKIVTNPSLRETLEYTHRVYNEDLKYMVEVLLAMRKGKHGDGYVEFWREHLQKAANPSGLLEPLTTPDGPKADAKSK
KSKIIRQLFKEHGMLFDRDERWPHWNARHRRHLFDDAKKWARQLDERLERWRSEHAQWLKEKEKFENENPLYMAVEPLFGGFFK
EFGQGGKRRERWAAYYHFLLAHPELANWRGQADAIVPLTPEEEAACLRDRRRGVKKRLDLFLEKNPELEALHKLDAKYGRNMVR
RSARHRNIDGFRHPPTMTLPHSRLHPEWITFQAGETYKDLDLNGGTVRLNILPPDAETSKLHSVEFRAEKRLRRITPRETPLRK
GRNEFNKNYRDAADRVHPARVKGIRLILRPRGAYLKITVELDRPESRTGLKQQTCDKYSSNWCYQEIVKRREGKPLKCLSLDLG
VRHLAVGVVVQETRANGDRETVIEGVRFFKEVGGRPFPSLDEIRRHKDRLREKRSQTRRAPKGRRTCIALQDHVTKMGQDRYRK
GAAAIVRYAVERDVDVILLEKLAGFIPDAAKQRGINKALMEWNRRKIADFVKLLAADHGISVVEVAPHHLSQLCSKCGELGRRF
SWKDGLPKFGTVEKLFVCPACGYQANSDYNAAVNLYKAFSGAYEERPKKAKKGVYKYRDRTICLKKLDGQLAERLTQEQTPF
(SEQ ID NO: 328)
>330001808|Ga0180433_10080815_1|M
[aquatic-non marine saline and alkaline-hypersaline lake sediment]
MSVKTIQAKLECDRETLETLWRTHTVFNEGLLPILALFFKMKRRETGSTEEEKDLYKRICEFILRTNASNPGYLLNSVCLENWK
PDSARKTKAEVVRDGKKEKISAEEWIDDLLNMKEESGLLFLKQELLNGLPIMFFEKLVAESKAILSAYEELQYQWEVDHNKWLK
EKEGWEKKHKEYLAVRSLFQKFEQEVKGKITKKRRRWHLYKDWMKRTPELVAWRGKETKVYGLTEEDREAIKKAPLKKKIGVEV
GLFFKRNPELSALDKLHSYYEEKFVRRGKKKRNCDGYEHRPTFTLPHSVKHPRWYVFSGPQTGNGYKNLNIPERKKDFGQLDLL
VLTDDKVDGEYPREYRTVKFRGDHRFLDIRGATRTRLVTKGKNKGQEVEEKFAYEMYDRYLKIWRPLKISGAKLAFRDIKINQD
GSLKSAVPYLWFTLNLEDLPISEKAKEIKWVETDEVKDNGDKRKKIQIPEGLITCAVNIGIRNLGFGTLAVGEKGKARILRSRN
IWIGEEEKTGKHKGRWQKGPTLRELRDHKQEIRRLRKLRGKVVKGERSHIKLQNHITNFADDRLKKAARKILNFALNVENHIDK
KTGKPYPRADVLLLEDLSTLVPTTKKRRDQNQLLIEFMHRGTLSKIEEMAKDAGLKVLRIKPGGISQVCPKCGGIGRRYEIRID
QETKKPDIHFTDVIDDIDCQKLFVCASCSYRGNGDHVASVNLHRKFYDWSVVDKYFTLLRSDNYKEQKKVIEEELREFLRKEHF
PQMALN (SEQ ID NO: 329)
>3300009503|Ga0123519_10002165_1|M
[aquatic-thermal springs-hot spring]
MATKSFEAKIVCKPDEKYTAEQKKQFLWFTHQVFNDGVRKVIPYVFKMKRGELGPEFQAIYYAITSSQDAIGKLEAVINPDWTS
GKIGKSDPNKWKELLKYQELEKGFRQRLKEEGIKSTKKFRKELEDEKKKLAKEIGQKDIWADAAAILRNKNLLLFNRDELLPNL
PSEFRRKIYEMTIQLIHGHQELVANWEDEHAEWLIEKDKWEEEHPEYMNVRPIFEKFEKEQGKVKGSRIRWLAYLDFLSSKPEL
ANWRGKAKETIPLTKEERAGFRKPGQHFAAFFNKNPELQELDRLHKEYQEKFARTQSKRTPHPDGFKHRPTFTLPDAMRHPVWY
SFKGATDPTKGSTYRNLDLENCTLDLKVLTAMEGEGRNPGGMIQYAFEPDERIKGFRYVGTTEKGKRAKGYIYYDPILEKERPA
KIQGIKLVFRPPRPDGTAYLIFSCQIEDEKPKIKIWKDKEEESPGEITKRKKTEVYPPELITLAIDFGQRHLGAITICKNNNGR
PEPIRFIPAYPKRRKDRESKPVSAWLAKIPGLTFNAVGMHEKEISAGMSRRFQDPKSIRQAGEKEGRKSKGQHIPETETPWAHL
REHIANMKEDHYKKAANLIIRTALQNGAQVILIENLRNYRPMLERTNLENRRRMQWAVRQTAKFLEDTARPLGLIVRQVSSAYT
SRFCSSCGHPGARVSLPGQKNWEKFYAEKYGKERKMIAVAGGQFFCCPACKKIINADINASLNMHKVFYQNFIWPGKIDKKDTK
NFIWQGKNYNWDQIADDVQSFLDQKAGIKKEDDIPY (SEQ ID NO: 330)
>CAAACB010000011_94|M
[bioreactor metagenome]
MSTRAIQARVVADKALLQKLWLTHRAFNEHLPHFSQTLFRMMRGELGDSDQDKAQWAGFVKGVLAQPSKNAYDCLIKATNQTTR
SDTTAAKALSKMEAAARKDIDALNAVPEAKRDPEKFAEASNRLEICRLYNAAFEEFRKGNRPLAHSRQQLDALPNFIERKVIEE
SVAILRSHLELVELWKAEWRQWSDDKAKWEADEVNRRYLAIRPIFEQFEESSGRSAHRSFRWKAYFEFLKSHPELAAWRGGAAV
VHPITDAGLRAIQRARLRKKDQIFAEEFFKANPELEALHRRHMEYEKRFVRRRKSKRSKAWITGFKNRPTFTLPDALRHPRWFV
FNAPQTAPAGYRKLRLPNTPGGEGSMELELLSGTMEKGEHKTVRTTIRFRGDPRLALFKPVMVKRKAIKGAIKGQETDKAAYVF
SDPQLHKDRPAKIGGAKLILRNIKLDEAGQLVSATPYIYFTVDVESPRWSPLAAAIKSVDDVPGGDGRKSRRRSLPDGLVVCTV
DLGIRNLGFATLARIEDGKPRILRSRNLWLGFEETGKHEGRWASGPDLTHISNHKQELRRLRRMRGRPVAGEESHAELQAHIDH
MAEDRFKKAARLITDFALNTDRNSPKGQETPYPSADILVVERLAGLVPQADKERGINRALIEWNRGQLVERLKLVTESVGLRLF
EIGAAGTSQVCSRCGALGRRYSIVPDPHPLFRPAPHVIRFSEVEKLFACPACGYRANADHNAAVNLQRRFALGETAIAAAMDLW
HRPKPEKEAMRKSIEAQLRSKLEIEHKLAEPLP (SEQ ID NO: 331)
>SRR5251193_megahit_k177_201695_120|P
[bioreactor metagenome]
MGIKSWRVKLWVESPEIRATLEYCLSLWNTSAALLCRHVLAMRRGDFGPEGQAIWEHLKKTPHNFHVLYPISNGSGIEKTTRYY
PDARAFVNKYGYGALGPVRSASGCLWKQLTKAVRERTHSYDKTCEKWSEEHSKWLEERAKWEAEHPEYMAIRPLIDTFHAKHGV
TRGRRNRWALWLQFLMETPELVRWRNKTAKVVPLTDSELKRIRSKRRGAARREQALFYEKNPELAELNRIHGIYERRFARSAAK
RKNPDGFRHRPTFTLPSEAKYPDWPRFQAGEGWRDLDLDRRTICLKLPTDGGSARWVQVHFVPDPRLQRLARLKQPVKEGATRY
DYSLDMPSGHTVLAQPQEVRLIRRGSSYFATIRLSLHSAPSAIPVPQTAASSPAGMLRKIRTDAPELKLITCAVDLGVRHLGAA
TIAREGEIIGRRILHNRFLAPGADGGVINIPSLPQIAQVRRQIRCAQRRSGRISPKVAGCRHLWTHYRHLCEDRYKKAVAAIFG
FARAHGAHIVLFEDLKMLNPDSANERGVNRALQNWNRGRIVEFAKNTAEDAGLCVVTVPAYWTSRICARCNAFGVRFNQGQRRK
PGDDGHVSRVACELDFLGHWFLCPSCGRSVHADLNASENLHRVFLGTFPTVQTIQRQPRIYEINGQRVALDEIQQRAVEILASR
RAGDTPF (SEQ ID NO: 332)
>SRR5251487_megahit_k177_199995_1|P
[bioreactor metagenome]
MGIKSWRVKLWVESPEIRATLEYCLSLWNTSAALLCRHVLAMRRGDFGPEGQAIWEHLKKTPHNFHVLYPISNGSGIEKTTRYY
PDARAFVNKYGYGALGPVRSASGCLWKQLTKAVRERTHSYDKTCEKWSEEHSKWLEERAKWEAEHPEYMAIRPLIDTFHAKHGV
TRGRRNRWALWLQFLMETPELVRWRNKTAKVVPLTDSELKRIRSKRRGAARREQALFYEKNPELAELNRIHGIYERRFARSAAK
RKNPDGFRHRPTFTLPSEAKYPDWPRFQAGEGWRDLDLDRRTICLKLPTDGGSARWVQVHFVPDPRLQRLARLKQPVKEGATRY
DYSLDMPSGHTVLAQPQEVRLIRRGSSYFATIRLSLHSAPSAIPVPQTAASSPAGMLRKIRTDAPELKLITCAVDLGVRHLGAA
TIAREGEIIGRRILHNRFLAPGADGGVINIPSLPQIAQVRRQIRCAQRRSGRISPKVAGCRHLWTHYRHLCEDRYKKAVAAIFG
FARAHGAHIVLFEDLKMLNPDSANERGVNRALQNWNRGRIVEFAKNTAEDAGLCVVTVPAYWTSRICARCNAFGVRFNQGQRRK
PGDDGHVSRVACELDFLGHWFLCPSCGRSVHADLNASENLHRVFLGTFPTVQTIQRQPRIYEINGQRVALDEIQQRAVEILASR
RAGDTPF (SEQ ID NO: 332)
>SRR5576005_megahit_k177_1292294_1|M
[bioreactor metagenome]
MSTRAIQARVVADKALLQKLWLTHRAFNEHLPHFSQTLFRMMRGELGDSDQDKAQWAGFVKGVLAQPSKNAYDCLIKATNQTTR
SDTTAAKALSKMEAAARKDIDALNAVPEAKRDPEKFAEASNRLEICRLYNAAFEEFRKGNRPLAHSRQQLDALPNFIERKVIEE
SVAILRSHLELVELWKAEWRQWSDDKAKWEADEVNRRYLAIRPIFEQFEESSGRSAHRSFRWKAYFEFLKSHPELAAWRGGAAV
VHPITDAGLRAIQRARLRKKDQIFAEEFFKANPELEALHRRHMEYEKRFVRRRKSKRSKAWITGFKNRPTFTLPDALRHPRWFV
FNAPQTAPAGYRKLRLPNTPGGEGSMELELLSGTMEKGEHKTVRTTIRFRGDPRLALFKPVMVKRKAIKGAIKGQETDKAAYVF
SDPQLHKDRPAKIGGAKLILRNIKLDEAGQLVSATPYIYFTVDVESPRWSPLAAAIKSVDDVPGGDGRKSRRRSLPDGLVVCTV
DLGIRNLGFATLARIEDGKPRILRSRNLWLGFEETGKHEGRWASGPDLTHISNHKQELRRLRRMRGRPVAGEESHAELQAHIDH
MAEDRFKKAARLITDFALNTDRNSPKGQETPYPSADILVVERLAGLVPQADKERGINRALIEWNRGQLVERLKLVTESVGLRLF
EIGAAGTSQVCSRCGALGRRYSIVPDPHPLFRPAPHVIRFSEVEKLFACPACGYRANADHNAAVNLQRRFALGETAIAAAMDLW
HRPKPEKEAMRKSIEAQLRSKLEIEHKLAEPLP (SEQ ID NO 331)
>UOUK01008448_2|M
[bioreactor metagenome]
MHLWRTHCVFNQRLPALLKRLFAMRRGEVGGNEAQRQVYQRVAQFVLARDAKDSVDLLNAVSLRKRSANSAFKKKATILRNGQR
REVTGEEVFAEAVALASKGVFAYDKADMRAGLPDSLFQPLTRDAVACMRSHEELVETWKKECREWLDRKSKWEAEPEHALYLKL
RPKFEKGEAERGGRFRKRAERDRAYLDWLEANPQLAAWRGKAPPAVVPIDEAGKRRIARAKAWKQASVRAEEFWKRNPELHALH
KIHVQYLREFVRFKQRPTFTMPDPVRHPRWCLFNAPQTSPQGYRLLRLPQSRRTVGSVELRLLTGPSDGAGFPDAWVNVRFKAD
PRLAQLRPVKVARTVTRGKNKGAKVEADGFRYYDDQLLIERDAQVSGVKLLFRDIRMAPFADKPIEDRLLSATPYLVFAVEIKD
EARTERAKAIRFDETSELTKSGKKRKTLPAGLVSVAVDLDTRGVGFLTRAVIGVPEIQQTHHGVRLLQSRYVAVGQVEARASGE
AEWSPGPDLAHIARHKREIRRLRQLRGKPVKGERSHVRLQAHIDRMGEDRFKKAARKIVNEALRGSNPAAGDRYTRADVLLYES
LETLLPDAERERGINRALLRWNRAKLIEHLKRMCDDAGIRHFPVSPFGTSQVCSKCGALGRRYSLARENGRAVIRFGWVERLFA
CPNPECPGRRPDRPDRPFTCNSDHNASVNLHRVFALGDQAVAAFRALPPRDSPARTLAVKRVEDTLRPQLMRVHKLADAGVDSP
F (SEQ ID NO: 333)
>U0UL01003058_1|M
[bioreactor metagenome]
MHLWRTHCVFNQRLPALLKRLFAMRRGEVGGNEAQRQVYQRVAQFVLARDAKDSVDLLNAVSLRKRSANSAFKKKATISCNGQA
REVTGEEVFAEAVALASKGVFAYDKDDMRAGLPDSLFQPLTRDAVACMRSHEELVATWKKEYREWRDRKSEWEAEPEHALYLNL
RPKFEEGEAARGGRFRKRAERDHAYLDWLEANPQLAAWRGKAPPAVVPIDEAGKRRIARAKAWKQASVRAEEFWKRNPELHALH
KIHVQYLREFVRPRRTRRNKRREGFKQRPTFTMPDPVRHPRWCLFNAPQTSPQGYRLLRLPQSRRTVGSVELRLLTGPSDGAGF
PDAWVNVRFKADPRLAQLRPVKVPRTVTRGKNKGAKVEADGFRYYDDQLLIERDAQVSGVKLLFRDIRMAPFADKPIEDRLLSA
TPYLVFAVEIKDEARTERAKAIRFDETSELTKSGKKRKTLPAGLVSVAVDLDTRGVGFLTRAVIGVPEIQQTHHGVRLLQSRYV
AVGQVEARASGEAEWSPGPDLAHIARHKREIRRLRQLRGKLVKGERSHVRLQAHIDRMGEDRFKKAARKIVNEALRGSNPAAGD
PYTRADVLLYESLETLLPDAERERGINRALLRWNRAKLIEHLKRMCDDAGIRHFPVSPFGTSQVCSKCGALGRRYSLARENGRA
VIRFGWVERLFACPNPECPGRRPDRPDRPFTCNSDHNASVNLHRVFALGDQAVAAFRALAPRDSPARTLAVKRVEDTLRPQLMR
VHKLADAGVDSPF (SEQ ID NO: 334)
>3300009652|Ga0123330_1010394_3|M
[bioreactor-anaerobic-anaerobic biogas reactor]
MHLWRTHCVFNQRLPALLKRLFAMRRGEVGGNEAQRQVYQRVAQFVLARDAKDSVDLLNAVSLRKRSANSAFKKKATISCNGQA
REVTGEEVFAEAVALASKGVFAYDKDDMRAGLPDSLFQPLTRDAVACMRSHEELVATWKKEYREWRDRKSEWEAEPEHALYLNL
RPKFEEGEAARGGRFRKRAERDHAYLDWLEANPQLAAWRRKAPPAVVPIDEAGKRRIARAKAWKQASVRAEEFWKRNPELHALH
KIHVQYLREFVRPRRTRRNKRREGFKQRPTFTMPDPVRHPRWCLFNAPQTSPQGYRLLRLPQSRRTVGSVELRLLTGPSDGAGF
PDAWVNVRFKADPRLAQLRPVKVPRTVTRGKNKGAKVEADGFRYYDDQLLIERDAQVSGVKLLFRDIRMAPFADKPIEDRLLSA
TPYLVFAVEIKDEARTERAKAIRFDETSELTKSGKKRKTLPAGLVSVAVDLDTRGVGFLTRAVIGVPEIQQTHHGVRLLQSRYV
AVGQVEARASGEAEWSPGPDLAHIARHKREIRRLRQLRGKPVKGERSHVRLQAHIDRMGEDRFKKAARKIVNEALRGSNPAAGD
PYTRADVLLYESLETLLPDAERERGINRALLRWNRAKLIEHLKRMCDDAGIRHFPVSPFGTSQVCSKCGALGRRYSLARENGRA
VIRFGWVERLFACPNPECPGRRPDRPDRPFTCNSDHNASVNLHRVFALGDQAVAAFRALAPRDSPARTLAVKRVEDTLRPQLMR
VHKLADAGVDSPF (SEQ ID NO: 335)
>3300012949|Ga0153798_10025413_2|P
[bioreactor-switchgrass degrading]
MRRGDFGPEGQAIWEHLKKTPHNFHVLYPISNGSGIEKTTGYYPDARAFVKKYGYGALGPVRSASGCLWYQLTKAVRERSHSYD
KTCEKWSEEHSKWLEERAKWEAEHPEYMAIRPLIDTFHAKHGVTRGRRNRWALWLQFLMETPELVRWRNKTAKVVPLTDSELKR
IRSKRRGAARREQALFYEKNPELAELNRIHGIYERRFARSAAKRKNPDGFRHRPTFTLPSEAKHPDWPRFQAGEGWRDLDLDRR
TICLRLPTDGGSARWVQVHFVPDPRLQRLARLKQPVKEGATRYDYSLDMPSGHTVLAQPQEVRLIRRGSSYFATIRLSLHSAPS
AIPVPQTAASSPAGMLRKIRTDAPELKLITCAVDLGVRHLGAATIAREGEIIGRRILHNRFLAPGAAGGVINIPSLPQIAQVRR
QIRCAQRRSGRISPKVAGCRHLWTHYRHLCEDRYKKAVAAIFGFARAHGAHIVLFEDLKMLNPDSANERGVNRALQNWNRGRIV
EFAKNTAEDAGLCVVTVPAYWTSRICARCNAFGVRFNQGQRRKPGDDGHVSRVACELNFLGHWFLCPSCGRSVHADLNASGNLH
RVFLGTFPTVQTIQRQPRIYEINGQRVALDEIQQRAVEILASRRAGDTPF (SEQ ID NO: 336)
>SRR7811943_megahit_k177_406423_2|M
[cave metagenome]
MGVRAIRARIECDRETLLSLWRTHRVFNERLPSLIAKLFAMRRGEFGSNAAQRKLYERAVRFILARDSKDAPYLLNSVSIKGWK
HATALKMKARVPDGAGDVQEVTGDSWAAEAATLSANGTLAYDKETERDGLPDSLFQPLIRDAVAYISGHDELVRNWRKEHEDWL
KKRAEWETKDERKKYLALRPQFDQFEEAVGGKAGKRRERWHLYLKWLRDNPDLAAWRGGAAVVRPLDARAQGRVARAKPWKQRS
VEAEEFWKANPELSALDRLHGFYEREFVRRRKTKRNPDGFDHRPTFTLPDPVLHPRWALFNAPQTSPSGYRALSLPMGRAGFGS
VELCLLTANRDGREWPRAWKSIRFRSDPRLAQFCEHTETTTFVKGKKKGQAKTRQSFSYTDNQLGGINRPAQISGAKLILKDLR
LNDDGSLKSATPYVVFACTIDDQPLTDRARAIQWSETGEVTKTGKKRKAKKLPEGLVACAVDLGVRNLGFLTVAVASDNPGDGV
RVLRSRNLWLGEFEGDGQWSPGPHLAHIGAHKREIKRLRRLRGKPVKGEESHIELQQHIDGMGKDRFKKAARAIVNFALNVEDR
ASPRTGEVYPRADVLIVEKLAGFIPDAERERGINRALVAWNRGQLVTRLKEVAKDAGLKLFEVHPAGTSQCCSKCGALGRRYSI
VRDDETRAPVIRFGWVEKLFACPNLNCAGRKSMHCDQPFVCNADHNASVNLHRRFVMDDKAVAAFFAWKARPDGEREQSVRSID
DALRPQLERIHGLSASPLETPF (SEQ ID NO: 337)
>SRR5818194_megahit_k177_802550_7|M
[freshwater metagenome]
MKLLFKLRRGKIGKNKQQRDDYKRWFEYVIYNGAKNADYNLNALSINNWTPNTAKKYITKLMAGKNKIHAKGLDLVVSRIKSLS
DGGILAFDKDKIFIEIPYSFSQLIIRDAVAGISGHDALVKIWKEDHKKWRDEKKSWEDKTENKQYLKLQHIFKQFSDTAGGVGS
KKRGRWFTYLCWLSKQSALANWRGNGSFIEPFGEENCGKPSCNKGFKSKLIKESKKFFDANPELFEIHKLHQFYEKNFIRPKAK
KHNIDGFEHPPTFTLPDPVRHPIWACYNAPQTNPIGYKDLVLPKKYSGKSAGSFALNLLTGDIIDGKYPKEWVPFKFIADKRLS
CFKGKIKVVNNKIRTEYIFEDPHLSKKRQATIGGIKIILKDIKIDNLGHLLSATPYVVFACDVDNEPISEQAKKLKSEDTGETG
NSGLPIRKRILPEGCLISCAVDLGIRNLGFATLAESGGIPSECTHGDIKLLRSYNLLLEDGPSLEKIAKQKKLLGKFRRLRGKP
IKGEKSHVFLQNHITKMGVDRFNKAARLIVNFALNTEKKKNVQGIEYPRADVIVLENLAGLIPDAAKRKGINKALMNWNRGHLV
KRIQEIARDSGYLYRVYIMSPWGSSQVCCKCKGLGRRYSIIKDENGRQIQFGNGEKLFACANCRYMANADHNASINLHHIFKDK
DAVGFYKDYANKNEEEKQKIIDNLEAILRPHLQKMHFTTKKERSLDVPEHPLDVPF (SEQ ID NO: 305)
>SRR5818194_megahit_k177_802550_6|P
[freshwater metagenome]
MGTRAIKAKIICDKTTLGYLWRSHCMYNQKLSEYMKLLFKLRRGKIGKNKQQRDDYKRWFEYVIYNGAKNADYNLNALSINNWT
PNTAKKYITKLMAGKNKIHAKGLDLVVSRIKSLSDGGILAFDKDKIFIEIPYSFSQLIIRDAVAGISGHDALVKIWKEDHKKWR
DEKKSWEDKTENKQYLKLQHIFKQFSDTAGGVGSKKRGRWFTYLCWLSKQSALANWRGNGSFIEPFGEENCGKPSCNKGFKSKL
IKESKKFFDANPELFEIHKLHQFYEKNFIRPKAKKHNIDGFEHPPTFTLPDPVRHPIWACYNAPQTNPIGYKDLVLPKKYSGKS
AGSFALNLLTGDIIDGKYPKEWVPFKFIADKRLSCFKGKIKVVNNKIRTEYIFEDPHLSKKRQATIGGIKIILKDIKIDNLGHL
LSATPYVVFACDVDNEPISEQAKKLKSEDTGETGNSGLPIRKRILPEGCLISCAVDLGIRNLGFATLAESGGIPSECTHGDIKL
LRSYNLLLEDGPSLEKIAKQKKLLGKFRRLRGKPIKGEKSHVFLQNHITKMGVDRFNKAARLIVNFALNTEKKKNVQGIEYPRA
DVIVLENLAGLIPDAAKRKGINKALMNWNRGHLVKRIQEIARDSGYLYRVYIMSPWGSSQVCCKCKGLGRRYSIIKDENGRQIQ
FGNGEKLFACANCRYMANADHNASINLHHIFKDKDAVGFYKDYANKNEEEKQKIIDNLEAILRPHLQKMHFTTKKERSLDVPER
PLDVPF (SEQ ID NO: 306)
>SRR6481359_megahit_k177_1860565_4|M
[freshwater sediment metagenome]
MSVRSFQAKVECDKQIMEHLWRTHTVFNERLPVIIKILFQMKRGECGQNDKQKALYQNIAKSILDTNAQNADYLLNSVSIKGWK
PGAARKYNKGSFTWLDEAANLSSKGILVYDKDQVLGDIPLMMRQMICRQSVETIKSQQEILKNWENEYKEWLKKKEEWESQEDN
KKYLALRDKFEQFRKSYGGKKSKKRGRWHLYLKWLRDNPDLAAWRGGKAEINPISEKAQSRINKAKPNKKNAIELEEFLEANPE
IKKLDELHGYYERTFIRRRETRKGKELTGFKQKPTFTLPHSTTHPRWFVFNAPQTNPAGYRNLILPQKTGALGSLEMRLLNGDK
NNGDYPDDWINVRFKADPRLALMRGIKIQRIVRRGKDAGQTKESDSYKYFDKFLNKWRPATLSGVKLIFKFNPDKSVNTAYLYF
TCDITNEPLTETAKKIQWIETGEVTKKGKIRKKKVLPEGLISCAVDLGMRKGTTGFGTLCKYKNGKPYILRSRNLWIRYKEEKG
SHSGRWAEGPDLGHIAKHKREIRFLRRKRGKPIKGEKSHIKLQQHIDDMGEDRFKKAARAIVNFALNTENVAGKNGIYPRADIL
LLENLEGLIPDAERERGINRALAGWNRRHLVERIKEMAEDAGFRRRVFEIPPYGTSQVCSKCGSLGRRYTLGINEETGKREIHF
GYVEKLFACPNCGYCANADHNASVNLHRRFLIDDALKNYYEFKKLPEKKQKEAIAAIENSLKEKLSTIHKI (SEQ ID NO:
338)
>SRR6049666_megahit_k177_256604_1|P
[hot springs metagenome]
MATKSFEAKIVCKPDEKYTAEQKKQFLWFTHQVFNDGVRKVIPYVFKMKRGELGPEFQAIYYAITSSQDAIGKLEAVINPDWTS
GKIGKSDPNKWKELLKYQELEKGFRQRLKEEGIKSTKKFRKELEDEKKKLAKEIGQKDIWADAAAILRNKNLLLFNRDELLPNL
PSEFRRKIYEMTIQLIHGHQELVANWEDEHAEWLIEKDKWEEEHPEYMNVRPIFEKFEKEQGKVKGSRIRWLAYLDFLSSKPEL
ANWRGKAKETIPLTKEERAGFRKPGQHFAAFFNKNPELQELDRLHKEYQEKFARTQSKRTPHPDGFKHRPTFTLPDAMRHPVWY
SFKGATDPTKGSTYRNLDLENCTLDLKVLTAMEGEGRNPGGMIQYAFEPDERIKGFRYVGTTEKGKRAKGYIYYDPILEKERPA
KIQGIKLVFRPPRPDGTAYLIFSCQIEDEKPKIKIWKDKEEESPGEITKRKKTEVYPPELITLAIDFGQRHLGAITICKNNNGR
PEPIRFIPAYPKRRKDRESKPVSAWLAKIPGLTFNAVGMHEKEISAGMSRRFQDPKSIRQAGEKEGRKSKGQHIPETETPWAHL
REHIANMKEDHYKKAANLIIRTALQNGAQVILIENLRNYRPMLERTNLENRRRMQWAVRQTAKFLEDTARPLGLIVRQVSSAYT
SRFCSSCGHPGARVSLPGQKNWEKFYAEKYGKERKMIAVAGGQFFCCPACKKIINADINASLNMHKVFYQNFIWPGKIDKKDTK
NFIWQGKNYNWDQIADDVQSFLDQKAGIKKEDDIPY (SEQ ID NO: 330)
>SRR2080392_megahit_k177_207267_1|M
[hydrothermal vent metagenome]
MLVLPHGARGAASSIICPFGIKNISAKWDSPEYHGQTYYIMPIMKRRVIQAKISDTDPDTRQKIHLTHQIFNEYVKQTIKLFLS
IMNGTYDHPAYKTNKELYRRWMASIIFAGSGGGSINYDYLINSICKTKSKRHWEGASIPEKVTIPTVNQITDQKGKKTAKIEKE
EHDFKSFSSVDQIVELAKQQIYLFDKSEAFPLPSQIISAVISEAIAIIKQHRTLITKWYEDNGDFETEQAEFLNSPRYKSFLKI
KYIFDAFEQENKLRAERKRSQRKRSLRWERWDKYVNLLIDNSELMQWREKNTTLTIPPALNDPAKNKKSRLGILLRANPELGAL
HRTYVETWKDIPPQRPTFCWPDINEHPRFFTASSPPTSPYAYQRLSIDHKKSKATVEFFLLDGKRDKNGRYIGNFYLLRVVFDP
RINRFEHNPNAITKKGNKKPNSYYFLDPRTKKKREAHIKGCMLFLKKQKTSNHTSAYLTFNYAIERSNISEQAKKVRYDEKAKG
WKAPIGAIVASVDMMYKEIAFVTIAEKIKPQEFTILASRKIKLHRNQDNGFKGPQAKQIEQHQISLRDRKRNLPRKQYPQKLQN
HLNNMREEFSKRSARRILDFVMNLNKTIKDSQDKIIEQVDVILLERLKGLKPQKKNPRGLNRVLSNKRRPKVVTAIQEGCEIAG
ISCREIMPHYTSQICSKCGKLGRRFYENRKDVHEVERKMSPFGGKLFICPHCGYQANAEFNATINLLNIVLLNKRMTPEDKTNL
SPAEKRNYYTQLDDLAMKKLSPQKSPVKV (SEQ ID NO: 339)
>SRR2080392_megahit_k177_207267_2|P
[hydrothermal vent metagenome]
MKRRVIQAKISDTDPDTRQKIHLTHQIFNEYVKQTIKLFLSIMNGTYDHPAYKTNKELYRRWMASIIFAGSGGGSINYDYLINS
ICKTKSKRHWEGASIPEKVTIPTVNQITDQKGKKTAKIEKEEHDFKSFSSVDQIVELAKQQIYLFDKSEAFPLPSQIISAVISE
AIAIIKQHRTLITKWYEDNGDFETEQAEFLNSPRYKSFLKIKYIFDAFEQENKLRAERKRSQRKRSLRWERWDKYVNLLIDNSE
LMQWREKNTTLTIPPALNDPAKNKKSRLGILLRANPELGALHRTYVETWKDIPPQRPTFCWPDINEHPRFFTASSPPTSPYAYQ
RLSIDHKKSKATVEFFLLDGKRDKNGRYIGNFYLLRVVFDPRINRFEHNPNAITKKGNKKPNSYYFLDPRTKKKREAHIKGCML
FLKKQKTSNHTSAYLTFNYAIERSNISEQAKKVRYDEKAKGWKAPIGAIVASVDMMYKEIAFVTIAEKIKPQEFTILASRKIKL
HRNQDNGFKGPQAKQIEQHQISLRDRKRNLPRKQYPQKLQNHLNNMREEFSKRSARRILDFVMNLNKTIKDSQDKIIEQVDVIL
LERLKGLKPQKKNPRGLNRVLSNKRRPKVVTAIQEGCEIAGISCREIMPHYTSQICSKCGKLGRRFYENRKDVHEVERKMSPFG
GKLFICPHCGYQANAEFNATINLLNIVLLNKRMTPEDKTNLSPAEKRNYYTQLDDLAMKKLSPQKSPVKV (SEQ ID NO:
340)
>3300025517|Ga0207869_1001046_11|M
[lab enrichment-defined media-ionic liquid and high solid enriched]
MGVKAWKAKIIVADPDLAAALDHIVRQWNLWAEQACIAIMRLRRGDYGDEGQAVWRFLSEKGTFGTSILDKITRVTGRGIKNDE
ERTIASNFLSRHGPFEPLRAARDALGPNFWYQFQRAVRERIESHDAKLRQWREDHAEWIETRRTWEEEHRAYMNVRPVIEAFEA
AEGQSGRRRGRWRRWLDFLSSHPELAAWRGGPATIVPLSKEELAACRRRRRRAVAEQARLFFEKNPELRALNREHDEYQEKFAR
PWAKRRNPDGFRHRPTFTLPHPDSHPDWPRFKGNAGWRDLSVPDRIVSLELLCPDGSRRWFNVRFVPDPRLASLRPATQPVRQG
NTTYHYVSDALGVAEQPVQPQGIKLILRPDGAYLSVSVSVLAPPCGFDVRQSAIEKYGSIWACRKYKQDHPDRPLVTCAVDLAV
RHNGALTVARDGEVFARRLLHNRFFLPGDDRERAMNIPSLLEIAAVKRQLRRARRQTGRIAPGKPSCARLQAHYRALCEDRYKK
LVAAIFAYARHHGAEVVIFEDLKNLLPNAVNERGVNRALQSWNRGAIVRFARQSAEDVGLRVATVPAFHTSIVCARCGALGCRF
EQTREGVRIAKLGPWFGCRACGRRIHADLNASENLHKVLIGTFPQVRRISRDPPVLSIADVRVDQREADRLAAAWLSELSVGPT
PF (SEQ ID NO: 301)
>3300025572|Ga0207864_1000213_47|M
[lab enrichment-defined media-ionic liquid and high solid enriched]
MGVKAWKAKIIVADPDLAAALDHIVRQWNLWAEQACIAIMRLRRGDYGDEGQAVWRFLSEKGTFGTSILDKITRVTGRGIKNDE
ERTIASNFLSRHGPFEPLRAARDALGPNFWYQFQRAVRERIESHDAKLRQWREDHAEWIETRRTWEEEHRAYMNVRPVIEAFEA
AEGQSGRRRGRWRRWLDFLSSHPELAAWRGGPATIVPLSKEELAACRRRRRRAVAEQARLFFEKNPELRALNREHDEYQEKFAR
PWAKRRNPDGFRHRPTFTLPHPDSHPDWPRFKGNAGWRDLSVPDRIVSLELLCPDGSRRWFNVRFVPDPRLASLRPATQPVRQG
NTTYHYVSDAIGVAEQPVQPQGIKLILRPDGAYLSVSVSVLAPPCGFDVRQSAIEKYGSIWACRKYKQDHPDRPLVTCAVDLAV
RHNGALTVARDGEVFARRLLHNRFFLPGDDRERAMNIPSLLEIAAVKRQLRRARRQTGRIAPGKPSCARLQAHYRALCEDRYKK
LVAAIFAYARHHGAEVVIFEDLKNLLPNAVNERGVNRALQSWNRGAIVRFARQSAEDVGLRVATVPAFHTSIVCARCGALGCRF
EQTREGVRIAKLGPWFGCRACGRRIHADLNASENLHKVLIGTFPQVRRISRDPPVLSIADVRVDQREADRLAAAWLSELSVGPT
PF (SEQ ID NO: 341)
>3300025572|Ga0207864_1014515_2|P
[lab enrichment-defined media-ionic liquid and high solid enriched]
MGIKSWRVKLWVESPEIRATLEYCLSLWNTSAALLCRHVLAMRRGDFGPEGQAIWEHLKKTPHNFHVLYPISNGSGIEKTTRYY
PDARAFVNKYGYGALGPVRSASGCLWKQLTKAVRERTHSYDKTCEKWSEEHSKWLEERAKWEAEHPEYMAIRPLIDTFHAKHGV
TRGRRNRWALWLQFLMETPELVRWRNKTAKVVPLTDSELKRIRSKRRGAARREQALFYEKNPELAELNRIRGIYERRFARSAAK
RKNPDGFRHRPTFTLPSEAKYPDWPRFQAGEGWRDLDLDRRTICLKLPTDGGSARWVQVHFVPDPRLQRLARLKQPVKEGATRY
DYSLDMPSGHTVLAQPQEVRLIRRGSSYFATIRLSLHSAPSAIPVPQTAASSPAGMLRKIRTDAPELKLITCAVDLGVRHLGAA
TIAREGEIIGRRILHNRFLAPGADGGVINIPSLPQIAQVRRQIRCAQRRSGRISPKVAGCRHLWTHYRHLCEDRYKKAVAAIFG
FARAHGAHIVLFEDLKMLNPDSANERGVNRALQNWNRGRIVEFAKNTAEDAGLCVVTVPAYWTSRICARCNAFGVRFNQGQRRK
PGDDGHVSRVACELDFLGHWFLCPSCGRSVHADLNASENLHRVFLGTFPTVQTIQRQPRIYEINGQRVALDEIQQRAVEILASR
RAGDTPF (SEQ ID NO: 332)
>3300025572|Ga0207864_1014515_3|M
[lab enrichment-defined media-ionic liquid and high solid enriched]
MRRGDFGPEGQAIWEHLKKTPHNFHVLYPISNGSGIEKTTRYYPDARAFVNKYGYGALGPVRSASGCLWKQLTKAVRERTHSYD
KTCEKWSEEHSKWLEERAKWEAEHPEYMAIRPLIDTFHAKHGVTRGRRNRWALWLQFLMETPELVRWRNKTAKVVPLTDSELKR
IRSKRRGAARREQALFYEKNPELAELNRIHGIYERRFARSAAKRKNPDGFRHRPTFTLPSEAKYPDWPRFQAGEGWRDLDLDRR
TICLKLPTDGGSARWVQVHFVPDPRLQRLARLKQPVKEGATRYDYSLDMPSGHTVLAQPQEVRLIRRGSSYFATIRLSLHSAPS
AIPVPQTAASSPAGMLRKIRTDAPELKLITCAVDLGVRHLGAATIAREGEIIGRRILHNRFLAPGADGGVINIPSLPQIAQVRR
QIRCAQRRSGRISPKVAGCRHLWTHYRHLCEDRYKKAVAAIFGFARAHGAHIVLFEDLKMLNPDSANERGVNRALQNWNRGRIV
EFAKNTAEDAGLCVVIVPAYWTSRICARCNAFGVRFNQGQRRKPGDDGHVSRVACELDFLGHWFLCPSCGRSVHADLNASENLH
RVFLGTFPTVQTIQRQPRIYEINGQRVALDEIQQRAVEILASRRAGDTPF (SEQ ID NO: 342)
>SRR6505097_megahit_k177_377355_35|M
[permafrost metagenome]
MGVRALRARIECDRETLLHLWRTHCAFNQRLLSILTKLFGMRRGELGETAARRALYQRVARFLLARDSKDAPYLLNSVSIQGWK
PATALKMKVRMPAPDGTLAEVSADTWAPEAAKLSAHGTLAYDKEAVRDGLPDSLFQPMLRDAVAYISGHIELARNWEAEHADWL
KAKAEWEEKDERKKYLALRPRFDAFEQSVGGKAGKRRERWHLYLTWLRENPALAAWRGGPPAVHPLDAKATARVAKAKPWKKRS
VEADEFWKANPELRELDKLHGYYEREFVRRRKTKKNPDGFDHRPTFTLPHAVLHPRWTLFNAPQTSPQGYADLRLPQRPGTLGS
VKLRLLTGEKAPDGCPNEWVGVQFRADPRLSQFAERAEAQEIRKGKKKGTQKTVRVFRFVDQQLNCERPAQISGAKLIFKNVKL
NDDGSLKSATPYLVFACTIDNLPLTEHARAIQWSETGEVTKTGKKRKAKKLPTGLVTCAVDLGLRNLAFLTLATSGEGPQNNTH
DGIRVLRSRNQWLALEETAGTHPGRWSAGPDLAHIAAHKREIRRLRRLRGKPVKGQQSHVELQGHIDHMGQDRFKKAARAIVNF
ALNVDRAAGQSGQPYPRADLLIVEKLEGFIPDAARERGINRALVAWNRGQLVTRIKEVAADAGLKLFPVHPAGTSQCCSRCGAL
GRRYSIVRDQQTGVPAIRFGWVEKLFACPNRECPGRAPDKPDQPFTCNADHNASLNLHRRFVLDDRAVAAFFAWKNKTNAERER
ELTQMDQALRPELEKLHRLPLAELATPF (SEQ ID NO: 343)
>SRR6962291_megahit_k177_1754533_5|M
[permafrost metagenome]
MKRGDSGTTDAQKHLYSQIARFVLARSSRDAPYLMNSISIRGWTPNTAKKKMKATLMGPDGQEVETSGDDWADAAAALSAEGTL
LYDKSELLGDVPDTLRQVVVRECAAIISGHEELVRAWELSHEEWLEQKAKWESDPEHERYMVLRPQFETFEQSVGGKAGKRRGR
WQRYLDWLRSNPELAAWRGGPATVVELPAAARERVRKARPSKVRSVEAEEFWKVNPELFELDRLHGFYEREFVRRRKTKKHPDG
FDHRPTFTLPDPVRHPRWFVFNAPQTSPQGYRKLSLPKKNGDFGTIELLLLTGEKTDAKYPNGWNQVRFAADPRLSDFKQISKK
RQIRKGKTKGQETDSNAFTFLDRQISMERPAQISGAKLLFKKVRLKSDGALLSADPYLVFTCNITDVPFTDAARAIKWEDAGER
RKRAVIPAGLITCAVDLGIRHVGFATIAAKDEARPEGLRVLRSRNVWMGHEEQKGRHPGRWSPGPELPHLASHKRSLRRLRRLR
GKPVKDESSHAELQGHITNMANDRFKKAAREIVNFALNVERKVDPRTGEPYSRADLLVVENLANLIPDSERERGINRALIEFNR
GHLVDRIAEVAKDCGLKLMKVSPVGTSQVCCRCGALGRRYSIQRDRDSGYADIRFGFVEALFACPHCGYRANADHNASINLHRR
FLLGEAAIRGFKEWQQGSRQERADTVRRIEESLVAPLRLMHRLEVADMPF (SEQ ID NO: 344)
>SRR6962291_megahit_k177_1754533_5|P
[permafrost metagenome]
MSTRAIQSRIQCDHQTLEHLWRTHVVFNRRLPPLISLLFRMKRGDSGTTDAQKHLYSQIARFVLARSSRDAPYLMNSISIRGWT
PNTAKKKMKATLMGPDGQEVETSGDDWADAAAALSAEGTLLYDKSELLGDVPDTLRQVVVRECAAIISGHEELVRAWELSHEEW
LEQKAKWESDPEHERYMVLRPQFETFEQSVGGKAGKRRGRWQRYLDWLRSNPELAAWRGGPATVVELPAAARERVRKARPSKVR
SVEAEEFWKVNPELFELDRLHGFYEREFVRRRKTKKHPDGFDHRPTFTLPDPVRHPRWFVFNAPQTSPQGYRKLSLPKKNGDFG
TIELLLLTGEKTDAKYPNGWNQVRFAADPRLSDFKQISKKRQIRKGKTKGQETDSNAFTFLDRQISMERPAQISGAKLLFKKVR
LKSDGALLSADPYLVFTCNITDVPFTDAARAIKWEDAGERRKRAVIPAGLITCAVDLGIRHVGFATIAAKDEARPEGLRVLRSR
NVWMGHEEQKGRHPGRWSPGPELPHLASHKRSLRRLRRLRGKPVKDESSHAELQGHITNMANDRFKKAAREIVNFALNVERKVD
PRTGEPYSRADLLVVENLANLIPDSERERGINRALIEFNRGHLVDRIAEVAKDCGLKLMKVSPVGTSQVCCRCGALGRRYSIQR
DRDSGYADIRFGFVEALFACPHCGYRANADHNASINLHRRFLLGEAAIRGFKEWQQGSRQERADTVRRIEESLVAPLRLMHRLE
VADMPF (SEQ ID NO: 345)
>SRR6962291_megahit_k177_3367137_15|M
[permafrost metagenome]
MFNRRLPDLISLLFRMRRGECGETEQASRLYSQVASFILARGSKDAPYLLNSVSVRGWKPHTAKKMKARALGPDGQQIELAGED
WADPAAALSAEGRLLYDKTELLGDVPDTLRQMAARECVAIISGHEELVRAWEKAHEEWLASKAKWESDPENQFYLGLRARFDEF
EQSVGGKVGKRRGRWQRYLDWLRANPDLAAWRGGPAAVVELSAGAKERIRKAKPRKVRSVEAEEFWKVNSELAALDRLHGYYER
EFVRRRKTKKHPDGFDHRPTFTLPHPVRHPRWFVFNAPQTSPQGYRKLCLPSSGGHTGTVELLLLTGEKADNKYPSGWETVRFA
ADPRLSDFKAITKRRQVLKGKTKGQETDTKAFTFLDRQINLERPAQISGVKLIFKGVRQNHDGSLLSAHPYLVFTCNVADVPLT
DGAKKIKWEDTGERRKRVVVPAGLIACAVDLGIRHVGFATVATQDAESGGLHVLRSRNIWIGQEEEKGRNPGRWSPGPDLAHLS
AHKRSLRHLRGLRGKPVKGENSHIELQDHITNMASDRFKKAAREIVNFALNADSKVDPRTGEPYPRADVLVLENLANLLPDSER
ERGINRALIEFNRGHLADRIVEVAKDCGLKVMMVSPVGTSQVCCRCGALGRRYSLRRHQDGGYTDIHFGFVEPLFACPSCGYRA
NSDHNASVNLHRRFRLGEAAIAKFKEWQKGTKPERDEVLRRIEQSLVGPLREMHRLAVADTPF (SEQ ID NO: 346)
>SRR6962291_megahit_k177_3367137_14|P
[permafrost metagenome]
MSTRALQSRIQCDHQTLEHLWRTHLVFNRRLPDLISLLFRMRRGECGETEQASRLYSQVASFILARGSKDAPYLLNSVSVRGWK
PHTAKKMKARALGPDGQQIELAGEDWADPAAALSAEGRLLYDKTELLGDVPDTLRQMAARECVAIISGHEELVRAWEKAHEEWL
ASKAKWESDPENQFYLGLRARFDEFEQSVGGKVGKRRGRWQRYLDWLRANPDLAAWRGGPAAVVELSAGAKERIRKAKPRKVRS
VEAEEFWKVNSELAALDRLHGYYEREFVRRRKTKKHPDGFDHRPTFTLPHPVRHPRWFVFNAPQTSPQGYRKLCLPSSGGHTGT
VELLLLTGEKADNKYPSGWETVRFAADPRLSDFKAITKRRQVLKGKTKGQETDTKAFTFLDRQINLERPAQISGVKLIFKGVRQ
NHDGSLLSAHPYLVFTCNVADVPLTDGAKKIKWEDTGERRKRVVVPAGLIACAVDLGIRHVGFATVATQDAESGGLHVLRSRNI
WIGQEEEKGRNPGRWSPGPDLAHLSAHKRSLRHLRGLRGKPVKGENSHIELQDHITNMASDRFKKAAREIVNFALNADSKVDPR
TGEPYPRADVLVLENLANLLPDSERERGINRALIEFNRGHLADRIVEVAKDCGLKVMMVSPVGTSQVCCRCGALGRRYSLRRHQ
DGGYTDIHFGFVEPLFACPSCGYRANSDHNASVNLHRRFRLGEAAIAKFKEWQKGTKPERDEVLRRIEQSLVGPLREMHRLAVA
DTPF (SEQ ID NO: 347)
>AQT69685.1
[Phycisphaerae bacterium ST-NAGAB-D1]
MATKSYRARILTDSRLAAALDRTHVVFVESLKQMINTYLRMQNGKFGPDHKKLAQIMLSRSNTFAHGVMDQITRDQPTSTLDEE
WTDLARRIHKTTGPLFLQAERFATVKNRAIHTKSRGKVIPSPETLAVPAKFWHQVCDSASAYIRSNRELMQQWRKDRAAWLKDK
NEWQQKHPEFMQFYNGPYQNFLKLCDDDRITSQLAAEQQPTASKNNRPRKTGKRFARWHLWYKWLSENPEIIEWRNKASASDFK
TVTDDVRKQIITKYPQQNKYITRLLDWLEDNNPELKTLENLRRTYVKKFDSFKRPPTLTLPSPYRHPYWFTMELDQFYKKADFE
NGTIQLLLIDEDDDGNWFFNWMPASLKPDPRLVPSWRAETFETEGRFPPYLGGKIGKKLSRPAPTDAERKAGIAGAKLMIKNNR
SELLFTVFEQDCPPRVKWAKTKNRKCPADNAFSSDGKTRKPLRILSIDLGIRHIGAFALTQGTRNDSAWQTESLKKGIINSPSI
PPLRQVRRHDYDLKRKRRRHGKPVKGQRSNANLQAHRTNMAQDRFKKGASAIVSLAREHSADLILFENLHSLKFSAFDERWMNR
QLRDMNRRHIVELVSEQAPEFGITVKDDINPWMTSRICSNCNLPGFRFSMKKKNPYREKLPREKCTDFGYPVWEPGGHLFRCPH
CDHRVNADINAAANLANKFFGLGYWNNGLKYDAETKTFTVHTDKKTPPLIFKPRPQFDLWADSVKTRKQLGPDPF (SEQ ID
NO: 348)
>PMBN01000097_4|M
[Phycisphaerales bacterium]
MGVRALRARIECDRETLLHLWRTHCAFNQRLLSILTKLFGMRRGELGETAARRALYQRVARFLLARDSKDAPYLLNSVSIQGWK
PATALKMKVRMPAPDGTLAEVSADTWAPEAAKLSAHGTLAYDKEAVRDGLPDSLFQPMLRDAVAYISGHIELARNWEAEHADWL
KAKAEWEEKDERKKYLALRPRFDAFEQSVGGKAGKRRERWHLYLTWLRENPALAAWRGGPPAVHPLDAKATARVAKAKPWKKRS
VEADEFWKANPELRELDKLHGYYEREFVRRRKTKKNPDGFDHRPTFTLPHAVLHPRWTLFNAPQTSPQGYADLRLPQRPGTLGS
VKLRLLTGEKAPDGCPNEWVGVQFRADPRLSQFAERAEAQEIRKGKKKGTQKTVRVFRFVDQQLNCERPAQISGAKLIFKNVKL
NDDGSLKSATPYLVFACTIDNLPLTEHARAIQWSETGEVTKTGKKRKAKKLPTGLVTCAVDLGLRNLAFLTLATSGEGPQNNTH
DGIRVLRSRNQWLALEETAGTHPGRWSAGPDLAHIAAHKREIRRLRRLRGKPVKGQQSHVELQGHIDHMGQDRFKKAARAIVNF
ALNVDRAAGQSGQPYPRADLLIVEKLEGFIPDAARERGINRALVAWNRGQLVTRIKEVAADAGLKLFPVHPAGTSQCCSRCGAL
GRRYSIVRDQQTGVPAIRFGWVEKLFACPNRECPGRAPDKPDQPFTCNADHNASLNLHRRFVLDDRAVAAFFAWKNKTNAERER
ELTQMDQALRPELEKLHRLPLAELATPF (SEQ ID NO: 343)
>PMDC01000375_4|M
[Phycisphaerales bacterium]
MGVRALRARIECDRETLLHLWRTHCAFNQRLLSILTKLFGMRRGELGETAARRALYQRVARFLLARDSKDAPYLLNSVSIQGWK
PATALKMKVRMPAPDGTLAEVSADTWAPEAAKLSAHGTLAYDKEAVRDGLPDSLFQPMLRDAVAYISGHIELARNWEAEHADWL
KAKAEWEEKDERKKYLALRPRFDAFEQSVGGKAGKRRERWHLYLTWLRENPALAAWRGGPPAVHPLDAKATARVAKAKPWKKRS
VEADEFWKANPELRELDKLHGYYEREFVRRRKTKKNPDGFDHRPTFTLPHAVLHPRWTLFNAPQTSPQGYADLRLPQRPGTLGS
VKLRLLTGEKAPDGCPNEWVGVQFRADPRLSQFAERAEAQEIRKGKKKGTQKTVRVFRFVDQQLNCERPAQISGAKLIFKNVKL
NDDGSLKSATPYLVFACTIDNLPLTEHARAIQWSETGEVTKTGKKRKAKKLPTGLVTCAVDLGLRNLAFLTLATSGEGPQNNTH
DGIRVLRSRNQWLALEETAGTHPGRWSAGPDLAHIAAHKREIRRLRRLRGKPVKGQQSHVELQGHIDHMGQDRFKKAARAIVNF
ALNVDRAAGQSGQPYPRADLLIVEKLEGFIPDAARERGINRALVAWNRGQLVTRIKEVAADAGLKLFPVHPAGTSQCCSRCGAL
GRRYSIVRDQQTGVPAIRFGWVEKLFACPNRECPGRAPDKPDQPFTCNADHNASLNLHRRFVLDDRAVAAFFAWKNKTNAERER
ELTQMDQALRPELEKLHRLPLAELATPF (SEQ ID NO: 343)
>pMMM01000072_9|M
[Phycisphaerales bacterium]
MGVRALRARIECDRETLLHLWRTHCAFNQRLLSILTKLFGMRRGELGETAARRALYQRVARFLLARDSKDAPYLLNSVSIQGWK
PATALKMKVRMPAPDGTLAEVSADTWAPEAAKLSAHGTLAYDKEAVRDGLPDSLFQPMLRDAVAYISGHIELARNWEAEHADWL
KAKAEWEEKDERKKYLALRPRFDAFEQSVGGKAGKRRERWHLYLTWLRENPALAAWRGGPPAVHPLDAKATARVAKAKPWKKRS
VEADEFWKANPELRELDKLHGYYEREFVRRRKTKKNPDGFDHRPTFTLPHAVLHPRWTLFNAPQTSPQGYADLRLPQRPGTLGS
VKLRLLTGEKAPDGCPNEWVGVQFRADPRLSQFAERAEAQEIRKGKKKGTQKTVRVFRFVDQQLNCERPAQISGAKLIFKNVKL
NDDGSLKSATPYLVFACTIDNLPLTEHARAIQWSETGEVTKTGKKRKAKKLPTGLVTCAVDLGLRNLAFLTLATSGEGPQNNTH
DGIRVLRSRNQWLALEETAGTHPGRWSAGPDLAHIAAHKREIRRLRRLRGKPVKGQQSHVELQGHIDHMGQDRFKKAARAIVNF
ALNVDRAAGQSGQPYPRADLLIVEKLEGFIPDAARERGINRALVAWNRGQLVTRIKEVAADAGLKLFPVHPAGTSQCCSRCGAL
GRRYSIVRDQQTGVPAIRFGWVEKLFACPNRECPGRAPDKPDQPFTCNADHNASLNLHRRFVLDDRAVAAFFAWKNKTNAERER
ELTQMDQALRPELEKLHRLPLAELATPF (SEQ ID NO: 343)
>QBM02859.1
[Planctomycetes bacterium RBG_13_46_10]
MSVRSFQARVECDKQTMEHLWRTHKVFNERLPEIIKILFKMKRGECGQNDKQKSLYKSISQSILEANAQNADYLLNSVSIKGWK
PGTAKKYRNASFTWADDAAKLSSQGIHVYDKKQVLGDLPGMMSQMVCRQSVEAISGHIELTKKWEKEHNEWLKEKEKWESEDEH
KKYLDLREKFEQFEQSIGGKITKRRGRWHLYLKWLSDNPDFAAWRGNKAVINPLSEKAQIRINKAKPNKKNSVERDEFFKANPE
MKALDNLHGYYERNFVRRRKTKKNPDGFDHKPTFTLPHPTIHPRWFVFNKPKTNPEGYRKLILPKKAGDLGSLEMRLLTGEKNK
GNYPDDWISVKFKADPRLSLIRPVKGRRVVRKGKEQGQTKETDSYEFFDKHLKKWRPAKLSGVKLIFPDKTPKAAYLYFTCDIP
DEPLTETAKKIQWLETGDVTKKGKKRKKKVLPHGLVSCAVDLSMRRGTTGFATLCRYENGKIHILRSRNLWVGYKEGKGCHPYR
WTEGPDLGHIAKHKREIRILRSKRGKPVKGEESHIDLQKHIDYMGEDRFKKAARTIVNFALNTENAASKNGFYPRADVLLLENL
EGLIPDAEKERGINRALAGWNRRHLVERVIEMAKDAGFKRRVFEIPPYGTSQVCSKCGALGRRYSIIRENNRREIRFGYVEKLF
ACPNCGYCANADHNASVNLNRRFLIEDSFKSYYDWKRLSEKKQKEEIETIESKLMDKLCAMHKISRGSISK (SEQ ID NO:
349)
>SRR6963308_megahit_k177_1876783_1|M
[sediment metagenome]
MSQRALAVRLLVDSEDDRAALWRTHRIFNERLGWVLRQMHRMKRGEPDARYAEIFNAITTSQRATAALEAVTSLAWTGTKEDRW
CQLARALIQEGTLLFDREARLAGLSKEFRRKLFEAAFQMISGHRELLAQWERDHNEWQQARETWEAENPEYMAIRPALQRFEAE
HGQAAKRRARWHKWRAFLQGSSQLAGWRGGPQVVNEISADARRRIERARRNKRNKIEAEEFFRVNPELKELDAKHGYYQREFLR
PWAKRRNADGFKHRPTFTEPSAENHPFWFQFKKHETYKDLNLKVRSLRLKLLASDSAVPGSQWRDFRFRVDPRLLDLSEAQSPA
KIGIARFSLLFNDRALAMEDRPAEIRGAKLIFRPAQRDGRVYLSFTCDVADLPCRVPLKQESWDKYSAEWLCKKSREALDGEDP
VTCAVDLGIRRLGAASVRRDGKIVRARIIREEQQRGCGPRLAAIKNHKRALKAGRRKRGKPVRGEQSLVELQEHVDKMGRDRFK
KGARRIVNFAHQNHCDVIVMEKLAGLIPDAERERGINSALVNWNRGHLVKWVKQLANDAGMRVVEVYPHWTSQLCSRCGAMGAR
FSADHGHVAFEYVGSLFACADCGYVANADHNASVNLHRRFYGELPKVKKLREGVYELAKPDHAPKEVKLAELRSAVSVAAAAMC
RGQASPF (SEQ ID NO: 325)
>SRR6963352_megahit_k177_1225001_5|P
[sediment metagenome]
MSVKTIQAKLECDRETLETLWRTHTVFNEGLLPILALFFKMKRRETGSTEEEKDLYKRICEFILRTNASNPGYLLNSVCLENWK
PDSARKTKAEVVRDGKKEKISAEEWIDDLLNMKEESGLLFLKQELLNGLPIMFFEKLVAESKAILSAYEELQYQWEVDHNKWLK
EKEGWEKKHKEYLAVRSLFQKFEQEVKGKITKKRRRWHLYKDWMKRTPELVAWRGKETKVYGLTEEDREAIKKAPLKKKIGVEV
GLFFKRNPELSALDKLHSYYEEKFVRRGKKKRNCDGYEHRPTFTLPHSVKHPRWYVFSGPQTGNGYKNLNIPERKKDFGQLDLL
VLTDDKVDGEYPREYRTVKFRGDHRFLDIRGATRTRLVTKGKNKGQEVEEKFAYEMYDRYLKIWRPLKISGAKLAFRDIKINQD
GSLKSAVPYLWFTLNLEDLPISEKAKEIKWVETDEVKDNGDKRKKIQIPEGLITCAVNIGIRNLGFGTLAVGEKGKARILRSRN
IWIGEEEKTGKHKGRWQKGPTLRELRDHKQEIRRLRKLRGKVVKGERSHIKLQNHITNFADDRLKKAARKILNFALNVENHIDK
KTGKPYPRADVLLLEDLSTLVPTTKKRRDQNQLLIEFMHRGTLSKIEEMAKDAGLKVLRIKPGGISQVCPKCGGIGRRYEIRID
QETKKPDIHFTDVIDDIDCQKLFVCASCSYRGNGDHVASVNLHRKFYDWSVVDKYFTLLRSDNYKEQKKVIEEELREFLRKEHF
PQMALN (SEQ ID NO: 329)
>SRR6963480_megahit_k177_1813084_1|M
[sediment metagenome]
MSVKTIQAKLECDRETLETLWRTHTVFNEGLLPILALFFKMKRRETGSTEEEKDLYKRICEFILRTNASNPGYLLNSVCLENWK
PDSARKTKAEVVRDGKKEKISAEEWIDDLLNMKEESGLLFLKQELLNGLPIMFFEKLVAESKAILSAYEELQYQWEVDHNKWLK
EKEGWEKKHKEYLAVRSLFQKFEQEVKGKITKKRRRWHLYKDWMKRTPELVAWRGKETKVYGLTEEDREAIKKAPLKKKIGVEV
GLFFKRNPELSALDKLHSYYEEKFVRRGKKKRNCDGYEHRPTFTLPHSVKHPRWYVFSGPQTGNGYKNLNIPERKKDFGQLDLL
VLTDDKVDGEYPREYRTVKFRGDHRFLDIRGATRTRLVTKGKNKGQEVEEKFAYEMYDRYLKIWRPLKISGAKLAFRDIKINQD
GSLKSAVPYLWFTLNLEDLPISEKAKEIKWVETDEVKDNGDKRKKIQIPEGLITCAVNIGIRNLGFGTLAVGEKGKARILRSRN
IWIGEEEKTGKHKGRWQKGPTLRELRDHKQEIRRLRKLRGKVVKGERSHIKLQNHITNFADDRLKKAARKILNFALNVENHIDK
KTGKPYPRADVLLLEDLSTLVPTTKKRRDQNQLLIEFMHRGTLSKIEEMAKDAGLKVLRIKPGGISQVCPKCGGIGRRYEIRID
QETKKPDIHFTDVIDDIDCQKLFVCASCSYRGNGDHVASVNLHRKFYDWSVVDKYFTLLRSDNYKEQKKVIEEELREFLRKEHF
PQMALN (SEQ ID NO: 329)
>SRR6963491_megahit_k177_4304123_3|P
[sediment metagenome]
MTIKSFEAKIVTNPSLRETLEYTHRVYNEDLKYMVEVLLAMRKGKHGDGYVEFWREHLQKAANPSGLLEPLTTPDGPKADAKSK
KSKIIRQLFKEHGMLFDRDERWPHWNARHRRHLFDDAKKWARQLDERLERWRSEHAQWLKEKEKFENENPLYMAVEPLFGGFFK
EFGQGGKRRERWAAYYHFLLAHPELANWRGQADAIVPLTPEEEAACLRDRRRGVKKRLDLFLEKNPELEALHKLDAKYGRNMVR
RSARHRNIDGFRHPPTMTLPHSRLHPEWITFQAGETYKDLDLNGGTVRLNILPPDAETSKLHSVEFRAEKRLRRITPRETPLRK
GRNEFNKNYRDAADRVHPARVKGIRLILRPRGAYLKITVELDRPESRTGLKQQTCDKYSSNWCYQEIVKRREGKPLKCLSLDLG
VRHLAVGVVVQETRANGDRETVIEGVRFFKEVGGRPFPSLDEIRRHKDRLREKRSQTRRAPKGRRTCIALQDHVTKMGQDRYRK
GAAAIVRYAVERDVDVILLEKLAGFIPDAAKQRGINKALMEWNRRKIADFVKLLAADHGISVVEVAPHHLSQLCSKCGELGRRF
SWKDGLPKFGTVEKLFVCPACGYQANSDYNAAVNLYKAFSGAYEERPKKAKKGVYKYRDRTICLKKLDGQLAERLTQEQTPF
(SEQ ID NO: 328)
>SRR6963587_megahit_k177_2272343_29|M
[sediment metagenome]
MATKTYRARIVQISPRLGAALERTHYLFAEALNQMVRRYIDMRKGKYGQECKKLADIMLTRSNTFAHGVMDQLTREISTSGLTD
EWVDLARAVHKSHGPLFLQHDGFAEVDGVMLHTKRHGKVSPMPGRLPVNAKFWHQVCDSASAFLKSNNELLENWRQGRKEWLQE
KADWESANADFMRFWNGSYQEFEETCEIKRKESQALEGQKVVEKKRHRRDRGKRMSRWHLWYNWVITHPEIIEWRNRAKAAEFR
PVPPTVHKKIKNKYPRQDKHITKFLDWLKENNPELKALDSRRRFYVRNYLRFRRPPTLTLPSANKHPYWFTFELNQFYKNVDFD
KGSIQLLLVDETEEGSWFFRWFDAKIQCDPRLRPCVRAGVFATKGRYPPYIGGKVGNKLNRPAERPENRKAGYAGAKLVLNKRR
KELFFTVIEQDCPRKVSWKKTKQRTCRGDNVFAKDGSQIPLKVMAIDLGIRHIGAYVIAEACRNRGVWDISWQHKGITGRHAVP
GLKEIRQHDRQLRKARSQRGKPVQGEESFIELQDHRTNMADDRFKKAANTIVELARQYDVHLILFERLGNLSPTAFDERWMNRQ
LRDMNRRQIVDTVKLQAREFGIECLEDISPWMTSHICSRCFKPGWRFSIKRKNPYGERFTRKNCRDYGYPVWDTGGHLFRCPHC
EYKCNADINAACNVAAKFFGLWSGLDGLKRDQWVYTWQENQPKTFDAKKTFLEWALDVRNRKLMGDSPF (SEQ ID NO:
326)
>SRR6963591_megahit_k177_1621237_2|P
[sediment metagenome]
MATKTYRARIVQISPRLGAALERTHYLFAEALNQMVRRYIDMRKGKYGQECKKLADIMLTRSNTFAHGVMDQLTREISTSGLTD
EWVDLARAVHKSHGPLFLQHDGFAEVDGVMLHTKRHGKVSPMPGRLPVNAKFWHQVCDSASAFLKSNNELLENWRQGRKEWLQE
KADWESANADFMRFWNGSYQEFEETCEIKRKESQALEGQKVVEKKRHRRDRGKRMSRWHLWYNWVITHPEIIEWRNRAKAAEFR
PVPPTVHKKIKNKYPRQDKHITKFLDWLKENNPELKALDSRRRFYVRNYLRFRRPPTLTLPSANKHPYWFTFELNQFYKNVDFD
KGSIQLLLVDETEEGSWFFRWFDAKIQCDPRLRPCVRAGVFATKGRYPPYIGGKVGNKLNRPAERPENRKAGYAGAKLVLNKRR
KELFFTVIEQDCPRKVSWKKTKQRTCRGDNVFAKDGSQIPLKVMAIDLGIRHIGAYVIAEACRNRGVWDISWQHKGITGRHAVP
GLKEIRQHDRQLRKARSQRGKPVQGEESFIELQDHRTNMADDRFKKAANTIVELARQYDVHLILFERLGNLSPTAFDERWMNRQ
LRDMNRRQIVDTVKLQAREFGIECLEDISPWMTSHICSRCFKPGWRFSIKRKNPYGERFTRKNCRDYGYPVWDTGGHLFRCPHC
EYKCNADINAACNVAAKFFGLWSGLDGLKRDQWVYTWQENQPKTFDAKKTFLEWALDVRNRKLMGDSPF (SEQ ID NO:
326)
>SRR8554444_megahit_k177_608234_1|M
[sediment metagenome]
MATKSFEAKIVCKPDEKYTAEQKKQFLWFTHQVFNDGVRKVIPYVFKMKRGELGPEFQAIYYAITSSQDAIGKLEAVINPDWTS
GKIGKSDPNKWKELLKYQELEKGFRQRLKEEGIKSTKKFRKELEDEKKKLAKEIGQKDIWADAAAILRNKNLLLFNRDELLPNL
PSEFRRKIYEMTIQLIHGHQELVANWEDEHAEWLIEKDKWEEEHPEYMNVRPIFEKFEKEQGKVKGSRIRWLAYLDFLSSKPEL
ANWRGKAKETIPLTKEERAGFRKPGQHFAAFFNKNPELQELDRLHKEYQEKFARTQSKRTPHPDGFKHRPTFTLPDAMRHPVWY
SFKGATDPTKGSTYRNLDLENCTLDLKVLTAMEGEGRNPGGMIQYAFEPDERIKGFRYVGTTEKGKRAKGYIYYDPILEKERPA
KIQGIKLVFRPPRPDGTAYLIFSCQIEDEKPKIKIWKDKEEESPGEITKRKKTEVYPPELITLAIDFGQRHLGAITICKNNNGR
PEPIRFIPAYPKRRKDRESKPVSAWLAKIPGLTFNAVGMHEKEISAGMSRRFQDPKSIRQAGEKEGRKSKGQHIPETETPWAHL
REHIANMKEDHYKKAANLIIRTALQNGAQVILIENLRNYRPMLERTNLENRRRMQWAVRQTAKFLEDTARPLGLIVRQVSSAYT
SRFCSSCGHPGARVSLPGQKNWEKFYAEKYGKERKMIAVAGGQFFCCPACKKIINADINASLNMHKVFYQNFIWPEKIDKKDTK
NFIWQGKNYNWDQIADDVQSFLDQKAGIKKEDDIPY (SEQ ID NO: 350)
>SRR4030068_megahit_k177_2163203_10|M
[soil metagenome]
MGVRAIKSKIICDKVVLTHLWRTHKVFNERLPLIISLLFKMRRGEAGETPERQDLYRRIAEFILMRSSKDAPYLFNSICIRNWK
PNSAKGYKVRLIDDGGVATETSGESWAEEAARLSAEGHLLYDKDDMVGDLPALLQQMVFRDSVSIISGHDGLVDLWKKDHAEWL
KRKADWEGREENKKYITMRPKFEEFEAAVGGKIGKRRGRWHLYHEWLRENPDLAGWRGGPPVVNPISTAGKKRIARAAPSKKMS
VEFEEFWKANPELQALDRLHGYYEREFVRRRKRKRNPDGFDHRPTFTLPHPVRHPRWLVFNAPQTNPVGYRNLILPTARSLRGS
VELQLVTGEKTEERYPHAWVPVGFKADPRLGDLRRVTAKSVYRKGKDKGKEKEVEAYEFLDRHLNRYRKAALSGAKLIFRLSPG
GSPKEAYLYFTLEVANEPLTDKAKAIKWTETGEVTKRGKARKRKTVSEGLVTCAVDLGIRNLGFATLAEYVDGVPRILRSRNLW
IGIEEEGGARPGRWSEGPTLHHISEHKAKIRSLRRLRGKPVKGEESHLELQSHITSMGEDRFKRSARAIINFALNAKKEAGKKG
AYPRADVLLLERLEGMIPDAERGRGINRALMAWNRGQLVEQVKEVARDAGLKVFEIHPIGTSQVCSKCGSLGRRYSIGRNKEDG
EKEITFGWVEKLFACPCGYTANADHNASVNLHRRFLFDDGAIPGFLEWSKRDKSGREAMVARIEAALEPRLRAMHGLEPISV
(SEQ ID NO: 351)
>SRR5487326_megahit_k177_254484_5|M
[soil metagenome]
MAVRAIQARLESDKNMLEHLWRTHKVFNERLPRIISILFKMRRGEYGTTDEDRALYKSIAKFVLGRNARDAPYLLNSISIKNWT
PSTAKKMKVTITTSDGSEQEVYGDTWANKAAELSSAGRLLYDKEEVLGDLPDSLRQIVVRECVAIISGHDELVKNWDKAHTEWL
KKKEEWEAKEEHKKYLAVRSHFDDFEKSVGGKAGKRRARWHKYLQWLRENPDLAGWRGGLAAVNELSKEAQERVRKAKPWRQRS
VEAEEFWKANPELQALDRLHGQYERDFVRRRKTKRNPDGFEHRPTFTLPHFLRHPRWLVFNAPQTSPAGYRNLKLPGKVGQYGE
VDLRLLTDEIRGAEFPVDWTRLRFLGDPRLADFKPVKIQKAATKGKVKGELRERPAYRFFDGELQMERPAQISGAKLIFKNVRL
DDGSLQSATPYLYFTCEAESLQWTENARKVKWNATGEVMKKGKKRGYRTLPDGLIACAVDLGIRNLGFATLARYENGVPRLLRS
RNLWIGHAEARGIHPGRWSEGPELAHLALHKRELRRLRSLRGDPIAGEESHIKLQQHITYFAEDRFKKAARAIVNFALNVKEFA
DKNTGEIYPRADVLVLENLGTLLPNAEREKGINRSLIEFNRGHLVDRVKELAKDVGLRVFEVSPVGTSQVCSRCGTLGRRYSIR
RSAETHRPEIQFGFVEKLFACPECGYRANADHNASVNLHRRLAQVPGAFAQWEELGKDQQSKRKRWEEIEAKLLPSLQKMHGLD
GAGKPHPFLSA (SEQ ID NO: 352)
>SRR5487761_megahit_k177_404752_1|M
[soil metagenome]
MAVRAIQARLESDKNMLEHLWRTHKVFNERLPRIISILFKMRRGEYGTTDEDRALYKSIAKFVLGRNARDAPYLLNSISIKNWT
PSTAKKMKVTITTSDGSEQEVYGDTWANKAAELSSAGRLLYDKEEVLGDLPDSLRQIVVRECVAIISGHDELVKNWDKAHTEWL
KKKEEWEAKEEHKKYLAVRSHFDDFEKSVGGKAGKRRARWHKYLQWLRENPDLAGWRGGLAAVNELSKEAQERVRKAKPWRQRS
VEAEEFWKANPELQALDRLHGQYERDFVRRRKTKRNPDGFEHRPTFTLPHFLRHPRWLVFNAPQTSPAGYRNLKLPGKVGQYGE
VDLRLLTDEIRGAEFPVDWTRLRFLGDPRLADFKPVKIQKAATKGKVKGELRERPAYRFFDGELQMERPAQISGAKLIFKNVRL
DDGSLQSATPYLYFTCEAESLQWTENARKVKWNATGEVMKKGKKRGYRTLPDGLIACAVDLGIRNLGFATLARYENGVPRLLRS
RNLWIGHAEARGIHPGRWSEGPELAHLALHKRELRRLRSLRGDPIAGEESHIKLQQHITYFAEDRFKKAARAIVNFALNVKEFA
DKNTGEIYPRADVLVLENLGTLLPNAEREKGINRSLIEFNRGHLVDRVKELAKDVGLRVFEVSPVGTSQVCSRCGTLGRRYSIR
RSAETHRPEIQFGFVEKLFACPECGYRANADHNASVNLHRRLAQVPGAFAQWEELGKDQQSKRKRWEEIEAKLLPSLQKMHGFD
GAGKPHPFLSA (SEQ ID NO: 353)
>SRR8554485_megahit_k177_439309_3|P
[soil metagenome]
MAVRAIQVRLESDKTLLEHLWRTHKVFNERLPMIISILFKMRRGECGTTDEERTLYKNIARFVLARSSKDAPYLLNSISIKDWT
PSTARKMKATITIPDGSEQEVCGDTWADKVAELSAAGRLLYSKQEVLGDLPDSLRQMIVRECVAIISGHDELVRNWDKSHTEWL
KKREEWEAKEEHKKYLAVRSRFDEFEKSVGGKAGKRRARWHKYLQWLRESPDLAGWRGSLAVVNELSREAQERVRKAKPWRQRG
VEAEEFWKANPELQALDRLHGQYERDFVRRGKTKRNPDGFEHRPTFTLPHFLRHPRWLVFNAPQTSPPGYRNLKLPGKAGQYGE
VELRLLTNEIRGAEFPIDWTTLRFLGDPRLADFKPVKIQKAATKGKVKGELRGRPAYRFFDRELQMERPAQISGAKLIFKNVRL
DDGPLQSATPYLYFACEAKSLRWTENARKVKWNATGEVTKKGKKRGYRTLPDGLIACAVDLGIRNLGFATLARYENGVPRLLRS
RNLWIGHAETRGRHPGRWCEGPELAHLALHKRELRRLRSLRGDPIAGEESHIELQQHITHFAEDRFKKAARAIVNFALNVKEFA
DKNTGEIYPRADVLVLENLGSLLPDAEREKGINRSLIEFNRGHLVDRVKEVAKDVGLRVFEVSPVGTSQVCSRCGTLGRRYSIR
RSAETHRPEIQFGFVEKLFACPECGYRANADHNASVNLHRRLAQVPGAFAQWEELGKDQQRKRKRWEEIEAKLLPSLQKMHGFD
GAGKPHPFLSA (SEQ ID NO: 354)
>SRR8554511_megahit_k177_4512460_1|M
[soil metagenome]
MCESPQQRAFLELTHRLFNESLRPLVPLLYAARKGRHGPDYQQILSTIKSGQGASAQVEAVSSLAARPGSRGGKEWTDLAKELV
ARGQILFDRHALMPNVHKEFRRKVFEMAFQIILSHDAKMKQWRTDHAKWLREKAEWERSYPEYLAVRPVIQEFEAREGQAGKRR
GRWHRWLDFLVLYPQLAAWRGGEPTVRQLTQQELQEARRSRRRAVPNTIDAFFAKNPELKELHGLHGEYQARFVRPWAKRRHQD
GFFLPPTFTLPSADRHPAWYSFKRGDTYRNLSLEQRTIELRVLKSADPNESGRHAYVQYQFVGDNRLGKLSPASKIVRSGRFPC
DLLVWDEGSDHARPARVQGAKLVIRKGQAYLYFSVYIEDAPSRLPVKQAQIDKYSPGWSINRIRGEPYDPPLRTMAIDLGIRHI
AAATVMENGNVLATRFLHSRPVSRVSGHVIEGIPTLPQISEMKQRLRRARRKFGKPLKGTMSCRRIQRHVTSMSEDRCKKSASA
IVDFGRAHRVDIILMERLEGLVPDAARERRINCGLINWNRGHLASWIRIIAEPYGIRVLELPPRWTSQVCSHCGSIGARFTCTR
GEMKISAGGKLFGCPTCAFEANADFNASLNLQRVFWGLFPQVSKAGRGRVQCNDRVIELASVNQAWENHWLRKHNSANDVF
(SEQ ID NO: 355)
>SRR8554511_megahit_k177_1767604_11|P
[soil metagenome]
MAAKSYQPKMSTKSYPSKVICGKSEFDDLRRTHRVFNAGVRKLVDTYIAMKKGAYGQTAVEIAMYLLGKKGSKGHDLMNPLTAE
DAKDTLDTEGMKLVRQYRRESGILFSRYEKIYEIDGKAVCAGSAKKGDSKGKLVISSIFWHFICNQAIEYLRSNNELMQAWRDD
RRKWLEEKQKFFDENAGFREFVQGYLADFDAHAEKMRQEFQTQAGQRAYLRKRNKSDSGKRFSRWHLWYEWICSHPEILEWRGR
AKAADFVLLTPDEVHEVEKQFPKRQDRRTGKMLDMLRRKNPELDEIEKLRKTYTNDFARFRRPPTLTLPSADKHPCWCRLEKDK
LYRKADFEKGTIELCVVCKDETDGEYDMKWRTFRLKPDPRLQPSHRAKAFAAEGRYPPYKEGKVGNTLNRPAESPDKRLAGLKG
AKLILKRNDNAQIVFTVVEQNEPLKAKFSKKEGAKSSADRLEILDGKEATRVMAVDLGARHVGAYVITEGKRSSDVWRLEHLKK
AFVDSPDLPDLRSITQHDWQLRKARRKRGKPVKNQRSFISLQDHRTKMSDDRFKKAANVIVENARRHSVHIIVFEDLRDLKPTA
YNERWLNRQVRGMNHRHIFDFTKKMAAEFGINVTDVRAYNTSRLCSKCGFPGYRFSMKQKSPYREKAPRKGCSDFGYPVWDSGG
HLFRCPHCGYRVQADLNAAANIAQRFFGSLMEWKCKDYRYSNAMHPSFDARKDFDKWASDVQRRKSMPDAPF (SEQ ID NO:
356)
>SRR8554643_4328113_1|P
[soil metagenome]
MCESPQQRAFLELTHRLFNESLRPLVPLLYAARKGRHGPDYQQILSTIKSGQGASAQVEAVSSLAARPGSRGGKEWTDLAKELV
ARGQILFDRHALMPNVHKEFRRKVFEMAFQIILSHDAKMKQWRTDHAKWLREKAEWERSYPEYLAVRPVIQEFEAREGQAGKRR
GRWHRWLDFLVLYPQLAAWRGGEPTVRQLTQQELQEARRSRRRAVPNTIDAFFAKNPELKELHGLHGEYQARFVRPWAKRRHQD
GFFLPPTFTLPSADRHPAWYSFKRGDTYRNLSLEQRTIELRVLKSADPNESGRHAYVQYQFVGDNRLGKLSPASKIVRSGRFPC
DLLVWDEGSDHARPARVQGAKLVIRKGQAYLYFSVYIEDAPSRLPVKQAQIDKYSPGWSINRIRGEPYDPPLRTMAIDLGIRHI
AAATVMENGNVLATRFLHSRPVSRVSGHVIEGIPTLPQISEMKQRLRRARRKFGKPLKGTMSCRRIQRHVTSMSEDRCKKSASA
IVDFGRAHRVDIILMERLEGLVPDAARERRINCGLINWNRGHLASWIRIIAEPYGIRVLELPPRWTSQVCSHCGSIGARFTCTR
GEMKISAGGKLFGCPTCAFEANADFNASLNLQRVFWGLFPQVSKAGRGRVQCNDRVIELASVNQAWENHWLRKHNSANDVF
(SEQ ID NO: 355)
>SRR8554643_megahit_k177_1281129_3|P
[soil metagenome]
MATKTYYARFVNTTPRLRAALERSHYVYVECLRQMIERYIAMRKGKLGEECRQLAKIILTTGNTEARGIMNQITRPVVTSGKDY
EWVKLAKQVHKLRGPLLLQDEGFARVDGVTIRTKHHGKVEPTRGELAVTAKFWMQICDMASSFLKSNAELMDDWREERTKWLRE
KAEWEERNSDFLKFWNGPYTEFENLYERMRLEAQTAAGQTPTVRKRQSRERGKRIDRWHLWYEWVISHPEIMAWRNQARASDFK
VMPIELQNQIKRKHPRQNKYIPEFLKWLRENNAEFDRLDSLRRVYVNNYCRFKRPPTLTLPSPKKHPYWFTFERDEFYKDADFE
TGAIRLLLIDQKDDGAWFFEWFDAQMKCDPRLKPSVRAEHFNKEGRYPPYIDGKPARKLNRSAASPEQRRAGYTGAKLVLKGAR
QELLFTVIEQDCPPKVKWQKVKMRACRADNAFSAEGERIPLKIMAIDLGIRHTAAYVIAEGTLQDGQWHLSWQKKGIVRDIHIS
PLMNIRQHDWELREGRSRQGKAAKGERTFVDLQDHRTDMADDRFKKAANTIVETAREHDVRLILFEQLKNWFPKAFDERWMNRQ
LRDMNRRKIVEMVEGQSKEFGIICADNISPWLTSRICSRCYKPGWRFSIKGKDPYKEKLSRRHCQDFGYPIWDRGGGHLFRCSH
CGYRANADINAAGNVAAKFFGAWPVLSCKKGVYTWQQDGQKHTFDARETFESWAQDVRRRKQIAESPF (SEQ ID NO:
357)
>SRR8554643_megahit_k177_1281129_3|M
[soil metagenome]
MIERYIAMRKGKLGEECRQLAKIILTTGNTEARGIMNQITRPVVTSGKDYEWVKLAKQVHKLRGPLLLQDEGFARVDGVTIRTK
HHGKVEPTRGELAVTAKFWMQICDMASSFLKSNAELMDDWREERTKWLREKAEWEERNSDFLKFWNGPYTEFENLYERMRLEAQ
TAAGQTPTVRKRQSRERGKRIDRWHLWYEWVISHPEIMAWRNQARASDFKVMPIELQNQIKRKHPRQNKYIPEFLKWLRENNAE
FDRLDSLRRVYVNNYCRFKRPPTLTLPSPKKHPYWFTFERDEFYKDADFETGAIRLLLIDQKDDGAWFFEWFDAQMKCDPRLKP
SVRAEHFNKEGRYPPYIDGKPARKLNRSAASPEQRRAGYTGAKLVLKGARQELLFTVIEQDCPPKVKWQKVKMRACRADNAFSA
EGERIPLKIMAIDLGIRHTAAYVIAEGTLQDGQWHLSWQKKGIVRDIHISPLMNIRQHDWELREGRSRQGKAAKGERTFVDLQD
HRTDMADDRFKKAANTIVETAREHDVRLILFEQLKNWFPKAFDERWMNRQLRDMNRRKIVEMVEGQSKEFGIICADNISPWLTS
RICSRCYKPGWRFSIKGKDPYKEKLSRRHCQDFGYPIWDRGGGHLFRCSHCGYRANADINAAGNVAAKFFGAWPVLSCKKGVYT
WQQDGQKHTFDARETFESWAQDVRRRKQIAESPF (SEQ ID NO: 358)
>SRR6056591_megahit_k177_2733919_1|P
[subsurface metagenome]
MATKSYRSRIVCTKAELDDLVRTHRVFNAGVRAMVDLYISMKKGTYGETAKEIARYLLDHSGTKGHDLMNPLTDRGGKDTRDDE
GMKLVRRHRQEHGILFPRHERICEIDGRKVRAGSSAKKEKSRDTLTISSIFWHFICNQAIEYLRSNNELMAAWREERGKWLRDK
EKFFAENPGFREFVEGYLAAFDAHAEKARQEFQTKAGQRAHARKRKKSGSGKRFSRWHLWCEWICDHPEIIEWRGKAKGEGFAF
LTAEEVRAIEEEFPKREDRRAGRMLDMLTRKNPELAEVEKLRKKYNNDYARFRRPPTLTLPSAKKHPCWYRLEKGKLYRNEDFE
KGTIEILVICHDEKEDAYYMDWRPFSIKPEPRLVPSHRARAFAAEGRYPPYKEGKVGNTLNRPAESAEERLAGLKGAKLILKPK
GNAQLVFTVVEQNEPLKAKFRKKAGATSSADRLDILDGNKATRVMGIDLGARHVGAYVITEGKKKGDGWKLAPLRKGFIDSPDL
PRLRSITRHENQLKKGRRKRGKPVKNERSFISLQGHRTKMSTDRFKKAANLIVEKAREHDVHIIVFEDLRRLKPTAYDERWLNR
QVRSMNHRHIFDLAKEMAPEFGIQVTDINPYNTSRLCSKCGFPGYRFSMKRKNPYREQAPRRECADFGYPVWDSGGHLFRCPHC
GYRVQADLNAAGNIARKFFGHFMDWECKEYTYRNAVQPSFDARKDFDQWASGVLKRRTMPDAPF (SEQ ID NO: 324)
>SRR6263259_megahit_k177_232832_6|M
[terrestrial metagenome]
MAVKTLQSRLHCKPKKLEALWRTHEIFNERLKDVLSLLFRMRRGECGRSPDERELYREIALFITACPANNAPYLLNSVCIQNWV
PTTARKIKAIVSRSSGELIKGTEESWTTRAAQLSAKGHLLFDKREVLGDLTPAMAQVIVREAAAYLSNYESHVRRWEREHEQWL
GEKAAWEAEPEHQLYLASRPRFTAFEAGIGGRLTERRHRWAQYLTWLSQNPELAAWRGGLATINPIGEEGRKRIAHAPRWRKAQ
VEAEEFFKLNPELKDLDALHGYYERKFVRRRKTKRHIDGFDHKPTFTQPDALVHPHWYLFNGPQTKPAGYRKLILPKSSKQAGS
IELSLMTCDKNDPNDTAWVPFRFHGDCRMSNFRSVVVTSKIKSGGKRGQTKEKEGYEMYDPALKTWRPAEIKGARLIFTVKGKR
PRAAYLYFTIKTKNTPFSEAARNIEWSETGETSVFGPPRKTKLVPEGLVTCAAHIGARDCVYATLAVGDGSTPHIIRQRNLWIE
HIEATGQHVGRRHRGPSLAHLASHKMELSRRRSQMGRIPRGETAHEGLQSHITNMAKDRYRQQARRIFDFALNVEGVGDPRAGK
SYPRADVLILESMENLIPNVERERGVNKALVAFNRAHLVSHLKEIVAEAGVMVFEISPVGTSQVCSRCGALGRPYSVRRRSKDH
PVAITFGPAEPLFACPSCHYRANSSHNASVNLHHRFYSDQALESFITYLRQPVERKLENLRRIEAELISPIAGPLSLYEMHGIK
IG (SEQ ID NO: 359)
>3300031539|Ga0307380_10018846_3|P
[terrestrial-soil]
MATKTYYARFVNTTPRLRAALERSHYVYVECLRQMIERYIAMRKGKLGEECRQLAKIILTTGNTEARGIMNQITRPVVTSGKDY
EWVKLAKQVHKLRGPLLLQDEGFARVDGVTIRTKHHGKVEPTRGELAVTAKFWMQICDMASSFLKSNAELMDDWREERTKWLRE
KAEWEERNSDFLKFWNGPYTEFENLYERMRLEAQTAAGQTPTVRKRQSRERGKRIDRWHLWYEWVISHPEIMAWRNQARASDFK
VMPIELQNQIKRKHPRQNKYIPEFLKWLRENNAEFDRLDSLRRVYVNNYCRFKRPPTLTLPSPKKHPYWFTFERDEFYKDADFE
TGAIRLLLIDQKDDGAWFFEWFDAQMKCDPRLKPSVRAEHFNKEGRYPPYIDGKPARKLNRSAASPEQRRAGYTGAKLVLKGAR
QELLFTVIEQDCPPKVKWQKVKMRACRADNAFSAEGERIPLKIMAIDLGIRHTAAYVIAEGTLQDGQWHLSWQKKGIVRDIHIS
PLMNIRQHDWELREGRSRQGKAAKGERTFVDLQDHRTDMADDRFKKAANTIVETAREHDVRLILFEQLKNWFPKAFDERWMNRQ
LRDMNRRKIVEMVEGQSKEFGIICADNISPWLTSRICSRCYKPGWRFSIKGKDPYKEKLSRRHCQDFGYPIWDRGGGHLFRCSH
CGYRANADINAAGNVAAKFFGAWPVLSCKKGVYTWQQDGQKHTFDARETFESWAQDVRRRKQIAESPF (SEQ ID NO:
357)
>3300031539|Ga0307380_10018846_4|M
[terrestrial-soil]
MIERYIAMRKGKLGEECRQLAKIILTTGNTEARGIMNQITRPVVTSGKDYEWVKLAKQVHKLRGPLLLQDEGFARVDGVTIRTK
HHGKVEPTRGELAVTAKFWMQICDMASSFLKSNAELMDDWREERTKWLREKAEWEERNSDFLKFWNGPYTEFENLYERMRLEAQ
TAAGQTPTVRKRQSRERGKRIDRWHLWYEWVISHPEIMAWRNQARASDFKVMPIELQNQIKRKHPRQNKYIPEFLKWLRENNAE
FDRLDSLRRVYVNNYCRFKRPPTLTLPSPKKHPYWFTFERDEFYKDADFETGAIRLLLIDQKDDGAWFFEWFDAQMKCDPRLKP
SVRAEHFNKEGRYPPYIDGKPARKLNRSAASPEQRRAGYTGAKLVLKGARQELLFTVIEQDCPPKVKWQKVKMRACRADNAFSA
EGERIPLKIMAIDLGIRHTAAYVIAEGTLQDGQWHLSWQKKGIVRDIHISPLMNIRQHDWELREGRSRQGKAAKGERTFVDLQD
HRTDMADDRFKKAANTIVETAREHDVRLILFEQLKNWFPKAFDERWMNRQLRDMNRRKIVEMVEGQSKEFGIICADNISPWLTS
RICSRCYKPGWRFSIKGKDPYKEKLSRRHCQDFGYPIWDRGGGHLFRCSHCGYRANADINAAGNVAAKFFGAWPVLSCKKGVYT
WQQDGQKHTFDARETFESWAQDVRRRKQIAESPF (SEQ ID NO: 358)
>3300031539|Ga0307380_10072822_2|M
[terrestrial-soil]
MCESPQQRAFLELTHRLFNESLRPLVPLLYAARKGRHGPDYQQILSTIKSGQGASAQVEAVSSLAARPGSRGGKEWTDLAKELV
ARGQILFDRHALMPNVHKEFRRKVFEMAFQIILSHDAKMKQWRTDHAKWLREKAEWERSYPEYLAVRPVIQEFEAREGQAGKRR
GRWHRWLDFLVLYPQLAAWRGGEPTVRQLTQQELQEARRSRRRAVPNTIDAFFAKNPELKELHGLHGEYQARFVRPWAKRRHQD
GFFLPPTFTLPSADRHPAWYSFKRGDTYRNLSLEQRTIELRVLKSADPNESGRHAYVQYQFVGDNRLGKLSPASKIVRSGRFPC
DLLVWDEGSDHARPARVQGAKLVIRKGQAYLYFSVYIEDAPSRLPVKQAQIDKYSPGWSINRIRGEPYDPPLRTMAIDLGIRHI
AAATVMENGNVLATRFLHSRPVSRVSGHVIEGIPTLPQISEMKQRLRRARRKFGKPLKGTMSCRRIQRHVTSMSEDRCKKSASA
IVDFGRAHRVDIILMERLEGLVPDAARERRINCGLINWNRGHLASWIRIIAEPYGIRVLELPPRWTSQVCSHCGSIGARFTCTR
GEMKISAGGKLFGCPTCAFEANADFNASLNLQRVFWGLFPQVSKAGRGRVQCNDRVIELASVNQAWENHWLRKHNSANDVF
(SEQ ID NO: 355)
>3300031539|Ga0307380_10072822_2|P
[terrestrial-soil]
MIQGTTSCASPTIAVKCFQAKLVCESPQQRAFLELTHRLFNESLRPLVPLLYAARKGRHGPDYQQILSTIKSGQGASAQVEAVS
SLAARPGSRGGKEWTDLAKELVARGQILFDRHALMPNVHKEFRRKVFEMAFQIILSHDAKMKQWRTDHAKWLREKAEWERSYPE
YLAVRPVIQEFEAREGQAGKRRGRWHRWLDFLVLYPQLAAWRGGEPTVRQLTQQELQEARRSRRRAVPNTIDAFFAKNPELKEL
HGLHGEYQARFVRPWAKRRHQDGFFLPPTFTLPSADRHPAWYSFKRGDTYRNLSLEQRTIELRVLKSADPNESGRHAYVQYQFV
GDNRLGKLSPASKIVRSGRFPCDLLVWDEGSDHARPARVQGAKLVIRKGQAYLYFSVYIEDAPSRLPVKQAQIDKYSPGWSINR
IRGEPYDPPLRTMAIDLGIRHIAAATVMENGNVLATRFLHSRPVSRVSGHVIEGIPTLPQISEMKQRLRRARRKFGKPLKGTMS
CRRIQRHVTSMSEDRCKKSASAIVDFGRAHRVDIILMERLEGLVPDAARERRINCGLINWNRGHLASWIRIIAEPYGIRVLELP
PRWTSQVCSHCGSIGARFTCTRGEMKISAGGKLFGCPTCAFEANADFNASLNLQRVFWGLFPQVSKAGRGRVQCNDRVIELASV
NQAWENHWLRKHNSANDVF (SEQ ID NO: 360)
>3300031566|Ga0307378_10072489_1|M
[terrestrial-soil]
MCESPQQRAFLELTHRLFNESLRPLVPLLYAARKGRHGPDYQQILSTIKSGQGASAQVEAVSSLAARPGSRGGKEWTDLAKELV
ARGQILFDRHALMPNVHKEFRRKVFEMAFQIILSHDAKMKQWRTDHAKWLREKAEWERSYPEYLAVRPVIQEFEAREGQAGKRR
GRWHRWLDFLVLYPQLAAWRGGEPTVRQLTQQELQEARRSRRRAVPNTIDAFFAKNPELKELHGLHGEYQARFVRPWAKRRHQD
GFFLPPTFTLPSADRHPAWYSFKRGDTYRNLSLEQRTIELRVLKSADPNESGRHAYVQYQFVGDNRLGKLSPASKIVRSGRFPC
DLLVWDEGSDHARPARVQGAKLVIRKGQAYLYFSVYIEDAPSRLPVKQAQIDKYSPGWSINRIRGEPYDPPLRTMAIDLGIRHI
AAATVMENGNVLATRFLHSRPVSRVSGHVIEGIPTLPQISEMKQRLRRARRKFGKPLKGTMSCRRIQRHVTSMSEDRCKKSASA
IVDFGRAHRVDIILMERLEGLVPDAARERRINCGLINWNRGHLASWIRIIAEPYGIRVLELPPRWTSQVCSHCGSIGARFTCTR
GEMKISAGGKLFGCPTCAFEANADFNASLNLQRVFWGLFPQVSKAGRGRVQCNDRVIELASVNQAWENHWLRKHNSANDVF
(SEQ ID NO: 355)
>3300031566|Ga0307378_10072489_1|M
[terrestrial-soil]
MIQGTTSCASPTIAVKCFQAKLVCESPQQRAFLELTHRLFNESLRPLVPLLYAARKGRHGPDYQQILSTIKSGQGASAQVEAVS
SLAARPGSRGGKEWTDLAKELVARGQILFDRHALMPNVHKEFRRKVFEMAFQIILSHDAKMKQWRTDHAKWLREKAEWERSYPE
YLAVRPVIQEFEAREGQAGKRRGRWHRWLDFLVLYPQLAAWRGGEPTVRQLTQQELQEARRSRRRAVPNTIDAFFAKNPELKEL
HGLHGEYQARFVRPWAKRRHQDGFFLPPTFTLPSADRHPAWYSFKRGDTYRNLSLEQRTIELRVLKSADPNESGRHAYVQYQFV
GDNRLGKLSPASKIVRSGRFPCDLLVWDEGSDHARPARVQGAKLVIRKGQAYLYFSVYIEDAPSRLPVKQAQIDKYSPGWSINR
IRGEPYDPPLRTMAIDLGIRHIAAATVMENGNVLATRFLHSRPVSRVSGHVIEGIPTLPQISEMKQRLRRARRKFGKPLKGTMS
CRRIQRHVTSMSEDRCKKSASAIVDFGRAHRVDIILMERLEGLVPDAARERRINCGLINWNRGHLASWIRIIAEPYGIRVLELP
PRWTSQVCSHCGSIGARFTCTRGEMKISAGGKLFGCPTCAFEANADFNASLNLQRVFWGLFPQVSKAGRGRVQCNDRVIELASV
NQAWENHWLRKHNSANDVF (SEQ ID NO: 360)
>3300031539|Ga0307380_10070813_2|P
[terrestrial-soil]
MAVRAIQVRLESDKTLLEHLWRTHKVFNERLPMIISILFKMRRGECGTTDEERTLYKNIARFVLARSSKDAPYLLNSISIKDWT
PSTARKMKATITIPDGSEQEVCGDTWADKVAELSAAGRLLYSKQEVLGDLPDSLRQMIVRECVAIISGHDELVRNWDKSHTEWL
KKREEWEAKEEHKKYLAVRSRFDEFEKSVGGKAGKRRARWHKYLQWLRESPDLAGWRGSLAVVNELSREAQERVRKAKPWRQRG
VEAEEFWKANPELQALDRLHGQYERDFVRRGKTKRNPDGFEHRPTFTLPHFLRHPRWLVFNAPQTSPPGYRNLKLPGKAGQYGE
VELRLLTNEIRGAEFPIDWTTLRFLGDPRLADFKPVKIQKAATKGKVKGELRGRPAYRFFDRELQMERPAQISGAKLIFKNVRL
DDGPLQSATPYLYFACEAKSLRWTENARKVKWNATGEVTKKGKKRGYRTLPDGLIACAVDLGIRNLGFATLARYENGVPRLLRS
RNLWIGHAETRGRHPGRWCEGPELAHLALHKRELRRLRSLRGDPIAGEESHIELQQHITHFAEDRFKKAARAIVNFALNVKEFA
DKNTGEIYPRADVLVLENLGSLLPDAEREKGINRSLIEFNRGHLVDRVKEVAKDVGLRVFEVSPVGTSQVCSRCGTLGRRYSIR
RSAETHRPEIQFGFVEKLFACPECGYRANADHNASVNLHRRLAQVPGAFAQWEELGKDQQRKRKRWEEIEAKLLPSLQKMHGFD
GAGKPHPFLSA (SEQ ID NO: 354)
>3300031965|Ga0326597_10006968_2|P
[terrestrial-soil]
MGVKAWKVKLFVDAPEKQAVLDDTLQQWNLYAAHVCKVLLAFRRNDYGDVGLEVWRQLAKTPQNYHVLYCITPRSDAAPDQGPR
VTREKDYHRAARAFVQRHGYEPELAAVRRDAGPLWKQIVKAARERVLSYEQKVADWREAREQWIVQRREWEGDNPPYMAVRPVI
EAFQRDHGATRGRRVRWRKWQAFLQSTPELAAWRGGAACVEPLSAQELRHAARNRRKAARRERELFLDKNPELAELDRVHFQYE
RLFARTAAKRRNPDGFRHRPTFTLPSLPNRPDWPRFQAMEGYKNLDITSRSVQLRLPTSDTRHWLSLSFVPDPRLARLVPLERS
VKEGRTRFDYRFPNRPGEFVLAQPQEIRLIRRGSSYFATIRLTLQPTPSPLPLTQTAAGKYSPQWVIRKIRDDAPDLSLVTCAV
DLAVRHLGAASVAREGRVFARRILHNRFFRPNDLRERALNIPSLPEIAAVRRRIRRAQRRSGRMSPAKDTCRRLWTHYRNLCED
HYKKAVAAIFAYALHHGAHLVIFEDLKMLNPDSANERGVNNALQNWNRGNIVRFAKMAAEDHGLRVVTVGAWWTSRLCARCGGM
GVRFNQGKRRPWGQRRHTAPRPVPIDRLTCNIAPLGHWFLCPGCRSCLHADINASENLHRQFLGSLPTVKRLGSSRPPIYEVNG
QRVEQARLEAAANELLLARTAPATPF (SEQ ID NO: 361)
>3300017540|Ga180217_1000254_36|M
[terrestrial-soil-compost]
MGTKAWKAKLILDSEDARAALLHTFSVWNAACAEVCEALLAFRRGEFGEEGRRVWTYLRDAGTQGYYILWCITKAPRPREVKEL
HRIAADFVARHGFHQGLLAVRERLPSALWKQVIHAARERVASYEEKVAIWNEDHAQWLADRAEWEAEHASYMAVRPLIEQFQSQ
FGATRGRRARWHAWLTFLGSTPALQSWRGGPPGVVSLTSGERQAARRNPRKAVAREAELFYAKNPELAELNRIHGEYERRFARS
WAKRKNPDGFRHRPTFTLPNEKSHPDWPRLQANEGWKALDPAARTIRLQLIAPDGTARLYPFRFVPDPRLSRLTPLAEPLKEGR
ITYAYSVPGPQGRPTPAQPQGIKVILRGDSAFISISLSLLPPPSAIPVTQAAMTKYPPTWALKQIREKAPELELVTCAVDLGVR
HIGAATIAREGEVIARRILHHRFFPPGGDPARPINIPSLAEIAEVKRQLRRARRRSGKPAPGKESCRRLQAHYRGLCEDRYKKV
VAAIFEFAAAHGAHIVIFEDLRMLNPDSANERGVNAALMNWNRGSIVRFARATAEDRGLRVATVGAWWTSRICCRCDALGVRFE
QGRRREWARAPVASPAPPDRTACTISRLGQWFFCPACRRRVHADINASENLHRVFLGTFPKVERVGREPPAYEVGGQHVDFREI
QRAAAAILSAPATPF (SEQ ID NO: 362)
>3300014494|Ga0182017_10000840_9|P
[terrestrial-soil-fen]
MGVRALRARIECDRETLLHLWRTHCAFNQRLLSILTKLFGMRRGELGETAARRALYQRVARFLLARDSKDAPYLLNSVSIQGWK
PATALKMKVRMPAPDGTLAEVSADTWAPEAAKLSAHGTLAYDKEAVRDGLPDSLFQPMLRDAVAYISGHIELARNWEAEHADWL
KAKAEWEEKDERKKYLALRPRFDAFEQSVGGKAGKRRERWHLYLTWLRENPALAAWRGGPPAVHPLDAKATARVAKAKPWKKRS
VEADEFWKANPELRELDKLHGYYEREFVRRRKTKKNPDGFDHRPTFTLPHAVLHPRWTLFNAPQTSPQGYADLRLPQRPGTLGS
VKLRLLTGEKAPDGCPNEWVGVQFRADPRLSQFAERAEAQEIRKGKKKGTQKTVRVFRFVDQQLNCERPAQISGAKLIFKNVKL
NDDGSLKSATPYLVFACTIDNLPLTEHARAIQWSETGEVTKTGKKRKAKKLPTGLVTCAVDLGLRNLAFLTLATSGEGPQNNTH
DGIRVLRSRNQWLALEETAGTHPGRWSAGPDLAHIAAHKREIRRLRRLRGKPVKGQQSHVELQGHIDHMGQDRFKKAARAIVNF
ALNVDRAAGQSGQPYPRADLLIVEKLEGFIPDAARERGINRALVAWNRGQLVTRIKEVAADAGLKLFPVHPAGTSQCCSRCGAL
GRRYSIVRDQQTGVPAIRFGWVEKLFACPNRECPGRAPDKPDQPFTCNADHNASLNLHRRFVLDDRAVAAFFAWKNKTNAERER
ELTQMDQALRPELEKLHRLPLAELATPF (SEQ ID NO: 343)
>3300014839|Ga0182027_10004505_2|P
[terrestrial-soil-fen]
MFNRRLPDLISLLFRMRRGECGETEQASRLYSQVASFILARGSKDAPYLLNSVSVRGWKPHTAKKMKARALGPDGQQIELAGED
WADPAAALSAEGRLLYDKTELLGDVPDTLRQMAARECVAIISGHEELVRAWEKAHEEWLASKAKWESDPENQFYLGLRARFDEF
EQSVGGKVGKRRGRWQRYLDWLRANPDLAAWRGGPAAVVELSAGAKERIRKAKPRKVRSVEAEEFWKVNSELAALDRLHGYYER
EFVRRRKTKKHPDGFDHRPTFTLPHPVRHPRWFVFNAPQTSPQGYRKLCLPSSGGHTGTVELLLLTGEKADNKYPSGWETVRFA
ADPRLSDFKAITKRRQVLKGKTKGQETDTKAFTFLDRQINLERPAQISGVKLIFKGVRQNHDGSLLSAHPYLVFTCNVADVPLT
DGAKKIKWEDTGERRKRVVVPAGLIACAVDLGIRHVGFATVATQDAESGGLHVLRSRNIWIGQEEEKGRNPGRWSPGPDLAHLS
AHKRSLRHLRGLRGKPVKGENSHIELQDHITNMASDRFKKAAREIVNFALNADSKVDPRTGEPYPRADVLVLENLANLLPDSER
ERGINRALIEFNRGHLADRIVEVAKDCGLKVMMVSPVGTSQVCCRCGALGRRYSLRRHQDGGYTDIHFGFVEPLFACPSCGYRA
NSDHNASVNLHRRFRLGEAAIAKFKEWQKGTKPERDEVLRRIEQSLVGPLREMHRLAVADTPF (SEQ ID NO: 346)
>3300031715|Ga0307476_10004505_2|P
[terrestrial-soil-fen]
MSTRALQSRIQCDHQTLEHLWRTHLVFNRRLPDLISLLFRMRRGECGETEQASRLYSQVASFILARGSKDAPYLLNSVSVRGWK
PHTAKKMKARALGPDGQQIELAGEDWADPAAALSAEGRLLYDKTELLGDVPDTLRQMAARECVAIISGHEELVRAWEKAHEEWL
ASKAKWESDPENQFYLGLRARFDEFEQSVGGKVGKRRGRWQRYLDWLRANPDLAAWRGGPAAVVELSAGAKERIRKAKPRKVRS
VEAEEFWKVNSELAALDRLHGYYEREFVRRRKTKKHPDGFDHRPTFTLPHPVRHPRWFVFNAPQTSPQGYRKLCLPSSGGHTGT
VELLLLTGEKADNKYPSGWETVRFAADPRLSDFKAITKRRQVLKGKTKGQETDTKAFTFLDRQINLERPAQISGVKLIFKGVRQ
NHDGSLLSAHPYLVFTCNVADVPLTDGAKKIKWEDTGERRKRVVVPAGLIACAVDLGIRHVGFATVATQDAESGGLHVLRSRNI
WIGQEEEKGRNPGRWSPGPDLAHLSAHKRSLRHLRGLRGKPVKGENSHIELQDHITNMASDRFKKAAREIVNFALNADSKVDPR
TGEPYPRADVLVLENLANLLPDSERERGINRALIEFNRGHLADRIVEVAKDCGLKVMMVSPVGTSQVCCRCGALGRRYSLRRHQ
DGGYTDIHFGFVEPLFACPSCGYRANSDHNASVNLHRRFRLGEAAIAKFKEWQKGTKPERDEVLRRIEQSLVGPLREMHRLAVA
DTPF (SEQ ID NO: 347)
>3300031715_Ga0307476_10010105_2|P
[terrestrial-soil-hardwood forest soil]
MSVRAIQARINCDRQTLLDLWRTHCVFNQRLPSILSVLFKMRRGECGDSPELQDLYQRISRFIIASSSQLADALLNSVSIRNWK
PDSARKRKIKVVSETGEIVEVTGETWADAAAAISKQGQLLYDKRALLFDLPNSMRQIVSREAVTIISGHDELTAIWHKEHAAWS
KQKAEWQNGAENQRYLALRPKFAAFEEEVGGAITKRRGRWHLYLQWLPERPELAAWRGGPAIVRKLDHAALLRVQRSRPSKQRT
VEAEEFWKINPELKVLDRLHGDYESRFVRRRKTKKNSDGFDHRPTFTLPHPVNHPRWFVFNAPQTSPQGYGDLKLPAEPNGFGT
ISLRLLTGEKSEGGYPSNWRTVRFKADPRLADFRQVSVKYRTNKGASKGTEREKRGYIFVDRQLKLERKATISGAKLVFRDIRL
NPDGSLSSATPYLIFTCNIDDELMTERAKAIKWSDTGEITKKGKPRKKKTLPSSLVSCAIDLGHRHLGFATIAQAEETGLRILR
SRNLWIGHKETTGHHPGRWCEGPDLAHLGQHKRKLRALRRLRGKPVKDERTHVELQGHIDDMAEDRFKKGARAIINFALNTDNR
IDEKTGKPIPRADVLLLENLENLIPDAERERGINRMLVQFNRGNLVKRVKEMAADAGLKVFEVSPFGTSQVCSKCGELGRRYSI
RRQQVPEDPTQTRPDIHFGLVEKLFACPKCNYRANADHNASVNLHRRFHNDGLALKSFAEFRALSDQDRLRSIEQIDAKLLEPL
RELHNVGPVTPF (SEQ ID NO: 363)
>3300031715|Ga0307476_10010105_1|M
[terrestrial-soil-hardwood forest soil]
MADALLNSVSIRNWKPDSARKRKIKVVSETGEIVEVTGETWADAAAAISKQGQLLYDKRALLFDLPNSMRQIVSREAVTIISGH
DELTAIWHKEHAAWSKQKAEWQNGAENQRYLALRPKFAAFEEEVGGAITKRRGRWHLYLQWLPERPELAAWRGGPAIVRKLDHA
ALLRVQRSRPSKQRTVEAEEFWKINPELKVLDRLHGDYESRFVRRRKTKKNSDGFDHRPTFTLPHPVNHPRWFVFNAPQTSPQG
YGDLKLPAEPNGFGTISLRLLTGEKSEGGYPSNWRTVRFKADPRLADFRQVSVKYRTNKGASKGTEREKRGYIFVDRQLKLERK
ATISGAKLVFRDIRLNPDGSLSSATPYLIFTCNIDDELMTERAKAIKWSDTGEITKKGKPRKKKTLPSSLVSCAIDLGHRHLGF
ATIAQAEETGLRILRSRNLWIGHKETTGHHPGRWCEGPDLAHLGQHKRKLRALRRLRGKPVKDERTHVELQGHIDDMAEDRFKK
GARAIINFALNTDNRIDEKTGKPIPRADVLLLENLENLIPDAERERGINRMLVQFNRGNLVKRVKEMAADAGLKVFEVSPFGTS
QVCSKCGELGRRYSIRRQQVPEDPTQTRPDIHFGLVEKLFACPKCNYRANADHNASVNLHRRFHNDGLALKSFAEFRALSDQDR
LRSIEQIDAKLLEPLRELHNVGPVTPF (SEQ ID NO: 364)

TABLE 13
Nucleotide sequences of Representative CLUST.089537 Direct Repeats

| Effector Protein Accession | Direct Repeat Nucleotide Sequence |
|---|---|
| CAAACX010000652_28\|M (SEQ ID NO: | CGTGCATCGGCCGACGAAGAGCGAAGGTCAGTGACAG (SEQ ID NO: |
| 301) | 401) |
| CAAACH010011569_2\|M (SEQ ID NO: | CGCCGCAGCGGCCGACGCGGCCCTGATCGATGGACAC (SEQ ID NO: |
| 302) | 402) |
| CAAACI010012331_3\|M (SEQ ID NO: 303) | GCCGCAGCGGCCGACGCGGCCCTGATCGATGGACAC (SEQ ID NO: 403) |
| UZXW01002767_11\|M (SEQ ID NO: 304) | GGAGCGCCCCCCGACGCGGCAATGATCGGCTGACAC (SEQ ID NO: 404) |
| 3300009161\|Ga0114966_10008307_8\|M | CTCTCAATGGCTAAAGATCTAGTTCTAATTGGACAC (SEQ ID NO: 405) |
| (SEQ ID NO: 305) | |
| 3300009161\|Ga0114966_10008307_7\|P | CTCTCAATGGCTAAAGATCTAGTTCTAATTGGACAC (SEQ ID NO: 405) |
| (SEQ ID NO: 306) | |
| 3300010885\|Ga0133913_10083255_2\|P | CTCTCAATGGCTAAAGATCTAGTTCTAATTGGACAC (SEQ ID NO: 405) |
| (SEQ ID NO: 306) | |
| 3300020109\|Ga0194112_10008260_1\|P | CCCCCCCCGCCATTCTCGGCTGTGTACAAC (SEQ ID NO: 406) |
| (SEQ ID NO: 307) | |
| 3300020109\|Ga0194112_10008260_1\|M | CCCCCCCCGCCATTCTCGGCTGTGTACAAC (SEQ ID NO: 406) |
| (SEQ ID NO: 308) | |
| 3300020222\|Ga0194125_10021011_3\|M | CGGGTGTACCCCCCCCGCCATTCTCGGCTGTGTACAAC (SEQ ID NO: |
| (SEQ ID NO: 309) | 407) |
| 3300027770\|Ga0209086_10006474_1\|P | TCTCTCAATGGCTAAAGATCTAGTTCTAATTGGACACC (SEQ ID NO: |
| (SEQ ID NO: 306) | 408) |
| 3300015360\|Ga0163144_10184575_1\|M | GCGGCGCTGGCCAGCGCCGCGCTGAA (SEQ ID NO: 409) |
| (SEQ ID NO: 310) | |
| 3300013130\|Ga0172363_10020541_1\|M | GTTGCACCTCGCGCGCACCTGCGGGTTGACAC (SEQ ID NO: 410) |
| (SEQ ID NO: 311) | |
| 3300013133\|Ga0172362_10044703_1\|M | GGCGCACCGCGTGGCGTGGAACTGACCGGCTGACAC (SEQ ID NO: 411) |
| (SEQ ID NO: 312) | |
| 3300031862\|Ga0315280_10005430_2\|M | CCTGTATCCGCGCGCGCGTTTGCGGCTGATGAACAC (SEQ ID NO: 412) |
| (SEQ ID NO: 313) | |
| 3300031952\|Ga0315294_10035890_2\|P | GTATTCATCCCCTTCAGAAAAGGAACTCTCTGGACAC (SEQ ID NO: |
| (SEQ ID NO: 314) | 413) |
| 3300031952\|Ga0315294_10041214_2\|M | GGCGCACCCGCTGGCGCGGAACAGACCGACTGACAC (SEQ ID NO: 414) |
| (SEQ ID NO: 315) | |
| 3300031952\|Ga0315294_10041214_2\|P | GGCGCACCCGCTGGCGCGGAACAGACCGACTGACAC (SEQ ID NO: 414) |
| (SEQ ID NO: 316) | |
| 3300031952\|Ga0315294_10058002_1\|M | CTCTCAACCGCTTGGGCAGAACCGACCGACTGACAC (SEQ ID NO: 415) |
| (SEQ ID NO: 317) | |
| 3300032046\|Ga0315289_10071835_2\|M | CTGTCCCCCCCGGACCTGACCAATAAGGGATTGACAC (SEQ ID NO: |
| (SEQ ID NO: 318) | 416) |
| 3300032053\|Ga0315284_10046226_2\|P | GTGACATCTTATGCGTGTGTACAGGTGGACAC (SEQ ID NO: 417) |
| (SEQ ID NO: 319) | |
| 3300032069\|Ga0315282_10002412_8\|P | CCTGTATCCGCGCGCGCGTTTGCGGCTGATGAACAC (SEQ ID NO: 412) |
| (SEQ ID NO: 320) | |
| 3300032118\|Ga0315277_10001015_12\|P | GAAGAAAAGACCTCTCAAAAAAAATTCTCCTGGACAC (SEQ ID NO: |
| (SEQ ID NO: 321) | 418) |
| 3300032118\|Ga0315277_10012802_3\|M | GTCGCGGCGGCCGATGCGATCCTGATCGATGGACAC (SEQ ID NO: 419) |
| (SEQ ID NO: 322) | |
| 3300009499\|Ga0114930_10003500_21\|M | GTGTGTTTCCTCTTAGAAAAACAAATGCTCTGGACAC (SEQ ID NO: |
| (SEQ ID NO: 323) | 420) |
| 3300011118\|Ga0114922_10000510_27\|M | GGGGGGCAAGCTCGCGCGTATGCACGGATTGGCAG (SEQ ID NO: 421) |
| (SEQ ID NO: 324) | |
| 3300017960\|Ga0180429_10052609_1\|M | GGTGGACGTCCCCCGCGATTCCCGGGTGTGTACCACGA (SEQ ID NO: |
| (SEQ ID NO: 325) | 422) |
| 3300017963\|Ga0180437_10045654_1\|M | GTTGCAATCTGTGCGTATGTATGGGTGGACAC (SEQ ID NO: 423) |
| (SEQ ID NO: 326) | |
| 3300017971\|Ga0180438_10003700_2\|P | GTTGCAATCTGTGCGTATGTATGGGTGGACAC (SEQ ID NO: 423) |
| (SEQ ID NO: 326) | |
| 3300017991\|Ga0180434_10065880_1\|P | ACTATAGGTACCCTAAAAATTCCGGGTAACTACACC (SEQ ID NO: 424) |
| (SEQ ID NO: 327) | |
| 3300017992\|Ga0180435_10008274_7\|P | GATGTAGCCGCACGTTGATTCACCGGCGATTACCAC (SEQ ID NO: 425) |
| (SEQ ID NO: 328) | |
| 3300018080\|Ga0180433_10080815_1\|M | ACTATAGGTACCCTAAAAATTCCGGGTAACTACACC (SEQ ID NO: 424) |
| (SEQ ID NO: 329) | |
| 3300009503\|Ga0123519_10002165_1\|M | GAAGAAAAAACCTCTTAAAAAAATTCTCCTGGACAC (SEQ ID NO: 426) |
| (SEQ ID NO: 330) | |
| CAAACB010000011_94\|M (SEQ ID NO: | GTTGCAGTTCGCGCGTAGGCGTGAGCGGACAC (SEQ ID NO: 427) |
| 331) | |
| SRR5251193_megahit_k177_201695_120\|P | GTCGCGATCCGCACACCCGAGCGAGTCGACAG (SEQ ID NO: 428) |
| (SEQ ID NO: 332) | |
| SRR5251487_megahit_k177_199995_1\|P | GTCGCGATCCGCACACCCGAGCGAGTCGACAG (SEQ ID NO: 428) |
| (SEQ ID NO: 332) | |
| SRR5576005_megahit_k177_1292294_1\|M | GTTGCAGTTCGCGCGTAGGCGTGAGCGGACAC (SEQ ID NO: 427) |
| (SEQ ID NO: 331) | |
| UOUK01008448_2\|M (SEQ ID NO: 333) | GCCGCCGCAGCGGCCGACGCGGCCCTGATCGATGGACAC (SEQ ID NO: |
| | 429) |
| UOUL01003058_1\|M (SEQ ID NO: 334) | GCCGCAGCGGCCGACGCGGCCCTGA (SEQ ID NO: 430) |
| 3300009652\|Ga0123330_1010394_3\|M | GCCGCAGCGGCCGACGCGGCCCTGA (SEQ ID NO: 430) |
| (SEQ ID NO: 335) | |
| 3300012949\|Ga0153798_10025413_2\|P | GTCGCGATCCGCACACCCGAGCGAGTCGACAG (SEQ ID NO: 428) |
| (SEQ ID NO: 336) | |
| SRR7811943_megahit_k177_406423_2\|M | GCCGCGGAGGCCGACGCGGCCCTGATGGATGGACAC (SEQ ID NO: 431) |
| (SEQ ID NO: 337) | |
| SRR5818194_megahit_k177_802550_7\|M | CTCTCAATGGCTAAAGATCTAGTTCTAATTGGACAC (SEQ ID NO: 405) |
| (SEQ ID NO: 305) | |
| SRR5818194_megahit_k177_802550_6\|P | CTCTCAATGGCTAAAGATCTAGTTCTAATTGGACAC (SEQ ID NO: 405) |
| (SEQ ID NO: 306) | |
| SRR6481359_megahit_k177_1860565_4\|M | ATAGCGCCCCCCGACGCAAATCTGATTAATGGACAC (SEQ ID NO: 432) |
| (SEQ ID NO: 338) | |
| SRR6049666_megahit_k177_256604_1\|P | GAAGAAAAAACCTCTTAAAAAAATTCTCCTGGACAC (SEQ ID NO: 426) |
| (SEQ ID NO: 330) | |
| 5RR2080392_megahit_k177_207267_1\|M | GTCGCAATGAGATAAAACATAGATATGTGTTGCAAC (SEQ ID NO: 433) |
| (SEQ ID NO: 339) | |
| SRR2080392_megahit_k177_207267_2\|P | GTCGCAATGAGATAAAACATAGATATGTGTTGCAAC (SEQ ID NO: 433) |
| (SEQ ID NO: 340) | |
| 3300025517\|Ga0207869_1001046_11\|M | GTGCATCGGCCGACGAAGAGCGAAGGTCAGTGACAG (SEQ ID NO: 434) |
| (SEQ ID NO: 301) | |
| 3300025572\|Ga0207864_1000213_47\|M | CGTGCATCGGCCGACGAAGAGCGAAGGTCAGTGACAG (SEQ ID NO: |
| (SEQ ID NO: 341) | 401) |
| 3300025572\|Ga0207864_1014515_2\|P (SEQ | GGCGTCGCGATCCGCACACCCGAGCGAGTCGACAGCA (SEQ ID NO: |
| ID NO: 332) | 435) |
| 3300025572\|Ga0207864_1014515_3\|M | GGCGTCGCGATCCGCACACCCGAGCGAGTCGACAGCA (SEQ ID NO: |
| (SEQ ID NO: 342) | 435) |
| SRR6505097_megahit_k177_377355_35\|M | GCCGCGTCCCCCGACGCGACCCTGATCGATGGACAC (SEQ ID NO: 436) |
| (SEQ ID NO: 343) | |
| SRR6962291_megahit_k177_1754533_5\|M | GGCGCAGCGGCCATCAGGATGGTGATCGGCTGACAC (SEQ ID NO: 437) |
| (SEQ ID NO: 344) | |
| SRR6962291_megahit_k177_1754533_5\|P | GGCGCAGCGGCCATCAGGATGGTGATCGGCTGACAC (SEQ ID NO: 437) |
| (SEQ ID NO: 345) | |
| SRR6962291_megahit_k177_3367137_15\|M | GGTGAAGCGGCCATCGGGATCGTGATCGGCTGACAC (SEQ ID NO: 438) |
| (SEQ ID NO: 346) | |
| SRR6962291_megahit_k177_3367137_14\|P | GGTGAAGCGGCCATCGGGATCGTGATCGGCTGACAC (SEQ ID NO: 438) |
| (SEQ ID NO: 347) | |
| AQT69685.1 (SEQ ID NO: 348) | GGCGCAACCCGCACACAACCGCGAATGGACAC (SEQ ID NO: 439) |
| PMBN01000097_4\|M (SEQ ID NO: 343) | GCCGCGTCCCCCGACGCGCCCCTGATCGAT (SEQ ID NO: 440) |
| PMDC01000375_4\|M (SEQ ID NO: 343) | GCCGCGTCCCCCGACGCGACCCTGATCGATGGACAC (SEQ ID NO: 436) |
| PMMM01000072_9\|M (SEQ ID NO: 343) | GCCGCGTCCCCCGACGCGACCCTGATCGATGGACAC (SEQ ID NO: 436) |
| QBM02859.1 (SEQ ID NO: 349) | ATTGCACCGGCCAACGCAAATCTGATTGATGGACAC (SEQ ID NO: 441) |
| SRR6963308_megahit_k177_1876783_1\|M | GGTGGACGTCCCCCGCGATTCCCGGGTGTGTACCAC (SEQ ID NO: 442) |
| (SEQ ID NO: 325) | |
| SRR6963352_megahit_k177_1225001_5\|P | TATAGGTACCCTAAAAATTCCGGGTAACTACACC (SEQ ID NO: 443) |
| (SEQ ID NO: 329) | |
| SRR6963480_megahit_k177_1813084_1\|M | ACTATAGGTACCCTAAAAATTCCGGGTAACTACACC (SEQ ID NO: 424) |
| (SEQ ID NO: 329) | |
| SRR6963491_megahit_k177_4304123_3\|P | GATGTAGCCGCACGTTGATTCACCGGCGATTACCAC (SEQ ID NO: 425) |
| (SEQ ID NO: 328) | |
| SRR6963587_megahit_k177_2272343_29\|M | GTTGCAATCTGTGCGTATGTATGGGTGGACAC (SEQ ID NO: 423) |
| (SEQ ID NO: 326) | |
| SRR6963591_megahit_k177_1621237_2\|P | GTTGCAATCTGTGCGTATGTATGGGTGGACAC (SEQ ID NO: 423) |
| (SEQ ID NO: 326) | |
| SRR8554444_megahit_k177_608234_1\|M | GAAGAAAAAACCTCTTAAAAAAATTCTCCTGGACACT (SEQ ID NO: |
| (SEQ ID NO: 350) | 444) |
| SRR4030068_megahit_k177_2163203_10\|M | CTCTCACCCCAGTTCGATCGAGGAAGGCGACTGACAC (SEQ ID NO: |
| (SEQ ID NO: 351) | 445) |
| SRR5487326_megahit_k177_254484_5\|M | GGCGTACCGCCTGACGGGTTTCTCATCGATGGACAC (SEQ ID NO: 446) |
| (SEQ ID NO: 352) | |
| SRR5487761_megahit_k177_404752_1\|M | GGCGTACCGCCTGACGGGTTTCTCATCGATGGACAC (SEQ ID NO: 446) |
| (SEQ ID NO: 353) | |
| SRR8554485_megahit_k177_439309_3\|P | GGCGCACCGCCTGACGGGTTTCTCATCGATGGACAC (SEQ ID NO: 447) |
| (SEQ ID NO: 354) | |
| 5RR8554511_megahit_k177_4512460_1\|M | ACCGCGGCGATACCGGCTTTCCCGGGTGACTGACAC (SEQ ID NO: 448) |
| (SEQ ID NO: 355) | |
| SRR8554511_megahit_k177_1767604_11\|P | GTCGCACCTCGCGCGTATGCACGGATTGGCAG (SEQ ID NO: 449) |
| (SEQ ID NO: 356) | |
| SRR8554643_megahit_k177_4328113_1\|P | ACCGCGGCGATACCGGCTTTCCCGGGTGACTGACAC (SEQ ID NO: 448) |
| (SEQ ID NO: 355) | |
| SRR8554643_megahit_k177_1281129_3\|P | GTTGCAACCTGTGCGTATGTGCGGATGGACAC (SEQ ID NO: 450) |
| (SEQ ID NO: 357) | |
| SRR8554643_megahit_k177_1281129_3\|M | GTTGCAACCTGTGCGTATGTGCGGATGGACAC (SEQ ID NO: 450) |
| (SEQ ID NO: 358) | |
| SRR6056591_megahit_k177_2733919_1\|P | GGGGGGCAAGCTCGCGCGTATGCACGGATTGGCAG (SEQ ID NO: 421) |
| (SEQ ID NO: 324) | |
| SRR6263259_megahit_k177_232832_6\|M | GGAGAGCGAAAAAAAGCGGGGGAGAT (SEQ ID NO: 451) |
| (SEQ ID NO: 359) | |
| 3300031539\|Ga0307380_10018846_3\|P | GTTGCAACCTGTGCGTATGTGCGGATGGACAC (SEQ ID NO: 450) |
| (SEQ ID NO: 357) | |
| 3300031539\|Ga0307380_10018846_4\|M | GTTGCAACCTGTGCGTATGTGCGGATGGACAC (SEQ ID NO: 450) |
| (SEQ ID NO: 358) | |
| 3300031539\|Ga0307380_10072822_2\|M | GGTACCGCGGCGATACCGGCTTTCCCGGGTGACTGACAC (SEQ ID NO: |
| (SEQ ID NO: 355) | 452) |
| 3300031539\|Ga0307380_10072822_2\|P | GGTACCGCGGCGATACCGGCTTTCCCGGGTGACTGACAC (SEQ ID NO: |
| (SEQ ID NO: 360) | 452) |
| 3300031566\|Ga0307378_10072489_1\|M | GGTACCGCGGCGATACCGGCTTTCCCGGGTGACTGACAC (SEQ ID NO: |
| (SEQ ID NO: 355) | 452) |
| 3300031566\|Ga0307378_10072489_2\|P | GGTACCGCGGCGATACCGGCTTTCCCGGGTGACTGACAC (SEQ ID NO: |
| (SEQ ID NO: 360) | 452) |
| 3300031670\|Ga0307374_10070813_2\|P | GGCGCACCGCCTGACGGGTTTCTCATCGATGGACAC (SEQ ID NO: 447) |
| (SEQ ID NO: 354) | |
| 3300031965\|Ga0326597_10006968_2\|P | GGCGCAACCCCGCACATGCGGGCGGGATGACAG (SEQ ID NO: 453) |
| (SEQ ID NO: 361) | |
| 3300017540\|Ga0180217_1000254_36\|M | GTCGCACCCCGCGCGTGCGAGCGTGTTGACAG (SEQ ID NO: 454) |
| (SEQ ID NO: 362) | |
| 3300014494\|Ga0182017_10000840_9\|P | GCCGCGTCCCCCGACGCGACCCTGATCGATGGACAC (SEQ ID NO: 436) |
| (SEQ ID NO: 343) | |
| 3300014839\|Ga0182027_10004505_3\|M | GGTGAAGCGGCCATCGGGATCGTGATCGGCTGACAC (SEQ ID NO: 438) |
| (SEQ ID NO: 346) | |
| 3300014839\|Ga0182027_10004505_2\|P | GGTGAAGCGGCCATCGGGATCGTGATCGGCTGACAC (SEQ ID NO: 438) |
| (SEQ ID NO: 347) | |
| 3300031715\|Ga0307476_10010105_2\|P | GGAGCGCCCCCCCGACGCGGCCATGATCGACTGACGG (SEQ ID NO: |
| (SEQ ID NO: 363) | 455) |
| 3300031715\|Ga0307476_10010105_1\|M | GGAGCGCCCCCCCGACGCGGCCATGATCGACTGACGG (SEQ ID NO: |
| (SEQ ID NO: 364) | 455) |

TABLE 14
Non-coding Sequences of Representative CLUST.089537 Systems

>3300009161|Ga0114966_10008307_7|P
ACTTTTTCTACCATGAAAAAAAACCCATCTGATAAAACAATAATGAACCTAGAATTTATGTTTTTTTATATAGAATGGATGAAA
AGATTAGAAACTAGTCCTGATTATAATAAACCTTTTATTGAAGAACTGTTTGGGATTACTATTTAGATTATCGTTAGCTAAAGA
CAATTGAAAAAATGACTTGAAGATTCTGAGCAAAAGATTAGGATTTTAAAAGGAGAAATTCCGTGGGCACAAGAGCCATAAAAG
CAAAGATTATTTGTGACAAAACAACACTTGGTTATTTGTGGCGATCTCACTGTATGTACAATCAGAAACTCTCTGAGTATATGA
AATTACTTTTTAAACTTCGACGTGGTAAGATTGGTAAAAATAAAGATTATGCGAATAAAAATGAGGAAGAAAAGCAGAAAATAA
TTGACAATCTTGAAGCAATACTTAGACCTCATCTGCAAAAAATGCACTTTACCACCAAAAAAGAACGCTCACTTGACGTTCCAG
AACACCCACTTGACGTTCCTTTTTGAGCTTGTTAAGATATGGTCATTGAGGAGTTTTGGTGGAGTTACGACCATCCCACAGACA
TGGCAAGCCATAGCTGAAAGCCTTGATAGCCTTCGTTTTGTGTCCAAAGTCTGCTGATCTGACTGCTTTCGGGCATTTAGGATA
TTACAAAAAAGGAAACTTTTTTGGCGGCGGGCAAGGCGAGGGCAATTAGAAGTAGATCTTTTGCTGTTTGACAAATTTAGTCAA
TTTTCGTCGTATTCTATTGATGCGAGTGACGAGGTATGATCGTTTTTGCCGTAGCTGCTCGCGAGCTAGGTGTAGTAACGACTT
AAGCACAGCTAAGCATTGTAAAAATTTGAATTTAAACCCTTATTTTTCAAGGTGCTCGTCAAGTGTGTTCTAAGTTGTTGTAGA
GACTTCGTTAAGGACTAGGGCCCCGTTGGAAAAAGAAAGAGTTAGATCATCTAATCCATAAGCATTGGAATATTTCCAAAGATC
AAAGATAGGAATCCAAGTGGGAAAAAAGAAAGTAGGGGGAAAAGGAAAAATAAAACAAAAATAATTTTCAAAAGAGATGTTCTG
GATTCAGAAAAAAACCAGAAGAATTTAAAATTGTGAAGGGTAAAATCTGTTTGGGGAGGAGGGTATTTCTCCACCGAACTACTC
AAGATTCTCTAAAATTAGGAAAAGTTTATGAAGACATCTAAGGCAGAATTTAAAATTATTAAATTAACATTTGCTGATAAAAAA
TGGGGACTAGTATTGATTTTTGTTGGTATTTGTTTGGGTCCTGTGATTGTAGGTATTCCATTAATTATACTTGGTGTGGGGATG
TTATGCATA (SEQ ID NO: 500)
>SRR6481359_megahit_k177_1860565_4|M
CTGCAATCGGACCTCGAAGCTGAAATGAACCTCAAAGACGACCAGACAATGATTATCGATATGGGTCCGAACGAAAAAGAGGCC
AGAGACGCAGCCGCTGTAATCGGCCAGTCCCTGCCGAAATTCGAAACCAGAATGATCGTCGTATAAAATTGACATCCCGGTTAT
GTTTTATATGATTAGGCATAATAGAGGATTACGAAAGGAGCTAATATGTCGGTGAGGTCTTTTCAAGCAAAAGTGGAATGCGAC
AAACAAATAATGGAACATCTTTGGCGGACGCACACGGTTTTCAATGAACGGCTTCCGGTAATTATCAAAATTTTGTTCCAGATG
AAACGGGGCGAATGCGGCCAAAATGATTCGGTCAATTTGCACAGGCGATTTCTGATAGATGATGCGTTAAAGAATTATTATGGA
TTCAAAAAGCTGCCTGAGAAAAAGCAAAAAGAGGCAATAGCAGCTATTGAGAACTCGCTGAAAGAAAAATTATCTACAATTCAC
AAAATATAAAATATTTTCCTTGTGTCTTAACATAGTTGAACATTTTTACTTGTCTTTGCCGATATTCTGAAAAAGGGACAAATT
GTTGTCCCATAAAGGATAACGTTCGGGAGGCAAGACAATGCAAGCTCTTTCAAAATTCTGTTGTCAAAATAAAAGATGCACGGA
TTACGGAAAACGTGGTGCAAATAATTTATCGGTGTGCGGCAGATTCGGCAAAAATGACCATATCCGGCTGCTATACTGCCGAAC
ATGCAAGAAGCGATTTTCCTATAATTTTTGCTGGCCGGTACGGACACTTCGTTTACAAGGCCAAGATGAGCGGTGGCACGCAAG
GACACCTGCGATGGCCGCGGATTTGACAGATCATGTCTGGTCTTTGCATGAATGGTTGAGTTATCCGGCTTTTCAATTAACATA
AGACACAAGGAATATTTTTCTTGCAGCATAAATATGCAAAAAGTACAATAATCTCGTGGAGTAAGGTAGGAGTGACGCCCGCCC
GTTTAGCGTGCTATAGGCCGCTGAATGCCACGTAGCGATATGTTTTGAGTGTCAAGAGCTGCTGACCCAAAGGCCAAAAGCTGA
GTAGGGCTTGATAGTCGTGGTTATTACCATGCAATGCGGCAGCGACACATATCGCGTTAATCAAATTTGCGTTGTCGTCAAGAT
GGATTCAGGCCATTGCGCAAGTGTCCTCTTTGACAAGTTCAGACGCGAGTGCGACGGTGGTGAAGGGGGGCACTGGGAATGCTC
GCAGCGGGAATTTGTGCAACTGCTTCCAGGATTTGAGGAACTATTGACAATATGGTTTGGCGCGAATTTTTACAGTTTTTTTAC
CTTGCTCGTAAACCAAACGCTAACCGCAGATGCAAAAAGAACTTGCAGAAGAAGCCATCAAGCCACAGAGAACAGAGGAATTTT
CATAATTACGCCACTGCATATAAATCCTTATGTGCCAAATACTGATGAATAAAAAATATCATAACTTTCAAATCCATCCAAGGG
CGAACCCTTTTCAGCCGCCTAACATGATAAGGCAGCATACTAACACGACTACGACCTTTGAACAAGCTATACCAACGCTCCACC
ACACTACGTCTTTCCAGCAGCCGTTGTCCCTCAACACTCTGCAACATACAATACGAAGCATTACGACCGGGTTGT (SEQ ID
NO: 501)
>UOUK01008448_2|M
CTGGAGAATGCATTGCGGGATGTGGTTAACCTCAAGCAGGATCAGGTGCTAATTGTCGATTTGGGGTCGAATGAGGAGGCGGCG
CGCGAGTCGGCGACGACGCTCGGGCCGGCGTTGCCTTCCCAAGACGAGGGGATCGTGGTAATCTAATCTGAGCGGTTCAGCACC
GGTCCCGCACCCGCGGCGATAGCTATTCTTGGAGGTGTAGACGTGGGAGTCCGGGCACTTCGAGCGCGAATCGAGTGCGATCGA
GAAACGCTTATGCACCTTTGGCGCACGCACTGCGTCTTCAATCAACGCCTACCCGCGCTGCTGAAACGCTTGTTCGCAATGCGG
CGAGGGGAAGTGGGGGGGAACGAGGCGCAGCGGCAGGTTTACCAACGTGTTGCGCAGTTTGTTCTTGCGCGCGACGGCGATCAA
GCCGTCGCCGCCTTCCGTGCTCTCCCACCGCGGGATTCCCCCGCCAGGACCTTAGCCGTTAAAAGGGTCGAAGATACCCTTCGC
CCACAGTTGATGCGCGTGCACAAACTTGCGGATGCAGGGGTTGACAGCCCCTTCTGAGGCTCTGATATACTGGGTTTGTGGGGC
GAGCCTTCGGGCGGTATATCCGAGCCGAAGTCCACGTAGTCGTCGCCCCTGTGTGTCAACACGTGCTGACTCCAAGGCGAACAG
CCGAAGGAGGCCCCGCAAGGGTGACATCGTGGCGGGCTCGCACGCCCCGGCGGCACGGACAGCGACGGCATTGATCAGGTCCGC
GTCGGCGCGGGCGGCGCTTCGCGGCGGTGTTCTTTGAAAAGTCGGTCGTCGCGGCCTTGGCCGAGGATAAAGTCGGTGCGACGC
AGCCAACTGCGACCAACGCCGTGGGCGCGTCAGGGCGCCCGCGAGTGCGACGGTGGTGAAGGGGGGGCCTGGGCACGCTCGCGA
GGGCGTAAGTCTTTGAGAATTGAGGACTTGCAGCTTGCTGGTTTTCGGATGCGACCGAACAGTTGCCGTACGTGCGGGTCACGC
TCGCAAACGCACGCGCAAGTTCGGTATTTACAGCAACTTGCGAGTGCG (SEQ ID NO: 502)
>3300012949|Ga0153798_10025413_2|P
CAAGCCGGCGACGTCGAGCGCACCTATGCCGACCTGACGCGGTCGAAGGCAGAACTCGGCTACGACCCGCAGGTGGAGATTACG
GAAGGCCTGCGGCGGTTCGTGGCGTGGTATCGGGAGAATCGGGAGAGGTATCGGCTGGAGGTGTGAGGGGGATTCGGGAGCGAC
GAAGCGACGGAGCGACGAAGGGGAAGGCGTATGATGCATAGGGGCAGGCAACTAGCCCGCCCTACTGCCTGATCTTCTCGACCG
GCCACATAATAGTGGGCGCATCTCCACGGGCACAACAGTGAGCGAGTCAGGTCGGCATGACTTCCACAGATCTGATTGTCTGTT
CTTGCATGGGAAATTGCACAGTCAACTGAATCCACGTTATCCTTGATCTTGCTGCCAAAGGAGGACCAGGTGGGGATCAAATCT
TGGCGCGTTAAATTATGGGTGGAATCGCCTGAGATACGCGCGACTCTGGAATATTGTCTCAGCCTGTGGAACACTTCGGCTGCT
TTACTTTGTCGGCATATACTAGCGATGCGTCGTGGCGACTTTGGCCCGGAAGGGCAAGCGATCTGGGAGCACCTAAAGAAAACC
CCACATAATTTTCATGTCCTTTATCCTATCTCCAATGGCAGCGGCATAGAAAAGACTACCGGATATTACCCCGATGCACGAGCA
TTCGTTGTCTTCCTTGGAACATTTCCCACTGTGCAGACCATCCAGCGCCAACCGCGGATCTACGAGATCAATGGCCAGCGCGTT
GCTCTGGATGAGATACAGCAGCGCGCTGTTGAAATCCTTGCCAGTCGCCGCGCAGGTGATACACCCTTTTAAGATTTGTTTGCG
GTATCAGACAGGAATAGTTAACATTTGCGGTGGATCAGGTCCGAAAGGGCCGGCCCGAGGTTCGGCTATAGGCCCAACCTCGCA
CCAGCGGGCGCTGTCTAGCCGAAATTGGCGAGCGAGGCTCTTCTCAGGGCCCGCTCGAAACCTGTGGCTACGATTACGTGGCTG
CGTGGGCTTTTCGGCACCCCGTTGGACACGCTTCGGTATGCGGAACGCGAACCACCAGCGCGCACCAGTTGCATAGCGGATTCG
CGTTTAGCGCAAGTTCTTTCTCAAGCGAACTGTGCAAACAGTGGGTCAGCAAATCGCTTCTGTTGCATTTGCCTGGACGAGACG
ATTCGCGAAAGTGGACTGTAGCTCTTGGTCCTGCCTTTAACTAGAGCAAGGCCGCCGATACCACTGGCGACGGCTTGCACGCCG
ACAGGGTCGCGATCGGTAACGTCCTGAATTGTGCGCACATTTGAAAAGGCAGCTCGTCCGCTGGAAGGTGTTGGTGTGTAGACA
ACCCTTACCAGACAGGAGAGCTGCATGGACAGCGTAACAGAAGCGGCGCAGG (SEQ ID NO: 503)
>CAAACB010000011_94|M
CAACACGGCCTGGGCTACGCCCCGTTCCCCGCCTCCGTGATTTCCCCCAGGAACTGGACCGACCACTGCCACCTGAACCCCAAC
GGCGAGCGCGAAAAAGCCACGCACATCGCCGACCATGTCCGCCCCTTCATCCGCGCTCCGCGATAAGCATTTTCGGACGGAATA
TGTCTCATCGGATGCACTTGACGGTGGCTGCGCCGCCGAGCGGAGTGCCCGTGCTTGCCCATCCATCGACCCGTTGGCATCCTG
TGAACAGGAACCAGGCACATGTCCACACGCGCTATTCAGGCCAGAGTTGTCGCCGACAAGGCACTGCTGCAAAAGCTCTGGCTG
ACCCACAGGGCGTTCAACGAACATTTGCCCCATTTTTCGCAAACCCTGTTCCGCATGATGCGGGGCGAGTTGGGCGACTCCGAT
CGCTTCGCCCTTGGTGAAACCGCCATCGCTGCCGCCATGGACTTGTGGCATCGCCCCAAGCCCGAAAAAGAGGCTATGCGAAAA
TCAATCGAAGCCCAACTCCGATCAAAACTGGAAATCGAACACAAGCTCGCCGAACCGCTGCCCTAAGGCTCTTCAAGGAGCTTG
CGCTGGGCACCCAGGTTTTCATACGTTGATCGCGTGGAGCAAGGGACCGGGTCGCGTCGGTTTCCACCATTTGGCTATAGGCCA
GGCGGTTGCCACATGGCGCCATGTTTTTGAGTGTCAAGATGTGCTGGTCCTAAGGCGACACGCCGAAGGGACTTTACCCTCCGG
GCAACTGGAAGGGGCGGCACAGACCCATGGCGCGTTCACTCCTACGATCTTTGAAGCGCGAGCTGCGACGTTCGCGAACCTATA
GCGGACAACCATCTACAGAGAGGTCCGCGGGCGGCGCAACCACCGCCCCCACGAGGACTTGTCACGCTCGTTCTCCGCTCGCCT
CTTGCGGGATCGGGAATCGCGTTTCGCTTCGCGAACTGGCGATGTGCGCACTTGTGCTTCAATCCTTTGCACTGCAAGG (SEQ
ID NO: 504)
>3300020109|Ga0194112_10008260_1|M
GCCGGCTGGACCTCCGAGTTCCCCGGCCAGCCGCCCCCCACCCGCATCGCCCTGGCCGGGCCGGACGCCCTCCTGTTCTTCTAC
CTCACGCTCCTCCAGGACATCAACACCAAGACCCTCCTCCCGCCCGACCTCGAATTTTACTGGTAGCCGGGCTAGATAGTCTCT
CTTGCGGAAAGTGCGATCACAGGCGGGACGCCTGTGCCACCGCTGAGACAAGGGGCTTGGTGGCACAGGCCTCTGGCCTGTGAT
TGGGGCACCGAGGGCGTGGATACGATGGACATGGTGGACCCCGAGGCCCGCGCCGCCCTCTGGCGCACCCATGGGGTCTACAAC
GAGTGCCTCCGGTTCGCCCTGCGCCACATGCACAAGATGAAGCGCGGGGAGGCCGACCCGCGCTACGCGGAGATATTCGGCGCG
ATCCGCGTGGAGAGGCCCAACAAGGACAAGCGGGTCTTCCGCCTGACTCCCAGGGGCCAGCCAACCGTGGAAGTGGACATGGCC
GACGTGGAGAAGCGCATCCTCGCGCGGGGAAGGGCCGAAGACCTCTGCCGCGGCAAGCCCACGCCATTTTGAGCTTGACTTTCC
CGGCCAAACTGTCATGAATGGTGTTGGCCCTGCGTGGAATAGCGCGGGCCGCCGGCGGGGCTGCTATAGGCCGCGTCGGACTAC
GTCGGGCATCCAGTTGTTACGGCCGCGTGGGCCTACGGGTTTCGGCCCAAAGGGCACAAGACCGGATGGGTCACTGGACCTGTC
AGGGCGGCGGCCAGGCCCGGCGACACGACCGAGAATGTTGGGGAAAAAGGAGGCCCGGACCGCCAAAGAATGGCCCGGATGGGG
CGCTTTTTTCAGCCGAAATGCGGGCTAACTCCTTGCGCCCTCTCGACTTCGGCGGTATCTGGTGTAGGCGCGGGTAGACTTTTC
GGACGATGTCAGGGCAAGGGCGAAGGGGTCAGACCTCAATTCTCTACTTTCCTGCCCCGGGTGGTGTGCCGACAGCGGCGTCGA
GCCGCCGCAGTCCGACGGGCTGACGCACGCTCGGGTGCCCGTGACGGTGCGAGAGGCATGGGGTACGGCGGGTGGCTGGGAAAG
GACAACAGGGACCACAGGGACAAC (SEQ ID NO: 505)
>UOUL01003058_1|M
CTGGAGAATGCATTGCGGGATGTGGTTAACCTCAAGCAGGATCAGGTGCTAATTGTCGATTTGGGGTCGAATGAGGAGGCGGCG
CGCGAGTCGGCGACGACGCTCGGGCCGGCGTTGCCTTCCCAAGACGAGGGGATCGTGGTAATCTAATCTGAGCGGTTCAGCACC
GGTCCCGCACCCGCGGCGATAGCTATTCTTGGAGGTGTAGACGTGGGAGTCCGGGCACTTCGAGCGCGAATCGAGTGCGATCGA
GAAACGCTTATGCACCTTTGGCGCACGCACTGCGTCTTCAATCAACGCCTACCCGCGCTGCTGAAACGCTTGTTCGCAATGCGG
CGAGGGGAAGTGGGGGGGAACGAGGCGCAGCGGCAGGTTTACCAACGTGTTGCGCAGTTTGTTCTTGCGCGCGACGGCGATCAA
GCCGTCGCCGCCTTCCGTGCTCTCGCACCGCGGGATTCGCCCGCCAGGACCTTAGCCGTTAAAAGGGTCGAAGATACCCTTCGC
CCACAGTTGATGCGCGTGCACAAACTTGCGGATGCAGGGGTTGACAGCCCCTTCTGAGGCTCTGATATACTGGGTTTGTGGGGC
GAGCCTTCGGGCGGTATATCCGAGCCGAAGTCCACGTAGTCGTCGCCCCTGTGTGTCAACACGTGCTGACTCCAAGGCGAACAG
CCGAAGGAGGCCCCGCAAGGGTGACATCGTGGCGGGCTCGCACGCCCCGGCGGCACGGACAGCGACGGCATTGATCAGGTCCGC
GTCGGCGCGGGCGGCGCTTCGCGGCGGTGTTCTTTGAAAAGTCGGTCGTCGCGGCCTTGGCCGAGGATAAAGTCGGTGCGACGC
AGCCAACTGCGACCAACGCCGTGGGCGCGTCAGGGCGCCCGCGAGTGCGACGGTGGTGAAGGGGGGGCCTGGGCACGCTCGCGA
GGGCGTAAGTCTTTGGGAATTGCGGACTTACAGCTTGCTGGTTTTGGGATGCGACCGAACAGTTGCCGTACGTGCGGGTCACGC
TCGCAAACGCACGCGCAAGTTCGGTATTTACAGCAACTTGCGAGTGCGGCA (SEQ ID NO: 506)
>3300025572|Ga0207864_1014515_3|M
GCGTCTACGGCAATGCCAGCAAGGTCCCCTTCAGCGAAGAAGACCCCGTCGATCAGCCCATCAGCCCCTACGCAGCCACCAAAC
GCGCCGGGGAGCTGATCTGCCATACGTTCTGGCATCTGTACAAAATGCCGATCTTCTGCCTGCGGTTTTTCACCGTCTACGGCC
CCCGCCAGCGGCCGGACCTGGCCATTCACAAGTTCACGCGGCTGATCGACGCCGGCAAGCCGATCCCCGTCTTCGGCGACGGCA
GCATGTCGCGCGATTACACGTATGTGTCGGACACGGTGGCTGGGATCATGAGCGCCCTGGACCGATGCGACCGCTACCGCATCT
ACAACCTCGGCGGCTCGAATCCGGTGACGCTCACGCAGTTGATCCAGGAGATCGGGACCGCCCTCGGCAAGCAGGCCATCATCG
ACCGCAAACCCACCCAAGCCGGCGACGTCGAGCGCACCTATGCCGACCTGACGCGGTCGAAGGCAGAACTCGGCTACGACCCGC
AGGTGGAGATTACGGAAGGCCTGCGGCGGTTCGTGGCGTGGTATCGGGAGAATCGGGAGAGGTATCGGCTGGAGGTGTGAGGGG
GATTCGGGAGCGACGAAGCGACGGAGCGACGAAGGGGAAGGCGTATGATGCATAGGGGCAGGCAACTAGCCCGCCCTACTGCCT
GATCTTCTCGACCGGCCACATAATAGTGGGCGCATCTCCACGGGCACAACAGTGAGCGAGTCAGGTCGGCATGACTTCCACAGA
TCTGATTGTCTGTTCTTGCATGGGAAATTGCACAGTCAACTGAATCCACGTTATCCTTGATCTTGCTGCCAAAGGAGGACCAGG
TGGGGATCAAATCTTGGCGCGTTAAATTATGGGTGGAATCGCCTGAGATACGCGCGACTCTGGAATATTGTCTCAGCCTGTGGA
ACACTTCGGCTGCTTTACTTTGTCGACATGTACTGGCGATGCGTCGTGGCGACTTTGGCCCGGAAGGGCAAGCGATCTGGGAGC
ACCTAAAGAAAACCCCACATAATTTTCATGTCCTTTATCCTATCTCCAATGGCAGCGGCATTGAAAAGACTACCCGGTATTATC
CCGATGCACGAGCATTCGTTGTCTTCCTTGGAACATTTCCCACTGTGCAGACCATCCAGCGCCAACCGCGGATCTACGAGATCA
ATGGCCAGCGCGTTGCTCTGGATGAGATACAGCAGCGCGCTGTTGAAATCCTTGCCAGTCGCCGCGCAGGTGATACACCCTTTT
AAGATTTGTTTGCGGTATCAGACAGGAATAGTTAACATTTGTGGTGGATCAGGTCCGAAAGGGCCGGCCCGAGGTTCGGCTATA
GGCCCAACCTCGCACCAGCGGGCGCTGTCTAGCCGAAATTGGCGAGCGAGGCTCTTCTCAGGGCCCGCTCGAAACCTGTGGCTA
CGATTACGTGGCTGCGTGGGCTTTTCGGCACCCCGTTGGACACGCTTCGGTATGCGGAACGCGAACCACCAGCGCGCACCAGTT
GCATAGCGGATTCGCGTTTAGCGCAAGTTCTTTCTCAAGCGAACTGTGCAAACAGTGGGTCAGCAAATCGCTTCTGTTGCATTT
GCCTGGACGAGACGATTCGCGAAAGTGGACTGTAGCTCTTGGTCCTGCCTTTAACTAGAGCAAATGTCTCGGCTGGCGATATCG
TCGTCGTCGGCGTCGCGATCCG (SEQ ID NO: 507)
>CAAACX010000652_28|M
GAGGATGGACCAGACGGCGCCGCCGCGCTCCTTGAGCATCGCGGCAAGAACGTGCAGGCCGGTGACCTCCGGGTTCTGACGCGC
CGCGGCGTCGGACTGGGCGTCGGCAAGGGTCTGCTGGGCGAGCGTGGTGAAGCGGTCGGTGCGCATGGGTCGTGCTCCCGAGGG
AGTCTGGCTGACTCGGTCTGCCGGATTGTTGGGCAAAACGGGGGCCAGTCGTGAGAGGCTTTGGAGGGGGGTTTTACGGAGCGC
GGACTGCCGGAATGGCAGGGGGACGCCGGCCGTCGGCCAACCGCCCCCCGGGCGCCTCCCGGCAAGTTTCGTTAGGATGGTCGC
CGTGGAGGCCGCCTCAGATGGGTGTTAAAGCCTGGAAGGCGAAGATCATCGTGGCCGACCCGGACCTGGCGGCCGCTCTGGACC
ACACCGTCCGGCAATGGAACCTCTGGGCGGAACAGGCCTGCATCGCCATCATGCGCCTGCGCCGCGGCGACTACGGGGACGAGG
TCCTGATCGGGACGTTCCCGCAGGTCCGGCGCATCTCGCGCGACCCGCCGGTGCTGTCGATCGCCGATGTCCGGGTGGACCAGC
GGGAGGCGGATCGCCTGGCCGCGGCGTGGCTGAGCGAACTGTCGGTCGGCCCGACGCCCTTCTGAGGTGGCGTCGGATGGGGGC
TTCCGGTATACTGCCGTCGCTACCGCCTGCACAGGGCGGCGCGGCGGACCGGCTATAGGCCCGTCCGTCGTACGTCGGGAACTG
TCGAGCCGGGAGTGACGTGGCGGGGCCTCGGCCCTGTTCAAGACCTTCACCCCTCTCGTGGGGGTGGTGGGCTTCCCGGCAACC
CGGCGGCTGATCCATCACGCGGCCGCCGCGAATATCGAGCGCCCACGCCGACGCCGGGAGGTTCGCGGAAGTCGCAACGCTCTT
TGACAGCCCAAGTTGCGCTCAGGAGGCCCGATGACCGGCTTGGCTCTCCGTTCAAAAACAGCCGATTCGCGGAGGAACGCTCCG
AAGTGCCGACCGGTCAACCTCTTACGGGCGGGAGCTACTACGAGGCGATGCGGATCGGCGGGAACGCCCGCTGCATCGGCCG
(SEQ ID NO: 508)
>3300018080|Ga0180433_10080815_1|M
TTCGAGATCGACACCGAATCACGCATAGGGTGTCAGGAGATAGTACCCTAAAAATTCTGAGTAATCTACACCTGTCGCGTTCTC
CTTGTTGACCATGATTTTATTAGAGATTGGTAAAAATATTAGATTGAAGATCGAATATATTAGATATTAGAGAAAAGTTATTGG
AGATAGATAATGTCAGTGAAAACAATTCAAGCAAAATTAGAATGTGACAGAGAGACTTTAGAGACTCTATGGAGAACTCATACG
GTTTTTAATGAAGGTCTTTTACCTATTTTAGCTTTATTTTTTAAGATGAAGCGTAGAGAAACAGGATCAACAGAATTACACAGA
AAGTTCTATGATTGGTCAGTAGTAGACAAGTACTTTACTTTGCTTAGAAGTGATAATTATAAGGAACAGAAAAAAGTGATTGAA
GAAGAACTTCGAGAGTTTTTGAGGAAAGAGCATTTTCCTCAAATGGCTCTGAATTAAAAAGTGGAGAATTTATCCAATGCCTAT
AGGGAAATTGGATAACCACACAGGCCTTGGTTTTGATGGTGAATATCTGTTGATTATTTGGTCTGAGGGCCTTAAATAAATTAC
ATGGATAGTA (SEQ ID NO: 509)
>3300025572|Ga0207864_1000213_47|M
GAGGATGGACCAGACGGCGCCGCCGCGCTCCTTGAGCATCGCGGCAAGAACGTGCAGGCCGGTGACCTCCGGGTTCTGACGCGC
CGCGGCGTCGGACTGGGCGTCGGCAAGGGTCTGCTGGGCGAGCGTGGTGAAGCGGTCGGTGCGCATGGGTCGTGCTCCCGAGGG
AGTCTGGCTGACTCGGTCTGCCGGATTGTTGGGCAAAACGGGGGCCAGTCGTGAGAGGCTTTGGAGGGGGGTTTTACGGAGCGC
GGACTGCCGGAATGGCAGGGGGACGCCGGCCGTCGGCCAACCGCCCCCCGGGCGCCTCCCGGCAAGTTTCGTTAGGATGGTCGC
CGTGGAGGCCGCCTCAGATGGGTGTTAAAGCCTGGAAGGCGAAGATCATCGTGGCCGACCCGGACCTGGCGGCCGCTCTGGACC
ACACCGTCCGGCAATGGAACCTCTGGGCGGAACAGGCCTGCATCGCCATCATGCGCCTGCGCCGCGGCGACTACGGGGACGAGG
TCCTGATCGGGACGTTCCCGCAGGTCCGGCGCATCTCGCGCGACCCGCCGGTGCTGTCGATCGCCGATGTCCGGGTGGACCAGC
GGGAGGCGGATCGCCTGGCCGCGGCGTGGCTGAGCGAACTGTCGGTCGGCCCGACGCCCTTCTGAGGTGGCGTCGGATGGGGGC
TTCCGGTATACTGCCGTCGCTACCGCCTGCACAGGGCGGCGCGGCGGACCGGCTATAGGCCCGTCCGTCGTACGTCGGGAACTG
TCGAGCCGGGAGTGACGTGGCGGGGCCTCGGCCCTGTTCGAAACCTTCACCCCTCTCGTGGGGGTGGTGGGCTTCCCGGCAACC
CGGCGGCTGATCCATCACGCGGCCGCCGCGAATATCGAGCGCCCACGCCGACGCCGGGAGGTTCGCGGAAGTCGCAACGCTCTT
TGACAGCCCAAGTTGCGCTCAGGAGGCCCGATGACCGGCTTGGCTCTCCGTTCAAAAACAGCCGATTCGCGGAGGAACGCTCCG
AAGTGCCGACCGGTCAACCTCTTACGGGCGGGAGCAGATGCAGCCAAGGGGCGGACTCGAACCGCCCCTCGCACGTATCCGCCG
GAAAAGGGCGAATGCCAGTGACCGAAGAACGGGTCCGGCCGTTCCGCAACACTCAATCGCCGCCGCGGCAATCAGGCAGAGCCC
GCCTCAGCAACATCATCCCCCGCGCCCCCTGCTCCGCCCTTCTGAGATCGCGCAGGCCCTCAACCCGTCTACGCCACCACGTCC
AGCCGCTCCCCCACTCCCGGCGGGCTCGCGCCCTGGGCGACCTTCGCCGCGGCCTGGATCATCTGCACCGCGGCCTGGCCCTGC
TGCTCGGCGTGGTCGAGGACCTTCCGGGCGACGCGGATGCTCGCGGCGGA (SEQ ID NO: 510)
>3300009652|Ga0123330_1010394_3|M
CTGGAGAATGCATTGCGGGATGTGGTTAACCTCAAGCAGGATCAGGTGCTAATTGTCGATTTGGGGTCGAATGAGGAGGCGGCG
CGCGAGTCGGCGACGACGCTCGGGCCGGCGTTGCCTTCCCAAGACGAGGGGATCGTGGTAATCTAATCTGAGCGGTTCAGCACC
GGTCCCGCACCCGCGGCGATAGCTATTCTTGGAGGTGTAGACGTGGGAGTCCGGGCACTTCGAGCGCGAATCGAGTGCGATCGA
GAAACGCTTATGCACCTTTGGCGCACGCACTGCGTCTTCAATCAACGCCTACCCGCGCTGCTGAAACGCTTGTTCGCAATGCGG
CGAGGGGAAGTGGGGGGGAACGAGGCGCAGCGGCAGGTTTACCAACGTGTTGCGCAGTTTGTTCTTGCGCGCGACGGCGATCAA
GCCGTCGCCGCCTTCCGTGCTCTCGCACCGCGGGATTCGCCCGCCAGGACCTTAGCCGTTAAAAGGGTCGAAGATACCCTTCGC
CCACAGTTGATGCGCGTGCACAAACTTGCGGATGCAGGGGTTGACAGCCCCTTCTGAGGCTCTGATATACTGGGTTTGTGGGGC
GAGCCTTCGGGCGGTATATCCGAGCCGAAGTCCACGTAGTCGTCGCCCCTGTGTGTCAACACGTGCTGACTCCAAGGCGAACAG
CCGAAGGAGGCCCCGCAAGGGTGACATCGTGGCGGGCTCGCACGCCCCGGCGGCACGGACAGCGACGGCATTGATCAGGTCCGC
GTCGGCGCGGGCGGCGCTTCGCGGCGGTGTTCTTTGAAAAGTCGGTCGTCGCGGCCTTGGCCGAGGATAAAGTCGGTGCGACGC
AGCCAACTGCGACCAACGCCGTGGGCGCGTCAGGGCGCCCGCGAGTGCGACGGTGGTGAAGGGGGGGCCTGGGCACGCTCGCGA
GGGCGTAAGTCTTTGGGAATTGCGGACTTACAGCTTGCTGGTTTTGGGATGCGACCGAACAGTTGCCGTACGTGCGGGTCACGC
TCGCAAACGCACGCGCAAGTTCGGTATTTACAGCAACTTGCGAGTGCGGCA (SEQ ID NO: 506)
>3300015360|Ga0163144_10184575_1|M
GAATTGAATGCAACCGCGAAGAATTACAACATCTTTGGCGGACTCACTGCGCCTTCAACGAAAGGTTGCCATCAATCCTTTCGG
TCCTGTTCAAAATGCGTCGTGGAGAATGGGGTGACACCGAACAGATAAAGAAAAAAATGCAGGCTTTCTTCTTTCTCGATGGGC
AGGCCGTCCAGGCATTCTACGATTGGAAGGGCAAGCCGGAGCCGGAAAGAAAAACGTTTTTCGCGGCAATGGACGGCAAGCTGC
AGCCAGCGCTGAGAGTGGAACATAAATTGGATGGTCCGACACCGTTTTAAAATTCGAAGACTTGCGGCAGGATGCAATCAGGTT
ACGTTTACGTTTGTGGGTTGTACCCGCTCGGCGTGCTATAGGCAGGCCGGGACCACAAAAGCACTGCGTTTGAGTGTCAAGACC
TGCTGACCGAACGGCCAAAAGCCTAGTAGGGCCAGACTGAGGAGGGGCAACCCACCAAGGGCGGCAGGAACACGCAGTGAGTTG
TTCAGCTTGCGTTGTGAGGACTGTTGGTGGGTCTTTGACAACTCATTTTGCGCGGCTTTTTGCCGCGAGCGCGGAGGTGGTGGT
GCGGGTGCGTCGTTGCGCTCGCACCTACTTAACCTCTTACGGTGCAAGGGCCTCGTCAACGGAAATGGTGAGTCGCATGTGTCT
CGATCAGTTGATTCCACGCAGGTGCTCGTTATGGGACTGCCAAGTCCCGGTCAAATCATGGTGTGCGAGCGCGACA (SEQ ID
NO: 511)
>3300017960|Ga0180429_10052609_1|M
TTCTACAGCAAGACTTAGCCGAAATGCCCCCAAGATGTTGCGGTGTCAGCAAAGCAGGCAATACAATATCCGGCACCAAATCGC
CCGAAGTCTTGCTGTAGAGAAAATGTACGCCTGACCCCGTTGTCCCCCCTTGCCCGCCCCAAGCGATGACGGCAAGCGGAGTAC
GTCTTCTACTGAATCGAAAGGACCCGAAATGTCACAAAGGGCCCTTGCCGTACGACTTCTGGTTGACTCTGAAGACGATCGCGC
GGCGCTGTGGCGAACGCACCGCATATTCAATGAGCGCCTCGGATGGGTGCTGCGTCAGATGCATCGGATGAAACGGGGCGAACC
GGACGCGCGCGAACTCCCGAAGGTCAAGAAGCTGCGCGAGGGCGTGTATGAGCTAGCGAAGCCTGATCACGCGCCCAAAGAAGT
GAAGCCTGCCGAGCTTAGGAGTGCGGTTAGCGTCGCGGCGGCCGCGATGTGCCGCGGGCAGGCGAGCCCCTTCTAGGCATTGAC
TTCCCTTCTGTACCTGATACGATATGGATCAGAGTTGCGAGATTACCGTAACCTGACCGGCAGGGCTATAGGCCGTGTCGGTGA
GCGCCGGGAACCAGTGGTTATGGCCGCATGGGCCTACGGGTTACCCCAATGGGCACGAAACCGACCCGGTCGGCTTCGCCGATC
CAGCGAGGGCGGCGGCCTTTGCCCGGCGACACAATCGGGAATTTCGGGGAAAACTGCCGCTTCGCACCCCGGGAAAGGGGCTGG
AAAGAGTGTTTTTTCGAGGCGTTTTCGTTCTAAGTCCTTTTGCAGCGAGGAGTTCGGTCCGGAACTTATTGTCCAA (SEQ ID
NO: 512)

### Example 8—Functional Validation of Engineered CLUST.089537 CRISPR-Cas Systems (FIGS. 19-21)

Having identified the minimal components of CLUST.089537 CRISPR-Cas systems, we selected one loci for functional validation, from the metagenomic source designated CAAACX010000652.

### DNA Synthesis and Effector Library Cloning

To test the activity of an exemplary CLUST.089537 CRISPR-Cas system, we designed and synthesized systems containing the pET28a(+) vector. Briefly, E. coli codon-optimized nucleic acid sequences encoding the CLUST.089537 CAAACX010000652 effector (amino acid sequence provided in Table 12) were synthesized (Genscript) and cloned into a custom expression system derived from the pET-28a(+) (EMD-Millipore). The vector included a nucleic acid encoding CLUST.089537 CAAACX010000652 effector protein under the control of a lac promoter and an E. coli ribosome binding sequence. The vector also included an acceptor site for a CRISPR array library driven by a J23119 promoter following the open reading frame for the CLUST.089537 CAAACX010000652 effector.

We computationally designed an oligonucleotide library synthesis (OLS) pool containing “repeat-spacer-repeat” sequences, where “repeat” represents the consensus direct repeat sequence found in the CRISPR array associated with the effector, and “spacer” represents sequences tiling the pACYC184 plasmid as well as E. coli essential genes. The spacer length was determined by the mode of the spacer lengths found in the endogenous CRISPR array. The repeat-spacer-repeat sequence was appended with restriction sites enabling the bi-directional cloning of the fragment into the aforementioned CRISPR array library acceptor site, as well as unique PCR priming sites to enable specific amplification of a specific repeat-spacer-repeat library from a larger pool.

We next cloned the repeat-spacer-repeat library into the plasmid using the Golden Gate assembly method. Briefly, we first amplified each repeat-spacer-repeat from the OLS pool (Agilent Genomics) using unique PCR primers, and pre-linearized the plasmid backbone using BsaI to reduce potential background. Both DNA fragments were purified with Ampure XP (Beckman Coulter) prior to addition to Golden Gate Assembly Master Mix (New England Biolabs) and incubated per the manufacturer's instructions. We further purified and concentrated the Golden Gate reaction to enable maximum transformation efficiency in the subsequent steps of the bacterial screen.

The plasmid library containing the distinct repeat-spacer-repeat elements and Cas proteins was electroporated into E. Cloni electrocompetent E. coli (Lucigen) using a Gene Pulser Xcell® (Bio-rad) following the protocol recommended by Lucigen. The library was either co-transformed with purified pACYC184 plasmid, or directly transformed into pACYC184-containing E. Cloni electrocompetent E. coli (Lucigen), plated onto agar containing chloramphenicol (Fisher), tetracycline (Alfa Aesar), and kanamycin (Alfa Aesar) in BioAssay® dishes (Thermo Fisher), and incubated for 10-12 hours at 37° C. After estimation of approximate colony count to ensure sufficient library representation on the bacterial plate, the bacteria were harvested and plasmid DNA extracted using a QIAprep Spin Miniprep® Kit (Qiagen) to create an “output library.” By performing a PCR using custom primers containing barcodes and sites compatible with Illumina sequencing chemistry, we generated a barcoded next generation sequencing library from both the pre-transformation “input library” and the post-harvest “output library,” which were then pooled and loaded onto a Nextseq 550 (Illumina) to evaluate the effectors. At least two independent biological replicates were performed for each screen to ensure consistency.

### Bacterial Screen Sequencing Analysis

Next generation sequencing data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source (pACYC184 or E. Cloni) or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

To identify specific parameters resulting in enzymatic activity and bacterial cell death, we used next generation sequencing (NGS) to quantify and compare the representation of individual CRISPR arrays (i.e., repeat-spacer-repeat) in the PCR product of the input and output plasmid libraries. We defined the array depletion ratio as the normalized output read count divided by the normalized input read count. An array was considered to be “strongly depleted” if the depletion ratio was less than 0.1 (more than 10-fold depletion). When calculating the array depletion ratio across biological replicates, we took the maximum depletion ratio value for a given CRISPR array across all experiments (i.e. a strongly depleted array must be strongly depleted in all biological replicates). We generated a matrix including array depletion ratios and the following features for each spacer target: target strand, transcript targeting, ORI targeting, target sequence motifs, flanking sequence motifs, and target secondary structure. We investigated the degree to which different features in this matrix explained target depletion for CLUST.089537 systems, thereby yielding a broad survey of functional parameters within a single screen.

FIG. 19 shows the degree of interference activity of the engineered compositions by plotting for a given target the normalized ratio of sequencing reads in the screen output versus the screen input. The results are plotted for each DR transcriptional orientation. In the functional screen for each composition, an active effector complexed with an active RNA guide will interfere with the ability of the pACYC184 to confer E. coli resistance to chloramphenicol and tetracycline, resulting in cell death and depletion of the spacer element within the pool. Comparing the results of deep sequencing the initial DNA library (screen input) versus the surviving transformed E. coli (screen output) suggest specific target sequences and DR transcriptional orientation that enable an active, programmable CRISPR system. The screen also indicates that the effector complex is only active with one orientation of the DR.

FIGS. 20A-B depict the location of strongly depleted targets for CLUST.089537 CAAACX010000652 effector targeting pACYC184 and E. coli E. Cloni essential genes.

FIG. 21 depicts a weblogo of the sequences flanking depleted target, indicating a 5′ PAM of TTC.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered, non-naturally occurring Clustered Regularly Interspaced Short Palindromic Repeat (CRISPR)-Cas system of CLUST.121143, CLUST.196682, or CLUST.089537, comprising:
an RNA guide comprising a direct repeat sequence and a spacer sequence capable of hybridizing to a target nucleic acid; and
a CRISPR-associated protein or a nucleic acid encoding the CRISPR-associated protein, wherein the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence provided in Table 2, Table 8, or Table 12;
wherein the CRISPR-associated protein is capable of binding to the RNA guide and of targeting the target nucleic acid sequence complementary to the spacer sequence.

2. The system of claim 1, wherein the system is the CRISPR-Cas system of CLUST.121143 and the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence provided in Table 2.

3. The system of claim 2, wherein the CRISPR-associated protein is a CLUST.121143 3300014839 effector protein.

4. The system of claim 2 or 3, wherein the CRISPR-associated protein is capable of recognizing a protospacer adjacent motif (PAM), and the target nucleic acid comprises a PAM comprising the nucleic acid sequence 5′-TTG-3′.

5. The system of any one of claims 2-4, wherein the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence provided in Table 3.

6. The system of any one of claims 2-5, wherein the spacer sequence of the RNA guide comprises between about 22 nucleotides to about 40 nucleotides.

7. The system of any one of claims 2-6, wherein the spacer sequence of the RNA guide comprises between about 26 nucleotides to about 35 nucleotides.

8. The system of claim 1, wherein the system is the CRISPR-Cas system of CLUST.196682 and the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence provided in Table 8.

9. The system of claim 8, wherein the CRISPR-associated protein is a CLUST.196682 3300025638 effector protein.

10. The system of claims 8 or 9, wherein the CRISPR-associated protein is capable of recognizing a protospacer adjacent motif (PAM), and the target nucleic acid comprises a PAM comprising the nucleic acid sequence 5′-CG-3′.

11. The system of any one of claims 8-10, wherein the direct repeat sequence comprises a nucleotide sequence that is at least 80% ((e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence provided in Table 9.

12. The system of any one of claims 8-11, wherein the spacer sequence of the RNA guide comprises between about 17 nucleotides to about 44 nucleotides.

13. The system of any one of claims 8-12, wherein the spacer sequence of the RNA guide comprises between 26 and 38 nucleotides.

14. The system of claim 1, wherein the system is the CRISPR-Cas system of CLUST.089537 and the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to an amino acid sequence provided in Table 12.

15. The system of claim 14, wherein the CRISPR-associated protein is a CLUST.089537 CAAACX010000652 effector protein.

16. The system of claim 14 or 15, wherein the CRISPR-associated protein is capable of recognizing a protospacer adjacent motif (PAM), and the target nucleic acid comprises a PAM comprising the nucleic acid sequence 5′-TTC-3′.

17. The system of any one of claims 14-16, wherein the direct repeat sequence comprises a nucleotide sequence that is at least 80% ((e.g., 81%, 82%, 83%, 84%, 85%, 86%, 87%, 88%, 89%, 90%, 91%, 92%, 93%, 94%, 95%, 96%, 97%, 98%, 99% or 100%) identical to a nucleotide sequence provided in Table 13.

18. The system of any one of claims 14-17, wherein the spacer sequence of the RNA guide comprises between about 20 nucleotides to about 40 nucleotides.

19. The system of any one of claims 14-18, wherein the spacer sequence of the RNA guide comprises between 25 and 37 nucleotides.

20. The system of any one of claims 1-19, wherein the CRISPR-associated protein comprises at least one (e.g., one, two, or three) RuvC domain.

21. The system of any one of claims 1-20, wherein the CRISPR-associated protein comprises a split RuvC domain.

22. The system of any one of claims 1-21, wherein the CRISPR-associated protein comprises a catalytic residue (e.g., aspartic acid or glutamic acid).

23. The system of any one of claims 1-22, wherein the CRISPR-associated protein cleaves the target nucleic acid.

24. The system of any one of claims 1-23, wherein the CRISPR-associated protein further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

25. The system of any one of claims 1-24, wherein the nucleic acid encoding the CRISPR-associated protein is codon-optimized for expression in a cell.

26. The system of any one of claims 1-25, wherein the nucleic acid encoding the CRISPR-associated protein is operably linked to a promoter.

27. The system of any one of claims 1-26, wherein the nucleic acid encoding the CRISPR-associated protein is in a vector.

28. The system of claim 27, wherein the vector comprises a retroviral vector, a lentiviral vector, a phage vector, an adenoviral vector, an adeno-associated vector, or a herpes simplex vector.

29. The system of any one of claims 1-28, wherein the target nucleic acid is a DNA.

30. The system of any one of claims 1-29, wherein the target nucleic acid comprises a PAM.

31. The system of any one of claims 1-30, wherein the CRISPR associated protein has non-specific nuclease activity.

32. The system of any one of claims 1-31, wherein the targeting of the target nucleic acid by the CRISPR-associated protein and RNA guide results in a modification in the target nucleic acid.

33. The system of claim 32, wherein the modification in the target nucleic acid is a double-stranded cleavage event.

34. The system of claim 32, wherein the modification in the target nucleic acid is a single-stranded cleavage event.

35. The system of claim 32, wherein the modification in the target nucleic acid results in an insertion event.

36. The system of claim 32, wherein the modification in the target nucleic acid results in a deletion event.

37. The system of any one of claims 32-36, wherein the modification results in cell toxicity or cell death.

38. The system of any one of claims 1-37, further comprising a donor template nucleic acid.

39. The system of claim 38, wherein the donor template nucleic acid is a DNA.

40. The system of claim 38, wherein the donor template nucleic acid is an RNA.

41. The system of any one of claims 1-40, wherein the RNA guide comprises a tracrRNA, a modulator RNA, or both.

42. The system of any one of claims 1-41, wherein the system further comprises a tracrRNA.

43. The system of any one of claims 1-42, wherein the system further comprises a modulator RNA.

44. The system of any one of claims 1-43, wherein the system is present in a delivery composition comprising a nanoparticle, a liposome, an exosome, a microvesicle, or a gene-gun.

45. The system of any one of claims 1-43, which is within a cell.

46. The system of claim 45, wherein the cell is a eukaryotic cell.

47. The system of claim 45, wherein the cell is a prokaryotic cell.

48. A method of binding the system of any one of claims 1-47 to the target nucleic acid in a cell comprising:
(a) providing the system; and
(b) delivering the system to the cell,
wherein the cell comprises the target nucleic acid, wherein the CRISPR-associated protein binds to the RNA guide, and wherein the spacer sequence binds to the target nucleic acid.

49. A method of editing a target nucleic acid, the method comprising contacting the target nucleic acid with a system of any one of claims 1-47.
